SEQUENCE LISTING (34 sequences)

SEQ ID NO: 1 - DNA, 534 bp, Mus musculus
tctccagtcacagagtgttgagggtgtgccacctcccctttgggaccaccttgggttgccctcttaacaaagttggccttaccaaggagcagtcatcttggattgtataatttgaatgagccaaggaccagagtgagggcagcacaaactactcagccacaatgtcttcagaggtggagacctcggagggggtagatgagtcagagaagaactctatggcaccagaaaaggaaaaccataccaaaatggcagacctttctgagctcctgaaggaagggaccaaggaatcacatgaccgagcagaaaatacccagtttgtcaaagacttcttgaaaggcgacattaagaaggagctatttgagctggccaccactgcactttacttcacatactcagcgcttgaggaggaaatggaccgcaacgagggccacgcagccttcgcccccttatatgtgcccacggagcttcaccggaagcagcactggtcaggacatgaagtatttcttgtggaaaactgggggagccggtaagtgc

SEQ ID NO: 2 - DNA, 1349 bp, Mus musculus
taaccttcattttttgtccatttatttagaaaaaaattaacatgagcaaatgaaatacctcagtgttacaacagagtatagaaatgtctagcaataatcaaataatttgatctttaaatacaaaataaccacatgaacacctaatatacaggtttcatctgaatacatatttattagataaatattagaggtcacatcatctaactgcatacagctttgcaagactagaaatcacaattagttttttttttctgaccagtcaaaagtatgaaatgattgcagtgtacatacgatgtacaaagacaagggcgggttctgtggacgtcacttcaggctgcacgtgtgggtgtggatgtgtgtacgtgtgaatcacctgtgatcatgatatcaaaaacttatacaaagtatatgaatttggttacaattttcttctgaaatccccgtttctcttcattgtttccatagcaccctaaaaatacacaggtggcagggccaggacacagaaggtaaatagtacatgtaggtaaaaataaaaacaaaagggaacaaaaacgcctctgcacacagggtcagtatattacaggagacaaggacggagtcacgaaggctaacaaacgggatctagtattccacgtagaatgaaggagttccaagccttttgttgtttctctgttttgtaaaataaaaacaatacacattccgggagaaatgaatgtatcttgttgacatgtctatttctcatttacatatgtacacacggcccttgagtcgctgctgctctctgcctcgtctggattggtcaggccgagggcccatggggagcagacctgtagctctctgggatttagggcttccgttagggagaaagtgttaggaatcttttaaaaaataaaatggctacaggatacgtgagacatgaataaagcttcaaaccaagaagatgagggtgatcgcctgtgcgggggcggggcctttcccatctgcgcatgctctctcccagcccagccgcttagctgagtggcggctggtacgtgcctatgggtgggttgctcgttgtggtaaagtggcttgctgagacctcatttcggaggttactatggctccaagttgttgtaagaaaggactgaggatctttccagagcctaggcctgcctctgtttatggatgtcacctttacctgcgtctgtcactaccaaggcatgctccagcccccgatgtctttgtagctctctcaaaccctggatcggctccaacatttctctggaaggagacatttccggagtgtgggcttcaggctctgtggtgaacttgctggtgggcacttgcctgggagggagccttaggaaatc

SEQ ID NO: 3 - DNA, 844 bp, Mus musculus
tctgtataagtctctcaatttataaccatacataatagtcaccaaaacacagaatgcttgctcctctggcatatgcaacagtagtacagcagcaagaaaagactgtccttgacagtacccaatgtcttcatcgaacaccgagtaggccttgcagattttgtaaagggattcttgaccatcgcctccagtgtctttgaagtaatcgtgtgcaggaaatgtacgatgaatatctcgagtaataacactctcctgtgcagagtcctttgtgataaggattcggtatttatccagcatttcctgattgtcatggcatcctgctaatagttgccatacttctgcccttagtgcctcaggaacaccactctttaccaaggtgaatagtccttttggtcggccaccaaggttattgtgccatcttcctaacaattctccccaagaatagagaatcttctcaggacagtcttttgacacatcacctgttccactcgagagttcattatcactctcctcttctgcttcatcctcctgtggtgacattgggcccccagcactagtaggggtgatgggctcctccttgtcagattctctctgcagactcaccacttcatatattgcatctccagcactgctgtggccttttccctcagactgtttcaacctcatgaagaaagtctctgtgaaagtctttctgctgaaataccaaaatctctcatttgcagggtatacacgaactactgtctccaggagaaaacggactggctccaccacctctgtgaccaccatgtccactgcaacggtcatgtacacccgtttatctttgggcgtttcttcattaagagccaga

SEQ ID NO: 4 - DNA, 1439 bp, Mus musculus
aggggcgccgggagcaggcgtgtgggactcctgaccggagagccggaggctgcgccttccccgcaccgggaccttcacgacacaccagatcctagtccttgccccgtgcgaacgcccacgatgaccaccaccctcgtgtccgccaccatttttgacttgagcgaagttttatgcaagggtaacaagatgctcaactacagcactcccagcgctgggggctgcctgctggacaggaaggcagtgggcacccctgctggcgggggcttccctcgcaggcactcggtcactctgcccagctccaagttccatcagaaccagcttctcagcagccttaagggtgagccggccccgtccctgagctcacgcgacagccgctttcgagaccgctctttctccgaagggggcgagcggctgctgcccacccagaagcagcctgggagcggccaggtcaactccagccgctacaagacggagctgtgccgtcccttcgaagaaaacggtgcctgtaagtacggggacaagtgccagttcgcgcatggcatccacgagctccgcagcctgacccgccaccccaagtacaagacggagctgtgccgcaccttccacaccatcggcttttgcccgtacgggccccgctgccacttcattcataacgccgaggagcgacgcgccctggcggggggccgagacctctccgctgaccgtccccgcctccagcatagctttagctttgctgggtttcccagtgccgctgccaccgccgctgccacggggctgctggacagccccacatccatcaccccaccccctatcctgagcgccgatgacctcttgggctcacctactctgcccgatggcaccaataaccccttcgccttttccagccaggagctggcgagcctctttgctcctagcatggggctgcctgggggaggctcccccaccactttcctcttccggcccatgtccgaatcccctcacatgtttgactctccccccagccctcaggattctctctcggaccacgagggctatctgagcagctccagctccagccacagtggctcagactcccctaccttggacaactcaagacgcctgcccattttcagcagactctccatctcagatgactaagccagggtagggagggacccccccccatgcctccttcacctctccaccccatctcttccctccacctccccaccccctaactttccctcaaaccccacattgatacatttaagctcagcccctttcccagaaccttggtatgttaccctccccccacataaggacaagtcaatttgttggtagcttctggcttgaaaccctctccctccatttcatagccacttaaccacgcataacagagttccatctttttgtcagtagatagcctttttttacccaccccccccccggcttaagcctta

SEQ ID NO: 5 - DNA, 3120 bp, Mus musculus
ggcggaggcgccctcggtacttcccgctcggcccgggcgcccggagatgaactgatcgtcggacccgctccccagctccgcgcgtctccgcccgctgcctcccctccccctctgccgtccgcggcgcgggtcccgcggcctgggcatccaggatcgcgggcccccgcgcggggcatcctccgcccgaggcgccggcccgcgccacccttcgccctgtgcccgccggtgacacagagagagccccaggaaacccgtgaatgttgaagaaaattcatctttgaaattttaatattcgaggaaatctgcattcatactcatcttttattaatctgaggggatttttgttttatttaaaacttcttgatatttacaatgaatggacacagtgatgaagaaagtgttagaaatggcagcggagaatcaagtcagtcaggtgatgattgtgggtcagcatcaggctctggatctggctcgagttctggcagcagcagtgacggaagcagcagccaatccgggagcagcgactctgattctggctctgactcaggaagtcaatcagagtctgaatcagacacatcccgagagaacaaggttcaagcaaaaccaccaaaagtcgacggagccgagttttggaaatctagccccagtattctggctgtccagagatctgcaatgcttaggaagcagccacagcaggcccagcagcagcgcccagcttcatctaatagtggatccgaagaagactcgtccagcagtgaagactccgacgactcgtccagcggtgccaagaggaagaagcacaatgatgaagactggcagatgtctgggtccggatctccatctcagctcggttcagactcagaatctgaagaagagcgagataaaagcagctgcgacgggacagagtccgactacgagccgaaaaacaaagtcagaagccgaaagcctcagaatagatctaagtcaaaaaatgggaaaaaaattcttggacaaaaaaagagacagattgattcatctgaggatgaagatgatgaagattatgataatgataaacgaagctctcgccgccaagccaccgtcaatgtgagctacaaggaggatgaagaaatgaaaactgactccgatgacctgctggaggtctgcggcgaggacgtccctcagcctgaggacgaggagtttgagacaatagagagggttatggattgcagagtggggcggaaaggagctactggtgctactacaaccatttatgctgtcgaagcagatggtgacccaaatgcaggatttgaaagaaacaaagagccaggagacatacagtatttaattaagtggaaaggatggtctcacatccacaacacatgggagacagaagagaccctgaagcagcagaacgttagagggatgaaaaaattggataattataagaaaaaagatcaagagacgaaacgatggctgaaaaatgcttctccagaagatgtggaatattataattgccagcaagagcttacagatgatctacacaaacagtatcagatagtggagcgcataattgctcattccaatcaaaaatcagcagctggtcttcctgattattattgcaaatggcaggggcttccatactcagagtgcagctgggaggatggagctctcatttccaaaaagtttcagacatgcatcgatgaatattttagcaggaatcagtcaaaaacgacaccttttaaagattgcaaagtgttgaaacaaagaccaagatttgtagctctgaagaaacaaccatcctatattggaggacatgagggcttagaactgagagactatcagctgaatggtttaaactggctcgctcactcttggtgcaaaggaaatagttgcatacttgctgatgaaatgggccttgggaaaacaatacagacgatctcatttttgaactatttgttccatgaacatcagttatatgggccttttctactagttgtcccgctctccacactgacttcctggcagagggagattcagacgtgggcgtctcagatgaatgctgtggtttacttaggcgacattaacagcagaaacatgataagaactcatgaatggatgcatccccagaccaaacggttaaaatttaatatacttttaacaacgtatgaaattttattgaaggataaggcattccttggtggtctgaattgggcattcataggtgttgatgaagcgcatcgattaaagaatgatgattcccttctgtacaaaactttaatcgactttaaatctaaccatcgccttctgatcactggaacccctctacagaactccctgaaggagctctggtcactgctgcacttcattatgccggagaagttttcttcatgggaagattttgaagaagaacatggcaaaggcagagaatatggttatgcaagcctccacaaggagcttgagccatttctgttacgcagagttaagaaagatgtggaaaaatctcttcctgccaaggtggagcagattttaagaatggagatgagtgctttacagaagcaatattacaagtggattttaactaggaattacaaagccctcagcaaaggttccaagggcagtacctcaggctttttgaacattatgatggagctaaagaaatgttgtaaccattgctacctcattaaaccaccagataataatgaattctataataaacaggaggccttacaacacttaatccgtagtagcggaaaactcatccttctcgacaagctgttgattcgcctaagagaaagaggcaaccgagtgctcattttctctcagatggtgcggatgttagacatactcgcagagtatttgaagtaccgccagttcccctttcaaagattagatggatcgataaaaggggagctgaggaagcaggctttggatcactttaatgccgagggctcagaggatttctgctttttgctttctacccgagctgggggtttaggcattaacctagcctctgctgacaccgttgttatattcgattctgattggaatccacaaaatgatcttcaggctcaggcaagagctcaccgaattggacagaagaaacaggtgaatatatatcgcttggttacaaagggatcagtt

SEQ ID NO: 6 - DNA, 2000 bp, Mus musculus
tgaaatttgaaatcaggacagttgttggctatggtacttactagttttgtagtaatgttttgctagcctgactttccttactggtttttatgtccatggtccccggtgactgttactcttgtttggggtgtgtgcatagtagtgtctcatctgtgtgtaggcagtcagtgtatgcacacggtgtgacagtggaacggggtgtggctgggagtggggtgctggagctttaagagcatttgtttttattcagaacagtatttcccatcttttgcctgcaggcagggaaagtgtacagtatttattttgtttctgttttactctgaatttgtaagtctctaagtagcttatattattattatagggaagataagtgacttgcttaaagttgtatttagtattcttactttctaatttctgtattttaaaatattgaaattaaaattgtattacttttgttctgagtgccaaatcacaaaagaaaaaaagcagtaaccgtttacagaagcaacttagtgccttgtaatctaactttgtcactgtgactacattacctcttcaacgccagggggcacccgtgggcctcccagagcctctgcccgtggggttgggggtgggggtggggtgaccgcaaccagcagctcccccagctggccacacaagtgcacctttctttcggtctcccgcacactcttcctcttccttatagttactcacctccctcagagagttgttgctggacttggggtttttttggggggaggggtctattttgactttcaaaaccttttacttcccagcccgaacccctgttgactaatcttgcctgggtttgtgtaggtctccagggacaaaggttgaacaagttggacgaaggttttgacataagtgggacttcgtgatttaatttctttctttcttttctttttttttttaagtggggaggaaggggaaactagatggactatgagagacttgattttggtgctaaagttccccaattcatatgtgacatcttaaaaatgacaacaaatgagagaaaagctaaaaacaacaaaaaaaaacatgtaaggggtgagcagttaatggtctgcattccacatacaatatctgtgtaaaacgatttcctgtagaagtagctttaatggattttgctctagaataccttaggtctaccttagagcccccacacaatgcttttttccctgggtttaaacttcatctaactttcagaaattggagagcaaaaatgttgcttatcactgcacatcaatataaaaaagcttatttaacttatcaaaacgtatttattgccaaactatgctttttttgttaattttgttcatatttatcgggatgacaaatccatagaatatattcttttatgttaaattatgatcttcatattaatcttaaaattttgtgacgtgtctttccttttttccacagttttaatatattattcttcaacaacatttttgtaactttacactttttttggttattttattttaaaaaaatgaaaaattaatttaaaaaaatgcaaaaaactgttggattatttattttagaaattctcccctttgtgttggactgcaaattgagtttctttctccttaggcctttcacaggtaggactgagaatgtatgtataagttctgtgacagtacagaaggaaaaccaccattttatgtatagcttctaaaagggaaaacaaaaaaagagaaaaaccctttgaattccatgtgcccatctcaagacattccgctcgcagatttgtggttctggattccaggttggagttttccaatgttgacataaacaactggcgcacacacataaagatgaatgtaattattattcctcttgctggtcactaccgtcgctttctatttctctttctttgtgtgaatttatttaaaagaaaaaaaaaactttttgtaacgactatttgcagtttaaaaatcaataaaccccgtttttcaagcgagaagcacgaagag

SEQ ID NO: 7 - DNA, 284 bp, Mus musculus
attcggcacagggcagttgttaagtatgaaatagcactgataggaaatgcatgtgcatctttgcagactgtatttcctttggaaaagacttttctacttttaatataattaagccataacagtttcatgctgtggaaaggatgaaaaggttcattttaagagattatatagtatgaactttcacatttattgtgaaacatctaactttgccagtgttcagcaagttttcttttggggtgttggagggtggtaatggggaggggtagtgttggttttaggggttt

SEQ ID NO: 8 - DNA, 1586 bp, Mus musculus
ggactgactccttgggcagattgcctctcctccttctcatgccagaggctgctgatgaggaaaggtccaggggactgtccatgctgtcttcatcctcagagtcactgcctgatgctgcaacaagacccttcttgtttagcaatagtggttggaacactctcttgtaagttaccggagcactagtataggaggaggatcatcgactaccctcccgccactccacggctgctggctcctagaaaccccagcttcacctctcactgggactcgagttccagaatgaaaagcaagaagggtcttgttgcagcatcaggcagtgactctgaggatgaagacagcatggacagtcccctggacctttcctcatcagcagcctctggcatgagaaggaggagaggcaatctgcccaaggagtcagtccagattctgcgagactggctgtatgaacacagatacaacgcctatccctcagagcaagagaaagcactgctgtcccagcagacacacctgtccacactacaggtctgtaactggttcatcaacgcccgccgcagtccttcctgacatgctgagaaaggatggcaaagatccaaatcagttcacgatttcccgccgtggggccaagatttcagaagctagctctattgaagctgcaatgggtatcaaaaacttcatgccaactctagaagagagcccatttcattcctgcgtagttggacccaacccaaccctagggagaccagtgtctcccaaacctccctccccaggatccattttggctcgcccgtcagtgatctgccataccactgtgactgcattgaaggatgggcctttctctctctgtcagccgattggtgtgggacagagtacagatgtaccgcaaatagcacccagcaactttacagacacctctctcgtgtacccagaggacacttgcaaatctggacccagtccaaaccctcagagtggtcttttcaacactcctccccctactccaccagacctcaaccaggattttagtggattccagcttctagtggatgttgcactcaaacgagcggcagagatggagcttcaggccaaactcacagcttaaccgttttttcaaacaaaacagttctccaaaatacggtcctgattgccgggggtgatggcaagagatgcattattttatatatttttctattaatatttgcacatgggattgctcagacgaagcttcctgttactaagatgtcttcagtggaatagagtcattccaagaactacaaactaaagctactgtagaaacaaagggttttcttttcgaatgtttcttggtagtttctcataatgtgagacggttcccagtatcatgtgatcttctcctccagactcctcttctttatgttccaagactgtgcaatactttagacgccctcgcacctctttcttcccatgtggaatgggacgcccacctacagtctaatgagtaaactttcagttttttgtttgtttgtttttttttagattcaagcaagtatgaatctagttgttggataccttttttcatgatgtaataaagtattttctttaaaagttattgc

SEQ ID NO: 9 - DNA, 1754 bp, Mus musculus
ttgaagttatagggatccaatgtttttatgatataaatatgagacacagaaaacataaaataggtataaaatatcttacagtttgtacagttctttcgaagcttttacagtatacatctttagacaatttcacaagtggtcagtaaatgggtgagtcactccacagagaagaagagcagagacattgctgttcacagacaggatcagtccttgctttggcagacacccatctgcccagacatttcaagacagcctggtaaacaaaacctcatgtgacaggccaaactttgttccagattgtatcaggagatcccacctgctcctaccattctgcaggaacagtaccaactcagacttcacacatcctttgtcttggctggaatgtaagaatttgtgaggaaaactactgtctcccactatctttcctgtcctttctgctgtctgtaaggtcatctagctgtgataaatgactgaggccctctgaattcctatattacagattatttacacaaaatctcactgggagatctttattggggatgagaaataagaaaaaggcattgtctaaagaatcaagctctacacttgtactagtgcataacagagtgaaaatacaaattttgaggtcttccattagtcctatcactccataaaagtttccacataatatagacacctccagaggcgtagtaccaggtgctatgcttttcacgtttctgagatgggatgtagaagaaggataactgtgtgtaaagtagttcattaaagtttagagagacactcaccacaagccagctctttgtcactgaaatgctccaaagaggtgtgcaaacaggcagtggtctttcatcaaaggaatatcttaaatgatttgagtcctttagggggcagcaccatgtacagttcatattcctctgtagactttatgctcttggtcgtagggttgaaacttttaaacactttttcctttgatttttccagacagagtttctcagcatatcccttgctgtcctggaactcattctgtagaccaggctggcctcaaactcacagagatcagcctgcctctacccaccgagtgctgggattaaaggcatgtgtccccattgcccagctaggtttggaacttatttagttcgatgagactcttaaattgcttgtcttgcatattaaattacaagttagtgtggtgccagaccttcctagcatggaaacagtctcctcaaaaggacacaatcagatgagatgttttttgaaagctaaaaggaagctgacaagagagtttgtgaattgcttcacaggcaacaagacaaacccactgattttaaccttcacgaaagaatactgggacactgttactcacggttttgttaagcagaacaatttacattgttagcagtttgccttataatgagcagaactatacttgcaaatttgtgtaagtgactaaaaggtgtttgattcacgtctgcattttctaagggagaaggtccaagctccatgcatgtctgatactatatatatacattctacacacatgtaaatactgccagctatgaaagccgtcacacccattagcttatttgtagataataaaagtgaagacaacttaagcacaaattcagttcaaaagagaaccttatgatagattacttgagagagagagagagagagagactagaacatgaagaattaaacactgttcacagaccatttcttttaaaaagtcacagattctgaaccgacagcaacgg

SEQ ID NO: 10 - DNA, 4265 bp, Mus musculus
caaatgctgggcgtacactggacaaggcccactggacaacactggttcctgggaggtgacggtgccccccagctttcttcttgtcacacactggacgccgccagctacagcggtcctcagccctggcttccttctgttccgccccctgacacactggacctgggggcgatccctgccgttgatcctcagtcacacacccctccttctcacctgccatgctgcagctagaacttcgcaaagcctctatgtttctgtggagtaaatattaggatggggaacagagggagcaatagcttggaagaagatattcttgaaagggccaaaaagaaaatggttttggatcatcttgttattcaaagaatggataccactgggaagacagtgctgcacacaggctcggctccgtcaagttccacccccttcaataaagaggagttatccgccattttaaaattcggtgctgaggagctctttaaggagcctgaaggagaggagcaggagccacaggaaatggatatcgatgaaatcctgaagagagccgagactcacgagaacgagccaggcccactgagtgtgggggatgagctgctttctcagtttaaggttgccaatttttcaaatatggatgaagatgacattgaattggaacctgaaagaaattcaaagaactgggaggagatcattccagaagagcaaagacggcgactagaagaggaggagagacaaaaggaactggaggaaatttatatgctcccgagaatgagaaactgtgcaaagcagataagtttcaatggaagtgaagggaggcggagtagaagcaggagatattctggatctgatagtgattcaatctcggaaaggaaacggccgaagaaacgtgggcgaccccgcactatccctcgggagaatattaaaggatttagtgatgccgagattcggcggtttatcaagagctataagaaatttggtggccccctggagaggttagatgcaattgctcgagatgctgaattggttgataagtcagaaacagatctcagaagactaggagaactggtgcataatggttgtgttaaagctttaaaagacagttcttcaggaacagagcgagcaggtggcagacttggaaaagtgaaggggccaacattccgcatctctggagtccaagtgaatgccaagctggtcattgcccatgaggatgagctgatccctctgcataagtccatcccttcggacccggaggagaggaagcagtatacaatcccctgccatacaaaggctgcgcactttgatatagactggggcaaagaagatgattctaatttgttaattggtatctatgagtatggctatggaagctgggaaatgattaaaatggatccagacctcagtttaacacacaagattcttccagatgatcctgataaaaaaccacaagcaaaacagttacagacccgtgcagactacctcatcaaactacttagcagagatcttgcaaaaagagaggctcagagactttgtggtgcgggaggttcaaagaggagaaaaacgagagctaaaaagagtaaggcaatgaagtccatcaaagtgaaagaggaaataaagagtgactcgtcgcccctgccttcagagaagtctgacgaggatgacgataaactgaatgactccaagcctgaaagtaaagaccgatccaaaaagtctgtagtgtccgatgctcccgttcacatcactgcgagtggagagcccgttcccatagctgaagagtctgaagagctggatcagaagacattcagtatttgtaaagaaagaatgagaccggtgaaagcagctttgaaacaacttgacaggcctgagaaaggcctttcagaaagagagcagctggaacacactagacagtgcttaatcaagatcggagaccatatcactgaatgcttgaaggaatattccaatcctgaacaaattaagcagtggaggaaaaacctgtggatttttgtatctaagtttactgagtttgatgcaaggaaattacataaattatataagcatgctattaaaaaacgacaagaatctcagcaaaacagtgaccagaatagcaatgttgctaccactcatgtgattaggaatccagatatggaaaggttaaaagagaatacaaatcatgatgacagtagcagggacagctattcttctgacagacacttatctcagtaccatgatcatcacaaggaccgccatcagggagattcttataaaaagagtgactctcggaagagaccctactcctcatttagcaatggcaaagaccaccgcgagtgggatcactacaggcaagacagcaggtactatagtgaccgagagaaacacagaaaactggatgaccacaggagtcgagagcacaggccaagtttggaaggaggcttaaaggacaggtgtcactctgaccaccgatctcactcggaccatcgaatgcactcagaccatcgctcaagctccgagcacacacatcataaatcctccagggattatcggtatctctcagattggcagttggaccaccgagctgccagcagtggccctaggtcacctttagatcagaggtctccatatggctccaggtccccatttgaacattcagctgaacacagaagtacgcctgaacacacctggagtagtcggaagacatgacagaagctcatgagtttgtcttccgggactttgttttagccatagatcataaaccaacacagtaattgccttacatgacttgaaagattcaaacagctcttctgtcagtagcagtattgttacttctctccaggacgcaaggtctattatcccaacagaagagaaaatatttttatatttaaggattatgctgcactgtactgcgatactgcagtaccttttttctcttcttttaaagaaatggaaaatgtttactatgccagggacctcagcactgccctcccgtgtaggctgtataaaactgtttttatgtcagtgattttagactgactccatttaaattatgtttgtatatgaactttactctgacctgtgatcatgtttcaggaaggaataaaagagaggtcttttcttaataaagaaaaatcactcaaggactttgttcactttccaaagctacttgtttacattgtacactgcgaccaccttgccgctcccatcacaagcttgaatatttaaatcctgtacttaagtttgtaatatagccgggaatttcctgtctgtgattattattatgcctttttacagaagaagatggctgtaaattattgtaaatggattaaatgagctgccctgccctgcccttctcaggcttcttttgactgttcctttccctaccaactcaggccttcttattaaaaaaaaaaatcagtgtaataacactttttaatgatttgtcttgatggaatcattgtttagaatgtaaaaatggggaaaggggccacttatttcttttagtcctttttatattgagtatttttattagatacatgttttgcccctcctttttaaagtcaatattgtgtttgtagttttacagaagcagtggcgagggttacatgtgagacaaactcggtgctctgggagagtccccagtgcattgggttgaaagggtggtaggtgtatgaacacttaaaatccaaacggccaaacgttcttgtaaatgcattgcttttccctttcatgtgggcaataatgtcaaacgtgctatgcagccaggttaacatttgaggtaaacttgattggctttaatataaaaactgttacagtacacactgattgtatataaaaaccttatatatgacaaattaaattttaagaaaaaggatatgtgggctcctgtagttttctgctgcattcttgtttccacgagcatttaatctttgctttaagtaacagctgccagaaagaaaggtattttggtcagcaactgagaactgttgatatgtaatcctaaaactgcctacgtctgtatagatgaatcagtcttctatgaagtaaaacagattctagcagtctcggcttcctgtagacattcctctaggatgtcttactcgtcacctcgtttctccttatattcagtgggaattttggatgtatgttgtgtaaccattaataacctttactcctggtcatgtctccatggagtagctgtggggctacctaaagttagtctgtggctttgtattttcatatctttttttttacatggataaatataaaatttatgctgtgtatac

SEQ ID NO: 11 - DNA, 3442 bp, Mus musculus
gagaagacgacagaaggtttatgccagtacttttattaagacagagtctaactgtacagccctgcctggcttcaaactgcttcctgagtggactttcacatccagcatatatgccagcattttattagtcactccccatgtttcatttgttcaaccttttagccagtgatttttctccctacaaaactgatagcagtcataatccttgagctatcaccattcacaaaggagatcaagaggctttcagaattttatttaaaaaatccagagtggatgaaaaaggaacaatcatcaagaatacagatgccagacattggccaaccaagagggtggctagagaagtatgcctcaggcccatgagaggcttatatcattctcatatcatactttaagctatggctaacacaactgctatccactgacctagcttctgcacctacagaccatggtacaggtagctcagtcttaaaggcatcagttgtaggagcaaatatacagaggtcattctttatagtagtgctcttggccaacaagcatctgcatatgatctcaactcaaggtcaatgacagactacctccactgcatgtgaaatgggcttcccaccttgaaatcacagccatttcagcacaaaccaaatctaccttaatgattggttcacagccccatcccacccctttacatgcagctgaaaataacaggctagtgacagaacagtatgaacctgctattgctgactttattacccattaaatgagttagcaattgtcactaagtttatatattaaggaaattatatatagaatactgcaaaaatacagtaaaaagactgaagtctgccccttttctgctcaggaagtccctttagtcccaagcttcatagtatgtccttctggctccacaatgcactgccacgattactgtttctcttccttctgatcttccttctgttccccagtgccagaacttccagaaccttcccgttcagatgccatctttttgtacgccatttcgaagagtttcaatgacgcctgctgtagggaagatgctgcctgcctgatgttctctcctgtctcactgtcctttccagcaaggagcgctctcattttggaaatctcttcctttagcttgttgcactcatcagcaggcaactggtccttaaattcttccatcttggtttctgtgtcatgaataattccttcagccatattaactgcttcaacacgttccttcttcctgcggtcttcctcagcgtacttctctgcatttttaaccatattttcaatatcatctttgcttaatccaccagaagactggattacaatctgttgctcacgaccagtgcctttatctttggcagaaacgtgcacaatcccattggcatcaatgtcaaatgtaacttcaatctggggcactccacgaggggctgggggaattccaatcaaagtgaactgtcctagaagtttgttgtctccagccatctctcgttccccctgacacactttaatctctacttgagtttgtccatcagcagcagtagaaaacacctggctctttttggttggaatagtggtgttcctattaataagtttggtaaagacgcctcccagagtctcaatacccagagagaggggagtgacatccaggagcagcacgtctgtaacgtcaccagccaacacacctccctggatggcagctccgatggctacagcctcatcaggattaacagctttactcggggctctgccaaaaagatcttgtacagtctgctgaaccttgggcatccttgtcatgccaccaaccagaatcacttctcctatgtcactcttgctgacttctgcatcctgcatagctttctgacacggagcaatagttctcttgattagatctgtgacaatgccttcaaactgagctcgagtcagcttcatattcaaatgctttggtccagaagcatccatggtaaggtatggcaagttgatgtcagtctgcacagatgaggaaagttcacatttagccttctcagcagcttcccgaaccctctgaagcgccatgttgtctttggtcaaatcaacccctgtctctctcttgaactccttgacaatgtgccgcaacaaagcttggtcaaagtcttcccctcctaagaaagtgtccccattggtagatttcacctcaaacactcctttctgaatttccaggatagaaatgtcaaaggttccaccacctaaatcatacacagcaatgactttatcttcagatttgtccagaccgtaagctagagcagcagctgtaggctcattgatcactcgaagcacatttagcccagatatctggccagcatccttagtggcctgtcgctgtgaatcattgaaataagcagggactgtgatcacagcattttttgctgtgtggcccaagtaattttctgcagtctctttcatcttcatcaacacaaatgctccaatctgacttggagaatagagttttccatgagcctcaacccaagcatcaccattggaggcacggacaattttaaaaggaacattcttagtgtctttctgtacttcagggtcatcatatcgtcgtccaataagacgcttagtagcatagaaggtattgtttggattggtgacagcttgccgttttgctggcataccaacaagtcgttctccatctgctgtaaaggcaaccacagaaggggtagttctggcaccttcagcattctccaggacctttgcttgtttgccctccataacagccacacaggagttagtagtacccaaatcaataccaaccactgcacccttgattgcttctgatgcataatctcttcttgaaacaaatctaaaagcctcatggctaaggccattccagccatcctggggacgggcggctgcggggctccgggacgcagcggtgcccacgagacgcgcggccgcggctctgctggcgcttatcatggtggctggacagagggggttacgagggcaagaaccaacaccccacgggcccggagctgcgtgcacggtggtggtacgcttctggaaacctccaaccacgtggggtgggggcgggggctggccgctgcagcaatgaagcccgctcttctgctgaggcgcccgacgtgctgcctgactagagactacgtgtccgctgctcccgggatcggcgcacgccgccagcacgcctctcagcattaatcgttgaaagttcccgttctctcatcacggtttccagggctgtggaagcagggagaagggaaagagagaagagaggtggacaggcatcgcttcggacagtcccggtccttaggacgggaaagactcaaggtcacacgggataaattacgaacagattacagtctccttgcgtcagttgcgcagaaggaggtgcgttctgcgcctgcgttctccgctccggacagaggtcgtgaagcgcatgtgtgag

SEQ ID NO: 12 - DNA, 1200 bp, Mus musculus
tgctggtgggatcaaagcgcagtgtcctgcggcggggagcttggaacgctaagaaaagtgaccatggagaacaacaaaacctcagtggattcaaaatccattaataattttgaagtaaagaccatacatgggagcaagtcagtggactctgggatctatctggacagtagttacaaaatggattatcctgaaatgggcatatgcataataattaataataagaacttccataagagcactggaatgtcatctcgctctggtacggatgtggacgcagccaacctcagagagacattcatgggcctgaaataccaagtcaggaataaaaatgatcttactcgtgaagacattttggaattaatggatagtgtttctaaggaagatcatagcaaaaggagcagctttgtgtgtgtgattctaagccatggtgatgaaggggtcatttatgggacaaatgggcctgttgaactgaaaaagttgactagcttcttcagaggcgactactgccggagtctgactggaaagccgaaactcttcatcattcaggcctgccggggtacggagctggactgtggcattgagacagacagtgggactgatgaggagatggcttgccagaagataccggtggaggctgacttcctgtatgcttactctacagcacctggttactattcctggagaaattcaaaggacgggtcgtggttcatccagtccctttgcagcatgctgaagctgtacgcgcacaagctagaatttatgcacattctcactcgcgttaacaggaaggtggcaacggaattcgagtccttctccctggactccactttccacgcaaagaaacagatcccgtgtattgtgtccatgctcacgaaagaactgtacttttatcactagaggaatgattgggggtgggggggggcgtgtttctgttttgttatgccaaatgagaaagctgtcagggagactctcatttaaatctaatctgacggtcctcctggtctttgtacgctaccactgcctagcaatgcagccagccacagtgcagctacctcaacttcgacatcaggtagttgaaatgaaatttaatttaataaggagcaagtaactgtcaatgatggtactatcatcctagatgaaattacaaagttgcccttttataattagcaagatttggcgatactatgaattttgaagtca

SEQ ID NO: 13 - DNA, 1158 bp, Mus musculus
cttctgcataaggtttgatgggtggaaagggcctcaaagaagccacagaaatcgccattctaaatgccaactacatggccaaacgactagagaaacactacagagtcctctttagaggtgcaagagggtatgtggctcatgagtttatcttggacacccgacccttcaaaaagtctgccaatgttgaggctgtggatgttgccaagaggctccaggattatggatttcacgcccctaccatgtcctggcctgtggcagggactctcatgattgagcccaccgagtcagaagacaaggcagagctcgacagattctgtgatgctatgatcagcatcaggcaagaaatcgctgacatagaggagggccgcatcgacccgagggtcaaccccctgaagatgtctccacactccttgacctgtgtcacatccttcctgctgggatcggccgtattctagagaggtagcagcatttccactgccctttgtgaaaccagagaacaaattctggccaaccattgcccggatcgatgacatctacggagatcagcacttggtctgcacctgcccgcccatggaggtctatgagtctccattttctgaacagaagagggcttcttcttagtcctccctcctacgttcaaagggctgacccgatgtctcttgctggagcatttgacaagcaaggatatttcttctcctttacgtggctcacacatgagttttatacgctgtatatttttataatctttcaaggtaatgtaagcacaattagcatggtgagtggcggccacctgctgtacggcaaggcagtggctgttcgtgttgccttgattgggagccatttagtttgccggatagaaaatgtggggtgcgctagcaattttaacattttaattcaagaagtttgcagcagtcagagcctatgctgggaattcaatagacattctttttgttccaaataagtcctcgtggactgtgccctctgtgggaaaccccagggcaaatgtttacattttatacactgaagaattctcctcactaatgtgcctgatctgtcacagcataagtgtcctcctttcactgtgcggacttttttttttatctgcttttattagtgtcctaataaaactgagtttgagtaaaaaaccttatgcagaag

SEQ ID NO: 14 - DNA, 442 bp, Mus musculus
attcagctaagtgacccttaacctgggatctactgtactgtaattttcaacactatagaatattatgctccccagtattggtgaatggttagcaatacaaaactggcagcttagtagttcaggatctttggaatacattgaaattcataaatgaagttcatttttgaagcacacaattcaaatcattaactcagacgagacaagtccatttatatggctaaatacttagcttgaatactcttctgtattttactaatcctaattaattccttttccatgtattttactgtacttatccataagagatcagcaggtattatcagacactcactgagtgctcagaaatagtgaggactgtgaggaacctcaagttcagttttgtctgtccgtttgtctgtccgtgaaggtgtcttctccaggaggtttgcagtgttgttttgac

SEQ ID NO: 15 - DNA, 1405 bp, Mus musculus
tgtggcccttagagacagaatggtttattgaatccaaaaggtagagtgtcaaaaacttaatagaatcctgtccagtaggcactctgggccaggaggtttgcatcttacatcagtctccctgaaggtctccgtgttcttggaaagtaaaggccctttgctgagcaccccgtccttgccacagggtccctgtgactcaggggacagtgttgtagacactgtagttgtccttctgtgtgacatgcagcttccaggggaagaggcgcttctgggaagggaggcctcgggcttgcttcaccatgagtcccacatcctcatctgacagcagcctaaccaagtccccctcagcatcctggtagctaagggcaatgtcttctctctggaactcacgcctcatgagcgctagcaggtctttgaacaggggcgtgctgctgaggtcctcctccaccgcaatgtccttgatggttttgcctgtgtcttcatagaagtagcatcgtagccagttggtggtgtcctcgtcctcgggaaagtccttaaggatcttcacgaaggaccctgggaagatgcctgtggctccctgggaggtgccctccagccagtctttgttgatcttactgagaaggaagatcacatctccagctttgaaacttagctccaatttgctgttcccagtgaagtcaaacaaggcctctgctcttggcgcttccatgcgatccatgatggccccttgtggagacacacccttgattttgcgcgtgcgcggtcggagcctgcggagtgcctggggcacctgctcagcatcgtatgcagactgatagaagaagatcctgacgtcggggtccatcagcacgcagaccggcaggctcaggaggttcttcatgtaggcattgagggccgggatccgagtctcagcgatctcttgttttgcgcccatgtagactttggctggcaatgtgggcaggttgcaggtgaaagggctgttcttgctctctggcccaaaccgctcctccagcttgctctgcagggcgtagaactggcgatagcggcggtagatgagatacttggaccctccttttgttttgacctcgatgacaaaaacaaagtggctggtgaagcctctcttctcctcgatgtcagcgatgttggctgagacggccacatcgtctggaagctgctcaaagtcgctctctgatcgcagctgctgggccagggccatggctgtgggtagtcagaaaagcagatggatgggggccttgcccaaccagatccaggccgagttcacctctcacttcctcctatgtgcatctgagagcttcctgaagcttggggtgtccccgggggcagcctttgaggaggtcccgccctgcccagcttctctggaccctccttgaagtacctccttcaggcg

SEQ ID NO: 16 - DNA, 745 bp, Mus musculus
ttaggattctaataaaaccacagaattcttttaattaagctcaaagttgcaagtttgtctcacgtatctttcatttgactaatgaacttttcgacaattccccccagtctttcaggttccatcagtttatttaccagttcctgctcaacagctatcagagccaggctactaagtttctcttgtcccatggtacgaagtaagtatgttttaagatgagacagcgtggaaaatgacttctcattatttgctgaagtaattggccaagataaagcaatatgcaacagcttcgtgatacaaggaatattattgtggagagcatgctgaataaacaaagagcctaggtcaatgaagcttaaggacccatcctctgcgatgaagttgaaacctgcatactgccggtaaaatcggagctcgggaatgatgtctgcatcaagcttatagaattcctggatgtgcttggctgttgcttcatttaatggctcattccacttaagtaacagctctgaaatttgtttcattttacaatagtcaaactctgaaaaatataactttaaatgttttaataccgtgtccagaccttggtagtaaatattaaatttatattgttcatctgctgaagtaggaaaaaacatatgctctgatttgccaggatctactgttctttgaagtttgcttctgttttgaaaaaaaggtttctcaacatcataacccttagtggtgatcttttaacatatatcatctgcat

SEQ ID NO: 17 - DNA, 1542 bp, Mus musculus
ccccgccgggcgaccacttcaccctctctacgtcggtctctcaaagatgccgctctacgagggccttgggagcggcggcgagaagacagcagtcgtgatcgacctaggagaagctttcaccaagtgtggattcgcaggagaaactggtccacgatgtataattcctagtgtgataaaaagagctggcatgtctaagccaatcaaagttgttcagtataatatcaatacagaagaattatattcctacctaaaggaattcatccacatactgtatttcaggcatctgttggtgaatcccagagaccgccgcgttgtggttatcgagtcggtgttatgtccttcccacttcagagagactctgactcgtgttctttttaaatattttgaggttccatctgtcctacttgctccaagtcatctgatggcactgctgacgcttggaattaattcggccatggtcctggattgtggatatagggaaagcctggtgttgcctatctatgaaggcatcccaatactgaattgctggggagcactgccgttaggaggaaaagctctccacaaggagttggaaactcagctgttggaacaatgtactgttgacactggtgcagctaaaggacagagccttccctcagtgatgggttcagttccagaaggtgtgctagaagatattaaagtgcgcacgtgctttgtcagtgatctgaagcgtggactgcaaatccaagcagcaaaatttaatattgatgggaataatgagcgtcccactccacctccaaatgttgactatccattggatggagagaaaattttacatgtgcttggatcaatcagagattcagttgtggaaatcctttttgaacaagataatgaggagaagtcagttgccactttaattttggattcccttttacagtgcccaatagacaccaggaagcaactggcagagaatttggtaatcatcggtggcacatctatgttgccaggctttctccacagattgcttgcagaaatacggtatttggtagaaaagccaaagtacaaaaagacacttggcaccaagaacttccgaattcatactccacctgcaaaagctaattgtgtggcctggttgggaggggctgtttttggagcattgcaagacattcttgggagccgctccatttcaaaggaatactataaccagacgggccgcatacctgactggtgctctttcaacaaccctcccctggaaatgatgtttgatgtggggaaagcgcaaccaccgctgatgaaaagggcattttccactgagaaataaacgcttgaatacatccagccttgcttatttcaaatatttaatcaattagaggtaaattgtacaaagtatgtgggatgatttaatatatagaggtgacctttatgttacaaagcatttctgtattttctctttgcattaatatttaattcatctgactttgtctcttgtgtcgtatgtcgtagtggatgcttgtgagatatgttgtatttatatcaataaatatagttaagctattcaaaaaa

SEQ ID NO: 18 - DNA, 1343 bp, Mus musculus
aattcggatccttgtttatatcaaaagtggatgtgaagatgttccttaaaggagatacacggactttctggcacaattttgagcctcagctctccttactcatattctagcctccctccttccctgcccatctctcccccagagccatggctttcatggactttctctttctctgtgaatactataagccagttccatatttagacacctggttttcttgttcaaatagacactagatgattcttggctattctccactggtattagaatgtcccctgtccttagagctatcctggcctaccacacccacaacaggctccctggggctctttaacctatatgcagcaagccactctgggattggtcataaaagggctctgtcaagctctccatgaccccttgatgccctttgagggcacaagaagagtcatacaagactcagagctaaagataattctctacccattctaaaaaagtccactagttgggttgtggtaggacattgcttgccatagtggtgatgattctggtagcacaatctgactttgagttactccatgattttgtaaagtattaggagcccttaaagtgttcttttgccatcaactataaatttctcatatatcctggaggctttccttctctttgccttgggtaaaaatcaaatgaggtgaaagataccttcatgtccatattttgtatgttctagctagataaaatggatgtttcagaaaagcctttaaaaatctttatattgacatccataacaccaaaaactgtctttttagctaaaatcgacccaagactgtcacagcaaagtagaagaaacagccatcttctttggaaaagtaaaatgttcataagaacagggattgcccactatgatgataatgatgtactgtgtctttgtgtaagtcctatgtgttgacttttcaaatatagccaattgcttaggtatgtacaatgagtttttctattgttcctttgatttttcagagaccccccctttctttttccagtttttgtatatttattgattgactaataataatgaggaaaacctgatgtgtacattacccgattgaaagtgtgtattggaaaatattaaaaggttgtaaaaaatggaacctagggtctctggtgtgaagggtctgggaagtggagaggggcagaaagtgattgtctggatgtctgaataatatggaagaagtcagaggcagctggcatcctttgttttgttttgttttgttttgttcttttatagcccccccctcctatttttctttggtgtttttgttttgtttgtttgatttttgaaacaggatctcatagagctcagtctg

SEQ ID NO: 19 - DNA, 1747 bp, Mus musculus
gacagcgcaaaagctcgcaaaccttgtcagcctggatccacgtcttattcaaaacgtgtcgcgatcattcacagcttaagatgtacgggattcgcgcgtgagaagggagagagaagcgccggtttgccgacgccaccccctctcatcagggctggcgcggcgagtggcagctccttaggtcacggcttccgccggcgccagtggcctagctccctgcggctgtccttggtcgccttgtcaccaggctgaagcagtcaagatgaccttatttcagcttctacgtgagcactgggttcatatacttgtccctgcgggatttgtctttggatgttatctagacagaaaggatgatgaaaagctaactgccttcaggaataagagcatgctatttcaaagggaactgaggcccaatgaagaagttacttggaagtaaaaaatggttaaatcacagaatgttcacattataaaagttattggagaaataaaaacatttactatttaattagacttttgaagcagtacagcttttccactgatgattttatactccccactcatgttaagaatgttgttctagttttcgttaaacgtagaaacaataatgtcaaatgataatgtcttagaacttgaatccatgagcagagtcaaaggctggaacccttgttttggacctgatttatggaagtgaagagttggaccaccatagcatgcattatagctactggttttgtgacagttgtccacaacacgtatttgttattttaaaaaaaaaaaatcatgttttattactggaacaaaaataacttgagtatttatgtgagaggaaaaccgtgtgcgaacttggttgactcattgaaggcaaggtgctaaaatccatcagcatttgtttcttacttatccttatacaaatcagctcggtcttacagaccagcaaaatatctcaagagctgttatgtaacagcctcaaatggttgattattagaatgaagtacaggaattctataggaatatactataataccagttgtcaaacagagcttaagaaaaggagagatgaaatctactgtagttggagagaatgtggtttgagccagagcagagacttggcagagcatgggcacatcttcagaaaaggcctgccagctggactccccacccctcaccccaccccccttgtgagttctggtttgtgtggatgatgtccaagttaccacgtgggaaagtgaaccagatcacaaacttctgcaaaaaggctttcccagagggctgagagtctgaagaaagttcactggactccatgtccagagtctgtagcatgctgacttttctgttgacgtttacaaagaaaggaaaaataatggtgatatttatactgtgctagaaacgaaagggccagggcacacaggacttggaaagcatccagcaatagccctgcagcccatgtggagaagctgcgggctgcagtccatcctcagggagactgtgctctgctcctggcctccttatgggggtgctgagtcactgctggctctcgcagcatttctcctaagcttccttgttgcacaaagaaaggaaggaactcacattgtaacggatagattcttgtttagactggctttgttgttaacgctttctaagtgaatgcagcatgtgatggtattttaaagtgtgttcccatgtgacaattttataaaagtttgttaccttgcaattttatttcaaaacataaattcagatttaaaattag

SEQ ID NO: 20 - DNA, 2387 bp, Mus musculus
aattgatattttttgatctgcaaaattttattaagcaatagctggacaaatcttacaatttcaaatcatcaagaaaaaaatgagattaacccatttcaattttaaaggcaaaataatttggaaaattacatcattttgaatgcaaaccattactcccttctagaatacttcctggacaatctagtatacaaaatacatacaaagataggcaaataagggagagctcattgagatgcaatggtgactctcaacagatcttgtctggaaggttaaagcaaaatgctgacttttcaaaggaaatgaagaaaccaacactgcagggaccgatgtgtatgaaacagtgcacgctgttcccaaagtgtggacaagaacatgctggtggtcttgtttggtctgtgacatatgcaccccacattgcacatttatggggataaaaaagatgacagagaaggagaaagacagatgccagcagtagcaatagtagtaaactcggaaattatgcccaaatttacagtactaaaataatgcatttataaacaaatgcaaacaatacacctttggtaaataatacagtgcgtttccactcaaatgtataacccatgcttttctacaacacgagaaagaatatctacaataaactagatcttagcatcctacccttcccctgaaaattgacttgtaaacagaaaatacggatacaaagtcacaggagaactgcacaaagctgaagaaaatggaaaccaagtcagtcacaatcctccagagttcatctgccagagatgaggcgatacacagccgtgtagtcttctgaactgtatgttaccaaaaactcccaggagcaaatgccagctcaaacctagctactgttgccaacctgtccccagctcacaggacagcacaccaaaggcggcaacccacacccagttttacagccacacagtgccttgttttacttgaggaccccccactccttggggactccttggggacttccactacacttggatttgatgcttccatcattttattagaataagcattctaaacacatttaggggaaaacttctggtttcttttcaggagacaatagacagcttacagactcatgaaaacttttgtctacacaggtgccacgttttgacacatactcccaccctcgtgaaagggaagacaagctgctgaattctgagctctcaggctaccagagccagaatgcagtgtttaagcttctccaggtatgacttttcatttaagagatggacaaaaggaggccaggcaccctaggggcctggccaaagtcacccctatccccgtggcacgacaagtggacctggagtccctggcctttgactacagagtgagtattacacagaaggccagtctctggatggacaaaaccaacatgcaaaagctttcgctttgtatgctcattagagccgagtatttctagctgtggtggtcccagtttttctcaggctatacattcaaataagtatattcacccataacttttgttttgatgatactagagccaaataaatctaacactagttttcatgttggttaatatactcttttcctatgggtctctatatagttgactgtttttaatttcagtatccaatgagacattgggacattcacaaattgctattaataaccatgaagtacccaaaattctgttacttaaaagtcttcatttgaggagaatttaatcaagtgatattgactggaagaagccattatcttaaaagatacgatgccacaactgctgcagccagcagctttctgcccatgtggaacctgagactcactctccgcctgggggaggaggctggtgagtggctgctgttcctgcagctcacaggtcaagcaactcaaggccagaagcctgcatctccacagacaaagagtatcttttagggatgttcttatttagcaaagttgtaagtataacttttgcagtctggaaaaggtaaattttaactaaaaaaaacaagacaaacaaacaaaacaaacaacaacaaaaaaaaaaacccctaaaacttgaagatcatttactaagggactcttttgagactcttatctcagatgtcttgcccttcctgtgtcttatcacattttattttaaattaacattttcaaaataagaaatactgtcttagggtatgctatctcaaagatattgcctattttgcatataatcaatactgagataaagagtgaatttatttttttaggggatcaataagattttctccaactctcaagaaagttgcaaagcacctagcctccccgtcccccacaaaaaccttctgtcatattctctttatttatttagaaaaatagtacctttgtaatgccttgtttggtggcaatatattttg

SEQ ID NO: 21 - DNA, 683 bp, Mus musculus
attcggatccttgagcggagcacggcgctgcttccaccgctgctcaggaggggagcatgtctgcaactcgagccaagaaagtgaagatggccaccaaatcgtgccccgagtgcgaccaacagattcctgttgcgtgtaaatcatgtccctgtggttacatatttattagcagaaaactactaaatgcaaaacattcagagaaatcaccgccgtctacagaaaacaagcacgaggccaagaggaggcgaacagagagagttaggcgagagaagataaattctacagtaaataaagatttagaaaacagaaagagatctcgaagtaacagccattcagatcatatcagacgagggagaggacgacctaaaagttcatccgccaaaaaacacgaggaagagagagaaaaacaggaaaaggaaatcgacatctatgctaacctctctgatgagaaggctttcgtgttttcagttgccttggcagaaataaatagaaaaattatcaatcaaagacttattctctgatatttgtcttcaaactctgttggttgccactttcttcaggattgcatcagcagatcctcaggcagtcgtggactctgaggagcaccagactgtgtgaatgagggcctttggttgctttctctgtttcctagtctgctgccacgctttgggagcaaagctgtg

SEQ ID NO: 22 - DNA, 1520 bp, Mus musculus
acagcaccccagtccacagggaagatactcacgttagttgatggatgttggaaggcattggaggaaaacaccacagttaggactgttaatgactccacactgccgttaagactgaccttgaacatgcgcaaacgttcatcagtcatggtggtggtggttagtgccatagtcaaataaaaatatttctatacaatatgttcatttggtcatgtgctatttacagattttacaaaacatacacacacataaacacacacttgcccttacaccaacctacatacaacgggccagcagaaacttgagtaatgtatatcacatgacagatcgtgcctatacattataaataaaatcctactatgcggcagaacaggactcttctcagaacaggaaactaaggctctaagctgagaagctcagaaatttgaactgattatacgtaattcaaggccattaagaacaagacagttatttttctctgtccatttaaaatgttttatttttcttttaaactagattgtgaagtgccattgaataggcaatgttggcaaaacaatgtctgttacaataaaatacattagacatttaaataaataaccttaaaaactaggcgaagggacagaaacccagtcgattggatctggagcaatgttttctgcacaagcgagacaggcaaacctctcgtgaagacggatgtaaacagaaccatcagacctacagaagaagccgagcaggctggggtgggctgggggtgacagaggctgagggtgcaggaagtggcaaaccaaaaatactgactgaggtagcaggactctgctcagtgcagaaaaattggcttatgaataaaaattagactgtgataccaaattaaatccagagaatcatgcaaaagacacaatacatgctatttagttttttttcttttaaaaaccacacacattcatttcctaagtctgctgccaattaatttgctgacaatgtctgctggctcaactatctttgtacaagtatgagtattaaacagaacactgtacatgctttttcatctacaaaaaaatatctgcataaaatagtgttagttctctgtacacgtcaatggacactttaattatgtgtataaaaaaaggaaaatcttgtcccactggagtcagaaaaagcaaaacaaaatctagatatgaaacctccttcaccagccaatatgttcatgtgaaaattcagcagaaatagacagttgctttaaacaataaaaaattaattgattaagttaaacatcttctgtatgtagcgctgtcagtcaagaccagaacatatcgctaaagttagtgtctgtgctgtagacccagaccaccaggggcataatgagcagcacaaacggccaggaaatgccatcggtgcatgccgacagggagcaggaagatttggtggcaggctgcacacactctttaccgggttgttccaggtagtttcgggccacaaacttgagcgtctcatccatgaggatcacaggcaaggagattttcagc

SEQ ID NO: 23 - DNA, 596 bp, Mus musculus
cggctctgctctccggcttcgcaatggtagcgatcggtggaagtccagctggacacagaccatgactacccaccagggttgctcatcgtctttagtgcctgcaccacagtgctagtggccgtgcacctgtttgccctcatgatcagcacctgcatcctgcccaacatcgaggctgtgagcaacgtccacaacctcaactcggtcaaagagtcaccccacgagcgcatgcatcgccacatcgagctggcctgggcttctccacggtcatcgggacgctgcttttcctagcagaggtcgtgctgctctgctgggtcaagttcttacctctcaagaggcaagcgggacagccaagccccaccaagcctcccgctgaatcagtcatcgtcgccaaccacagcgacacgagcggcatcaccccgggtgaggcggcacgattgcctccaccgccatcatggttccctgtggcctggtttttatcgtctttgctgttcacttctaccgctccctggtcagccataagacggaccggcagttccaggagctcaatgagctggccgagtttgcccgcttgcaggaccagctggaccacagagggg

SEQ ID NO: 24 - DNA, 571 bp, Mus musculus
attcgaaaatcgattctttatctaaacctactttagaagatttaatatgtagtacgactgaagaaggaaaatatcaggcttaccgaggagacagaaaaattgcgtgctgaaaatctatttttgaaagaaaaaaagaaggaattctaaggatcccagctatcttcatacacatcactttgtttagattgattctttaaaagtgggattgtatttgctcacatggtagaactaaactaaaagaccttaactatgaatcatggtgcgcactaatcccgagtgttccgatggatagtgtgcatgcaaactcagcagcctgttaagtaacaatgcattccaagtcatgtgtacattctggaagacccttaaaagtctgtagctgaggtctctggtgaattctagtcaggtccttttcttctttttttatttactttattttattttttttttttttggttttttgagacagggtttctctgtatagctctggctggtcttggaactaactaactctgtagtccagcctggcctcgaactcagaaatctgcctgcctctgcctcccgagtgctggga

SEQ ID NO: 25 - DNA, 761 bp, Mus musculus
aaaagtcttttcactttaatacaaacattgtataaagaaaagttgaatgagagagatcaagtattcatcaaatatcagtcaaggcgatgattccatatgttcctgcttttccatcgctcccggccacgtgctaaggaccagataatcacggaggcaggaacgagccacaggccagaggcctctgtccatgtgtggaggaggcgggatgcagccacacactctgttcctttgctttctccagttctcagtattggcctcatttggcctggccaaagcttactgactgtccaggtctgtccccagagagcaggtgagccatgggagggagcatcttcctctgactgctctgtgcaggagcaagccaaggcacattcctgacacaggaaggtggttccagaaggcatattggcaattgtcttctcaagtcaagagggtcaccatcttggtgatggttttcagtctaggtatgcttccctgaccggaaagagaacagcttcttcataatggtcctctcctccttctcgccgttgcccatggcaaccacagcatccttctctcttcttcctcctggagtttctctgggtggaagtagaccactcctgtagcaccgcagttcctcctgcaccagcagcagctgcgacttgagcttgtttcgctcctgcagcacctccctcagctcttgcagagtgaagcggggacggttggggtctgtcagatccactaccatcttgtccggtcccaagtttacctgggctccggac

SEQ ID NO: 26 - DNA, 1418 bp, Mus musculus
taggagctaggctccaatgagcaagatccaacaactttatttttctcacacatattgtatgtcccagcaaaatctttccagtagtacaaccatcataatgtggctatattctatttttctttaagtggatgattacaatgactatacataagaaaaaacatctttcccaataccctcacatgataaaatgttttatgtattctggataagcaaaataaaaacaaattattctcaagaacccttatacatttgtaactaaccaacttgtgatattgccaaagtgtaagcaaccaagataattctctcaaaattattgacacacggcaattattttattattcatcttaaaatgtaatcttataaaaaaatcttaagagtatcacaaacttaaaccttcatataaaaatcatttttcctataaatccttagggtgcacatctacttattaacaattttttattaaatcctgtcaggaatctgagggtaagatgggtataagatcttagtcatcaccttgatcaccagcgctgcttcagccaaacaggcatacccacctcagccagtattagctgggttgccattgttcttgtccctgacatcaaaagggtctagctcaactatcaagagtgtgcctctctgaatttccaattgttccccacggttttctacttccaagccgctagcaaaagcatcttagcctaactttactacaaccgacatttccacattctttctaagagtgccggacattaccagcaatctacagctctatgtcctttctgagggtggtggttgaatcagtaatctgctccagctgcaacacttgaaaaagagcaatcactattcttgataccaatgatactgtccagtgaggggctggcagcctttttggagttttcatgcaactcacggaccaagagggatgtggtgatcttgaagacggtgagcagagcaaaactccacaacagcaagaagtcctcaggacagtcggggttctgcctcttgatgtggttgtgctaaccggtgggacaaattccatgagttctaaggctatttggttttcctcctgggaccagtcctcttcctttctttttatttctttttctcatacacatggcgccgaaaaccaggaccagtcatgttttctttctttctttctctttttcattcagccaagatgaaggattttgcccagaaccactccgggtccccagaaaatggcggcagaggtagtttgcagagtcatcaactgggggcaggctcctagaagaaacttccatgccgatgtttcaatgtttcgttttggtaacacaaactaacaggcagccacgaaaactcttgaccaaactcaatgcagtaaagagtgcatgttatacagggctacgaatatgaagaggcctccccccccagtgccgaat

SEQ ID NO: 27 - DNA, 741 bp, Mus musculus
aattcggcactgagggaagaaatctctgtaataccttttcttccaagctagacacctcacatcaggagtgcctatctgggccaggattggtctacttctgcctcttcagtgttctttcccaagcccacctagtcaggcgccgcaggggctgcctcctactgtaccctgctctgtggcccagcagaagaacactgtcaccatcacttacgaagtaggaatcacagcgcagagtgaccacatgaccaacagcagcagaaatgaatgaatctgaacagcatccctgaatcttcacaaaaacatactccaatccctaattgtctgcatctggcaacctcagggtcagaaccggggatttaagagaaaatgctaagagctcacacccatttcgcagcacttgggaggcagatgcagggggattaccatgagttcgaagccagtctgaactacatcgtttgttttatggtagtctgtgtaaaacagaatacttacgccaccattcacacttcctatcttactttctaaataatggttttaagagaaacacagtgtttgctttactctgaaagcgtgtttttattaattggacacacacaggtgaatgtttgatcccggggctgtaactgtatttgtgtcagtgtccatggagaggtgatgcctgggaggtcttgactttcctcctagttgtcttctgctttctctgcacagttgtcatatatctacctaaatataatcacaaatgta

SEQ ID NO: 28 - DNA, 4129 bp, Mus musculus
aatatataaaattatgtaaaagctatactatattttgatttagattttcctgctgtttgttaccaaaaaatttgtattttaaatttgtttagctttagtatggttttgtctacaatgagtatgtaattcttccttattaaatgaaaataagggagcgaattttgagatgtgattttaatgctcccctatggattcagtagatcaggccattctctgctgaagcctgtctgaactagaattcatccaaatttatctctaccaagagtggggttatttgataggttcgtttaggaaaaaaatttgcccctttgttttttttcttgagtcttttaagtaggaggctttaaaaaaaattgagcatttttttaagatcagaaataatgggcgtttctcctgaaagcatgattttctccctctgtgaaacggcagaacattccagtgcttttatttctttccttctctcacgcgtgctttaaaaaaaagaaaatgccttaaatcatttattttgcttatgccttcttgtaaatatgcaaaggaaggatacgaattttaaggcccaggtctacggttcgaaagaacacacccagtaaatgtactataatgttgttaaagaccaaaaagcacttcaaagggagttgatagctcagaactgttcttggtacgaggtggtcattgccctgtgagcatgcgtggtctgatgtcagttttaaagcaatgaaggaataaattgaaaaaaatcgctgctgagtagatctgtgcactaaaggggacatttaaatggaagcaccctcccgatcccggtccagaccggatgtacacaacggtctgtctgcaaccggatgtgaaactggaaggaccccacgcaccctcccagggtaccgcccagccctctcatgtgtgattctgaatttaagtttccagaagcctactggctttttgtctcgttgagattcttagatatcgattgtcttccaggctgcttgagaatgggcaaggactagttaaatggattcaccatcagccactgggtcagattcagcggtgtgatctggaaaatgagcggcaaaggttccacgtagaggatcaagaagtgaagtgacatggacaagcagatggagcccacgagccagatattctcccaggggggcatcctcagcaaagactggttttcagacaagctgttgagggcattacacatctctatggttactagaacagaaagtgccattgtcattggatatggggactcaaagattgcacaatccactccatcgaagtctgggttgtcctccttacactgtaggaaatgactcagctggtagaaggagactcttggaccgccgtcagcagcgatgaaccaccatgcagcagcacccacggtggcagcgccaacataacagccaatagccaggtaacggaaaaagagccacccgctgatcagtggttcttttgggttccgggggggtttgttcatgatgtccaggtctggaggattgaaccccagcgcagtggcaggcagaccatctgtcaccagattgacccagagtaactggacaggaattaaagcctcaggaaacccaagggctgccgtcaggaagatacagaccacttcccccacgttggatgagatgaggtagcggatgaactgcttcatgttgttgtagatggctcgcccctcctcaacagcagccacaatggtggagaagttgtcatctgccaggaccatctcagaagcagtcttagccactgcagtccctgagcccatggcaatcccgatttcagatttcttcagagcaggagcatcattcacaccatcaccagtcatagctgtgatctcatcaaaggactgaaggaactcaacaatcttagacttgtgggaaggttcaactcgagcaaaacagcgggcatttaagcaggcatctctctgggctgaagggcttaattcatcaaactctcgccctgtaaaagcctttgatgtcacatcctcatcctgcccaaagatgccaatgcggcgacagatggccacagcggtgcctttgttgtctccagtgatcatgatgacccggatgcctgcttgccggcacagcttcacagaagaggctacttcaatcctgggaggatccagcatgcccacacagccgacgaaagtcaggttggtctcgtatttgatgaagttagcagagtcttccaggtgcatctcctctctcttcagtgggttgtcatgagtggccagagccaggcaccgtagcgtgtcgctgccactgccccactcccgaatgacagacataatcttctgtttgacaccaggagtcatggggaccttggtacttccaactcggatgtgggtgcacctatcgatgacaccttctggagcccccttcacaaacatcttgctcatggatgtccggcttggcttgtttggggtacaatagacggacattgattttctatcccgtgaaaactccagagtgaactccttcttcatcagctgctttatgaccgagttgcaggcgtttgcacgctctattttagaaagccccttcagctcagtatcaaatacattcatcttctccaccaggcacgtgagagcagtctcggtagcttctccaactttctcatacacaccctttgcctcattataatccaaagcagagtcattacacagagcacagatggtggctaactctacaagcccgtcatactgatggcacttcactggcttatcatccttttgcacttctccaattggtgcatatgtggatccagttatgctgaactcattaagggaacaagtgtcaccttctactttgtccagaatgaacatcctgcacacggacatctggtttgtggtaagtgtgcctgtcttatctgagcagataacagaagtacaaccaagggtctccacagaaggcagacttcgaacgatagcatttttctttgccatcctacgagttccaagagctaagcaggtggtgatgacagcaggcagaccctcagggattgcggcaacagccagggccacggcaatcttaaagtagtagatggcacccctgatccaggagccaccatgaactgggtcattgaaatgcccaatgttgatgatccagactgcaatgcaaatgagggagataactttggaaagctgctccccaaactcgtctagcttctggtgtaggggtgttctctcctgttctgttgccaccatttcatcccggatcttgccgatctcagtattaactccagttgccaccaccactcccatagctttcccagcagcaatgtttgtaccagaaaagagcatgttctttttgtcttgattaacagctcgggggtcagggacagggtcagtatgcttgatgacggagacagattcacctgtaagaattgactggtcgactcttagagttgtagacttgatggatgtcaatctaatatcagcaggaactttgtcaccaacagcaatttccactatatcaccaggaactatgtctttagctttaattcgttgcacactctttctgtcctgtcgatacactttgcccatttcaggctcatattccttaagagcttctattgcattttcagcatttctttcctgccacacacccacgattgcattggctaccaagataagcagaattacaaacggctctacaaaggctgtaatcgtttcttccccttcctcgaaccaagccaaaacgaaagatatacatgctgccagcagtaaaattctaactagtaagtcttcaaactgctcaatcacaagttccagcaaggtttttccttcttcagccggcaattcgttggagccccatctctccttgagcttcttgacctgctccaagctcagccccgtgctctcgttgaccccgaagtggcccagcacctcctccacggtctttgtgtgagcgttctccatggctgcggggccccggccggcctcgcctcgcgtccccgcggcctcctcgcctaccgcctcgcactccggccgcgggctcgtgccaccgcgggcgcccgggcgcggacagctgtcccctcctttttcttctcctcctcctcctccgcggcggcggcggccgcttccgcctgaccggggcgctgaatcacccgagccccctcccccagaaag

SEQ ID NO: 29 - DNA, 454 bp, Mus musculus
tttttagagttgctctcaactgtttattgctaagctgtcatcatataagccgtataaaaatactttacatatagcaaaaataaactgcaggaaacaggagattaaaatcgttttgcataggaatgtgatatatcccgactcctcaggtagtggggtggagaacttcccaaaccccggccttcagagcccgatgagcaggctaccatgagggctgagaaatgacagggggacggggatgtctgatgtctgggatgctctcacagaatgctggggttcctactcacatagcaacagcactgtctgcacccagtccatctaaagcctgagagggcagagccaggcagggccaggcagggcagggacgtttattcccatctgaaagcattaacacttttgactccagttcccaggacttcattgtgatctcagaggggtcctgctcagggaagcacca

SEQ ID NO: 30 - DNA, 2439 bp, Mus musculus
tgctggtgggatcaaagcgcagtgtcctgcggcggggagcttggaacgctaagaaaagtgaccatggagaacaacaaaacctcagtggattcaaaatccattaataattttgaagtaaagaccatacatgggagcaagtcagtggactctgggatctatctggacagtagttacaaaatggattatcctgaaatgggcatatgcataataattaataataagaacttccataagagcactggaatgtcatctcgctctggtacggatgtggacgcagccaacctcagagagacattcatgggcctgaaataccaagtcaggaataaaaatgatcttactcgtgaagacattttggaattaatggatagtgtttctaaggaagatcatagcaaaaggagcagctttgtgtgtgtgattctaagccatggtgatgaaggggtcatttatgggacaaatgggcctgttgaactgaaaaagttgactagcttcttcagaggcgactactgccggagtctgactggaaagccgaaactcttcatcattcaggcctgccggggtacggagctggactgtggcattgagacagacagtgggactgatgaggagatggcttgccagaagataccggtggaggctgacttcctgtatgcttactctacagcacctggttactattcctggagaaattcaaaggacgggtcgtggttcatccagtccctttgcagcatgctgaagctgtacgcgcacaagctagaatttatgcacattctcactcgcgttaacaggaaggtggcaacggaattcgagtccttctccctggactccactttccacgcaaagaaacagatcccgtgtattgtgtccatgctcacgaaagaactgtacttttatcactagaggaatgattgggggtgggggggggcgtgtttctgttttgttatgccaaatgagaaagctgtcagggagactctcatttaaatctaatctgacggtcctcctggtctttgtacgctaccactgcctagcaatgcagccagccacagtgcagctacctcaacttcgacatcaggtagttgaaatgaaatttaatttaataaggagcaagtaactgtcaatgatggtactatcatcctagatgaaattacaaagttgcccttttataattagcaagatttggcgatactatgaattttgaagtcattttgaagcagtacagcttttccactgatgattttatactccccactcatgttaagaatgttgttctagttttcgttaaacgtagaaacaataatgtcaaatgataatgtcttagaacttgaatccatgagcagagtcaaaggctggaacccttgttttggacctgatttatggaagtgaagagttggaccaccatagcatgcattatagctactggttttgtgacagttgtccacaacacgtatttgttattttaaaaaaaaaaaatcatgttttattactggaacaaaaataacttgagtatttatgtgagaggaaaaccgtgtgcgaacttggttgactcattgaaggcaaggtgctaaaatccatcagcatttgtttcttacttatccttatacaaatcagctcggtcttacagaccagcaaaatatctcaagagctgttatgtaacagcctcaaatggttgattattagaatgaagtacaggaattctataggaatatactataataccagttgtcaaacagagcttaagaaaaggagagatgaaatctactgtagttggagagaatgtggtttgagccagagcagagacttggcagagcatgggcacatcttcagaaaaggcctgccagctggactccccacccctcaccccaccccccttgtgagttctggtttgtgtggatgatgtccaagttaccacgtgggaaagtgaaccagatcacaaacttctgcaaaaaggctttcccagagggctgagagtctgaagaaagttcactggactccatgtccagagtctgtagcatgctgacttttctgttgacgtttacaaagaaaggaaaaataatggtgatatttatactgtgctagaaacgaaagggccagggcacacaggacttggaaagcatccagcaatagccctgcagcccatgtggagaagctgcgggctgcagtccatcctcagggagactgtgctctgctcctggcctccttatgggggtgctgagtcactgctggctctcgcagcatttctcctaagcttccttgttgcacaaagaaaggaaggaactcacattgtaacggatagattcttgtttagactggctttgttgttaacgctttctaagtgaatgcagcatgtgatggtattttaaagtgtgttcccatgtgacaattttataaaagtttgttaccttgcaattttatttcaaaacataaattcagatttaaaattag

SEQ ID NO: 31 - DNA, 1090 bp, Mus musculus
ggaagtcgaccagcacaaggcagagtcctggacagaacaaaacctgcctgctacaggacagagcttgtgtttgaatgcatgtacatataggcatatatttatgtatagatatccaaggacagaggtggcaatgagcagtctgtgcccaccaggggccttccttccatgttagcaataaccagtatccaccttgagagtgcacctcagagtccagaaccggcttcccaccatcatggtctttccttccatcagggtctacctctggacaggcaggtggccttttcttgcctcacgcagtacctgagcactccagagctgtgctcaaatgcccatcagctaccaaatggcaaaatctgaaagtggttgtaaataaccattacagaatgagtgtagtatatttgttcaattataagattattctttcacagaagccttatagctctctgcttcatctaagaaaacaattaccaaaaaaaacaacgtttctaactgcaatctgtgaactgtgcgttttcagattggttactggtaacagataagctggtgtctgctctgtgtaattagctgcttacttcagttactagcagtgacctattatttcttataaccaaaaaaagcatggtttaattaaaacatgtttaatgatcgtgccttaggagttaatgcccccttatggaacacgcctgaattgcacctgtggctggaagttttaagttactcccagacagatggactcatgacaggaaaagctctctcacaggaagatgcatctttaaaatttttgtcagtctgtatgatggtggcttacctttcccaacgcacagaaagaaacaactgtctgaaagcatactgaatgatttcgcacgactgtgaagagctggcgcgaactgccttgtacacacatagctcctggccgcctgcaggctgcctcccgcctgcctctcgtctgtaccccatgtttattagcatcatggagttgcatgaaccattcttagtagactgtcatctgaaagcaagcgtctgatatttgtgtcagctatctttgtagttaggagatgaatccaataaagcagtatttttttcttttttt

SEQ ID NO: 32 - DNA, 968 bp, Mus musculus
tttgggggtcactcgacttgattaattttattctacaaaatgctactcagtggaagtaggaaagctaacaaaacaacaacaacaaaaacataaaacgagaacaaacccgagggaaaataagtttttaatatgttctttcctccatagcagcaagctcctaccaagcttttcttagtgcaaatcctgtaggcttgtgtcacatacagtacacagaacaacacatcacaccaccacagatgcttccgagcagagatactcctcaaaaatttaaaactatacaaagatttctgagcataaggtcctgcctggagagttcaactagagcgaccctcctagggccgtttcaccgttaatttaaaagtaggggacaaaaggtgcccagaaaggaaattaaattccccgcggagccataaaaccttgtacaacccatttgcctccaggatctaatagcaaatttcactccacgtcattgacatataccaaatacagatgcatgaagcttgggtcctactctataccaaaatacgatatatacacctcccactgcaaaaggaatctgatacctagtctttatacaaagctgaatattttcttcctcaaaatcaagtaaccacaaagtaaaataaatggaatatttttttaaaaaaaaaatcatacagagaaagttaagttttgagagacagggccagggtctttcattgtcttctcttacaatgtagatttctcagtagccactgtccccacaggaatgacaattgatctttaaaaacagagcctttttaatacagtttatacagcacaagtccacaagtcacttgagaaacaaacaaaatagagatcattatcctaagtcagaacaagtgggggagagagagccagagaaaggagtgggaaggaagtactttaatgctatctgtttctattcaggcttggaacaacacaaagaaatgtacttctgtcgtcttctc

SEQ ID NO: 33 - DNA, 668 bp, Mus musculus
ggaagcccaagatggaccaaccactgctgtgggctgatggccttagcagccacataatcctccgtgtgatgtccagggtcagtgcagtcctggcccaccagggctaggagaaggtatatgagaatctcactgtgatctgtcctctttagaacagcatttcagtcaccacctgggccgatgctctgtccatgtgttccttcaccagctcaaagcacctgtgggctagctgctgcagcctggcgtcctcactcaccatcagctccccatacaggtatatgcccatggcctggtcctccagtaggaacctctccacctgattgtatgcctttgtgtacttgtgcttaataaccagttcacaccatcgatggcgaacctctgcatcctgctctgggagatggtaagtctgctggagacagtgcagtgtctgagggctcagcgtcttctgctctaagagccactccaaaagcaagacgatctggtctggagaaagcttttcaaaggcgacttctcgcttccctcgtttgcgttttcggggtctgcggttgactcgaatccatttcgccacctcggagcctacttgccgtgcgagctactcggcgagcagtcctcggccttgcgctcctcctgcagcgcctgtggggagaacaagtcaagttcaccgagcccag

SEQ ID NO: 34 - DNA, 932 bp, Mus musculus
agcccagatcctagcagtagtcacacttcaaagtccagccatttctatgagataaaaggacacaccacagatggagaactttagaagaagtttgactagaaaaatcattggctgtgtagcgtgaatttttatcagtgaagaaatatacctggattctttcatagtggctatattaactggctaaaagttttgtcatttagtccattaataggctaaaatgattttttaaaatgaatccacaaaactgtcatttaaaacactacatatgtggcaacccaaaattagtgttcatcagcctaagggcacagggaagatactggccataattttaattcataagaataaattttaacttttaaggccaaaaagcaaaataattaaaatattttgagttgtttaaaacaaatctagatcataactggaaaaagaagtcatcactattattttttaacccaaataaaaatatgtgatctaacaataagacaggaaactaagacatcacagtgatatattgtggaaaggtgagtgatcagtcacattcagatttctgcatgttgaaatacaacaaccagctgactgtatccctctctccactagttatggcattgtgtaggcaggggctagggatgctatctacagatatgaagaactccttaaaaatccttcttccggacactcaaaatacaatggaaactttggtccagacaatctggaccagaaggtgaattcgtaacttccttatcccattaaatattgtaaaagagacagaataagaaatgtttctctttggaaatacgtattttgaatgcaactatgaaagataatggtgcataattatttcctttcaacttaatgcttaatacagaagtaaaagtctacctgccttgctgaattgaaaagaacaaatccattatatagccatatttctccat

(19) United States
(12) Patent Application Publication
(10) Pub. No.: US 2003/0064380 A1
(43) Pub. Date: Apr. 3, 2003

(54) Anergy-regulated molecules

(75) Inventors: Anjana Rao, Cambridge, MA (US); Michael Byrne, Brookline, MA (US); Fernando Macian, Quincy, MA (US)

Correspondence Address: FITZPATRICK CELLA HARPER & SCINTO, 30 ROCKEFELLER PLAZA, NEW YORK, NY, 10112, US

(73) Assignee: The Center For Blood Research, Inc., Boston, MA

(21) Appl. No.: 10/058,024

(22) Filed: Jan. 29, 2002

Related U.S. Application Data
(60) Provisional application No. 60/264,876, filed on Jan. 29, 2001.

(51) Int. Cl. C12Q 1/68; G01N 33/53; G01N 33/543

(52) U.S. Cl. 435/6; 435/7.1; 436/518

(57) ABSTRACT

Methods and compositions for the identification of novel targets for diagnosis, prognosis, therapeutic intervention and prevention of an immune disorder. In particular, the present invention is directed to the identification of novel targets which are anergy markers. The present invention is further directed to methods of high-throughput screening for test compounds capable of modulating the activity of proteins encoded by the novel targets. Moreover, the present invention is also directed to methods that can be used to assess the efficacy of test compounds and therapies for the ability to inhibit an immune disorder. Methods for determining the long term prognosis in a subject are also provided.

This application claims benefit of U.S. Provisional Patent Application No. 60/264,876, filed Jan. 29, 2001.

This invention was made with Government support under NIH Grants CA42471 and AI48213. The Government has certain rights in this invention.

### BACKGROUND OF THE INVENTION

1. Field of the Invention

The present invention is directed to novel methods of diagnosis, treatment and prognosis of immune disorders using differentially expressed polynucleotides. The present invention is further directed to novel therapeutics and therapeutic targets and to methods of screening and assessing test compounds for the treatment and prevention of immune disorders. In particular, the present invention is directed to a method of modulating the expression levels of anergy polynucleotides associated with immune disorders.

2. Related Background Art

One of the salient features of the normal immune system is its ability to mount responses against foreign antigens while not attacking self antigens. This discrimination is imposed largely during development in the thymus where many autoreactive T cells are triggered to undergo apoptosis in a process known as clonal deletion; cells that survive this process are rendered tolerant to self antigens in the periphery. There are at least two mechanisms for inducing tolerance outside the thymus in the periphery. The first mechanism is anergy induction, an intracellular process in which antigen receptors become uncoupled from their downstream signaling pathways. The second mechanism involves regulatory T cells which limit the responses of other lymphocytes to self and environmental antigens, in part by producing immunosuppressive cytokines such as TGFβ and IL-10.

Activation of the cell-intrinsic mechanism of lymphocyte tolerance is closely linked to the cell surface stimulus received. In both T and B cells, combined activation of antigen and costimulatory receptors leads to full activation of all signaling pathways and culminates in a productive immune response. Costimulation is necessary for a productive response to antigen. In T cells, a predominant costimulatory receptor is CD28, which binds the costimulatory ligands B7-1 (CD80) and B7-2 (CD86) expressed on the surface of antigen-presenting cells (APC). Combined engagement of TCR and CD28 results in full activation of a number of signaling pathways that ultimately lead to IL-2 production and cell proliferation.

In contrast, tolerance is evoked, both ex vivo and in vivo, by unbalanced stimulation through antigen receptors without engagement of costimulatory receptors or by stimulation with weak agonist antigens in the presence of full costimulation. In each system, the process of tolerance induction may be conceptualized as occurring in two stages. The tolerising stimulus first elicits partial or suboptimal activation; next, the partially-activated lymphocytes enter a long-lasting unresponsive state, in which they paradoxically become refractory to subsequent full stimulation with antigen and costimulatory ligands.

TCR engagement in the absence of costimulation results in a partial response. The incompletely stimulated T cells enter a long-lived unresponsive state, known as tolerance or anergy. Critically, once tolerance is induced, the anergic T cell is blocked from the response evoked by exposure to an antigen presented by an APC. In such cells, the combined engagement of the T cell receptor (TCR) and CD28 does not trigger the level of IL-2 production and the extent of proliferation that occurs in fully activated T cells.

The most consistent feature of tolerising stimuli is their ability to induce elevation of intracellular free calcium. One of the simplest methods of inducing tolerance (anergy) in T cells is treatment with the calcium ionophore ionomycin; conversely, anergy induction is blocked by the extracellular calcium chelator EGTA and by the calcineurin inhibitor cyclosporin A (“CsA”). Calcium has also been implicated in a well-established model of B cell tolerance in vivo. B cells bearing an anti-hen egg lysozyme (HEL) Ig transgene, that have been tolerised to circulating antigen in vivo, show a small but significant elevation in their basal levels of intracellular free calcium, and a concomitant increase in resting nuclear levels of the calcium-regulated transcription factor NFAT. Lastly, calcium is implicated in anergisation by weak agonist antigens (altered peptide ligands), which dissociate more rapidly than agonist peptide-MHC complexes from the T cell receptor. Measurements of calcium transients in single cells show that these weak agonist peptides elicit much lower levels of calcium mobilization than full agonist peptides, but increased calcium levels are maintained for much longer times.

A major consequence of calcium mobilization is activation of the transcription factor NFAT. NFAT is a family of highly-phosphorylated proteins residing in the cytoplasm of resting cells; when cells are activated, these proteins are dephosphorylated by the calcium/calmodulin-dependent phosphatase calcineurin, translocate to the nucleus, and become transcriptionally active. In the nucleus, they cooperate with an unrelated transcription factor, AP-1 (Fos-Jun), to induce a large number of cytokine genes and other genes that are central to the productive immune response. Notably, NFAT activation does not require strong stimulation of antigen receptors on B and T cells: substantial nuclear localization of NFAT can be achieved with low, sustained levels of calcium mobilization, such as those achieved by low concentrations of calcium ionophores, self-antigens, and low-affinity peptide-MHC complexes. Costimulatory receptors are not coupled to calcium mobilization, and so contribute weakly, if at all, to activation of NFAT. Thus NFAT activation occurs in response to calcium signals or TCR stimulation alone, the precise conditions needed to evoke anergy. In contrast, costimulation is critical for optimal activation of NFκB and AP-1: combined TCR/CD28 stimulation activates cJun kinase (JNK), p38 MAP kinase and IκB kinase (IKK) pathways, and increases nuclear levels of NFκB/Rel and AP-1 proteins, more strongly than TCR stimulation alone.

This invention demonstrates that NFAT plays a central role in tolerance induction in T lymphocytes. Using a non-complex biochemical method of inducing anergy in T cells, it is shown that anergy induction is associated with expression of a novel set of anergy polynucleotides, distinct from those characteristic of the productive immune response. Among these are polynucleotides encoding caspase-3 and putative E3 ligases; and the data suggest that proteolytic mechanisms contribute to the long-lived anergic state. T cells lacking a major NFAT protein, NFAT1 (NFATp, NFATc2) are resistant to anergy induction, and show significantly lower expression of most anergy genes. Conversely, T cells expressing constitutively active NFAT1, engineered so as to be incapable of cooperation with AP-1, basally express caspase-3 and other anergy polynucleotides, and display an anergic phenotype of lowered TCR responsiveness. Thus depending on the signaling pathways and transcriptional partners available, a single transcription factor regulates two contrasting aspects of cellular behavior: in the absence of AP-1, NFAT mediates a genetic program of anergy in lymphocytes that opposes the program of productive activation mediated by the cooperative NFAT:AP-1 complex.

Understanding the molecular mechanisms of immunological response is critical for medical intervention in numerous conditions. Although regulation of autoreactivity normally focuses immune cell surveillance on foreign antigens, such as those expressed on pathogenic cells and organisms, in many disorders this regulation is impaired, and the immune system attacks the body's own tissues or elicits a hyperactive assault on a nonpathogenic antigen as in allergic reactions. In transplant medicine, the body is frequently subject to non-self antigens on the donor tissue. There is also considerable evidence that tumors can induce immune tolerance by functional inactivation of T cells that may mount a tumor-specific response.

### SUMMARY OF THE INVENTION

It is believed that imbalanced activation of the T cell receptor-activated transcription factor NFAT relative to the activation of other transcription factors also induced during the complete immune response, e.g., a CD28-activated transcription factor, such as AP-1 (e.g., Fos/Jun, Jun/Jun dimers) and NFκB/Rel, promotes or induces anergy or tolerance. The complete set of these transcription factors that are turned on during a productive immune response may hereafter be referred to as “productive transcription factors.” Because these transcription factors may also interact physically (e.g. AP-1) or functionally (e.g. NFκB/Rel) with NFAT, they may sometimes be referred to hereafter as “NFAT ligands.” The invention is based, in part, on the discovery that the expression of a set of nucleic acids is altered or modulated when immune cells are in such an anergic state, e.g., when the cells are treated with a compound that induces NFAT signaling, (e.g., a calcium ionophore such as ionomycin, or an anti-CD3 antibody) compared to fully stimulated immune cells, e.g., cells treated with compounds that induce NFAT-NFAT ligand signaling (e.g., a calcium ionophore such as ionomycin and a signaling activator such as the phorbol ester, phorbol 12-myristate 13-acetate (PMA), or cells treated with an antigen presenting cell and an antigen). These modulated nucleic acids are herein referred to as “anergy markers” or “anergy nucleic acids,” examples of which are listed in Group I, Group II, Group III, and Group IV. Among the nucleic acids turned on under these conditions, there are some whose products have a negative feedback effect on the production of an immune response, e.g., these gene products may uncouple an antigen receptor from the proximal signaling pathways. The anergy markers described herein are useful indicators of the anergic state of an immune cell, as well as candidate targets for identifying novel modulators of an immune response.

In one aspect, the invention features a method of evaluating or identifying an agent, e.g., a test compound, for its ability to interact with an anergy marker listed in Group I or Group II or Group III or Group IV, or a polypeptide encoded by an anergy marker listed in Group I or Group II or Group III or Group IV. The interaction can be (1) a physical interaction, e.g., binding, e.g., with a dissociation constant of less than 1 mM, 100 nM, 10 nM, 1 nM, or 0.1 nM, and/or (2) an interaction that alters the activity or expression of the marker polynucleotide or polypeptide (e.g., with or without binding the polypeptide). The method includes contacting a test compound and the anergy marker polynucleotide, or the polypeptide or a fragment thereof, e.g., under conditions that allow an interaction between the marker or the polypeptide and the test compound to occur; and determining whether the test compound interacts with (e.g., binds to or alters the activity or expression of the marker, or polypeptide or fragment thereof. Binding to the marker or polypeptide or a change, e.g., a decrease or increase, in the level of activity or expression of the marker or polypeptide can identify the test compound as a useful agent for altering an immune response.

In one embodiment, the anergy marker polynucleotide includes, or the polypeptide is encoded by, an anergy marker listed in Group I. In another embodiment, the anergy marker polynucleotide includes, or the polypeptide is encoded by, an anergy marker listed in Group II. In another embodiment, the anergy marker polynucleotide includes, or the polypeptide is encoded by, an anergy marker listed in Group III. In another embodiment, the anergy marker polynucleotide includes, or the polypeptide is encoded by, an anergy marker listed in Group IV. In another preferred embodiment, the anergy marker encodes a nucleotide binding protein, or a regulator of a nucleotide binding protein. Preferably, the anergy marker encoding the nucleotide binding protein, or the regulator of a nucleotide binding protein includes an anergy marker selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176. In still another preferred embodiment, the anergy marker includes, or the polypeptide is encoded by, an anergy marker selected from the group consisting of Mm. 116802, Hs. 248037, Mm. 10085, and Hs. 96149. In yet another preferred embodiment, the anergy marker includes, or the polypeptide is encoded by, one or more of the following: Z31202_s_at, aa144045_s_at, aa174748_at, c81206_rc_at, D86609_{—s}_at, ET63436_at k00083_s_at, MIP1-B_at, Msa.11439.0_s_at, Msa.15983.0_f_at, Msa.1669.0_f_at Msa.18713.0_g_at, U44731_s_at, x12531_s_at, and x67914_s_at. In yet another preferred embodiment, the anergy marker includes, or the polypeptide is encoded by, an anergy marker selected from the group consisting of GRG4, jumonji, RPTPσ, PTP-1B, RPTPκ, GBP-3, Rab10, caspase-3, SOCS-2, Traf5, DAGKα, LDHAα, phosphoglycerate mutase, CD98, 4-IBB-L, and FasL. In another preferred embodiment, the anergy marker encodes a protease, e.g., a caspase (e.g., caspase-3). In still another preferred embodiment, the anergy marker encodes a G protein, e.g., a guanylate binding protein, e.g., GBP-3.

In preferred embodiments, the test compound is a nucleic acid (e.g., an antisense nucleic acid or ribozyme), a polypeptide (e.g., an antibody or an antigen-binding fragment thereof), a peptide fragment, a peptidomimetic, or a small molecule (e.g., a small organic molecule with molecular weight less than about 2000 or 800 Daltons). In preferred embodiments, the test compound is a member of a combinatorial library, e.g., a peptide or organic combinatorial library, or a natural product library. In one preferred embodiment, a plurality of test compounds, e.g., library members, is tested. The plurality of test compounds, e.g., library members, can include at least 10, 10², 10³, 10⁴, 10⁵, 10⁶, 10⁷, or 10⁸ compounds. In a preferred embodiment, test compounds of the plurality, e.g., library members, share a structural or functional characteristic.

In a preferred embodiment, the method is performed in cell-free conditions (e.g., a reconstituted system or a binding assay with purified components, e.g., an NMR binding assay).

In a preferred embodiment, the method further includes contacting the test compound with a test cell, or a test animal, to evaluate the effect of the test compound on immune cell (e.g., T cell) function or an immune response (e.g., a normal or abnormal immune response). The test cell can be an immune cell, e.g., a T cell or a B cell or cell line. The test animal can be a transgenic animal. The test animal can have an autoimmune disorder. In still another embodiment, the method further includes obtaining a nucleic acid from the test cell and determining an expression profile for the test cell as described below.

In another embodiment, the contacting step between the test compound and the polypeptide occurs within a cell, e.g., a recombinant cell. For example, the test compound can be administered to a yeast cell that includes the polypeptide as a fusion protein in a two-hybrid assay. The ability of the test compound to alter an activity of the polypeptide can be assayed by the polypeptide function in the two-hybrid assay with a polypeptide ligand.

In another aspect, the invention features a method of evaluating or identifying a test compound, for the ability to modulate, e.g. increase or decrease, transcription of an anergy marker listed in Group I or Group II or Group III or Group IV. The method includes contacting a cell (e.g., an immune cell, e.g., a T cell or a B cell or cell line) with a test compound and determining whether the test compound modulates, e.g., activates or represses, transcription of the marker, wherein a change, e.g., an increase or decrease, in the level of expression of the marker is indicative of an alteration in marker expression, e.g., activation or repression of marker expression.

In a preferred embodiment, the anergy marker is listed in Group I. In another preferred embodiment, the anergy marker is listed in Group II. In another preferred embodiment, the anergy marker is listed in Group III. In another preferred embodiment, the anergy marker is listed in Group IV. In a preferred embodiment, the level of expression of more than one anergy marker listed in Group I or Group II or Group III or Group IV is determined. In another preferred embodiment, the anergy marker encodes a nucleotide binding protein, or a regulator of a nucleotide binding protein. Preferably, the anergy marker encoding the nucleotide binding protein, or the regulator of a nucleotide binding protein includes an anergy marker selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176. In still another preferred embodiment, the anergy marker is selected from the group consisting of Mm. 116802, Hs. 248037, Mm. 10085 and Hs. 96149. In another preferred embodiment, the anergy marker encodes a protease, e.g., a caspase (e.g., caspase-3). In still another preferred embodiment, the anergy marker encodes a G protein, e.g., a guanylate binding protein, e.g., GBP-3.

In a preferred embodiment, the level of expression of the anergy marker is evaluated after full stimulation of the cell, e.g., the immune cell, for example, after stimulating the cell with an antigen, an antigen presenting cell (APC), activators of NFAT-NFAT ligand signaling (e.g., ionomycin and PMA), a combination of anti-CD3 and anti-CD28 antibodies, and/or a combination of anti-TCR and anti-CD28 antibodies. In another preferred embodiment, the level of expression of the anergy marker is evaluated after stimulation of the cell with an activator of NFAT, e.g., ionomycin, e.g., before, during or after contact with the test compound.

The test compound can be a nucleic acid (e.g., an antisense, ribozyme), a polypeptide (e.g., an antibody or an antigen-binding fragment thereof) a peptide fragment, a peptidomimetic, or a small molecule (e.g., a small organic molecule with molecular weight less than about 2000 or 800 Daltons). In preferred embodiments, the test compound is a member of a combinatorial library, e.g., a peptide or organic combinatorial library, or a natural product library. In one preferred embodiment, a plurality of test compounds, e.g., library members, is tested. The plurality of test compounds, e.g., library members, can include at least 10, 10², 10³, 10⁴, 10⁵, 10⁶, 10⁷, or 10⁸ compounds. In a preferred embodiment, test compounds of the plurality, e.g., library members, share a structural or functional characteristic.

In a preferred embodiment, the ability of the test compound to alter transcription of the anergy marker is evaluated in a cell-based system, e.g., using a reporter nucleic acid operably linked to a regulatory region (e.g., the promoter) of the anergy marker.

In another preferred embodiment, the ability of the test compound to alter transcription of the anergy marker is evaluated in a second system, e.g., a cell-free, cell-based, or an animal system. In still another preferred embodiment, the method further includes contacting the test compound with a test cell, or a test animal, to evaluate the effect of the test compound on the transcription of the anergy marker.

Also within the scope of the invention are test compounds identified using the methods described herein. The invention features a composition, e.g., a pharmaceutical composition, which includes a test compound as identified and/or described herein, and a pharmaceutically acceptable carrier. In one embodiment, the compositions of the invention, e.g., the pharmaceutical compositions, are formulated for combination therapy, or are administered in combination therapy, i.e., combined with other test compounds, e.g., therapeutic agents, that are useful for treating disorders, such as, for example, cancers, immune cell mediated disorders, or infections.

In another aspect the invention features a method of modulating (e.g., increasing or decreasing) anergy in a cell (e.g., an immune cell), or tolerance in a subject. The method includes contacting a cell, or administering to a subject, a test compound (e.g., a test compound as identified and/or described herein) in an amount sufficient to modulate (e.g., increase or decrease) the activity or expression of one or more anergy markers listed in Group I or Group II or Group III or Group IV, thereby modulating tolerance in said subject.

In a preferred embodiment, the test compound increases the expression or activity of, one or more anergy markers listed in Group I or Group II or Group III or Group IV, or one or more polypeptides encoded by the aforesaid markers (e.g., a nucleotide binding protein, regulator of a nucleotide binding protein, or a protease (e.g., a caspase, e.g., caspase-3) or a G protein, e.g., a guanylate binding protein, e.g., GBP-3.).

In a preferred embodiment, the test compound decreases the expression or activity of, one or more anergy markers listed in Group I or Group II or Group III or Group IV, or one or more polypeptides encoded by the aforesaid markers (e.g., a nucleotide binding protein, regulator of a nucleotide binding protein, or a protease (e.g., a caspase, e.g., caspase-3) or a G protein, e.g., GDP Dissociation Inhibitor Beta).

In a preferred embodiment, the cell is an immune cell, e.g., a T cell. The cell can be in a subject as part of a therapeutic or prophylactic protocol. In another preferred embodiment, the cell, e.g., the immune cell, is removed from the subject prior to contacting the cell with the test compound, e.g., prior to introducing the anergy marker. The method can further include returning the immune cell to the subject.

In a preferred embodiment, the test compound induces, or promotes anergy or tolerance, in a subject, thereby inhibiting, or reducing, an unwanted or detrimental immune response in the subject.

In a preferred embodiment, the subject is a human, e.g., a patient suffering from an unwanted immune response, e.g., an autoimmune disorder (including, for example, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), allergy (e.g., atopic allergy), asthma (e.g., extrinsic or intrinsic asthma), a reaction to a xeno- or allo-antigen, graft-vs.-host disease, and transplant rejection.

In a preferred embodiment, the test compound blocks or reduces tolerance, e.g., ongoing tolerance, or the initiation of tolerance, in a subject, thereby enhancing the subject's immunity. Such test compounds may be useful in treating or preventing, in a subject, a cancer (e.g., a tumor, a soft tissue tumor, or a metastatic lesion), or a pathogenic infection, e.g., a viral, bacterial, or parasitic infection.

In a preferred embodiment, the subject is a human, e.g., a cancer patient, or a subject in need of heightened immune surveillance, e.g., a patient suffering from an autoimmune disorder or a subject suffering from a pathogenic infection, e.g., a viral (e.g., HIV), bacterial, or parasitic infection.

In a preferred embodiment, the test compound is a small molecule (e.g., a chemical agent having a molecular weight of less than 2500 Da, preferably, less than 1500 Da), a chemical, e.g., a small organic molecule, e.g., a product of a combinatorial library, a polypeptide (e.g., an antibody such as an intrabody), a peptide, a peptide fragment, a peptidomimetic, an antisense, a ribozyme, or an anergy marker listed in Group I or Group II or Group III or Group IV, or a fragment thereof.

The test compounds described herein can be administered by themselves, or in combination with at least one or more test compounds. In one embodiment, a combination of test compound(s) that modulate the activity or expression of one or more of the anergy markers listed in Groups I or Group II can be co-administered. In other embodiments, a modulator of a costimulatory receptor or its ligands (e.g., CD28/B7 or CD19/ligand) can be administered in combination with one or more of the test compounds described herein. In those embodiments where tolerance is increased, an inhibitor of a costimulatory pathway (e.g., at least one blocker, e.g., an inhibitor of the CD40 ligand-CD40 interaction (e.g., an anti-CD40L antibody), an inhibitor of the CD28-B7, or the CTLA4-B7 interaction (e.g., a soluble CTLA4, e.g., a CTLA4 fusion protein, e.g., a CTLA4 immunoglobulin fusion, e.g., CTLA4/Ig), or any combination thereof) can be co-administered. These costimulatory receptor modulators can be administered prior to, simultaneously with, or after the administration of one or more of the test compounds described herein.

The test compounds described herein may also be administered in combination therapy, i.e., combined with other test compounds, e.g., therapeutic agents, that are useful for treating disorders, such as cancers, immune cell mediated disorders, or infections.

In a preferred embodiment, the test compound is an anergy marker listed in Group I or Group II or Group III or Group IV. Preferably, the anergy marker is introduced into a cell, e.g., an immune cell, under conditions that allow the marker to be expressed, e.g., 2, 4, 6, 8, 10 or more fold greater than the expression level of its endogenous counterpart in the cell prior to introducing the anergy marker. In a preferred embodiment, the anergy marker is operably linked to an inducible promoter, e.g., a promoter that can be regulated by a small molecule, e.g., an organic molecule of molecular weight about 2000 Daltons or less. The anergy marker can be introduced using a vector as described below. The vector can be delivered to a cell by a membrane bound structure (e.g., a liposome) or a virus (e.g., a retrovirus, herpes virus, or adenovirus).

In another preferred embodiment, the test compound is a nucleic acid which regulates the expression of an endogenous anergy marker listed in Group I or Group II or Group III or Group IV. The introduced nucleic acid can be an inducible promoter, e.g., a promoter that can be regulated by a small molecule, e.g., an organic molecule of molecular weight about 2000 Daltons or less. For example, the introduced nucleic acid can recombine with a genomic sequence in order to regulate the endogenous marker.

In another aspect, the invention features an array. The array includes a substrate having a plurality of addresses. Each address of the plurality includes a capture probe, e.g., a unique capture probe. Preferably, an address has a single species of capture probe, e.g., each address recognizes a single species (e.g., a nucleic acid or polypeptide species). The addresses can be disposed on the substrate in a two-dimensional or three-dimensional configuration.

In a preferred embodiment, at least one address of the plurality includes a capture probe that hybridizes specifically to an anergy marker listed in Group I or Group II or Group III or Group IV. In one embodiment, the marker is listed in Group I. In another embodiment, the marker is listed in Group II. In one embodiment, the marker is listed in Group III. In one embodiment, the marker is listed in Group IV. In a preferred embodiment, the plurality of addresses includes addresses having nucleic acid capture probes for all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the plurality of addresses includes addresses having nucleic acid capture probes for all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the plurality of addresses includes addresses having nucleic acid capture probes for all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the plurality of addresses includes addresses having nucleic acid capture probes for all the markers listed in Group IV (i.e., 100% of the markers) or a fraction of the markers listed in Group IV, e.g. at least 20%, 40%, 50%, 60%, 80%, or 90% of the markers listed in Group IV. Preferably, the array has no more than 4000, 3000, 2000, 1000, 500, or 250 addresses.

In another preferred embodiment, at least one address of the plurality includes a capture probe that binds specifically to a polypeptide selected from the group of polypeptides encoded by the markers listed in Group I or Group II or Group III or Group IV. Preferably, the capture probe is an antibody or derivative thereof. In a preferred embodiment, the plurality of addresses includes addresses having polypeptide capture probes for all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the plurality of addresses includes addresses having polypeptide capture probes for all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the plurality of addresses includes addresses having nucleic acid capture probes for all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the plurality of addresses includes addresses having nucleic acid capture probes for all the markers listed in Group IV (i.e., 100% of the markers) or a fraction of the markers listed in Group IV, e.g. at least 20%, 40%, 50%, 60%, 80%, or 90% of the markers listed in Group IV. Preferably, the array has no more than 4000, 3000, 2000, 1000, 500, or 250 addresses.

In another aspect, the invention features a first method of evaluating a sample. The method includes determining the expression of at least one anergy marker listed in Group I or Group II or Group III or Group IV and comparing the expression to a reference to thereby evaluate the sample. In a preferred embodiment, the expression is determined as a value and compared to a reference value to thereby evaluate the sample. A change in the expression, e.g., the expression value, relative to the reference, e.g., the reference value, is an indication that the sample differs from a sample used to obtain the reference, e.g., the reference value. The expression, e.g., the expression value, can be a qualitative or quantitative assessment of the abundance of 1) an mRNA transcribed from the anergy marker, or of 2) the polypeptide encoded by the anergy marker.

In a preferred embodiment, the reference value is obtained by determining a value for the expression of the nucleic acid in a normal sample, a diseased sample, an anergic immune cell (e.g., T or B cell) population, or an immune cell (e.g., T or B cell) population treated with a calcium ionophore (e.g., ionomycin) and/or a phorbol ester, or a population treated with an anti-CD3 antibody or an APC and an antigen, or a combination of anti-CD3 and anti-CD28 antibodies, or a combination of anti-TCR and anti-CD28 antibodies.

In a preferred embodiment, the expression, e.g., a value for expression, can be determined by quantitative PCR, Northern blotting analysis, microarray analysis, serial analysis of nucleic acid expression, and other routine methods.

In a preferred embodiment, the anergy marker is listed in Group I. Most preferably, multiple expression values are determined, e.g., from all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the marker is listed in Group II. Most preferably, multiple expression values are determined, e.g., from all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the marker is listed in Group III. Most preferably, multiple expression values are determined, e.g., from all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the marker is listed in Group IV. Most preferably, multiple expression values are determined, e.g., from all the markers listed in Group IV (i.e., 100% of the markers) or a fraction of the markers listed in Group IV, e.g. at least 20%, 40%, 50%, 60%, 80%, or 90% of the markers listed in Group IV.

In another aspect, the invention features a second method of evaluating a sample. The method includes providing a sample expression profile and at least one reference expression profile; and comparing the sample expression profile to at least one reference expression profile to thereby evaluate the sample.

In a preferred embodiment, an expression profile includes a plurality of values, wherein each value corresponds to the level of expression of a different anergy marker, splice-variant or allelic variant of an anergy marker or a translation product thereof. The value can be a qualitative or quantitative assessment of the level of expression of the marker or the translation product of the marker, i.e., an assessment of the abundance of 1) an mRNA transcribed from the marker, or of 2) the polypeptide encoded by the marker.

In a preferred embodiment, the sample expression profile and the reference profile have a plurality of values, one or more of which correspond to an anergy marker listed in Group I or Group II or Group III or Group IV.

In a preferred embodiment, the profiles include values for all the anergy markers listed in Group I (i.e., 100% of the markers) or a fraction of the anergy markers listed in Group I, e.g., at least 20%, 40%, 50%, 60%, 80%, or 90% of the markers listed in Group I. In another preferred embodiment, the profiles include values for all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profiles include values for all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the profiles include values for all the markers listed in Group IV (i.e., 100% of the markers) or a fraction of the markers listed in Group IV, e.g. at least 20%, 40%, 50%, 60%, 80%, or 90% of the markers listed in Group IV.

In a preferred embodiment, a plurality of reference profiles is provided. A reference profile can be a profile obtained from a normal sample, a diseased sample, an anergic immune cell (e.g., T or B cell) or cell population, or an immune cell (e.g., T or B cell) population treated with a calcium ionophore (e.g., ionomycin) and/or a phorbol ester. A reference profile can also be an expression profile obtained from any suitable standard, e.g., a mixture of anergy markers.

In one preferred embodiment, the sample expression profile is compared to a reference profile to produce a difference profile. In a preferred embodiment, the sample expression profile is compared indirectly to the reference profile. For example, the sample expression profile is compared in multi-dimensional space to a cluster of reference profiles.

In a preferred embodiment, the sample expression profile is obtained from an array. For example, the method further includes providing an array as described above; contacting the array with a nucleic acid mixture (e.g., a mixture of nucleic acids obtained or amplified from a cell), and detecting binding of the nucleic acid mixture to the array to produce a sample expression profile. In another embodiment, the sample expression profile is determined using a method and/or apparatus that does not require an array (e.g., SAGE or quantitative PCR with multiple primers).

The method can further include harvesting mRNA from the sample and reverse transcribing the mRNA to produce cDNA, e.g., labeled or unlabelled cDNA. Optionally, the cDNA can be amplified, e.g., by a thermal cycling (e.g., polymerase chain reaction (PCR)) or an isothermal reaction (e.g., NASBA) to produce amplified nucleic acid for use as the nucleic acid mixture that is contacted to the array.

In one embodiment, the sample is a blood sample, a spleen sample, a lung sample, or a lymph sample. Preferably the sample includes immune cells (e.g., T cells or B cells). In a preferred embodiment, the method further includes fluorescent-activated cell sorting (FACS) of the sample prior to harvesting mRNA. For example, FACS can be used to isolate a subtype of T cells, e.g., Th1 cells, T cells with a particular T cell receptor, T cells of various stages of maturation, helper T cells, killer T cells, and the like. The sample can be obtained from a patient, e.g., a patient with an immunological disorder, a transplant patient, or an immuno-compromised patient.

Also featured is a method of evaluating a subject. The method includes providing a sample from the subject and determining a sample expression profile, wherein the profile includes one or more values representing the level of expression of one or more anergy markers listed in Group I or Group II or Group III or Group IV. In a preferred embodiment, the profile includes multiple values for the level of expression of markers listed in Group I, e.g., all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the profile includes multiple values for the level of expression of markers listed in Group II, e.g., all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profile includes multiple values for the level of expression of markers listed in Group III, e.g., all the markers listed in Group III (i.e., 100% of the markers) or a fraction of the markers listed in Group III, e.g. at least 20%, 40%, 50%, 60%, 80%, or 90% of the markers listed in Group III.

The method can further include comparing the value or the profile (i.e., multiple values) to a reference value or a reference profile.

An alteration in the expression of one or more nucleic acids of the profile is an indication that the subject has or is disposed to having an immune disorder, e.g., anergy or an immuno-compromised disorder. Preferably, expression of a plurality of anergy markers of the profile (e.g., at least about 5%, 10%, 15%, 20%, 40%, 50%, 60%, 70%, 80%, or 90%) is altered.

The method can be used to a) diagnose an immune disorder in a subject; b) monitor an infection, e.g., a viral, bacterial, fungal, or parasitic infection in a subject; c) monitor immunosuppression therapy in a subject (e.g., prior to, during, or following transplantation, or administration of cyclosporin A and FK506); d) monitor a treatment for an immune disorder (e.g., T cell anergy or T cell hyperstimulation) in a subject; and e) monitor a cancer or proliferative disorder. Non-limiting examples of immune disorders include myocardial hypertrophy, allergy, arthritis, and autoimmune disease.

The subject expression profile can be determined in a subject during treatment. The subject expression profile can be compared to a reference profile or to a profile obtained from the subject prior to treatment or prior to onset of the immune disorder. In a preferred embodiment, the subject expression profile is determined at intervals (e.g., regular intervals) during treatment.

The treatment can be an immuno-suppressive treatment, e.g., a treatment that inhibits calcineurin activity e.g., treatment with cyclosporin A or FK506. The treatment can be with a specific NFAT inhibitor.

In another aspect, the invention features a transactional method of evaluating a subject. The method includes: a) obtaining a sample from a caregiver; b) determining a subject expression profile for the sample; and c) transmitting a result to the caregiver.

Optionally, the method further includes either or both of steps: d) comparing the subject expression profile to one or more reference expression profiles; and e) selecting the reference profile most similar to the subject reference profile. The reference expression profiles can include one or more of: i) a profile from a like sample from a normal subject; ii) a profile from a like sample from a subject having a disease or disorder (e.g., a T cell disorder, an autoimmune disease, an immune-compromised state); iii) a profile from a like sample from a subject having a disease or disorder and undergoing a treatment; and iv) a profile from the subject being evaluated, e.g., an earlier profile or a normal profile of the same subject.

The result transmitted to the caregiver can be one or more of: information about the subject expression profile, e.g., raw or processed expression profile data and/or a graphical representation of the profile; a difference expression profile obtained by comparing the subject expression profile to a reference profile; a descriptor of the most similar reference profile; the most similar reference profile; and a diagnosis or treatment associated with the most similar reference profile. The result can be transmitted across a computer network, e.g., the result can be in the form of a computer transmission (e.g., across the Internet or a private network, e.g., a virtual private network). The result can be transmitted across a telecommunications network, e.g., using a telephone or mobile phone. The results can compressed and/or encrypted.

The expression profiles can be determined, e.g., using an array (e.g., a nucleic acid or polypeptide array) as described herein or using a method and/or apparatus that does not require an array (e.g., SAGE or quantitative PCR with multiple primers).

In a preferred embodiment, the subject expression profile and the reference profiles include one or more values representing the level of expression of one or more anergy markers listed in Group I or Group II or Group III or Group IV. In a preferred embodiment, the profiles include multiple values for the level of expression of markers listed in Group I, e.g., all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the profiles include multiple values for the level of expression of markers listed in Group II, e.g., all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profiles include multiple values for the level of expression of markers listed in Group III, e.g., all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the profiles include multiple values for the level of expression of markers listed in Group IV, e.g., all the markers listed in Group IV (i.e., 100% of the markers) or a fraction of the markers listed in Group IV, e.g. at least 20%, 40%, 50%, 60%, 80%, or 90% of the markers listed in Group IV.

In the context of expression profiles herein, “most similar” refers to a profile, which for more than one value of the profile, compares favorably to a given profile. A variety of routine statistical measures can be used to compare two reference profiles. One possible metric is the length (i.e. Euclidean distance) of a difference vector representing the difference between the two profiles. Each of the subject and reference profile is represented as a multi-dimensional vector, wherein the coordinate of each dimension is a value in the profile. The distance of the difference vector is calculated using standard vectorial mathematics. In another embodiment, values for different nucleic acids in the profile are weighted for comparison.

Also featured is a computer medium having encoded thereon computer-readable instructions to effect the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile and the reference profiles include one or more values representing the level of expression of one or more anergy markers listed in Group I or Group II or Group III or Group IV. In a preferred embodiment, the profiles include multiple values for the level of expression of markers listed in Group I, e.g., all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the profiles include multiple values for the level of expression of markers listed in Group II, e.g., all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profiles include multiple values for the level of expression of markers listed in Group III, e.g., all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the profiles include multiple values for the level of expression of markers listed in Group IV, e.g., all the markers listed in Group IV (i.e., 100% of the markers) or a fraction of the markers listed in Group IV, e.g. at least 20%, 40%, 50%, 60%, 80%, or 90% of the markers listed in Group IV. Preferably, the profiles include additional values for markers that are not members of any Group.

In a preferred embodiment, the instructions further include instructions to create a graphical user interface that can display a sample expression profile and/or a reference profile. For example, a subset of or all values of the profile can be depicted as a graphic having a color dependent on the magnitude of the value. The graphical user interface can also allow the user to select a reference profile from a plurality of reference profiles, and can depict a comparison between the sample expression profile and the selected reference profile.

In another embodiment, the computer medium can further include, e.g., have encoded thereon, data records for one or more reference profiles.

In another aspect, the invention features a computer medium having a plurality of digitally encoded data records. Each data record includes values representing the level of expression of one or more anergy markers listed in Group I or Group II or Group III or Group IV in a sample, and a descriptor of the sample.

In a preferred embodiment, the profiles include multiple values for the level of expression of anergy markers listed in Group I, e.g., all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the profiles include multiple values for the level of expression of markers listed in Group II, e.g., all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profiles include multiple values for the level of expression of markers listed in Group III, e.g., all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the profiles include multiple values for the level of expression of markers listed in Group IV, e.g., all the markers listed in Group IV (i.e., 100% of the markers) or a fraction of the markers listed in Group IV, e.g. at least 20%, 40%, 50%, 60%, 80%, or 90% of the markers listed in Group IV.

The descriptor of the sample can be an identifier of the sample, a subject from which the sample was derived (e.g., a patient), a diagnosis (e.g., a T cell disorder, an immunodeficiency, an autoimmune disease or an infection), or a treatment (e.g., a preferred treatment, an immunosuppressant). In a preferred embodiment, the records include records for one or more samples from a normal individual, an abnormal individual (e.g., an individual having a disease or disorder), and in vitro culture T cells. The abnormal individual can be an immune-compromised individual (e.g., an AIDS patient, an individual treated with an immunosuppressant (e.g., FK506, cyclosporin A)), an individual having an infection (e.g., viral, bacterial, fungal, or parasitic infection), an individual exposed to a superantigen, an individual having an autoimmune disease, or an individual having a proliferative disorder (e.g., cancer). In vitro cultured T cells can include T cells exposed in vitro to a drug (e.g., cyclosporin A or FK506), an antigen presenting cell, a cytokine, or a virus.

In one embodiment, the data record further includes a value representing the level of expression for each nucleic acid detected by a capture probe on an array described herein.

In another aspect, the invention features a second method of evaluating or identifying an agent, e.g., a test compound that alters an immune cell activity (e.g., a compound that induces anergy or a compound that stimulates immune cells to exit anergy). The method includes: providing one or more reference profiles; contacting the test compound to an immune (e.g., a T or B) cell; determining a test compound-associated expression profile, e.g., using a method described herein; and comparing the test compound-associated expression profile to at least one reference profile.

The test compound-associated expression profile and the reference profiles include the subject expression profile and the reference profiles include one or more values representing the level of expression of one or more anergy markers listed in Group I or Group II or Group III or Group IV. In a preferred embodiment, the profiles include multiple values for the level of expression of markers listed in Group I, e.g., all the markers listed in Group I (i.e., 100% of the markers) or a fraction of the markers listed in Group I, e.g., at least 20%, 40%, 50%, 60%, 80%, or 90% of the markers listed in Group I. In another preferred embodiment, the profiles include multiple values for the level of expression of markers listed in Group II, e.g., all the markers listed in Group II (i.e., 100% of the markers) or a fraction of the markers listed in Group II, e.g. at least 20%, 40%, 50%, 60%, 80%, or 90% of the markers listed in Group II. In another preferred embodiment, the profiles include multiple values for the level of expression of markers listed in Group III, e.g., all the markers listed in Group III (i.e., 100% of the markers) or a fraction of the markers listed in Group III, e.g. at least 20%, 40%, 50%, 60%, 80%, or 90% of the markers listed in Group III. In another preferred embodiment, the profiles include multiple values for the level of expression of markers listed in Group IV, e.g., all the markers listed in Group IV (i.e., 100% of the markers) or a fraction of the markers listed in Group IV, e.g. at least 20%, 40%, 50%, 60%, 80%, or 90% of the markers listed in Group IV.

In one embodiment, the reference profiles include one or more of a profile of an immune cell (e.g., a T cell) in an anergic state, a profile of an immune cell (e.g., a T cell) in a normal state, and a profile of an immune cell (e.g., a T cell) in an activated state. In a preferred embodiment, the contacted immune cell is in an anergic state. For example, prior, during, or after the immune cell is contacted with the test compound, the immune cell can be contacted with cyclosporin A or FK506.

In another preferred embodiment, the method further includes, e.g., prior to determining the expression profile, contacting an immune cell with an antigen and/or an antigen presenting cell, e.g., to stimulate the immune cell with antigen. The compound-associated expression profile can be determined at periodic intervals after contact with the antigen. In still another preferred embodiment, the method further includes, e.g., prior to determining the expression profile, contacting an immune cell with a compound which emulates costimulation, e.g., PMA or a combination of an antibody which crosslinks or engages TCR and CD28 or a combination of an antibody which crosslinks or engages CD3 and CD28.

In another preferred embodiment, the contacted immune cell is in a normal state. In still another preferred embodiment, the contacted immune cell is in an activated state (e.g., activated by a phorbol ester, a cytokine, or an antigen presenting cell).

In a preferred embodiment, the method includes comparing the agent expression profile to a plurality of reference profiles (e.g., all reference profiles), and identifying a most similar reference profile as an indication of the efficacy and/or utility of the agent. In another preferred embodiment, multiple test compound-associated expression profiles are determined at periodic intervals after contact with the agent.

In another aspect, the invention features an isolated or purified marker polynucleotide, and the purified protein product of a marker discovered by a method described herein. Such markers or marker proteins can be used to alter the state of an immune cell, in addition to providing screens for molecules that can alter immune responses.

In another embodiment, the invention provides a method of screening for test compounds capable of modulating the activity of an anergy marker protein encoded by a an anergy marker listed in Group I or Group II or Group III or Group IV. The method includes contacting the anergy marker protein with a plurality of test compounds; detecting binding of one of the test compounds to the anergy marker protein, relative to other test compounds; and correlating the amount of binding of the test compound to the anergy marker protein with the ability of the test compound to modulate the activity of the anergy marker protein, wherein binding indicates that the test compound is capable of modulating the activity of the anergy marker protein. In a preferred embodiment, the method of screening is high-throughput screening.

In another preferred embodiment, the test compound is from a library selected from a group of libraries of spatially addressable parallel solid phase or solution phase libraries or synthetic libraries made from deconvolution, ‘one-bead one-compound’ methods and/or by affinity chromatography selection. In still another preferred embodiment, the selected test compound prevents binding of the anergy marker protein with a bioactive agent selected from the group of naturally-occurring compounds, biomolecules, proteins, peptides, oligopeptides, polysaccharides, nucleotides and/or polynucleotides. In still another preferred embodiment, the test compound is a bioactive agent selected from the group of naturally-occurring compounds, biomolecules, proteins, peptides, oligopeptides, polysaccharides, nucleotides and/or polynucleotides. In yet another preferred embodiment, the test compound is a small molecule.

In another preferred embodiment, the anergy marker is one or more of the following: Msa.21745.0_s_at (also Mm. 21985), Hs. 129764, U44731_s_at (also Mm. 1909), Hs. 240849, Msa.1669.0_f_at (also Mm. 19123), or GenBank PID:g2853176. In another preferred embodiment, the anergy marker is one or more of the following: Mm. 116802, Hs. 248037, Mm. 10085 or Hs. 96149. In yet another preferred embodiment, the anergy marker is one or more of the following: Z31202_s_at, aa144045_s_at aa174748_at, c81206_rc_at, D86609_s_at, ET63436_at, k00083_s_at, MIP1-B_at, Msa.11439.0_s_at, Msa.15983.0_f_at, Msa.1669.0_f_at Msa.18713.0 _g_at, U44731_s_at, x12531_s_at, or x67914_s_at. In still another preferred embodiment, the anergy marker is one or more of the following: GRG4, jumonji, RPTPσ, PTP-1B, RPTPκ, GBP-3, Rab10, SOCS-2, Traf5, DAGKα, LDHAα, phosphoglycerate mutase, CD98, 4-IBB-L, or FasL. In yet another preferred embodiment, the anergy marker is GBP-3.

In another embodiment, the present invention provides a method of screening for test compounds capable of modulating the level of expression of an anergy marker. The method includes the steps of comparing a level of expression of an anergy marker listed in Group I or Group II or Group III or Group IV in a first sample of cells prior to providing a test compound to the first sample of cells; and a level of expression of the same anergy marker in a second sample of cells after providing the test compound to the second sample of cells, wherein a substantially modulated level of expression of the anergy marker in the second sample, relative to the first sample, is an indication that the test compound is capable of modulating the level of expression.

In a preferred embodiment, the test compound is from a library selected from a group of libraries of spatially addressable parallel solid phase or solution phase libraries or synthetic libraries made from deconvolution, ‘one-bead one-compound’ methods and/or by affinity chromatography selection.

In a preferred embodiment, the cell is an immune cell. In another preferred embodiment, the method further includes the step of stimulating the cells prior to providing the test compound. In another preferred embodiment, the step of stimulating the cells includes contacting the cells with a stimulant, such as, for example, an antigen, an antigen presenting cell, an activator of NFAT-NFAT ligand signaling, a combination of anti-CD3 and anti-CD28 antibodies, and/or a combination of anti-TCR and anti-CD28 antibodies. In a preferred embodiment, the activator of NFAT-NFAT ligand signaling is ionomycin and/or PMA.

In another preferred embodiment, the anergy marker is one or more of the following: Msa.21745.0_s_at (also Mm. 21985), Hs. 129764, U44731_s_at (also Mm. 1909), Hs. 240849, Msa.1669.0_f_at (also Mm. 19123), or GenBank PID:g2853176. In another preferred embodiment, the anergy marker is one or more of the following: Mm. 116802, Hs. 248037, Mm. 10085 or Hs. 96149. In yet another preferred embodiment, the anergy marker is one or more of the following: Z31202_S_at, aa144045_s_at, aa174748_at, c81206_rc_at, D86609_s_at, ET63436_at, k00083_s_at, MIP1-B_at, Msa.11439.0_s_at, Msa.15983.0_f_at Msa.1669.0_f_at, Msa.18713.0_g_at, U44731_s_at, x12531_s_at, or x67914_s_at. Instill another preferred embodiment, the anergy marker is one or more of the following: GRG4, jumonji, RPTPσ, PTP-1B, RPTPκ, GBP-3, Rab10, SOCS-2, Traf5, DAGKα, LDHAα, phosphoglycerate mutase, CD98, 4-IBB-L, or FasL. In yet another preferred embodiment, the anergy marker is GBP-3.

In one embodiment, the present invention provides a method of screening for test compounds capable of inhibiting an immune disorder. The method includes contacting a panel of anergy marker proteins with a plurality of test compounds, wherein the panel of anergy marker proteins comprise at least 2 anergy marker proteins encoded by anergy markers listed in Group I or Group II or Group III or Group IV; detecting binding of one of the test compounds to the panel of anergy marker proteins, relative to other test compounds; and correlating the amount of binding of the test compound to the panel of anergy marker proteins with the ability of the test compound to inhibit an immune disorder, wherein binding indicates that the test compound is capable of inhibiting an immune disorder.

In a preferred embodiment, the method of screening is high-throughput screening. In another preferred embodiment, the test compound is selected from a library of spatially addressable parallel solid phase or solution phase libraries or synthetic libraries made from deconvolution, ‘one-bead one-compound’ methods and/or by affinity chromatography selection. In another preferred embodiment, the selected test compound prevents binding of the anergy marker protein with a bioactive agent selected from naturally-occurring compounds, biomolecules, proteins, peptides, oligopeptides, polysaccharides, nucleotides and/or polynucleotides. In still another preferred embodiment, the test compound is a bioactive agent selected from naturally-occurring compounds, biomolecules, proteins, peptides, oligopeptides, polysaccharides, nucleotides and/or polynucleotides. In still another preferred embodiment, the test compound is a small molecule.

In another preferred embodiment, the anergy marker is one or more of the following: Msa.21745.0_s_at (also Mm. 21985), Hs. 129764, U44731_s_at (also Mm. 1909), Hs. 240849, Msa.1669.0_f_at (also Mm. 19123), or GenBank PID:g2853176. In another preferred embodiment, the anergy marker is one or more of the following: Mm. 116802, Hs. 248037, Mm. 10085 or Hs. 96149. In yet another preferred embodiment, the anergy marker is one or more of the following: Z31202_s_at, aa144045_s_at, aa174748_at, c81206_rc_at, D86609_s_at, ET63436_at, k00083_s_at, MIP1-B_at Msa.11439.0_s_at, Msa.15983.0_f_at, Msa.1669.0_f_at, Msa.18713.0_g_at, U44731_s_at, x12531_s_at, or x67914_s_at. In still another preferred embodiment, the anergy marker is one or more of the following: GRG4, jumonji, RPTPσ, PTP-1B, RPTPκ, GBP-3, Rab10, SOCS-2, Traf5, DAGKα, LDHAα, phosphoglycerate mutase, CD98, 4-IBB-L, or FasL. In yet another preferred embodiment, the anergy marker is GBP-3.

In another preferred embodiment, the immune disorder is selected from the group of T cell disorders, B cell disorders, autoimmune disorders, infectious disorders, proliferative disorders, transplant rejection and/or cancer. In still another preferred embodiment, the immune disorder is diabetes mellitus, rheumatoid arthritis, juvenile rheumatoid arthritis, osteoarhritis, psoriatic arthritis, multiple sclerosis, encephalomyelitis, myasthenia gravis, systemic lupus erythematosis, autoimmune thyroiditis, atopic dermatitis eczematous dermatitis, psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, graft-versus-host disease, and/or allergy. In another preferred embodiment, the immune disorder is diabetes mellitus, rheumatoid arthritis, multiple sclerosis, Crohn's disease, asthma, allergic asthma, graft-versus-host disease and/or allergy.

In a preferred embodiment, the cancer is lung cancer, breast cancer, lymphoid cancer, gastrointestinal cancer, genitourinary tract cancer, pharynx cancer, colon cancer, renal-cell carcinoma, prostate cancer, testicular cancer, non-small cell carcinoma of the lung, cancer of the small intestine, cancer of the esophagus, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and/or retinoblastoma. In another preferred embodiment, the cancer is renal cancer, melanoma, breast cancer, lymphoma, and multiple myeloma.

In another embodiment, the present invention provides a method of screening test compounds for inhibitors of an immune disorder in a subject. The method includes the steps of obtaining a sample comprising cells; contacting an aliquot of the sample with one of a plurality of test compounds; comparing a level of expression of an anergy marker listed in Group I or Group II or Group III or Group IV; and selecting one of the test compounds which substantially modulates the level of expression of the anergy marker in the aliquot containing that test compound, relative to other test compounds.

In a preferred embodiment, the test compound is from a library selected from a group of libraries of spatially addressable parallel solid phase or solution phase libraries or synthetic libraries made from deconvolution, ‘one-bead one-compound’ methods and/or by affinity chromatography selection.

In another preferred embodiment, the anergy marker is one or more of the following: Msa.21745.0_s_at (also Mm. 21985), Hs. 129764, U44731_s_at (also Mm. 1909), Hs. 240849, Msa.1669.0_f_at (also Mm. 19123), or GenBank PID:g2853176. In another preferred embodiment, the anergy marker is one or more of the following: Mm. 116802, Hs. 248037, Mm. 10085 or Hs. 96149. In yet another preferred embodiment, the anergy marker is one or more of the following: Z31202_s_at, aa144045_s_at, aa174748_at, c81206_rc_at, D86609_s_at, ET63436_at, k00083_s_at, MIP1-B_at, Msa.11439.0_s_at, Msa.15983.0_f_at, Msa.1669.0_f_at, Msa.18713.0_g_at, U44731_s_at, x12531_s_at, or x67914_s_at. In still another preferred embodiment, the anergy marker is one or more of the following: GRG4, jumonji, RPTPσ, PTP-1B, RPTPκ, GBP-3, Rab10, SOCS-2, Traf5, DAGKα, LDHAα, phosphoglycerate mutase, CD98, 4-IBB-L, or FasL. In yet another preferred embodiment, the anergy marker is GBP-3.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 includes graphs of nucleic acid expression data for nucleic acids induced by ionomycin alone, ionomycin with CsA or ionomycin with PMA.

FIGS. 2A and 2B include graphs of nucleic acid expression data for nucleic acids induced by ionomycin alone, ionomycin with CsA or ionomycin with PMA.

FIG. 3 includes graphs of the induction of caspase-3.

FIG. 4 is a schematic of a method for obtaining a sample profile and a reference profile from microarrays.

FIG. 5 is a schematic of a network for a transactional method of evaluating a sample.

FIG. 6: Stimulation With Calcium lonophores Activates A Specific Program Of Gene Expression

A: RNA was prepared from D5 cells stimulated for 0, 2, 6 or 16 hours with ionomycin, PMA plus ionomycin or CsA plus ionomycin, and used to evaluate gene transcription profiles using Affymetrix oligonucleotide arrays. Genes which were modulated by at least 3-fold in response to any of the treatments were selected for clustering analysis using the Self-Organizing Map (SOM) algorithm, a method for clustering genes on the basis of kinetic expression pattern. Hours of treatment are indicated on the x-axis and normalized mRNA frequency (a log transformation of absolute frequency values, which allows clustering independent of expression magnitude) is displayed on the y-axis. The number of genes in each panel is indicated.

B: Expression profiles of 18 specific genes chosen on the basis of their strong activation by ionomycin. The genes are grouped into six categories based on function. The numbers within the panels indicate the fold induction of each transcript after stimulation of D5 T cells with ionomycin for 2 hours, as confirmed by real time quantitative RT-PCR. n.d., not determined.

FIG. 7: NFAT1−/− Th1 Cells Show Reduced Expression Of Anergy-Associated Genes

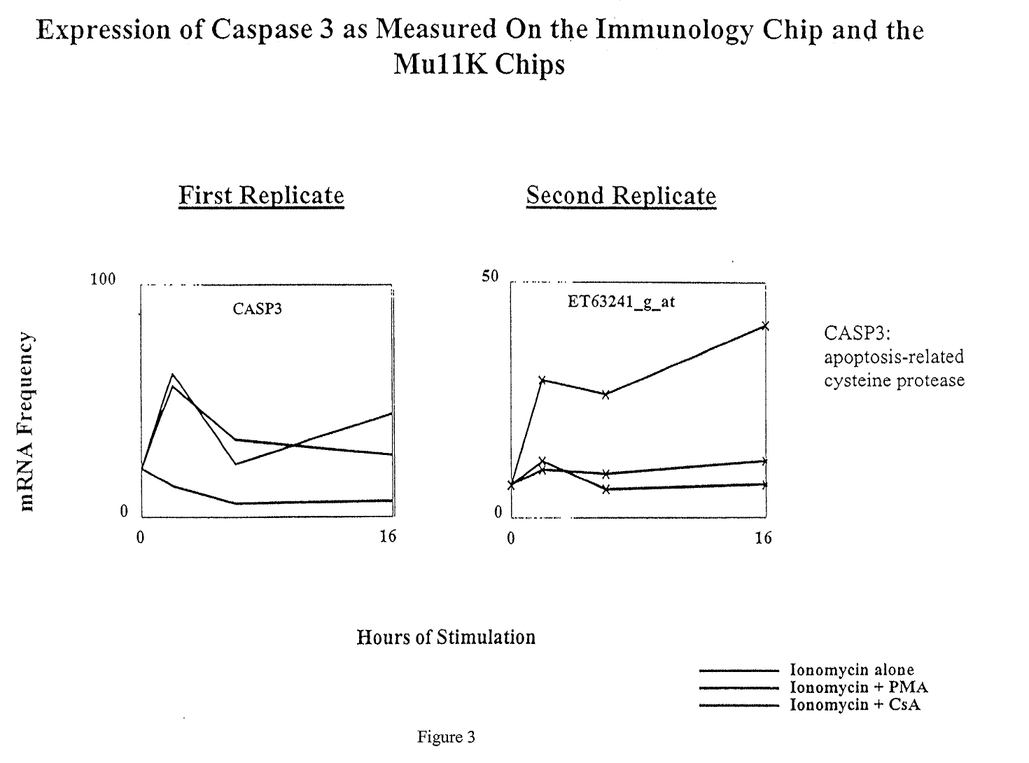

A: Expression of 15 of the ionomycin-induced genes shown in FIG. 3B was examined by real-time quantitative RT-PCR in wild type and NFAT1−/− Th1 cells. CD4+ cells were isolated from wildtype and NFAT1−/−DO 11.10 transgenic mice, differentiated under Th1 conditions for 1 week, and left unstimulated or stimulated with ionomycin for 2 or 6 hours. For each cell type and stimulation condition, results are represented as fold increase over the levels of mRNA present in resting cells (set to 1).

B: Gene transcription profiles of selected genes in wild type and NFAT1−/− Th1 cells, in response to stimulation with ionomycin, PMA/ionomycin or CsA/ionomycin. Panels show mRNA frequencies obtained using Affymetrix oligonucleotide arrays.

FIG. 8: A Model For Anergy Induction

### DETAILED DESCRIPTION OF THE INVENTION

The inventors have discovered that the physiological state of immune cells, characterized by desensitization and resistance to co-receptor engagement (as described below) is manifest in the expression pattern of numerous nucleic acids. These nucleic acids can be used as a molecular fingerprint indicative of this state. In addition, many of these nucleic acids can be effectors of the molecular processes characteristic of this state.

The tolerant state of the cell requires the function of the calcium calcineurin-dependent transcription factor NFAT. NFAT is activated by calcium mobilization via the T cell receptor (TCR). Tolerance is likely the result of imbalanced activation of NFAT, relative to the CD28-activated transcription factors AP-1 (Fos/Jun, Jun/Jun) and NFκB/Rel. Whereas NFAT can be activated by TCR engagement alone, cJun, RelA and cRel require engagement of both TCR and CD28 for maximal activity. Under conditions of full stimulation through both the TCR and CD28, activation of NFAT, AP-1, NFκB/Rel and other transcription factors results in transcription of the cytokine nucleic acids and other nucleic acids associated with a productive immune response. In contrast, when T cells are stimulated through the TCR in the absence of CD28 stimulation, NFAT becomes activated without significant activation of AP-1 or NFκB. As set forth herein, imbalanced NFAT activation activates a distinct genetic program associated with the anergic or tolerant state, as exemplified by differential expression of certain polynucleotides within the cell. For example, expression of the nucleic acids of Group I or Group II or Group III or Group IV (referred to throughout as “anergy markers”) are modulated as cells enter this state.

Definitions and Terms

In order that the present invention may be more readily understood, certain terms are first defined. Additional definitions are set forth throughout.

As used herein, “Group I” includes one or more of the anergy marker polynucleotides having the following identifiers: TC14671_g_at (also Mm. 710), TC16364_at, TC16828_at TC17132_at, TC17495_at, TC17559_at, TC18221_at, TC19211_at, TC21156_at, TC23346_s_at TC23450_s_at, TC24045_at, TC24067_at, TC25965_at, TC27326_g_at, TC29889_at, TC30384_g_at, TC30935_at, TC30992_s_at, TC31681_at, TC32225_at, TC33206_at, TC33833_at, TC34186_at, TC36089_at, TC36583_at, TC37631_at, TC38094_at, TC38978_at, TC39012_at, TC39080_at, TC39762_at, TC40487_g_at, TC41014_at; murine T cell receptor V beta chain, Z31202_s_at, aa144045_s_at, aa174748_at, c81206_rc_at, D86609_s_at, ET63436_at k00083_s_at, MIP1-B_at Msa.11439.0_s_at, Msa.15983.0_f_at, Msa.1669.0_f_at, Msa.18713.0_g_at, U44731_s_at, x12531_s_at, x67914_s_at, U61363_s_at (GRG4), D31967_s_at (jumonji), D28530_s_at (RPTPσ), u24700_s_at (PTP-1B), L10106_s_at (RPTPκ), U44731_s_at (GBP-3), Msa.21745.0_s_at (Rab10), ET63241_g_at (caspase-3), U88327_s_at (SOCS-2), d78141_s_at (Traf5), Msa.26042.0_s_at (DAGKα), Msa.358.0_f_at (LDHAα), aa161799_s_at (phosphoglycerate mutase), Msa.2134.0_f_at (CD98), Msa.907.0_at (4-IBB-L), and u06948_s_at (FasL).

As used herein, “Group II” includes one or more of the anergy marker polynucleotides having the following UniGene identifiers Mm. 638, 13146, 7398, 34570, 529, 1255, 29317, 873, 19123, 42255, 21985, 1909, 1282, 5024, 100579, 18571, 8137, 8155, 5021, 2849, 34405, 2068, 29317, 142543, 716, 28251, 3189, 116802, and 10085 as identified by their UniGene cluster number (http://www.ncbi.nlm.nih.gov/).

As used herein, “Group III” includes one or more of the following human anergy marker polynucleotides Hs. 284279, 170843, 24370, 94785, 106127, 856, 75703, 274369, 151787, 1526, 129764, 240849, 158297, 22670, 75562, 3069, 74552, 196352, 169610, 224961, 37268, 1526, 248037, 74552, 240849, and 96149 as identified by their UniGene cluster number, LOC64749 as identified by its LocusLink number (http://www.ncbi.nlm.nih.gov/), GenBank PID:g2853176, and human T cell receptor V beta chain.

As used herein, “Group IV” includes one or more of the following human anergy marker polynucleotides: human GRG4, human jumonji, human RPTPσ, human PTP-1B, human RPTPκ, human GBP-3, human Rab10, human caspase-3, human SOCS-2, human Traf5, human DAGKα, human LDHAα, human phosphoglycerate mutase, human CD98, human 4-IBB-L, and human FasL.

The nucleic acid sequences of the anergy markers identified by TC identifiers are listed in Table 1.

TABLE 1

| | |
|---|---|
| TC14671_g_at | |
| tctccagtca cagagtgttg agggtgtgcc acctcccctt tgggaccacc ttgggttgcc ctcttaacaa agttggcctt | SEQ ID NO. 1 |
| accaaggagc agtcatcttg gattgtataa tttgaatgag ccaaggacca gagtgagggc agcacaaact actcagccac | |
| aatgtcttca gaggtggaga cctcggaggg ggtagatgag tcagagaaga actctatggc accagaaaag gaaaaccata | |
| ccaaaatggc agacctttct gagctcctga aggaagggac caaggaatca catgaccgag cagaaaatac ccagtttgtc | |
| aaagacttct tgaaaggcga cattaagaag gagctatttg agctggccac cactgcactt tacttcacat actcagcgct | |
| tgaggaggaa atggaccgca acgagggcca cgcagccttc gcccccttat atgtgcccac ggagcttcac cggaagcagc | |
| actggtcagg acatgaagta tttcttgtgg aaaactgggg gagccggtaa gtgc | |
| TC16364_at | |
| taaccttcat tttttgtcca tttatttaga aaaaaattaa catgagcaaa tgaaatacct cagtgttaca acagagtata | SEQ ID NO. 2 |
| gaaatgtcta gcaataatca aataatttga tctttaaata caaaataacc acatgaacac ctaatataca ggtttcatct | |
| gaatacatat ttattagata aatattagag gtcacatcat ctaactgcat acagctttgc aagactagaa atcacaatta | |
| gttttttttt tctgaccagt caaaagtatg aaatgattgc agtgtacata cgatgtacaa agacaagggc gggttctgtg | |
| gacgtcactt caggctgcac gtgtgggtgt ggatgtgtgt acgtgtgaat cacctgtgat catgatatca aaaacttata | |
| caaagtatat gaatttggtt acaattttct tctgaaatcc ccgtttctct tcattgtttc catagcaccc taaaaataca | |
| caggtggcag ggccaggaca cagaaggtaa atagtacatg taggtaaaaa taaaaacaaa agggaacaaa aacgcctctg | |
| cacacagggt cagtatatta caggagacaa ggacggagtc acgaaggcta acaaacggga tctagtattc cacgtagaat | |
| gaaggagttc caagcctttt gttgtttctc tgttttgtaa aataaaaaca atacacattc cgggagaaat gaatgtatct | |
| tgttgacatg tctatttctc atttacatat gtacacacgg cccttgagtc gctgctgctc tctgcctcgt ctggattggt | |
| caggccgagg gcccatgggg agcagacctg tagctctctg ggatttaggg cttccgttag ggagaaagtg ttaggaatct | |
| tttaaaaaat aaaatggcta caggatacgt gagacatgaa taaagcttca aaccaagaag atgagggtga tcgcctgtgc | |
| gggggcgggg cctttcccat ctgcgcatgc tctctcccag cccagccgct tagctgagtg gcggctggta cgtgcctatg | |
| ggtgggttgc tcgttgtggt aaagtggctt gctgagacct catttcggag gttactatgg ctccaagttg ttgtaagaaa | |
| ggactgagga tctttccaga gcctaggcct gcctctgttt atggatgtca cctttacctg cgtctgtcac taccaaggca | |
| tgctccagcc cccgatgtct ttgtagctct ctcaaaccct ggatcggctc caacatttct ctggaaggag acatttccgg | |
| agtgtgggct tcaggctctg tggtgaactt gctggtgggc acttgcctgg gagggagcct taggaaatc | |
| TC16828_at | |
| tctgtataag tctctcaatt tataaccata cataatagtc accaaaacac agaatgcttg ctcctctggc atatgcaaca | SEQ ID NO. 3 |
| gtagtacagc agcaagaaaa gactgtcctt gacagtaccc aatgtcttca tcgaacaccg agtaggcctt gcagattttg | |
| taaagggatt cttgaccatc gcctccagtg tctttgaagt aatcgtgtgc aggaaatgta cgatgaatat ctcgagtaat | |
| aacactctcc tgtgcagagt cctttgtgat aaggattcgg tatttatcca gcatttcctg attgtcatgg catcctgcta | |
| atagttgcca tacttctgcc cttagtgcct caggaacacc actctttacc aaggtgaata gtccttttgg tcggccacca | |
| aggttattgt gccatcttcc taacaattct ccccaagaat agagaatctt ctcaggacag tcttttgaca catcacctgt | |
| tccactcgag agttcattat cactctcctc ttctgcttca tcctcctgtg gtgacattgg gcccccagca ctagtagggg | |
| tgatgggctc ctccttgtca gattctctct gcagactcac cacttcatat attgcatctc cagcactgct gtggcctttt | |
| ccctcagact gtttcaacct catgaagaaa gtctctgtga aagtctttct gctgaaatac caaaatctct catttgcagg | |
| gtatacacga actactgtct ccaggagaaa acggactggc tccaccacct ctgtgaccac catgtccact gcaacggtca | |
| tgtacacccg tttatctttg ggcgtttctt cattaagagc caga | |
| TC17132_at | |
| aggggcgccg ggagcaggcg tgtgggactc ctgaccggag agccggaggc tgcgccttcc ccgcaccggg accttcacga | SEQ ID NO. 4 |
| cacaccagat cctagtcctt gccccgtgcg aacgcccacg atgaccacca ccctcgtgtc cgccaccatt tttgacttga | |
| gcgaagtttt atgcaagggt aacaagatgc tcaactacag cactcccagc gctgggggct gcctgctgga caggaaggca | |
| gtgggcaccc ctgctggcgg gggcttccct cgcaggcact cggtcactct gcccagctcc aagttccatc agaaccagct | |
| tctcagcagc cttaagggtg agccggcccc gtccctgagc tcacgcgaca gccgctttcg agaccgctct ttctccgaag | |
| ggggcgagcg gctgctgccc acccagaagc agcctgggag cggccaggtc aactccagcc gctacaagac ggagctgtgc | |
| cgtcccttcg aagaaaacgg tgcctgtaag tacggggaca agtgccagtt cgcgcatggc atccacgagc tccgcagcct | |
| gacccgccac cccaagtaca agacggagct gtgccgcacc ttccacacca tcggcttttg cccgtacggg ccccgctgcc | |
| acttcattca taacgccgag gagcgacgcg ccctggcggg gggccgagac ctctccgctg accgtccccg cctccagcat | |
| agctttagct ttgctgggtt tcccagtgcc gctgccaccg ccgctgccac ggggctgctg gacagcccca catccatcac | |
| cccaccccct atcctgagcg ccgatgacct cttgggctca cctactctgc ccgatggcac caataacccc ttcgcctttt | |
| ccagccagga gctggcgagc ctctttgctc ctagcatggg gctgcctggg ggaggctccc ccaccacttt cctcttccgg | |
| cccatgtccg aatcccctca catgtttgac tctcccccca gccctcagga ttctctctcg gaccacgagg gctatctgag | |
| cagctccagc tccagccaca gtggctcaga ctcccctacc ttggacaact caagacgcct gcccattttc agcagactct | |
| ccatctcaga tgactaagcc agggtaggga gggacccccc cccatgcctc cttcacctct ccaccccatc tcttccctcc | |
| acctccccac cccctaactt tccctcaaac cccacattga tacatttaag ctcagcccct ttcccagaac cttggtatgt | |
| taccctcccc ccacataagg acaagtcaat ttgttggtag cttctggctt gaaaccctct ccctccattt catagccact | |
| taaccacgca taacagagtt ccatcttttt gtcagtagat agcctttttt tacccacccc ccccccggct taagcctta | |
| TC17495_at | |
| ggcggaggcg ccctcggtac ttcccgctcg gcccgggcgc ccggagatga actgatcgtc ggacccgctc cccagctccg | SEQ ID NO. 5 |
| cgcgtctccg cccgctgcct cccctccccc tctgccgtcc gcggcgcggg tcccgcggcc tgggcatcca ggatcgcggg | |
| cccccgcgcg gggcatcctc cgcccgaggc gccggcccgc gccacccttc gccctgtgcc cgccggtgac acagagagag | |
| ccccaggaaa cccgtgaatg ttgaagaaaa ttcatctttg aaattttaat attcgaggaa atctgcattc atactcatct | |
| tttattaatc tgaggggatt tttgttttat ttaaaacttc ttgatattta caatgaatgg acacagigat gaagaaagtg | |
| ttagaaatgg cagcggagaa tcaagtcagt caggtgatga ttgtgggtca gcatcaggct ctggatctgg ctcgagttct | |
| ggcagcagca gtgacggaag cagcagccaa tccgggagca gcgactctga ttctggctct gactcaggaa gtcaatcaga | |
| gtctgaatca gacacatccc gagagaacaa ggttcaagca aaaccaccaa aagtcgacgg agccgagttt tggaaatcta | |
| gccccagtat tctggctgtc cagagatctg caatgcttag gaagcagcca cagcaggccc agcagcagcg cccagcttca | |
| tctaatagtg gatccgaaga agactcgtcc agcagtgaag actccgacga ctcgtccagc ggtgccaaga ggaagaagca | |
| caatgatgaa gactggcaga tgtctgggtc cggatctcca tctcagctcg gttcagactc agaatctgaa gaagagcgag | |
| ataaaagcag ctgcgacggg acagagtccg actacgagcc gaaaaacaaa gtcagaagcc gaaagcctca gaatagatct | |
| aagtcaaaaa atgggaaaaa aattcttgga caaaaaaaga gacagattga ttcatctgag gatgaagatg atgaagatta | |
| tgataatgat aaacgaagct ctcgccgcca agccaccgtc aatgtgagct acaaggagga tgaagaaatg aaaactgact | |
| ccgatgacct gctggaggtc tgcggcgagg acgtccctca gcctgaggac gaggagtttg agacaataga gagggttatg | |
| gattgcagag tggggcggaa aggagctact ggtgctacta caaccattta tgctgtcgaa gcagatggtg acccaaatgc | |
| aggatttgaa agaaacaaag agccaggaga catacagtat ttaattaagt ggaaaggatg gtctcacatc cacaacacat | |
| gggagacaga agagaccctg aagcagcaga acgttagagg gatgaaaaaa ttggataatt ataagaaaaa agatcaagag | |
| acgaaacgat ggctgaaaaa tgcttctcca gaagatgtgg aatattataa ttgccagcaa gagcttacag atgatctaca | |
| caaacagtat cagatagtgg agcgcataat tgctcattcc aatcaaaaat cagcagctgg tcttcctgat tattattgca | |
| aatggcaggg gcttccatac tcagagtgca gctgggagga tggagctctc atttccaaaa agtttcagac atgcatcgat | |
| gaatatttta gcaggaatca gtcaaaaacg acacctttta aagattgcaa agtgttgaaa caaagaccaa gatttgtagc | |
| tctgaagaaa caaccatcct atattggagg acatgagggc ttagaactga gagactatca gctgaatggt ttaaactggc | |
| tcgctcactc ttggtgcaaa ggaaatagtt gcatacttgc tgatgaaatg ggccttggga aaacaataca gacgatctca | |
| tttttgaact atttgttcca tgaacatcag ttatatgggc cttttctact agttgtcccg ctctccacac tgacttcctg | |
| gcagagggag attcagacgt gggcgtctca gatgaatgct gtggtttact taggcgacat taacagcaga aacatgataa | |
| gaactcatga atggatgcat ccccagacca aacggttaaa atttaatata cttttaacaa cgtatgaaat tttattgaag | |
| gataaggcat tccttggtgg tctgaattgg gcattcatag gtgttgatga agcgcatcga ttaaagaatg atgattccct | |
| tctgtacaaa actttaatcg actttaaatc taaccatcgc cttctgatca ctggaacccc tctacagaac tccctgaagg | |
| agctctggtc actgctgcac ttcattatgc cggagaagtt ttcttcatgg gaagattttg aagaagaaca tggcaaaggc | |
| agagaatatg gttatgcaag cctccacaag gagcttgagc catttctgtt acgcagagtt aagaaagatg tggaaaaatc | |
| tcttcctgcc aaggtggagc agattttaag aatggagatg agtgctttac agaagcaata ttacaagtgg attttaacta | |
| ggaattacaa agccctcagc aaaggttcca agggcagtac ctcaggcttt ttgaacatta tgatggagct aaagaaatgt | |
| tgtaaccatt gctacctcat taaaccacca gataataatg aattctataa taaacaggag gccttacaac acttaatccg | |
| tagtagcgga aaactcatcc ttctcgacaa gctgttgatt cgcctaagag aaagaggcaa ccgagtgctc attttctctc | |
| agatggtgcg gatgttagac atactcgcag agtatttgaa gtaccgccag ttcccctttc aaagattaga tggatcgata | |
| aaaggggagc tgaggaagca ggctttggat cactttaatg ccgagggctc agaggatttc tgctttttgc tttctacccg | |
| agctgggggt ttaggcatta acctagcctc tgctgacacc gttgttatat tcgattctga ttggaatcca caaaatgatc | |
| ttcaggctca ggcaagagct caccgaattg gacagaagaa acaggtgaat atatatcgct tggttacaaa gggatcagtt | |
| TC17559_at | |
| tgaaatttga aatcaggaca gttgttggct atggtactta ctagttttgt agtaatgttt tgctagcctg actttcctta | SEQ ID NO. 6 |
| ctggttttta tgtccatggt ccccggtgac tgttactctt gtttggggtg tgtgcatagt agtgtctcat ctgtgtgtag | |
| gcagtcagtg tatgcacacg gtgtgacagt ggaacggggt gtggctggga gtggggtgct ggagctttaa gagcatttgt | |
| ttttattcag aacagtattt cccatctttt gcctgcaggc agggaaagtg tacagtattt attttgtttc tgttttactc | |
| tgaatttgta agtctctaag tagcttatat tattattata gggaagataa gtgacttgct taaagttgta tttagtattc | |
| ttactttcta atttctgtat tttaaaatat tgaaattaaa attgtattac ttttgttctg agtgccaaat cacaaaagaa | |
| aaaaagcagt aaccgtttac agaagcaact tagtgccttg taatctaact ttgtcactgt gactacatta cctcttcaac | |
| gccagggggc acccgtgggc ctcccagagc ctctgcccgt ggggttgggg gtgggggtgg ggtgaccgca accagcagct | |
| cccccagctg gccacacaag tgcacctttc tttcggtctc ccgcacactc ttcctcttcc ttatagttac tcacctccct | |
| cagagagttg ttgctggact tggggttttt ttggggggag gggtctattt tgactttcaa aaccttttac ttcccagccc | |
| gaacccctgt tgactaatct tgcctgggtt tgtgtaggtc tccagggaca aaggttgaac aagttggacg aaggttttga | |
| cataagtggg acttcgtgat ttaatttctt tctttctttt cttttttttt ttaagtgggg aggaagggga aactagatgg | |
| actatgagag acttgatttt ggtgctaaag ttccccaatt catatgtgac atcttaaaaa tgacaacaaa tgagagaaaa | |
| gctaaaaaca acaaaaaaaa acatgtaagg ggtgagcagt taatggtctg cattccacat acaatatctg tgtaaaacga | |
| tttcctgtag aagtagcttt aatggatttt gctctagaat accttaggtc taccttagag cccccacaca atgctttttt | |
| ccctgggttt aaacttcatc taactttcag aaattggaga gcaaaaatgt tgcttatcac tgcacatcaa tataaaaaag | |
| cttatttaac ttatcaaaac gtatttattg ccaaactatg ctttttttgt taattttgtt catatttatc gggatgacaa | |
| atccatagaa tatattcttt tatgttaaat tatgatcttc atattaatct taaaattttg tgacgtgtct ttcctttttt | |
| ccacagtttt aatatattat tcttcaacaa catttttgta actttacact ttttttggtt attttatttt aaaaaaatga | |
| aaaattaatt taaaaaaatg caaaaaactg ttggattatt tattttagaa attctcccct ttgtgttgga ctgcaaattg | |
| agtttctttc tccttaggcc tttcacaggt aggactgaga atgtatgtat aagttctgtg acagtacaga aggaaaacca | |
| ccattttatg tatagcttct aaaagggaaa acaaaaaaag agaaaaaccc tttgaattcc atgtgcccat ctcaagacat | |
| tccgctcgca gatttgtggt tctggattcc aggttggagt tttccaatgt tgacataaac aactggcgca cacacataaa | |
| gatgaatgta attattattc ctcttgctgg tcactaccgt cgctttctat ttctctttct ttgtgtgaat ttatttaaaa | |
| gaaaaaaaaa actttttgta acgactattt gcagtttaaa aatcaataaa ccccgttttt caagcgagaa gcacgaagag | |
| TC18221_at | |
| attcggcaca gggcagttgt taagtatgaa atagcactga taggaaatgc atgtgcatct ttgcagactg tatttccttt | SEQ ID NO. 7 |
| ggaaaagact tttctacttt taatataatt aagccataac agtttcatgc tgtggaaagg atgaaaaggt tcattttaag | |
| agattatata gtatgaactt tcacatttat tgtgaaacat ctaactttgc cagtgttcag caagttttct tttggggtgt | |
| tggagggtgg taatggggag gggtagtgtt ggttttaggg gttt | |
| TC19211_at | |
| ggactgactc cttgggcaga ttgcctctcc tccttctcat gccagaggct gctgatgagg aaaggtccag gggactgtcc | SEQ ID NO. 8 |
| atgctgtctt catcctcaga gtcactgcct gatgctgcaa caagaccctt cttgtttagc aatagtggtt ggaacactct | |
| cttgtaagtt accggagcac tagtatagga ggaggatcat cgactaccct cccgccactc cacggctgct ggctcctaga | |
| aaccccagct tcacctctca ctgggactcg agttccagaa tgaaaagcaa gaagggtctt gttgcagcat caggcagtga | |
| ctctgaggat gaagacagca tggacagtcc cctggacctt tcctcatcag cagcctctgg catgagaagg aggagaggca | |
| atctgcccaa ggagtcagtc cagattctgc gagactggct gtatgaacac agatacaacg cctatccctc agagcaagag | |
| aaagcactgc tgtcccagca gacacacctg tccacactac aggtctgtaa ctggttcatc aacgcccgcc gcagtccttc | |
| ctgacatgct gagaaaggat ggcaaagatc caaatcagtt cacgatttcc cgccgtgggg ccaagatttc agaagctagc | |
| tctattgaag ctgcaatggg tatcaaaaac ttcatgccaa ctctagaaga gagcccattt cattcctgcg tagttggacc | |
| caacccaacc ctagggagac cagtgtctcc caaacctccc tccccaggat ccattttggc tcgcccgtca gtgatctgcc | |
| ataccactgt gactgcattg aaggatgggc ctttctctct ctgtcagccg attggtgtgg gacagagtac agatgtaccg | |
| caaatagcac ccagcaactt tacagacacc tctctcgtgt acccagagga cacttgcaaa tctggaccca gtccaaaccc | |
| tcagagtggt cttttcaaca ctcctccccc tactccacca gacctcaacc aggattttag tggattccag cttctagtgg | |
| atgttgcact caaacgagcg gcagagatgg agcttcaggc caaactcaca gcttaaccgt tttttcaaac aaaacagttc | |
| tccaaaatac ggtcctgatt gccgggggtg atggcaagag atgcattatt ttatatattt ttctattaat atttgcacat | |
| gggattgctc agacgaagct tcctgttact aagatgtctt cagtggaata gagtcattcc aagaactaca aactaaagct | |
| actgtagaaa caaagggttt tcttttcgaa tgtttcttgg tagtttctca taatgtgaga cggttcccag tatcatgtga | |
| tcttctcctc cagactcctc ttctttatgt tccaagactg tgcaatactt tagacgccct cgcacctctt tcttcccatg | |
| tggaatggga cgcccaccta cagtctaatg agtaaacttt cagttttttg tttgtttgtt tttttttaga ttcaagcaag | |
| tatgaatcta gttgttggat accttttttc atgatgtaat aaagtatttt ctttaaaagt tattgc | |
| TC21156_at | |
| ttgaagttat agggatccaa tgtttttatg atataaatat gagacacaga aaacataaaa taggtataaa atatcttaca | SEQ ID NO. 9 |
| gtttgtacag ttctttcgaa gcttttacag tatacatctt tagacaattt cacaagtggt cagtaaatgg gtgagtcact | |
| ccacagagaa gaagagcaga gacattgctg ttcacagaca ggatcagtcc ttgctttggc agacacccat ctgcccagac | |
| atttcaagac agcctggtaa acaaaacctc atgtgacagg ccaaactttg ttccagattg tatcaggaga tcccacctgc | |
| tcctaccatt ctgcaggaac agtaccaact cagacitcac acatcctttg tcttggctgg aatgtaagaa tttgtgagga | |
| aaactactgt ctcccactat ctttcctgtc ctttctgctg tctgtaaggt catctagctg tgataaatga ctgaggccct | |
| ctgaattcct atattacaga ttatttacac aaaatctcac tgggagatct ttattgggga tgagaaataa gaaaaaggca | |
| ttgtctaaag aatcaagctc tacacttgta ctagtgcata acagagtgaa aatacaaatt ttgaggtctt ccattagtcc | |
| tatcactcca taaaagtttc cacataatat agacacctcc agaggcgtag taccaggtgc tatgcttttc acgtttctga | |
| gatgggatgt agaagaagga taactgtgtg taaagtagtt cattaaagtt tagagagaca ctcaccacaa gccagctctt | |
| tgtcactgaa atgctccaaa gaggtgtgca aacaggcagt ggtctttcat caaaggaata tcttaaatga tttgagtcct | |
| ttagggggca gcaccatgta cagttcatat tcctctgtag actttatgct cttggtcgta gggttgaaac ttttaaacac | |
| tttttccttt gatttttcca gacagagttt ctcagcatat cccttgctgt cctggaactc attctgtaga ccaggctggc | |
| ctcaaactca cagagatcag cctgcctcta cccaccgagt gctgggatta aaggcatgtg tccccattgc ccagctaggt | |
| ttggaactta tttagttcga tgagactctt aaattgcttg tcttgcatat taaattacaa gttagtgtgg tgccagacct | |
| tcctagcatg gaaacagtct cctcaaaagg acacaatcag atgagatgtt ttttgaaagc taaaaggaag ctgacaagag | |
| agtttgtgaa ttgcttcaca ggcaacaaga caaacccact gattttaacc ttcacgaaag aatactggga cactgttact | |
| cacggttttg ttaagcagaa caatttacat tgttagcagt ttgccttata atgagcagaa ctatacttgc aaatttgtgt | |
| aagtgactaa aaggtgtttg attcacgtct gcattttcta agggagaagg tccaagctcc atgcatgtct gatactatat | |
| atatacattc tacacacatg taaatactgc cagctatgaa agccgtcaca cccattagct tatttgtaga taataaaagt | |
| gaagacaact taagcacaaa ttcagttcaa aagagaacct tatgatagat tacttgagag agagagagag agagagacta | |
| gaacatgaag aattaaacac tgttcacaga ccatttcttt taaaaagtca cagattctga accgacagca acgg | |
| TC23346_s_at | |
| caaatgctgg gcgtacactg gacaaggccc actggacaac actggttcct gggaggtgac ggtgcccccc agctttcttc | SEQ ID NO. 10 |
| ttgtcacaca ctggacgccg ccagctacag cggtcctcag ccctggcttc cttctgttcc gccccctgac acactggacc | |
| tgggggcgat ccctgccgtt gatcctcagt cacacacccc tccttctcac ctgccatgct gcagctagaa cttcgcaaag | |
| cctctatgtt tctgtggagt aaatattagg atggggaaca gagggagcaa tagcttggaa gaagatattc ttgaaagggc | |
| caaaaagaaa atggttttgg atcatcttgt tattcaaaga atggatacca ctgggaagac agtgctgcac acaggctcgg | |
| ctccgtcaag ttccaccccc ttcaataaag aggagttatc cgccatttta aaattcggtg ctgaggagct ctttaaggag | |
| cctgaaggag aggagcagga gccacaggaa atggatatcg atgaaatcct gaagagagcc gagactcacg agaacgagcc | |
| aggcccactg agtgtggggg atgagctgct ttctcagttt aaggttgcca atttttcaaa tatggatgaa gatgacattg | |
| aattggaacc tgaaagaaat tcaaagaact gggaggagat cattccagaa gagcaaagac ggcgactaga agaggaggag | |
| agacaaaagg aactggagga aatttatatg ctcccgagaa tgagaaactg tgcaaagcag ataagtttca atggaagtga | |
| agggaggcgg agtagaagca ggagatattc tggatctgat agtgattcaa tctcggaaag gaaacggccg aagaaacgtg | |
| ggcgaccccg cactatccct cgggagaata ttaaaggatt tagtgatgcc gagattcggc ggtttatcaa gagctataag | |
| aaatttggtg gccccctgga gaggttagat gcaattgctc gagatgctga attggttgat aagtcagaaa cagatctcag | |
| aagactagga gaactggtgc ataatggttg tgttaaagct ttaaaagaca gttcttcagg aacagagcga gcaggtggca | |
| gacttggaaa agtgaagggg ccaacattcc gcatctctgg agtccaagtg aatgccaagc tggtcattgc ccatgaggat | |
| gagctgatcc ctctgcataa gtccatccct tcggacccgg aggagaggaa gcagtataca atcccctgcc atacaaaggc | |
| tgcgcacttt gatatagact ggggcaaaga agatgattct aatttgttaa ttggtatcta tgagtatggc tatggaagct | |
| gggaaatgat taaaatggat ccagacctca gtttaacaca caagattctt ccagatgatc ctgataaaaa accacaagca | |
| aaacagttac agacccgtgc agactacctc atcaaactac ttagcagaga tcttgcaaaa agagaggctc agagactttg | |
| tggtgcggga ggttcaaaga ggagaaaaac gagagctaaa aagagtaagg caatgaagtc catcaaagtg aaagaggaaa | |
| taaagagtga ctcgtcgccc ctgccttcag agaagtctga cgaggatgac gataaactga atgactccaa gcctgaaagt | |
| aaagaccgat ccaaaaagtc tgtagtgtcc gatgctcccg ttcacatcac tgcgagtgga gagcccgttc ccatagctga | |
| agagtctgaa gagctggatc agaagacatt cagtatttgt aaagaaagaa tgagaccggt gaaagcagct ttgaaacaac | |
| ttgacaggcc tgagaaaggc ctttcagaaa gagagcagct ggaacacact agacagtgct taatcaagat cggagaccat | |
| atcactgaat gcttgaagga atattccaat cctgaacaaa ttaagcagtg gaggaaaaac ctgtggattt ttgtatctaa | |
| gtttactgag tttgatgcaa ggaaattaca taaattatat aagcatgcta ttaaaaaacg acaagaatct cagcaaaaca | |
| gtgaccagaa tagcaatgtt gctaccactc atgtgattag gaatccagat atggaaaggt taaaagagaa tacaaatcat | |
| gatgacagta gcagggacag ctattcttct gacagacact tatctcagta ccatgatcat cacaaggacc gccatcaggg | |
| agattcttat aaaaagagtg actctcggaa gagaccctac tcctcattta gcaatggcaa agaccaccgc gagtgggatc | |
| actacaggca agacagcagg tactatagtg accgagagaa acacagaaaa ctggatgacc acaggagtcg agagcacagg | |
| ccaagtttgg aaggaggctt aaaggacagg tgtcactctg accaccgatc tcactcggac catcgaatgc actcagacca | |
| tcgctcaagc tccgagcaca cacatcataa atcctccagg gattatcggt atctctcaga ttggcagttg gaccaccgag | |
| ctgccagcag tggccctagg tcacctttag atcagaggtc tccatatggc tccaggtccc catttgaaca ttcagctgaa | |
| cacagaagta cgcctgaaca cacctggagt agtcggaaga catgacagaa gctcatgagt ttgtcttccg ggactttgtt | |
| ttagccatag atcataaacc aacacagtaa ttgccttaca tgacttgaaa gattcaaaca gctcttctgt cagtagcagt | |
| attgttactt ctctccagga cgcaaggtct attatcccaa cagaagagaa aatattttta tatttaagga ttatgctgca | |
| ctgtactgcg atactgcagt accttttttc tcttctttta aagaaatgga aaatgtttac tatgccaggg acctcagcac | |
| tgccctcccg tgtaggctgt ataaaactgt ttttatgtca gtgattttag actgactcca tttaaattat gtttgtatat | |
| gaactttact ctgacctgtg atcatgtttc aggaaggaat aaaagagagg tcttttctta ataaagaaaa atcactcaag | |
| gactttgttc actttccaaa gctacttgtt tacattgtac actgcgacca ccttgccgct cccatcacaa gcttgaatat | |
| ttaaatcctg tacttaagtt tgtaatatag ccgggaattt cctgtctgtg attattatta tgccttttta cagaagaaga | |
| tggctgtaaa ttattgtaaa tggattaaat gagctgccct gccctgccct tctcaggctt cttttgactg ttcctttccc | |
| taccaactca ggccttctta ttaaaaaaaa aaatcagtgt aataacactt tttaatgatt tgtcttgatg gaatcattgt | |
| ttagaatgta aaaatgggga aaggggccac ttatttcttt tagtcctttt tatattgagt atttttatta gatacatgtt | |
| ttgcccctcc tttttaaagt caatattgtg tttgtagttt tacagaagca gtggcgaggg ttacatgtga gacaaactcg | |
| gtgctctggg agagtcccca gtgcattggg ttgaaagggt ggtaggtgta tgaacactta aaatccaaac ggccaaacgt | |
| tcttgtaaat gcattgcttt tccctttcat gtgggcaata atgtcaaacg tgctatgcag ccaggttaac atttgaggta | |
| aacttgattg gctttaatat aaaaactgtt acagtacaca ctgattgtat ataaaaacct tatatatgac aaattaaatt | |
| ttaagaaaaa ggatatgtgg gctcctgtag ttttctgctg cattcttgtt tccacgagca tttaatcttt gctttaagta | |
| acagctgcca gaaagaaagg tattttggtc agcaactgag aactgttgat atgtaatcct aaaactgcct acgtctgtat | |
| agatgaatca gtcttctatg aagtaaaaca gattctagca gtctcggctt cctgtagaca ttcctctagg atgtcttact | |
| cgtcacctcg tttctcctta tattcagtgg gaattttgga tgtatgttgt gtaaccatta ataaccttta ctcctggtca | |
| tgtctccatg gagtagctgt ggggctacct aaagttagtc tgctggcttg tattttcata tctttttttt tacatggata | |
| aatataaaat ttatgtgtag tatac | |
| TC23450_s_at | |
| gagaagacga cagaaggttt atgccagtac ttttattaag acagagtcta actgtacagc cctgcctggc ttcaaactgc | SEQ ID NO. 11 |
| ttcctgagtg gactttcaca tccagcatat atgccagcat tttattagtc actccccatg tttcatttgt tcaacctttt | |
| agccagtgat ttttctccct acaaaactga tagcagtcat aatccttgag ctatcaccat tcacaaagga gatcaagagg | |
| ctttcagaat tttatttaaa aaatccagag tggatgaaaa aggaacaatc atcaagaata cagatgccag acattggcca | |
| accaagaggg tggctagaga agtatgcctc aggcccatga gaggcttata tcattctcat atcatacttt aagctatggc | |
| taacacaact gctatccact gacctagctt ctgcacctac agaccatggt acaggtagct cagtcttaaa ggcatcagtt | |
| gtaggagcaa atatacagag gtcattcttt atagtagtgc tcttggccaa caagcatctg catatgatct caactcaagg | |
| tcaatgacag actacctcca ctgcatgtga aatgggcttc ccaccttgaa atcacagcca tttcagcaca aaccaaatct | |
| accttaatga ttggttcaca gccccatccc acccctttac atgcagctga aaataacagg ctagtgacag aacagtatga | |
| acctgctatt gctgacttta ttacccatta aatgagttag caattgtcac taagtttata tattaaggaa attatatata | |
| gaatactgca aaaatacagt aaaaagactg aagtctgccc cttttctgct caggaagtcc ctttagtccc aagcttcata | |
| gtatgtcctt ctggctccac aatgcactgc cacgattact gtttctcttc cttctgatct tccttctgtt ccccagtgcc | |
| agaacttcca gaaccttccc gttcagatgc catctttttg tacgccattt cgaagagttt caatgacgcc tgctgtaggg | |
| aagatgctgc ctgcctgatg ttctctcctg tctcactgtc ctttccagca aggagcgctc tcattttgga aatctcttcc | |
| tttagcttgt tgcactcatc agcaggcaac tggtccttaa attcttccat cttggtttct gtgtcatgaa taattccttc | |
| agccatatta actgcttcaa cacgttcctt cttcctgcgg tcttcctcag cgtacttctc tgcattttta accatatttt | |
| caatatcatc tttgcttaat ccaccagaag actggattac aatctgttgc tcacgaccag tgcctttatc tttggcagaa | |
| acgtgcacaa tcccattggc atcaatgtca aatgtaactt caatctgggg cactccacga ggggctgggg gaattccaat | |
| caaagtgaac tgtcctagaa gtttgttgtc tccagccatc tctcgttccc cctgacacac tttaatctct acttgagttt | |
| gtccatcagc agcagtagaa aacacctggc tctttttggt tggaatagtg gtgttcctat taataagttt ggtaaagacg | |
| cctcccagag tctcaatacc cagagagagg ggagtgacat ccaggagcag cacgtctgta acgtcaccag ccaacacacc | |
| tccctggatg gcagctccga tggctacagc ctcatcagga ttaacagctt tactcggggct ctgccaaaa agatcttgta | |
| cagtctgctg aaccttgggc atccttgtca tgccaccaac cagaatcact tctcctatgt cactcttgct gacttctgca | |
| tcctgcatag ctttctgaca cggagcaata gttctcttga ttagatctgt gacaatgcct tcaaactgag ctcgagtcag | |
| cttcatattc aaatgctttg gtccagaagc atccatggta aggtatggca agttgatgtc agtctgcaca gatgaggaaa | |
| gttcacattt agccttctca gcagcttccc gaaccctctg aagcgccatg ttgtctttgg tcaaatcaac ccctgtctct | |
| ctcttgaact ccttgacaat gtgccgcaac aaagcttggt caaagtcttc ccctcctaag aaagtgtccc cattggtaga | |
| tttcacctca aacactcctt tctgaatttc caggatagaa atgtcaaagg ttccaccacc taaatcatac acagcaatga | |
| ctttatcttc agatttgtcc agaccgtaag ctagagcagc agctgtaggc tcattgatca ctcgaagcac atttagccca | |
| gatatctggc cagcatcctt agtggcctgt cgctgtgaat cattgaaata agcagggact gtgatcacag cattttttgc | |
| tgtgtggccc aagtaatttt ctgcagtctc tttcatcttc atcaacacaa atgctccaat ctgacttgga gaatagagtt | |
| ttccatgagc ctcaacccaa gcatcaccat tggaggcacg gacaatttta aaaggaacat tcttagtgtc tttctgtact | |
| tcagggtcat catatcgtcg tccaataaga cgcttagtag catagaaggt attgtttgga ttggtgacag cttgccgttt | |
| tgctggcata ccaacaagtc gttctccatc tgctgtaaag gcaaccacag aaggggtagt tctggcacct tcagcattct | |
| ccaggacctt tgcttgtttg ccctccataa cagccacaca ggagttagta gtacccaaat caataccaac cactgcaccc | |
| ttgattgctt ctgatgcata atctcttctt gaaacaaatc taaaagcctc atggctaagg ccattccagc catcctgggg | |
| acgggcggct gcggggctcc gggacgcagc ggtgcccacg agacgcgcgg ccgcggctct gctggcgctt atcatggtgg | |
| ctggacagag ggggttacga gggcaagaac caacacccca cgggcccgga gctgcgtgca cggtggtggt acgcttctgg | |
| aaacctccaa ccacgtgggg tgggggcggg ggctggccgc tgcagcaatg aagcccgctc ttctgctgag gcgcccgacg | |
| tgctgcctga ctagagacta cgtgtccgct gctcccggga tcggcgcacg ccgccagcac gcctctcagc attaatcgtt | |
| gaaagttccc gttctctcat cacggtttcc agggctgtgg aagcagggag aagggaaaga gagaagagag gtggacaggc | |
| atcgcttcgg acagtcccgg tccttaggac gggaaagact caaggtcaca cgggataaat tacgaacaga ttacagtctc | |
| cttgcgtcag ttgcgcagaa ggaggtgcgt tctgcgcctg cgttctccgc tccggacaga ggtcgtgaag cgcatgtgtg | |
| ag | |
| TC24045_at | |
| tgctggtggg atcaaagcgc agtgtcctgc ggcggggagc ttggaacgct aagaaaagtg accatggaga acaacaaaac | SEQ ID NO. 12 |
| ctcagtggat tcaaaatcca ttaataattt tgaagtaaag accatacatg ggagcaagtc agtggactct gggatctatc | |
| tggacagtag ttacaaaatg gattatcctg aaatgggcat atgcataata attaataata agaacttcca taagagcact | |
| ggaatgtcat ctcgctctgg tacggatgtg gacgcagcca acctcagaga gacattcatg ggcctgaaat accaagtcag | |
| gaataaaaat gatcttactc gtgaagacat tttggaatta atggatagtg tttctaagga agatcatagc aaaaggagca | |
| gctttgtgtg tgtgattcta agccatggtg atgaaggggt catttatggg acaaatgggc ctgttgaact gaaaaagttg | |
| actagcttct tcagaggcga ctactgccgg agtctgactg gaaagccgaa actcttcatc attcaggcct gccggggtac | |
| ggagctggac tgtggcattg agacagacag tgggactgat gaggagatgg cttgccagaa gataccggtg gaggctgact | |
| tcctgtatgc ttactctaca gcacctggtt actattcctg gagaaattca aaggacgggt cgtggttcat ccagtccctt | |
| tgcagcatgc tgaagctgta cgcgcacaag ctagaattta tgcacattct cactcgcgtt aacaggaagg tggcaacgga | |
| attcgagtcc ttctccctgg actccacttt ccacgcaaag aaacagatcc cgtgtattgt gtccatgctc acgaaagaac | |
| tgtactttta tcactagagg aatgattggg ggtggggggg ggcgtgtttc tgttttgtta tgccaaatga gaaagctgtc | |
| agggagactc tcatttaaat ctaatctgac ggtcctcctg gtctttgtac gctaccactg cctagcaatg cagccagcca | |
| cagtgcagct acctcaactt cgacatcagg tagttgaaat gaaatttaat ttaataagga gcaagtaact gtcaatgatg | |
| gtactatcat cctagatgaa attacaaagt tgccctttta taattagcaa gatttggcga tactatgaat tttgaagtca | |
| TC24067_at | |
| cttctgcata aggtttgatg ggtggaaagg gcctcaaaga agccacagaa atcgccattc taaatgccaa ctacatggcc | SEQ ID NO. 13 |
| aaacgactag agaaacacta cagagtcctc tttagaggtg caagagggta tgtggctcat gagtttatct tggacacccg | |
| acccttcaaa aagtctgcca atgttgaggc tgtggatgtt gccaagaggc tccaggatta tggatttcac gcccctacca | |
| tgtcctggcc tgtggcaggg actctcatga ttgagcccac cgagtcagaa gacaaggcag agctcgacag attctgtgat | |
| gctatgatca gcatcaggca agaaatcgct gacatagagg agggccgcat cgacccgagg gtcaaccccc tgaagatgtc | |
| tccacactcc ttgacctgtg tcacatcctt cctgctggga tcggccgtat tctagagagg tagcagcatt tccactgccc | |
| tttgtgaaac cagagaacaa attctggcca accattgccc ggatcgatga catctacgga gatcagcact tggtctgcac | |
| ctgcccgccc atggaggtct atgagtctcc attttctgaa cagaagaggg cttcttctta gtcctccctc ctacgttcaa | |
| agggctgacc cgatgtctct tgctggagca tttgacaagc aaggatattt cttctccttt acgtggctca cacatgagtt | |
| ttatacgctg tatattttta taatctttca aggtaatgta agcacaatta gcatggtgag tggcggccac ctgctgtacg | |
| gcaaggcagt ggctgttcgt gttgccttga ttgggagccat ttagtttgc cggatagaaa atgtggggtg cgctagcaat | |
| tttaacattt taattcaaga agtttgcagc agtcagagcc tatgctggga attcaataga cattcttttt gttccaaata | |
| agtcctcgtg gactgtgccc tctgtgggaa accccagggc aaatgtttac attttataca ctgaagaatt ctcctcacta | |
| atgtgcctga tctgtcacag cataagtgtc ctcctttcac tgtgcggact ttttttttta tctgctttta ttagtgtcct | |
| aataaaactg agtttgagta aaaaacctta tgcagaag | |
| TC25965_at | |
| attcagctaa gtgaccctta acctgggatc tactgtactg taattttcaa cactatagaa tattatgctc cccagtattg | SEQ ID NO. 14 |
| gtgaatggtt agcaatacaa aactggcagc ttagtagttc aggatctttg gaatacattg aaattcataa atgaagttca | |
| tttttgaagc acacaattca aatcattaac tcagacgaga caagtccatt tatatggcta aatacttagc ttgaatactc | |
| ttctgtattt tactaatcct aattaattcc ttttccatgt attttactgt acttatccat aagagatcag caggtattat | |
| cagacactca ctgagtgctc agaaatagtg aggactgtga ggaacctcaa gttcagtttt gtctgtccgt ttgtctgtcc | |
| gtgaaggtgt cttctccagg aggtttgcag tgttgttttg ac | |
| TC27326_g_at | |
| tgtggccctt agagacagaa tggtttattg aatccaaaag gtagagtgtc aaaaacttaa tagaatcctg tccagtaggc | SEQ ID NO. 15 |
| actctgggcc aggaggtttg catcttacat cagtctccct gaaggtctcc gtgttcttgg aaagtaaagg ccctttgctg | |
| agcaccccgt ccttgccaca gggtccctgt gactcagggg acagtgttgt agacactgta gttgtccttc tgtgtgacat | |
| gcagcttcca ggggaagagg cgcttctggg aagggaggcc tcgggcttgc ttcaccatga gtcccacatc ctcatctgac | |
| agcagcctaa ccaagtcccc ctcagcatcc tggtagctaa gggcaatgtc ttctctctgg aactcacgcc tcatgagcgc | |
| tagcaggtct ttgaacaggg gcgtgctgct gaggtcctcc tccaccgcaa tgtccttgat ggttttgcct gtgtcttcat | |
| agaagtagca tcgtagccag ttggtggtgt cctcgtcctc gggaaagtcc ttaaggatct tcacgaagga ccctgggaag | |
| atgcctgtgg ctccctggga ggtgccctcc agccagtctt gttgatctt actgagaagg aagatcacat ctccagcttt | |
| gaaacttagc tccaatttgc tgttcccagt gaagtcaaac aaggcctctg ctcttggcgc ttccatgcga tccatgatgg | |
| ccccttgtgg agacacaccc ttgattttgc gcgtgcgcgg tcggagcctg cggagtgcct ggggcacctg ctcagcatcg | |
| tatgcagact gatagaagaa gatcctgacg tcggggtcca tcagcacgca gaccggcagg ctcaggaggt tcttcatgta | |
| ggcattgagg gccgggatcc gagtctcagc gatctcttgt tttgcgcccat gtagacttt ggctggcaat gtgggcaggt | |
| tgcaggtgaa agggctgttc ttgctctctg gcccaaaccg ctcctccagc ttgctctgca gggcgtagaa ctggcgatag | |
| cggcggtaga tgagatactt ggaccctcct tttgttttga cctcgatgac aaaaacaaag tggctggtga agcctctctt | |
| ctcctcgatg tcagcgatgt tggctgagac ggccacatcg tctggaagct gctcaaagtc gctctctgat cgcagctgct | |
| gggccagggc catggctgtg ggtagtcaga aaagcagatg gatgggggcc ttgcccaacc agatccaggc cgagttcacc | |
| tctcacttcc tcctatgtgc atctgagagc ttcctgaagc ttggggtgtc cccgggggca gcctttgagg aggtcccgcc | |
| ctgcccagct tctctggacc ctccttgaag tacctccttc aggcg | |
| TC29889_at | |
| ttaggattct aataaaacca cagaattctt ttaattaagc tcaaagttgc aagtttgtct cacgtatctt tcatttgact | SEQ ID NO. 16 |
| aatgaacttt tcgacaattc cccccagtct ttcaggttcc atcagtttat ttaccagttc ctgctcaaca gctatcagag | |
| ccaggctact aagtttctct tgtcccatgg tacgaagtaa gtatgtttta agatgagaca gcgtggaaaa tgacttctca | |
| ttatttgctg aagtaattgg ccaagataaa gcaatatgca acagcttcgt gatacaagga atattattgt ggagagcatg | |
| ctgaataaac aaagagccta ggtcaatgaa gcttaaggac ccatcctctg cgatgaagtt gaaacctgca tactgccggt | |
| aaaatcggag ctcgggaatg atgtctgcat caagcttata gaattcctgg atgtgcttgg ctgttgcttc atttaatggc | |
| tcattccact taagtaacag ctctgaaatt tgtttcattt tacaatagtc aaactctgaa aaatataact ttaaatgttt | |
| taataccgtg tccagacctt ggtagtaaat attaaattta tattgttcat ctgctgaagt aggaaaaaac atatgctctg | |
| atttgccagg atctactgtt ctttgaagtt tgcttctgtt ttgaaaaaaa ggtttctcaa catcataacc cttagtggtg | |
| atcttttaac atatatcatc tgcat | |
| TC30384_g_at | |
| ccccgccggg cgaccacttc accctctcta cgtcggtctc tcaaagatgc cgctctacga gggccttggg agcggcggcg | SEQ ID NO. 17 |
| agaagacagc agtcgtgatc gacctaggag aagctttcac caagtgtgga ttcgcaggag aaactggtcc acgatgtata | |
| attcctagtg tgataaaaag agctggcatg tctaagccaa tcaaagttgt tcagtataat atcaatacag aagaattata | |
| ttcctaccta aaggaattca tccacatact gtatttcagg catctgttgg tgaatcccag agaccgccgc gttgtggtta | |
| tcgagtcggt gttatgtcct tcccacttca gagagactct gactcgtgtt ctttttaaat attttgaggt tccatctgtc | |
| ctacttgctc caagtcatct gatggcactg ctgacgcttg gaattaattc ggccatggtc ctggattgtg gatataggga | |
| aagcctggtg ttgcctatct atgaaggcat cccaatactg aattgctggg gagcactgcc gttaggagga aaagctctcc | |
| acaaggagtt ggaaactcag ctgttggaac aatgtactgt tgacactggt gcagctaaag gacagagcct tccctcagtg | |
| atgggttcag ttccagaagg tgtgctagaa gatattaaag tgcgcacgtg ctttgtcagt gatctgaagc gtggactgca | |
| aatccaagca gcaaaattta atattgatgg gaataatgag cgtcccactc cacctccaaa tgttgactat ccattggatg | |
| gagagaaaat tttacatgtg cttggatcaa tcagagattc agttgtggaa atcctttttg aacaagataa tgaggagaag | |
| tcagttgcca ctttaatttt ggattcccttt tacagtgcc caatagacac caggaagcaa ctggcagaga atttggtaat | |
| catcggtggc acatctatgt tgccaggctt tctccacaga ttgcttgcag aaatacggta tttggtagaa aagccaaagt | |
| acaaaaagac acttggcacc aagaacttcc gaattcatac tccacctgca aaagctaatt gtgtggcctg gttgggaggg | |
| gctgtttttg gagcattgca agacattctt gggagccgct ccatttcaaa ggaatactat aaccagacgg gccgcatacc | |
| tgactggtgc tctttcaaca accctcccct ggaaatgatg tttgatgtgg ggaaagcgca accaccgctg atgaaaaggg | |
| cattttccac tgagaaataa acgcttgaat acatccagcc ttgcttattt caaatattta atcaattaga ggtaaattgt | |
| acaaagtatg tgggatgatt taatatatag aggtgacctt tatgttacaa agcatttctg tattttctct ttgcattaat | |
| atttaattca tctgactttg tctcttgtgt cgtatgtcgt agtggatgct tgtgagatat gttgtattta tatcaataaa | |
| tatagttaag ctattcaaaa aa | |
| TC30935_at | |
| aattcggatc cttgtttata tcaaaagtgg atgtgaagat gttccttaaa ggagatacac ggactttctg gcacaatttt | SEQ ID NO. 18 |
| gagcctcagc tctccttact catattctag cctccctcct tccctgccca tctctccccc agagccatgg ctttcatgga | |
| ctttctcttt ctctgtgaat actataagcc agttccatat ttagacacct ggttttcttg ttcaaataga cactagatga | |
| ttcttggcta ttctccactg gtattagaat gtcccctgtc cttagagctatcctggccta ccacacccac aacaggctcc | |
| ctggggctct ttaacctata tgcagcaagc cactctggga ttggtcataa aagggctctg tcaagctctc catgacccct | |
| tgatgccctt tgagggcaca agaagagtca tacaagactc agagctaaag ataattctct acccattcta aaaaagtcca | |
| ctagttgggt tgtggtagga cattgcttgc catagtggtg atgattctgg tagcacaatc tgactttgag ttactccatg | |
| attttgtaaa gtattaggag cccttaaagt gttcttttgc catcaactat aaatttctca tatatcctgg aggctttcct | |
| tctctttgcc ttgggtaaaa atcaaatgag gtgaaagata ccttcatgtc catattttgt atgttctagc tagataaaat | |
| ggatgtttca gaaaagcctt taaaaatctt tatattgaca tccataacac caaaaactgt ctttttagct aaaatcgacc | |
| caagactgtc acagcaaagt agaagaaaca gccatcttct ttggaaaagt aaaatgttca taagaacagg gattgcccac | |
| tatgatgata atgatgtact gtgtctttgt gtaagtccta tgtgttgact tttcaaatat agccaattgc ttaggtatgt | |
| acaatgagtt tttctattgt tcctttgatt tttcagagac cccccctttc tttttccagt ttttgtatat ttattgattg | |
| actaataata atgaggaaaa cctgatgtgt acattaccg attgaaagtg tgtattggaa aatattaaaa ggttgtaaaa | |
| aatggaacct agggtctctg gtgtgaaggg tctgggaagt ggagaggggc agaaagtgat tgtctggatg tctgaataat | |
| atggaagaag tcagaggcag ctggcatcct ttgttttgtt ttgttttgtt ttgttctttt atagcccccc cctcctattt | |
| ttctttggtg ttttgttttt gtttgtttga tttttgaaac aggatctcat agagctcagt ctg | |
| TC30992_s_at | |
| gacagcgcaa aagctcgcaa accttgtcag cctggatcca cgtcttattc aaaacgtgtc gcgatcattc acagcttaag | SEQ ID NO. 19 |
| atgtacggga ttcgcgcgtg agaagggaga gagaagcgcc ggtttgccga cgccaccccc tctcatcagg gctggcgcgg | |
| cgagtggcag ctccttaggt cacggcttcc gccggcgcca gtggcctagc tccctgcggc tgtccttggt cgccttgtca | |
| ccaggctgaa gcagtcaaga tgaccttatt tcagcttcta cgtgagcact gggttcatat acttgtccct gcgggatttg | |
| tctttggatg ttatctagac agaaaggatg atgaaaagct aactgccttc aggaataaga gcatgctatt tcaaagggaa | |
| ctgaggccca atgaagaagt tacttggaag taaaaaatgg ttaaatcaca gaatgttcac attataaaag ttattggaga | |
| aataaaaaca tttactattt aattagactt ttgaagcagt acagcttttc cactgatgat tttatactcc ccactcatgt | |
| taagaatgtt gttctagttt tcgttaaacg tagaaacaat aatgtcaaat gataatgtct tagaacttga atccatgagc | |
| agagtcaaag gctggaaccc ttgttttgga cctgatttat ggaagtgaag agttggacca ccatagcatg cattatagct | |
| actggttttg tgacagttgt ccacaacacg tatttgttat tttaaaaaaa aaaaatcatg ttttattact ggaacaaaaa | |
| taacttgagt atttatgtga gaggaaaacc gtgtgcgaac ttggttgact cattgaaggc aaggtgctaa aatccatcag | |
| catttgtttc ttacttatcc ttatacaaat cagctcggtc ttacagacca gcaaaatatc tcaagagctg ttatgtaaca | |
| gcctcaaatg gttgattatt agaatgaagt acaggaattc tataggaata tactataata ccagttgtca aacagagctt | |
| aagaaaagga gagatgaaat ctactgtagt tggagagaat gtggtttgag ccagagcaga gacttggcag agcatgggca | |
| catcttcaga aaaggcctgc cagctggact ccccacccct caccccaccc cccttgtgag ttctggtttg tgtggatgat | |
| gtccaagtta ccacgtggga aagtgaacca gatcacaaac ttctgcaaaa aggctttccc agagggctga gagtctgaag | |
| aaagttcact ggactccatg tccagagtct gtagcatgct gacttttctg ttgacgttta caaagaaagg aaaaataatg | |
| gtgatattta tactgtgcta gaaacgaaag ggccagggca cacaggactt ggaaagcatc cagcaatagc cctgcagccc | |
| atgtggagaa gctgcgggct gcagtccatc ctcagggaga ctgtgctctg ctcctggcct ccttatgggg gtgctgagtc | |
| actgctggct ctcgcagcat ttctcctaag cttccttgtt gcacaaagaa aggaaggaac tcacattgta acggatagat | |
| tcttgtttag actggctttg ttgttaacgc tttctaagtg aatgcagcat gtgatggtat tttaaagtgt gttcccatgt | |
| gacaatttta taaaagtttg ttaccttgca attttatttc aaaacataaa ttcagattta aaattag | |
| TC31681_at | |
| aattgatatt ttttgatctg caaaatttta ttaagcaata gctggacaaa tcttacaatt tcaaatcatc aagaaaaaaa | SEQ ID NO.20 |
| tgagattaac ccatttcaat tttaaaggca aaataatttg gaaaattaca tcattttgaa tgcaaaccat tactcccttc | |
| tagaatactt cctggacaat ctagtataca aaatacatac aaagataggc aaataaggga gagctcattg agatgcaatg | |
| gtgactctca acagatcttg tctggaaggt taaagcaaaa tgctgacttt tcaaaggaaa tgaagaaacc aacactgcag | |
| ggaccgatgt gtatgaaaca gtgcacgctg ttcccaaagt gtggacaaga acatgctggt ggtcttgttt ggtctgtgac | |
| atatgcaccc cacattgcac atttatgggg ataaaaaaga tgacagagaa ggagaaagac agatgccagc agtagcaata | |
| gtagtaaact cggaaattat gcccaaattt acagtactaa aataatgcat ttataaacaa atgcaaacaa tacacctttg | |
| gtaaataata cagtgcgttt ccactcaaat gtataaccca tgcttttcta caacacgaga aagaatatct acaataaact | |
| agatcttagc atcctaccct tcccctgaaa attgacttgt aaacagaaaatacggataca aagtcacagg agaactgcac | |
| aaagctgaag aaaatggaaa ccaagtcagt cacaatcctc cagagttcat ctgccagaga tgaggcgata cacagccgtg | |
| tagtcttctg aactgtatgt taccaaaaac tcccaggagc aaatgccagc tcaaacctag ctactgttgc caacctgtcc | |
| ccagctcaca ggacagcaca ccaaaggcgg caacccacac ccagttttac agccacacag tgccttgttt tacttgagga | |
| ccccccactc cttggggact ccttggggac ttccactaca cttggatttg atgcttccat cattttatta gaataagcat | |
| tctaaacaca tttaggggaa aacttctggt ttcttttcag gagacaatag acagcttaca gactcatgaa aacttttgtc | |
| tacacaggtg ccacgttttg acacatactc ccaccctcgt gaaagggaag acaagctgct gaattctgag ctctcaggct | |
| accagagcca gaatgcagtg tttaagcttc tccaggtatg acttttcatt taagagatgg acaaaaggag gccaggcacc | |
| ctaggggcct ggccaaagtc acccctatcc ccgtggcacg acaagtggac ctggagtccc tggcctttga ctacagagtg | |
| agtattacac agaaggccag tctctggatg gacaaaacca acatgcaaaa gctttcgctt tgtatgctca ttagagccga | |
| gtatttctag ctgtggtggt cccagttttt ctcaggctat acattcaaat aagtatattc acccataact tttgttttga | |
| tgatactaga gccaaataaa tctaacacta gttttcatgt tggttaatat actcttttcc tatgggtctc tatatagttg | |
| actgttttta atttcagtat ccaatgagac attgggacat tcacaaattg ctattaataa ccatgaagta cccaaaattc | |
| tgttacttaa aagtcttcat ttgaggagaa tttaatcaag tgatattgac tggaagaagc cattatctta aaagatacga | |
| tgccacaact gctgcagcca gcagctttct gcccatgtgg aacctgagac tcactctccg cctgggggag gaggctggtg | |
| agtggctgct gttcctgcag ctcacaggtc aagcaactca aggccagaag cctgcatctc cacagacaaa gagtatcttt | |
| tagggatgtt cttatttagc aaagttgtaa gtataacttt tgcagtctgg aaaaggtaaa ttttaactaa aaaaaacaag | |
| acaaacaaac aaaacaaaca acaacaaaaa aaaaaacccc taaaacttga agatcattta ctaagggact cttttgagac | |
| tcttatctca gatgtcttgc ccttcctgtg tcttatcaca ttttatttta aattaacatt ttcaaaataa gaaatactgt | |
| cttagggtat gctatctcaa agatattgcc tattttgcat ataatcaata ctgagataaa gagtgaattt atttttttag | |
| gggatcaata agattttctc caactctcaa gaaagttgca aagcacctag cctccccgtc ccccacaaaa accttctgtc | |
| atattctctt tatttattta gaaaaatagt acctttgtaa tgccttgttt ggtggcaata tattttg | |
| TC32225_at | |
| attcggatcc ttgagcggag cacggcgctg cttccaccgc tgctcaggag gggagcatgt ctgcaactcg agccaagaaa | SEQ ID NO.21 |
| gtgaagatgg ccaccaaatc gtgccccgag tgcgaccaac agattcctgt tgcgtgtaaatcatgtccct gtggttacat | |
| atttattagc agaaaactac taaatgcaaa acattcagag aaatcaccgc cgtctacaga aaacaagcac gaggccaaga | |
| ggaggcgaac agagagagtt aggcgagaga agataaattc tacagtaaat aaagatttag aaaacagaaa gagatctcga | |
| agtaacagcc attcagatca tatcagacga gggagaggac gacctaaaag ttcatccgcc aaaaaacacg aggaagagag | |
| agaaaaacag gaaaaggaaa tcgacatcta tgctaacctc tctgatgaga aggctttcgt gttttcagtt gccttggcag | |
| aaataaatag aaaaattatc aatcaaagac ttattctctg atatttgtct tcaaactctg ttggttgcca ctttcttcag | |
| gattgcatca gcagatcctc aggcagtcgt ggactctgag gagcaccaga ctgtgtgaat gagggccttt ggttgctttc | |
| tctgtttcct agtctgctgc cacgctttgg gagcaaagct gtg | |
| TC33206_at | |
| acagcacccc agtccacagg gaagatactc acgttagttg atggatgttg gaaggcattg gaggaaaaca ccacagttag | SEQ ID NO.22 |
| gactgttaat gactccacac tgccgttaag actgaccttg aacatgcgca aacgttcatc agtcatggtg gtggtggtta | |
| gtgccatagt caaataaaaa tatttctata caatatgttc atttggtcat gtgctattta cagattttac aaaacataca | |
| cacacataaa cacacacttg cccttacacc aacctacata caacgggcca gcagaaactt gagtaatgta tatcacatga | |
| cagatcgtgc ctatacatta taaataaaat cctactatgc ggcagaacag gactcttctc agaacaggaa actaaggctc | |
| taagctgaga agctcagaaa tttgaactga ttatacgtaa ttcaaggcca ttaagaacaa gacagttatt tttctctgtc | |
| catttaaaat gttttatttt tcttttaaac tagattgtga agtgccattg aataggcaat gttggcaaaa caatgtctgt | |
| tacaataaaa tacattagac atttaaataa ataaccttaa aaactaggcg aagggacaga aacccagtcg attggatctg | |
| gagcaatgtt ttctgcacaa gcgagacagg caaacctctc gtgaagacgg atgtaaacag aaccatcaga cctacagaag | |
| aagccgagca ggctggggtg ggctgggggt gacagaggct gagggtgcag gaagtggcaa accaaaaata ctgactgagg | |
| tagcaggact ctgctcagtg cagaaaaatt ggcttatgaa taaaaattag actgtgatac caaattaaat ccagagaatc | |
| atgcaaaaga cacaatacat gctatttagt tttttttctt ttaaaaacca cacacattca tttcctaagt ctgctgccaa | |
| ttaatttgct gacaatgtct gctggctcaa ctatctttgt acaagtatga gtattaaaca gaacactgta catgcttttt | |
| catctacaaa aaaatatctg cataaaatag tgttagttct ctgtacacgt caatggacac tttaattatg tgtataaaaa | |
| aaggaaaatc ttgtcccact ggagtcagaa aaagcaaaac aaaatctaga tatgaaacct ccttcaccag ccaatatgtt | |
| catgtgaaaat tcagcagaa atagacagtt gctttaaaca ataaaaaatt aattgattaa gttaaacatc ttctgtatgt | |
| agcgctgtca gtcaagacca gaacatatcg ctaaagttag tgtctgtgct gtagacccag accaccaggg gcataatgag | |
| cagcacaaac ggccaggaaa tgccatcggt gcatgccgac agggagcagg aagatttggt ggcaggctgc acacactctt | |
| taccgggttg ttccaggtag tttcgggcca caaacttgag cgtctcatcc atgaggatca caggcaagga gattttcagc | |
| TC33833_at | |
| cggctctgct ctccggcttc gcaatggtag cgatcggtgg aagtccagct ggacacagac catgactacc caccagggtt | SEQ ID NO. 23 |
| gctcatcgtc tttagtgcct gcaccacagt gctagtggcc gtgcacctgt ttgccctcat gatcagcacc tgcatcctgc | |
| ccaacatcga ggctgtgagc aacgtccaca acctcaactc ggtcaaagag tcaccccacg agcgcatgca tcgccacatc | |
| gagctggcct gggcttctcc acggtcatcg ggacgctgct tttcctagca gaggtcgtgc tgctctgctg ggtcaagttc | |
| ttacctctca agaggcaagc gggacagcca agccccacca agcctcccgc tgaatcagtc atcgtcgcca accacagcga | |
| cacgagcggc atcaccccgg gtgaggcggc acgattgcct ccaccgccat catggttccc tgtggcctgg tttttatcgt | |
| ctttgctgtt cacttctacc gctccctggt cagccataag acggaccggc agttccagga gctcaatgag ctggccgagt | |
| ttgcccgctt gcaggaccag ctggaccaca gagggg | |
| TC34186_at | |
| attcgaaaat cgattcttta tctaaaccta ctttagaaga tttaatatgt agtacgactg aagaaggaaa atatcaggct | SEQ ID NO. 24 |
| taccgaggag acagaaaaat tgcgtgctga aaatctattt ttgaaagaaa aaaagaagga attctaagga tcccagctat | |
| cttcatacac atcactttgt ttagattgat tctttaaaag tgggattgta tttgctcaca tggtagaact aaactaaaag | |
| accttaacta tgaatcatgg tgcgcactaa tcccgagtgt tccgatggat agtgtgcatg caaactcagc agcctgttaa | |
| gtaacaatgc attccaagtc atgtgtacat tctggaagac ccttaaaagt ctgtagctga ggtctctggt gaattctagt | |
| caggtccttt tcttcttttt ttatttactt tattttattt tttttttttt tggttttttg agacagggtt tctctgtata | |
| gctctggctg gtcttggaac taactaactc tgtagtccag cctggcctcg aactcagaaa tctgcctgcc tctgcctccc | |
| gagtgctggg a | |
| TC36089_at | |
| aaaagtcttt tcactttaat acaaacattg tataaagaaa agttgaatga gagagatcaa gtattcatca aatatcagtc | SEQ ID NO. 25 |
| aaggcgatga ttccatatgt tcctgctttt ccatcgctcc cggccacgtg ctaaggacca gataatcacg gaggcaggaa | |
| cgagccacag gccagaggcc tctgtccatg tgtggaggag gcgggatgca gccacacact ctgttccttt gctttctcca | |
| gttctcagta ttggcctcat ttggcctggc caaagcttac tgactgtcca ggtctgtccc cagagagcag gtgagccatg | |
| ggagggagca tcttcctctg actgctctgt gcaggagcaa gccaaggcac attcctgaca caggaaggtg gttccagaag | |
| gcatattggc aattgtcttc tcaagtcaag agggtcacca tcttggtgat ggttttcagt ctaggtatgc ttccctgacc | |
| ggaaagagaa cagcttcttc ataatggtcc tctcctcctt ctcgccgttg cccatggcaa ccacagcatc cttctctctt | |
| cttcctcctg gagtttctct gggtggaagt agaccactcc tgtagcaccg cagttcctcc tgcaccagca gcagctgcga | |
| cttgagcttg tttcgctcct gcagcacctc cctcagctct tgcagagtga agcggggacg gttggggtct gtcagatcca | |
| ctaccatctt gtccggtccc aagtttacct gggctccgga c | |
| TC36583_at | |
| taggagctag gctccaatga gcaagatcca acaactttat ttttctcaca catattgtat gtcccagcaa aatctttcca | SEQ ID NO. 26 |
| gtagtacaac catcataatg tggctatatt ctatttttct ttaagtggat gattacaatg actatacata agaaaaaaca | |
| tctttcccaa taccctcaca tgataaaatg ttttatgtat tctggataag caaaataaaa acaaattatt ctcaagaacc | |
| cttatacatt tgtaactaac caacttgtga tattgccaaa gtgtaagcaa ccaagataat tctctcaaaa ttattgacac | |
| acggcaatta ttttattatt catcttaaaa tgtaatctta taaaaaaatc ttaagagtat cacaaactta aaccttcata | |
| taaaaatcat ttttcctata aatccttagg gtgcacatct acttattaac aattttttat taaatcctgt caggaatctg | |
| agggtaagat gggtataaga tcttagtcat caccttgatc accagcgctg cttcagccaa acaggcatac ccacctcagc | |
| cagtattagc tgggttgcca ttgttcttgt ccctgacatc aaaagggtct agctcaacta tcaagagtgt gcctctctga | |
| atttccaatt gttccccacg gttttctact tccaagccgc tagcaaaagc atcttagcct aactttacta caaccgacat | |
| ttccacattc tttctaagag tgccggacat taccagcaat ctacagctct atgtcctttc tgagggtggt ggttgaatca | |
| gtaatctgct ccagctgcaa cacttgaaaa agagcaatca ctattcttga taccaatgat actgtccagt gaggggctgg | |
| cagccttttt ggagttttca tgcaactcac ggaccaagag ggatgtggtg atcttgaaga cggtgagcag agcaaaactc | |
| cacaacagca agaagtcctc aggacagtcg gggttctgcc tcttgatgtg gttgtgctaa ccggtgggac aaattccatg | |
| agttctaagg ctatttggtt ttcctcctgg gaccagtcct cttcctttct ttttatttct ttttctcata cacatggcgc | |
| cgaaaaccag gaccagtcat gttttctttc tttctttctc tttttcattc agccaagatg aaggattttg cccagaacca | |
| ctccgggtcc ccagaaaatg gcggcagagg tagtttgcag agtcatcaac tgggggcagg ctcctagaag aaacttccat | |
| gccgatgttt caatgtttcg ttttggtaac acaaactaac aggcagccac gaaaactctt gaccaaactc aatgcagtaa | |
| agagtgcatg ttatacaggg ctacgaatat gaagaggcct ccccccccag tgccgaat | |
| TC37631_at | |
| aattcggcac tgagggaaga aatctctgta ataccttttc ttccaagcta gacacctcac atcaggagtg cctatctggg | SEQ ID NO. 27 |
| ccaggattgg tctacttctg cctcttcagt gttctttccc aagcccacct agtcaggcgc cgcaggggct gcctcctact | |
| gtaccctgct ctgtggccca gcagaagaac actgtcacca tcacttacga agtaggaatc acagcgcaga gtgaccacat | |
| gaccaacagc agcagaaatg aatgaatctg aacagcatcc ctgaatcttc acaaaaacat actccaatcc ctaattgtct | |
| gcatctggca acctcagggt cagaaccggg gatttaagag aaaatgctaa gagctcacac ccatttcgca gcacttggga | |
| ggcagatgca gggggattac catgagttcg aagccagtct gaactacatc gtttgtttta tggtagtctg tgtaaaacag | |
| aatacttacg ccaccattca cacttcctat cttactttct aaataatggt tttaagagaa acacagtgtt tgctttactc | |
| tgaaagcgtg tttttattaa ttggacacac acaggtgaat gtttgatccc ggggctgtaa ctgtatttgt gtcagtgtcc | |
| atggagaggt gatgcctggg aggtcttgac tttcctccta gttgtcttct gctttctctg cacagttgtc atatatctac | |
| ctaaatataa tcacaaatgt a | |
| TC38094_at | |
| aatatataaa attatgtaaa agctatacta tattttgatt tagattttcc tgctgtttgt taccaaaaaa tttgtatttt | SEQ ID NO. 28 |
| aaatttgttt agctttagta tggttttgtc tacaatgagt atgtaattct tccttattaa atgaaaataa gggagcgaat | |
| tttgagatgt gattttaatg ctcccctatg gattcagtag atcaggccat tctctgctga agcctgtctg aactagaatt | |
| catccaaatt tatctctacc aagagtgggg ttatttgata ggttcgttta ggaaaaaaat ttgccccttt gttttttttc | |
| ttgagtcttt taagtaggag gctttaaaaa aaattgagca tttttttaag atcagaaata atgggcgttt ctcctgaaag | |
| catgattttc tccctctgtg aaacggcaga acattccagt gcttttattt ctttccttct ctcacgcgtg ctttaaaaaa | |
| aagaaaatgc cttaaatcat ttattttgct tatgccttct tgtaaatatg caaaggaagg atacgaattt taaggcccag | |
| gtctacggtt cgaaagaaca cacccagtaa atgtactata atgttgttaa agaccaaaaa gcacttcaaa gggagttgat | |
| agctcagaac tgttcttggt acgaggtggt cattgccctg tgagcatgcg tggtctgatg tcagttttaa agcaatgaag | |
| gaataaattg aaaaaaatcg ctgctgagta gatctgtgca ctaaagggga catttaaatg gaagcaccct cccgatcccg | |
| gtccagaccg gatgtacaca acggtctgtc tgcaaccgga tgtgaaactg gaaggacccc acgcaccctc ccagggtacc | |
| gcccagccct ctcatgtgtg attctgaatt taagtttcca gaagcctact ggctttttgt ctcgttgaga ttcttagata | |
| tcgattgtct tccaggctgc ttgagaatgg gcaaggacta gttaaatgga ttcaccatca gccactgggt cagattcagc | |
| ggtgtgatct ggaaaatgag cggcaaaggt tccacgtaga ggatcaagaa gtgaagtgac atggacaagc agatggagcc | |
| cacgagccag atattctccc aggggggcat cctcagcaaa gactggtttt cagacaagct gttgagggca ttacacatct | |
| ctatggttac tagaacagaa agtgccattg tcattggata tggggactca aagattgcac aatccactcc atcgaagtct | |
| gggttgtcct ccttacactg taggaaatga ctcagctggt agaaggagac tcttggaccg ccgtcagcag cgatgaacca | |
| ccatgcagca gcacccacgg tggcagcgcc aacataacag ccaatagcca ggtaacggaa aaagagccac ccgctgatca | |
| gtggttcttt tgggttccgg gggggtttgtt catgatgtc caggtctgga ggattgaacc ccagcgcagt ggcaggcaga | |
| ccatctgtca ccagattgac ccagagtaac tggacaggaa ttaaagcctc aggaaaccca agggctgccg tcaggaagat | |
| acagaccact tcccccacgt tggatgagat gaggtagcgg atgaactgct tcatgttgtt gtagatggct cgcccctcct | |
| caacagcagc cacaatggtg gagaagttgt catctgccag gaccatctca gaagcagtct tagccactgc agtccctgag | |
| cccatggcaa tcccgatttc agatttcttc agagcaggag catcattcac accatcacca gtcatagctg tgatctcatc | |
| aaaggactga aggaactcaa caatcttaga cttgtgggaa ggttcaactc gagcaaaaca gcgggcattt aagcaggcat | |
| ctctctgggc tgaagggctt aattcatcaa actctcgccc tgtaaaagcc tttgatgtca catcctcatc ctgcccaaag | |
| atgccaatgc ggcgacagat ggccacagcg gtgcctttgt tgtctccagt gatcatgatg acccggatgc ctgcttgccg | |
| gcacagcttc acagaagagg ctacttcaat cctgggagga tccagcatgc ccacacagcc gacgaaagtc aggttggtct | |
| cgtatttgat gaagttagca gagtcttcca ggtgcatctc ctctctcttc agtgggttgt catgagtggc cagagccagg | |
| caccgtagcg tgtcgctgcc actgccccac tcccgaatga cagacataat cttctgtttg acaccaggag tcatggggac | |
| cttggtactt ccaactcgga tgtgggtgca cctatcgatg acaccttctg gagccccctt cacaaacatc ttgctcatgg | |
| atgtccggct tggcttgttt ggggtacaat agacggacat tgattttcta tcccgtgaaa actccagagt gaactccttc | |
| ttcatcagct gctttatgac cgagttgcag gcgtttgcac gctctatttt agaaagcccc ttcagctcag tatcaaatac | |
| attcatcttc tccaccaggc acgtgagagc agtctcggta gcttctccaa ctttctcata cacacccttt gcctcattat | |
| aatccaaagc agagtcatta cacagagcac agatggtggc taactctaca agcccgtcat actgatggca cttcactggc | |
| ttatcatcct tttgcacttc tccaattggt gcatatgtgg atccagttat gctgaactca ttaagggaac aagtgtcacc | |
| ttctactttg tccagaatga acatcctgca cacggacatc tggtttgtgg taagtgtgcc tgtcttatct gagcagataa | |
| cagaagtaca accaagggtc tccacagaag gcagacttcg aacgatagca tttttctttg ccatcctacg agttccaaga | |
| gctaagcagg tggtgatgac agcaggcaga ccctcaggga ttgcggcaac agccagggcc acggcaatct taaagtagta | |
| gatggcaccc ctgatccagg agccaccatg aactgggtca ttgaaatgcc caatgttgat gatccagact gcaatgcaaa | |
| tgagggagat aactttggaa agctgctccc caaactcgtc tagcttctgg tgtaggggtg ttctctcctg ttctgttgcc | |
| accatttcat cccggatctt gccgatctca gtattaactc cagttgccac caccactccc atagctttcc cagcagcaat | |
| gtttgtacca gaaaagagca tgttcttttt gtcttgatta acagctcggg ggtcagggac agggtcagta tgcttgatga | |
| cggagacaga ttcacctgta agaattgact ggtcgactct tagagttgta gacttgatgg atgtcaatct aatatcagca | |
| ggaactttgt caccaacagc aatttccact atatcaccag gaactatgtc tttagcttta attcgttgca cactctttct | |
| gtcctgtcga tacactttgc ccatttcagg ctcatattcc ttaagagctt ctattgcatt ttcagcattt ctttcctgcc | |
| acacacccac gattgcattg gctaccaaga taagcagaat tacaaacggc tctacaaagg ctgtaatcgt ttcttcccct | |
| tcctcgaacc aagccaaaac gaaagatata catgctgcca gcagtaaaat tctaactagt aagtcttcaa actgctcaat | |
| cacaagttcc agcaaggttt ttccttcttc agccggcaat tcgttggagc cccatctctc cttgagcttc ttgacctgct | |
| ccaagctcag ccccgtgctc tcgttgaccc cgaagtggcc cagcacctcc tccacggtct ttgtgtgagc gttctccatg | |
| gctgcggggc cccggccggc ctcgcctcgc gtccccgcgg cctcctcgcc taccgcctcg cactccggcc gcgggctcgt | |
| gccaccgcgg gcgcccgggc gcggacagct gtcccctcct ttttcttctc ctcctcctcc tccgcggcgg cggcggccgc | |
| ttccgcctga ccggggcgct gaatcacccg agccccctcc cccagaaag | |
| TC38978_at | |
| tttttagagt tgctctcaac tgtttattgc taagctgtca tcatataagc cgtataaaaa tactttacat atagcaaaaa | SEQ ID NO. 29 |
| taaactgcag gaaacaggag attaaaatcg ttttgcatag gaatgtgata tatcccgact cctcaggtag tggggtggag | |
| aacttcccaa accccggcct tcagagcccg atgagcaggc taccatgagg gctgagaaat gacaggggga cggggatgtc | |
| tgatgtctgg gatgctctca cagaatgctg gggttcctac tcacatagca acagcactgt ctgcacccag tccatctaaa | |
| gcctgagagg gcagagccag gcagggccag gcagggcagg gacgtttatt cccatctgaa agcattaaca cttttgactc | |
| cagttcccag gacttcattg tgatctcaga ggggtcctgc tcagggaagc acca | |
| TC39012_at | |
| tgctggtggg atcaaagcgc agtgtcctgc ggcggggagc ttggaacgct aagaaaagtg accatggaga acaacaaaac | SEQ ID NO. 30 |
| ctcagtggat tcaaaatcca ttaataattt tgaagtaaag accatacatg ggagcaagtc agtggactct gggatctatc | |
| tggacagtag ttacaaaatg gattatcctg aaatgggcat atgcataata attaataata agaacttcca taagagcact | |
| ggaatgtcat ctcgctctgg tacggatgtg gacgcagcca acctcagaga gacattcatg ggcctgaaat accaagtcag | |
| gaataaaaat gatcttactc gtgaagacat tttggaatta atggatagtg tttctaagga agatcatagc aaaaggagca | |
| gctttgtgtg tgtgattcta agccatggtg atgaaggggt catttatggg acaaatgggc ctgttgaact gaaaaagttg | |
| actagcttct tcagaggcga ctactgccgg agtctgactg gaaagccgaa actcttcatc attcaggcct gccggggtac | |
| ggagctggac tgtggcattg agacagacag tgggactgat gaggagatgg cttgccagaa gataccggtg gaggctgact | |
| tcctgtatgc ttactctaca gcacctggtt actattcctg gagaaattca aaggacgggt cgtggttcat ccagtccctt | |
| tgcagcatgc tgaagctgta cgcgcacaag ctagaattta tgcacattct cactcgcgtt aacaggaagg tggcaacgga | |
| attcgagtcc ttctccctgg actccacttt ccacgcaaag aaacagatcc cgtgtattgt gtccatgctc acgaaagaac | |
| tgtactttta tcactagagg aatgattggg ggtggggggg ggcgtgtttc tgttttgtta tgccaaatga gaaagctgtc | |
| agggagactc tcatttaaat ctaatctgac ggtcctcctg gtctttgtac gctaccactg cctagcaatg cagccagcca | |
| cagtgcagct acctcaactt cgacatcagg tagttgaaat gaaatttaat ttaataagga gcaagtaact gtcaatgatg | |
| gtactatcat cctagatgaa attacaaagt tgccctttta taattagcaa gatttggcga tactatgaat tttgaagtca | |
| ttttgaagca gtacagcttt tccactgatg attttatact ccccactcat gttaagaatg ttgttctagt tttcgttaaa | |
| cgtagaaaca ataatgtcaa atgataatgt cttagaactt gaatccatga gcagagtcaa aggctggaac ccttgttttg | |
| gacctgattt atggaagtga agagttggac caccatagca tgcattatag ctactggttt tgtgacagtt gtccacaaca | |
| cgtatttgtt attttaaaaa aaaaaaatca tgttttatta ctggaacaaa aataacttga gtatttatgt gagaggaaaa | |
| ccgtgtgcga acttggttga ctcattgaag gcaaggtgct aaaatccatc agcatttgtt tcttacttat ccttatacaa | |
| atcagctcgg tcttacagac cagcaaaata tctcaagagc tgttatgtaa cagcctcaaa tggttgatta ttagaatgaa | |
| gtacaggaat tctataggaa tatactataa taccagttgt caaacagagc ttaagaaaag gagagatgaa atctactgta | |
| gttggagaga atgtggtttg agccagagca gagacttggc agagcatggg cacatcttca gaaaaggcct gccagctgga | |
| ctccccaccc ctcaccccac cccccttgtg agttctggtt tgtgtggatg atgtccaagt taccacgtgg gaaagtgaac | |
| cagatcacaa acttctgcaa aaaggctttc ccagagggct gagagtctga agaaagttca ctggactcca tgtccagagt | |
| ctgtagcatg ctgacttttc tgttgacgtt tacaaagaaa ggaaaaataa tggtgatatt tatactgtgc tagaaacgaa | |
| agggccaggg cacacaggac ttggaaagca tccagcaata gccctgcagc ccatgtggag aagctgcggg ctgcagtcca | |
| tcctcaggga gactgtgctc tgctcctggc ctccttatgg gggtgctgag tcactgctgg ctctcgcagc atttctccta | |
| agcttccttg ttgcacaaag aaaggaagga actcacattg taacggatag attcttgttt agactggctt tgttgttaac | |
| gctttctaag tgaatgcagc atgtgatggt attttaaagt gtgttcccat gtgacaattt tataaaagtt tgttaccttg | |
| caattttatt tcaaaacata aattcagatt taaaattag | |
| TC39080_at | |
| ggaagtcgac cagcacaagg cagagtcctg gacagaacaa aacctgcctg ctacaggaca gagcttgtgt ttgaatgcat | SEQ ID NO. 31 |
| gtacatatag gcatatattt atgtatagat atccaaggac agaggtggca atgagcagtc tgtgcccacc aggggccttc | |
| cttccatgtt agcaataacc agtatccacc ttgagagtgc acctcagagt ccagaaccgg cttcccacca tcatggtcttt | |
| ccttccatc agggtctacc tctggacagg caggtggcct tttcttgcct cacgcagtac ctgagcactc cagagctgtg | |
| ctcaaatgcc catcagctac caaatggcaa aatctgaaag tggttgtaaa taaccattac agaatgagtg tagtatattt | |
| gttcaattat aagattattc tttcacagaa gccttatagc tctctgcttc atctaagaaa acaattacca aaaaaaacaa | |
| cgtttctaac tgcaatctgt gaactgtgcg ttttcagatt ggttactggt aacagataag ctggtgtctg ctctgtgtaa | |
| ttagctgctt acttcagtta ctagcagtga cctattattt cttataacca aaaaaagcat ggtttaatta aaacatgttt | |
| aatgatcgtg ccttaggagt taatgccccc ttatggaaca cgcctgaatt gcacctgtgg ctggaagttt taagttactc | |
| ccagacagat ggactcatga caggaaaagc tctctcacag gaagatgcat ctttaaaatt tttgtcagtc tgtatgatgg | |
| tggcttacct ttcccaacgc acagaaagaa acaactgtct gaaagcatac tgaatgattt cgcacgactg tgaagagctg | |
| gcgcgaactg ccttgtacac acatagctcc tggccgcctg caggctgcct cccgcctgcc tctcgtctgt accccatgtt | |
| tattagcatc atggagttgc atgaaccatt cttagtagac tgtcatctga aagcaagcgt ctgatatttg tgtcagctat | |
| ctttgtagtt aggagatgaa tccaataaag cagtattttt ttcttttttt | |
| TC39762_at | |
| tttgggggtc actcgacttg attaatttta ttctacaaaa tgctactcag tggaagtagg aaagctaaca aaacaacaac | SEQ ID NO. 32 |
| aacaaaaaca taaaacgaga acaaacccga gggaaaataa gtttttaata tgttctttcc tccatagcag caagctccta | |
| ccaagctttt cttagtgcaa atcctgtagg cttgtgtcac atacagtaca cagaacaaca catcacacca ccacagatgc | |
| ttccgagcag agatactcct caaaaattta aaactataca aagatttctg agcataaggt cctgcctgga gagttcaact | |
| agagcgaccc tcctagggcc gtttcaccgt taatttaaaa gtaggggaca aaaggtgccc agaaaggaaa ttaaattccc | |
| cgcggagcca taaaaccttg tacaacccat ttgcctccag gatctaatag caaatttcac tccacgtcat tgacatatac | |
| caaatacaga tgcatgaagc ttgggtccta ctctatacca aaatacgata tatacacctc ccactgcaaa aggaatctga | |
| tacctagtct ttatacaaag ctgaatattt tcttcctcaa aatcaagtaa ccacaaagta aaataaatgg aatatttttt | |
| taaaaaaaaa atcatacaga gaaagttaag ttttgagaga cagggccagg gtctttcatt gtcttctctt acaatgtaga | |
| tttctcagta gccactgtcc ccacaggaat gacaattgat ctttaaaaac agagcctttt taatacagtt tatacagcac | |
| aagtccacaa gtcacttgag aaacaaacaa aatagagatc attatcctaa gtcagaacaa gtgggggaga gagagccaga | |
| gaaaggagtg ggaaggaagt actttaatgc tatctgtttc tattcaggct tggaacaaca caaagaaatg tacttctgtc | |
| gtcttctc | |
| TC40487_g_at | |
| ggaagcccaa gatggaccaa ccactgctgt gggctgatgg ccttagcagc cacataatcc tccgtgtgat gtccagggtc | SEQ ID NO. 33 |
| agtgcagtcc tggcccacca gggctaggag aaggtatatg agaatctcac tgtgatctgt cctctttaga acagcatttc | |
| agtcaccacc tgggccgatg ctctgtccat gtgttccttc accagctcaa agcacctgtg ggctagctgc tgcagcctgg | |
| cgtcctcact caccatcagc tccccataca ggtatatgcc catggcctgg tcctccagta ggaacctctc cacctgattg | |
| tatgcctttg tgtacttgtg cttaataacc agttcacacc atcgatggcg aacctctgca tcctgctctg ggagatggta | |
| agtctgctgg agacagtgca gtgtctgagg gctcagcgtc ttctgctcta agagccactc caaaagcaag acgatctggt | |
| ctggagaaag cttttcaaag gcgacttctc gcttccctcg tttgcgtttt cggggtctgc ggttgactcg aatccatttc | |
| gccacctcgg agcctacttg ccgtgcgagc tactcggcga gcagtcctcg gccttgcgct cctcctgcag cgcctgtggg | |
| gagaacaagt caagttcacc gagcccag | |
| TC41014_at | |
| agcccagatc ctagcagtag tcacacttca aagtccagcc atttctatga gataaaagga cacaccacag atggagaact | SEQ ID NO. 34 |
| ttagaagaag tttgactaga aaaatcattg gctgtgtagc gtgaattttt atcagtgaag aaatatacct ggattctttc | |
| atagtggcta tattaactgg ctaaaagttt tgtcatttag tccattaata ggctaaaatg attttttaaa atgaatccac | |
| aaaactgtca tttaaaacac tacatatgtg gcaacccaaa attagtgttc atcagcctaa gggcacaggg aagatactgg | |
| ccataatttt aattcataag aataaatttt aacttttaag gccaaaaagc aaaataatta aaatattttg agttgtttaa | |
| aacaaatcta gatcataact ggaaaaagaa gtcatcacta ttatttttta acccaaataa aaatatgtga tctaacaata | |
| agacaggaaa ctaagacatc acagtgatat attgtggaaa ggtgagtgat cagtcacatt cagatttctg catgttgaaa | |
| tacaacaacc agctgactgt atccctctct ccactagtta tggcattgtg taggcagggg ctagggatgc tatctacaga | |
| tatgaagaac tccttaaaaa tccttcttcc ggacactcaa aatacaatgg aaactttggt ccagacaatc tggaccagaa | |
| ggtgaattcg taacttcctt atcccattaa atattgtaaa agagacagaa taagaaatgt ttctctttgg aaatacgtat | |
| tttgaatgca actatgaaag ataatggtgc ataattattt cctttcaact taatgcttaa tacagaagta aaagtctacc | |
| tgccttgctg aattgaaaag aacaaatcca ttatatagcc atatttctcc at | |

By “NFAT protein” or “NFAT” (nuclear factor of ctivated T cells) is meant a member of a family of transcription factors comprising the members NFAT1, NFAT2, NFAT3 and NFAT4, with several isoforms. Any other NFAT protein whose activation is calcineurin dependent is also meant to be included. NFAT proteins can be, e.g., mammalian proteins, e.g., human or murine. NFAT1, NFAT2 and NFAT4 are expressed in immune cells, e.g., T lymphocytes, and play a role in eliciting immune responses. NFAT proteins are involved in the transcriptional regulation of cytokine nucleic acids, e.g., IL-2, IL-3, IL-4, TNF-α and IFN-γ, during the immune response.

cDNA sequences for NFAT have been previously reported. The published sequences for human NFAT2 represent two isoforms differing by alternative splicing at the N and C termini, but having the same regulatory domain and DNA-binding domain. The two published sequences for murine NFAT4 are not identical.

NFAT proteins have been shown to be direct substrates of calcineurin. Calcineurin is a calmodulin-dependent, cyclosporin A (“CsA”)-sensitive and FK506-sensitive, phosphatase. Calcineurin is activated through its interaction with Ca²⁺ activated calmodulin when intracellular calcium levels are elevated as a result of receptor (e.g., TCR) crosslinking and phospholipase C activation. The activated calcineurin in turn activates NFAT from an inactive cytoplasmic pool. NFAT activation involves a protein-protein interaction between calcineurin and NFAT, dephosphorylation of NFAT by calcineurin, a conformational change in NFAT (resulting from the interaction between calcineurin and NFAT or the dephosphorylation of NFAT), and translocation of NFAT to the nucleus. NFAT activation results in induction of nucleic acid expression.

NFAT-mediated nucleic acid expression programs include at least two modes referred to herein as “NFAT signaling” and “NFAT-NFAT ligand signaling.” “NFAT signaling” or “NFAT-mediated immune response” refers to a calcium-triggered cascade of signal transduction events that leads to NFAT activation, without substantial expression and/or activity of an NFAT ligand, where an “NFAT-ligand” is defined as a protein or transcription factor that interacts physically or functionally with NFAT during the course of a complete or productive immune response. For instance, an NFAT ligand includes the complete set of transcription factors that are turned on during a productive immune response and cooperate physically or functionally with NFAT. Because these transcription factors may also interact physically (e.g. AP-1) or functionally (e.g. NFκB/Rel) with NFAT, they may sometimes be referred to hereafter as “NFAT ligands.” Such NFAT-specific activation may result from, e.g., activation of a T cell receptor in the absence of costimulatory receptor stimulation (e.g., CD28), or by an increase in intracellular calcium concentration (e.g., using a calcium ionophore, such as ionomycin). These events lead to calcineurin-mediated activation of NFAT. NFAT-specific activation gives rise to expression of one or more nucleic acids, some or most of which may encode polypeptide effectors of the anergic or tolerant state.

“NFAT-NFAT ligand signaling” refers to a cascade of signal transduction events that leads to NFAT and NFAT ligand activation. This coactivation results from, e.g., costimulation of a T or a B cell receptor and a costimulatory receptor (e.g., CD28 or CD19), which in turn activate calcium calcineurin- and protein kinase C-dependent pathways. Coactivation can be induced by administration of a calcium ionophore, such as ionomycin, and a phorbol ester, such as PMA. The term “NFAT ligand” refers to a protein, or a complex of proteins (e.g., a protein dimer) that interacts, e.g., binds to, NFAT and leads to a complete productive immune response, including expression of cytokine nucleic acids, cell proliferation, and prevention or minimization of anergy or tolerance. In one embodiment, the NFAT ligand is a CD28-activated transcription factor, such as AP-1 (e.g., Fos/Jun, Jun/Jun dimers which interact physically with NFAT on specific composite NFAT: AP-1 DNA elements) or NFκB/Rel, which interact functionally with NFAT at other gene regulatory regions).

The costimulatory receptors CD28 and CD19 are present on T and B cells, respectively. CD28 forms a transmembrane homodimer that is present on most T cells and binds to a B7 ligand, e.g., B7-1 (CD80) or B7-2 (CD86), present on antigen presenting cells (APC), such as B cells. B7 family members are typically produced in response to foreign infection. Stimuli that lead to upregulation of B7 proteins include structural components of bacteria, such as lipopolysaccharides, antigen binding to B cells, and TNFα. When a T cell is acutely stimulated by an antigen through its T cell receptor, and at the same time costimulated by a B7 protein through the CD28 co-receptor, the combined signal stimulates the T cell to produce IL-2 and to proliferate.

CD19 has a similar costimulatory role as CD28 in B cells. Like CD28 on T cells, activation of the CD19 costimulatory receptor complex changes the outcome of antigen-receptor ligation. CD19 is a transmembrane protein made constitutively by B cells. Alone CD19 may act as a receptor for an as yet unidentified ligand, but in association with the complement-binding chain CD21, CD19 forms the signaling subunit of the CR2 complement receptor. The complement system is a proteolytic cascade of interacting serum proteins that is selectively triggered by foreign microorganisms. Once triggered, cleavage products of the third complement component, C3b and C3d, are covalently attached to foreign antigens, tagging them for destruction (C3b) or for immune responses (C3d). When a B cell is acutely stimulated through its B cell receptor and simultaneously costimulated by attached C3d via its complement receptor complex, the combined signal synergistically augments B cell activation and antibody production.

A “costimulatory blocker” or a “costimulatory inhibitor” as used herein, refers to a molecule which binds a member of a ligand/counter-ligand pair (e.g., CD28/B7, CD19/ligand) and inhibits the interaction between the ligand and counter-ligand or which disrupts the ability of the bound member to transduce a signal. The blocker can be an antibody (or fragment thereof) to the ligand or counter ligand, a soluble ligand (soluble fragment of the counter ligand), a soluble counter ligand (soluble fragment of the counter ligand), or other protein, peptide or other molecule which binds specifically to the counter-ligand or ligand, e.g., a protein or peptide selected by virtue of its ability to bind the ligand or counter ligand in an affinity assay, e.g., a phage display system.

The term “tolerance,” as used herein, refers to a down-regulation of at least one element of an immune response, for example, the down-regulation of a humoral, cellular, or both humoral and cellular responses. The term tolerance includes not only complete immunologic tolerance to an antigen, but to partial immunologic tolerance, i.e., a degree of tolerance to an antigen which is greater than what would be seen if a method of the invention were not employed. “Cellular tolerance,” or “anergy,” refers to down-regulation of at least one response of an immune cell, e.g., a B a T cell. Such down-regulated responses may include: decreased proliferation in response to antigen stimulation; decreased cytokine, e.g., IL-2, production, among others.

As used herein, the term “anergy polynucleotides” or their corresponding polypeptide products are those whose expression is modulated (e.g., increased or decreased) in response to NFAT signaling, e.g., an ionomycin-induced and calcineurin-dependent response.

As used herein, the terms “marker” or “anergy marker” are used interchangeably, and include a polynucleotide or polypeptide molecule which is modulated (e.g., increased or decreased) in quantity or activity in subejcts afflicted with immune disorders (e.g., T cell disorders, B cell disorders, autoimmune disease, infectious disorders, transplant rejection, cancer and proliferative disorders) as compared to a subject not afflicted with the immune disorder. In certain embodiments, the anergy markers of the invention include the markers listed in Group I or Group II or Group III or Group IV, as well as homologs or isoforms thereof, particularly human homologs or human isoforms.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA. Additionally, the terms “polynucleotide,” “nucleic acid” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers. Polynucleotides of the invention may be naturally-occurring, synthetic, recombinant or any combination thereof. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) in place of guanine when the polynucleotide is RNA. Thus, the term “polynucleotide sequence” is the alphabetical representation of a polynucleotide molecule. This alphabetical representation can be inputted into databases in a computer and used for bioinformatics applications, such as functional genomics and homology searching.

The term “noncoding region” includes 5′ and 3′ sequences which flank the coding region that are not translated into amino acids (i.e., also referred to as 5′ and 3′ untranslated regions).

The term “isolated or purified nucleic acid molecule” or “biologically active portion thereof” includes nucleic acid molecules which are separated from or substantially free of cellular material or other nucleic acid molecules which are present in the natural source of the nucleic acid, or substantially free from chemical precursors or other chemicals when chemically synthesized. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

The language “substantially free of cellular material” includes preparations of marker protein in which the protein is separated from cellular components of the cells from which it is isolated or recombinantly produced. In one embodiment, the language “substantially free of cellular material” includes preparations of marker protein having less than about 30% (by dry weight) of non-marker protein (also referred to herein as a “contaminating protein”), more preferably less than about 20% of non-marker protein, still more preferably less than about 10% of non-marker protein, and most preferably less than about 5% non-marker protein. When the marker protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of marker protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or non-protein chemicals, more preferably less than about 20% chemical precursors or non-protein chemicals, still more preferably less than about 10% chemical precursors or non-protein chemicals, and most preferably less than about 5% chemical precursors or non-protein chemicals.

An “isolated” or “purified” protein or biologically active portion thereof is substantially free of cellular material or other contaminating proteins from the cell or tissue source from which the marker protein is derived, or substantially free from chemical precursors or other chemicals when chemically synthesized.

As used herein, a “comparison to a reference profile” also includes comparison to a plurality of reference profiles. Non-limiting examples of such comparisons include comparisons to an average of a plurality of reference profiles, a range formed by a plurality of reference profiles, or a region multi-dimensional space, e.g., occupied by a plurality of reference profiles.

As used herein, a marker “chimeric protein” or “fusion protein” comprises an anergy marker polypeptide operatively linked to a non-marker polypeptide. A “marker polypeptide” includes a polypeptide having an amino acid sequence encoded by an anergy marker set forth in Group I or Group II or Group III or Group IV, whereas a “non-marker polypeptide” includes a polypeptide having an amino acid sequence corresponding to a protein which is not substantially homologous to the marker protein, e.g., a protein which is different from the marker protein and which is derived from the same or a different organism.

As used herein, a “biologically active portion” of a marker protein includes a fragment of a marker protein comprising amino acid sequences sufficiently homologous to or derived from the amino acid sequence of the marker protein, which include fewer amino acids than the full length marker proteins, and exhibit at least one activity of a marker protein. Typically, biologically active portions comprise a domain or motif with at least one activity of the marker protein. A biologically active portion of a marker protein can be a polypeptide which is, for example, 10, 25, 50, 100, 200 or more amino acids in length. Biologically active portions of a marker protein can be used as targets for developing test compounds which modulate a marker protein-mediated activity.

“Differentially” or “abnormally” expressed, as applied to a gene, includes the differential production of mRNA transcribed from a gene. A differentially or abnormally expressed gene may be overexpressed or underexpressed as compared to the expression level of a normal cell or control cell. In one aspect, abnormal or differential expression refers to a level of expression that differs from normal levels of expression by one normal standard of deviation. In a preferred aspect, the differential is 2 times higher or lower than the expression level detected in a control sample. The term “differentially-” or “abnormally-” expressed also includes nucleotide sequences in a cell or tissue which are expressed where silent in a normal cell or control cell.

As used herein, the term “aberrant” includes a marker expression or activity which deviates from the normal marker expression or activity. Aberrant expression or activity includes increased or decreased expression or activity, as well as expression or activity which does not follow the normal developmental pattern of expression or the subcellular pattern of expression. For example, aberrant marker expression or activity is intended to include the cases in which a mutation in the marker polynucleotide causes the marker polynucleotide to be under-expressed or over-expressed and situations in which such mutations result in a non-functional marker protein or a protein which does not function in a normal fashion, e.g., a protein which does not interact with a marker ligand, e.g., NFAT, or one which interacts with a non-marker protein ligand. In certain embodiments the normal cell or control cell or sample is substantially free of an immune disorder.

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation or suppression.

A “probe” when used in the context of polynucleotide manipulation, includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set or primers” consisting of “upstream” and a “downstream” primer, and a catalyst of polymerization, such as a DNA polymerase, and typically a thermally-stable polymerase enzyme. Methods for PCR are well known in the art. All processes of producing replicate copies of a polynucleotide, such as PCR or gene cloning, are collectively referred to herein as “replication.” A primer can also be used as a probe in hybridization reactions, such as Southern or Northern blot analyses.

The term “cDNAs” includes complementary DNA, that is, mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A “gene delivery vehicle” includes a molecule that is capable of inserting one or more polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy as well as for simply polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above functions.

A “host cell” is intended to include any individual cell or cell culture which can be or has been a recipient for vectors or for the incorporation of exogenous polynucleotides and/or polypeptides. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, including but not limited to murine, rat, simian or human cells.

The term “genetically modified” includes a cell containing and/or expressing a foreign or exogenous gene or polynucleotide sequence which, in turn, modifies the genotype or phenotype of the cell or its progeny. This term includes any addition, deletion, or disruption to a cell's endogenous nucleotides.

As used herein, “expression” includes the process by which polynucleotides are transcribed into RNA and translated into polypeptides or proteins. Methods of measuring expression are known in the art and include, for example, detection of the presence of an RNA species transcribed from a specific gene. For example, expression of the marker caspase-3 would include detection of caspase-3 RNA transcripts in immune cells from a subject. If the polynucleotide is derived from genomic DNA, expression may include splicing of the RNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG. Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

As used herein, a “test sample” includes a biological sample obtained from a subject of interest. For example, a test sample can be a biological fluid (e.g., blood, T cells,), cell sample, or tissue (e.g., spleen, lymph nodes, lung tissue).

As used herein, “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table A below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE A
Stringency Conditions

| | Poly- | | | |
|---|---|---|---|---|
| Stringency | nucleotide | Hybrid | Hybridization Temperature and | Wash Temperature |
| Condition | Hybrid | Length (bp)¹ | Buffer^{H} | and Buff^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | DNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A ⁺ T bases) ⁺ 4(# of G ⁺ C bases). For hybrids between 18 | | | | |
| # and 49 base pairs in length, Tₘ(° C.) = 81.5 ⁺ 16.6 (log₁₀Na⁺) ⁺ 0.41(% G ⁺ C) − (600/N), where N is the number of bases in the hybrid, and Na⁺ is the concentration of sodium ions in the hybridization buffer (Na⁺ for 1xSSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

The term “immunospecific” refers to antibodies that have at least one hundred-fold greater affinity for the antigen of interest, (e.g., a protein encoded by an anergy marker listed in Group I or Group II or Group III or Group IV, or homologs thereof or fragment), than any other protein.

As used herein, the term “normal” refers to cells, tissues or other such samples taken either pre-disorder or from a subject who has not suffered an immune disorder, or from a cell, tissue or sample that is substantially free of an immune disorder. Control samples of the present invention are taken from normal samples. As used herein, a “control level of expression” refers to the level of expression associated with control samples thereof.

As used herein, the term “therapeutic target” refers to a polypeptide or polynucleotide or a biochemical complex, e.g., an enzyme-substrate complex, a receptor-ligand complex or a protein-antibody complex, which is the subject of diagnostic manipulation for treating or preventing injury caused by an immune disorder. In the present invention, the therapeutic targets are the subject of manipulation in assays or treatments for inhibiting immune disorders. More specifically, the therapeutic targets of the invention may include transcription factors and polynucleotides, cell surface receptors and their ligands, as well as molecules involved in antigen processing and presentation, calcium regulation or metabolism, carbohydrate metabolism, cell cycle regulation, cytoskeleton, lipid metabolism, general metabolism, nucleotide metabolism, protein metabolism, or signaling. The therapeutic targets of the invention may also include a molecule that is a small G protein, a secreted protein, a kinase, or a molecule with unknown function. In certain embodiments, the present invention is directed to orphan receptors where the cognate ligand has yet to be identified.

As used herein, the term “panel of markers” includes a group of markers, the quantity or activity of each member of which is correlated with the incidence or risk of incidence of an immune disorder described herein. A panel of markers comprises 2 or more markers. A panel may also comprise 2-5, 5-15, 15-35, 35-50, 50-100, or more than 100 markers. In certain embodiments, a panel of markers may include only those markers which are abnormally increased or abnormally decreased in quantity or activity in subjects having or suspected of having an immune disorder. In a preferred embodiment, the panel of markers comprises at least 2 markers, preferably 5, more preferably 10, still more preferably 15 of the markers listed in Group I or Group II or Group III or Group IV.

Various aspects of the invention are described in further detail in the following subsections. The subsections below describe in more detail the present invention. The use of subsections is not meant to limit the invention; subsections may apply to any aspect of the invention.

Anergy Markers

As shown in the Examples and Figures below, expression levels of polynucleotides indicative of anergy are detected and compared in tissue after treatment with ionomycin, or with a combination of ionomycin and/or PMA or CsA. The polynucleotides listed herein as referred to as “anergy markers.” While animal subjects are provided in the present invention for a more detailed analysis of immune disorders, it is well-appreciated in the art that expression levels of polynucleotides and genes in animal models can be interpreted to reflect expression levels from human subjects as well. It is specifically intended by the invention and understood that the anergy markers of the invention also specifically encompass human homologs of the anergy markers listed in Group I and Group II, some homologs of which are listed in Group III and Group IV. However, the invention is also intended to include homologs of the anergy markers that are not listed in Group III or Group IV, as well as homologs of the polynucleotides listed in Group III or Group IV. Markers from other organisms may also be useful in the use of animal models for the study of immune disorders and for drug evaluation. Markers from other organisms may be obtained using the techniques outlined below.

In one aspect, the present invention is based on the identification of a number of genetic markers, set forth in Group I or Group II or Group III or Group IV, which are differentially expressed in anergic or tolerised cells. These markers may, in turn, be components of disease pathways and thus may serve as novel therapeutic targets for treatment in immune disorders. The expression levels of polynucleotides that were differentially expressed between anergic tissues or cells are set forth in Group I or Group II or Group III or Group IV. In general, Group I and Group II provides anergy markers which are expressed at abnormally increased or decreased levels in anergic tissues or cells and represent anergy immune disorder-related polynucleotides. These polynucleotides may be a component in the disease mechanism and be novel therapeutic targets for the treatment and prevention of the immune disorders provided herein. In general, Group III and Group IV provide human homologs of anergy markers listed in Group I and Group II.

Polynucleotides listed in Group I or Group II or Group III or Group IV were found to be differentially expressed in anergic tissue or cells. These polynucleotides and their corresponding gene products (and detectable fragments thereof) are referred to herein as “anergy markers.”

The polynucleotides which are known in the art to be linked to anergy may also serve as validation in expression studies for anergy-related immune disorders in conjunction with the anergy markers of the invention. Two markers that were known prior to the invention to be associated with anergy-related immune disorders are RGS-2 and Ikaros. These markers are not to be considered as anergy markers of the invention. However, these markers may be conveniently used in combination with the markers of the invention (i.e., those anergy markers listed in Group I or Group II or Group III or Group IV) in the methods, panels, kits and compositions of the invention.

Accordingly, the present invention pertains to the use of the markers listed in Group I or Group II or Group III or Group IV, polynucleotides, and the encoded polypeptides as markers for anergy-related immune disorders. Moreover, the use of expression profiles of these genes may indicate the presence of or a risk of an immune disorder. With respect to an immune disorder, these markers are further useful to correlate differences in levels of expression with a poor or favorable prognosis. In particular, the present invention is directed to the use of markers and panels of markers set forth in Group I or Group II or Group III or Group IV, or homologs thereof, such as human homologs. For example, panels of the markers can be conveniently arrayed on solid supports, i.e., biochips, such as the GeneChip®, for use in kits. The anergy markers can also be useful for assessing the efficacy of a treatment or therapy of an immune disorder, or as a target for a treatment or therapeutic agent.

Therefore, without limitation as to mechanism, the invention is based in part on the principle that modulation of the expression of the anergy markers of the invention may ameliorate an immune disorder when they are expressed at levels similar or substantially similar to normal (non-diseased) tissue.

In one aspect, the invention provides anergy markers whose level of expression, which signifies their quantity or activity, is correlated with the presence of an immune disorder. The anergy markers of the invention may be polynucleotides (e.g., DNA, cDNA or mRNA) or peptide(s) or polypeptides. In certain preferred embodiments, the invention is performed by detecting the presence of a transcribed polynucleotide or a portion thereof, wherein the transcribed polynucleotide comprises the anergy marker. Alternatively, detection may be performed by detecting the presence of a protein which corresponds to (i.e., is encoded by) the marker gene or RNA species.

In another aspect of the invention, the expression levels of the anergy markers are determined in a particular subject sample for which either diagnosis or prognosis information is desired. The level of expression of a number of markers simultaneously provides an expression profile, which is essentially a “fingerprint” of the presence or activity of a marker or plurality of markers that is unique to the state of the cell. In certain embodiments, comparison of relative levels of expression is indicative of the severity of an immune disorder, and as such permits for diagnostic and prognostic analysis. Moreover, by comparing relative expression profiles of anergy markers from tissue samples taken at different points in time, e.g., pre- and post-therapy and/or at different time points within a course of therapy, information regarding which polynucletodies are important in each of these stages is obtained. The identification of markers that are abnormally expressed in tissue afflicted with an immune disorder versus normal tissue, as well as differentially expressed markers during a severe immune disorder, allows the use of this invention in a number of ways. For example, in the field of immunology, comparison of expression of anergy marker profiles of various disease progression states provides a method for long term prognosing, including survival. In another example, the evaluation of a particular treatment regime may be evaluated, including whether a particular drug will act to improve the long-term prognosis in a particular patient.

The discovery of these differential expression patterns for individual or panels of anergy markers allows for screening of test compounds with the goal of modulating a particular expression pattern; for example, screening can be done for compounds that will convert an expression profile for a poor prognosis to one for a better prognosis. In certain embodiments, this may be done by making biochips or arrays comprising sets of significant anergy markers, which can then be used in these screens. These methods can also be done on the protein level; that is, protein expression levels of the immune disorder-associated proteins can be evaluated for diagnostic and prognostic purposes or to screen test compounds. For example, in relation to these embodiments, significant anergy markers may comprise markers which are determined to have modulated activity or expression in response to a therapy regime. Alternatively, the modulation of the activity or expression of an anergy marker may be correlated with the diagnosis or prognosis of an immune disorder. In addition, the markers can be administered for gene therapy purposes, including the administration of antisense nucleic acids, or proteins (including marker polypeptides, antibodies to a marker polypeptide and other modulators of marker polypeptides) administered as therapeutic drugs.

For example, the anergy marker caspase-3 shows increased expression in anergic tissue samples, relative to control tissue samples. The presence of increased mRNA for this marker (or up-regulated anergy markers listed in Group I or Group II or Group III or Group IV), or increased levels of the protein products of this marker (and other up-regulated anergy markers set forth in Group I or Group II or Group III or Group IV) serve as markers for immune disorders. Accordingly, modulation of up-regulated anergy markers, such as caspase-3, to normal levels (e.g. levels similar or substantially similar to tissue substantially free of immune disorder) allows for amelioration or inhibition of an immune disorder. Preferably, for the purposes of the present invention, increased levels of the up-regulated anergy markers of the invention are increased by an abnormal magnitude, wherein the level of expression is outside the standard deviation for the same marker as compared to normal tissue or cells. Most preferably, the up-regulated anergy marker is enhanced or increased relative to normal tissue samples by at least 2-, 3-, or 4-fold or more. Alternatively, the up-regulated anergy marker is modulated to be similar to a control sample which is taken from a subject or tissue or cell which is substantially free of an immune disorder. One of skill in the art will appreciate the application of such control samples.

As another example, the polynucleotide designated Msa.1669.0_f_at (GDP Dissociation Inhibitor Beta) has decreased expression in anergic tissue samples relative to control tissue samples. The presence of decreased mRNA for this marker (and for other down-regulated anergy markers set forth in Group I or Group II or Group III or Group IV), or decreased levels of the protein products of this gene (and for other down-regulated anergy markers set forth in Group I or Group II or Group III or Group IV) serve as markers for immune disorders. Accordingly, modulation of down-regulated anergy markers to normal levels (e.g. levels similar or substantially similar to tissue substantially free of an immune disorder) or levels decreased as compared to control tissue allows for amelioration of immune disorders. Preferably for the purposes of the present invention, decreased levels of the down-regulated anergy markers of the invention are decreased by an abnormal magnitude, wherein the level of expression is outside the standard deviation for the same marker as compared to control tissue. Most preferably the marker is decreased relative to control samples by at least 2-, 3- or 4-fold or more. Alternatively, the down-regulated anergy marker is modulated to be similar to a control sample which is taken from a subject, tissue, or cell, which is substantially free of an immune disorder. For example, the polynucleotide Msa.1669.0_f_at (GDP Dissociation Inhibitor Beta), which is involved in modulating small G protein activity, is down-regulated in anergic tissue. One of skill in the art will appreciate the application of such control samples.

In another embodiment of the invention, an anergy marker can be used as a therapeutic compound of the invention, or may be used in combination with one or more other therapeutic compositions of the invention. Formulation of such compounds into pharmaceutical compositions is described in subsections below. Administration of such a therapeutic may induce suppressive bioactivity, and therefore may be used to ameliorate or inhibit an immune disorder.

One of the skill in the art will recognize other controls such as by using different time points, other polynucleotides, or the presence or absence of a test compound. One of ordinary skill in the art will appreciate that other post-activation time points may be used to assess expression levels of tissue afflicted with an immune disorder. For example, post-activation time points include but are not limited to 2 h, 6 h, 8 h, 12 h, 15 h, 16 h, 20 h, 24 h, 36 h, 48 h, 72 hours. One skilled in the art will be cognizant of the fact that a preferred detection methodology is one in which the resulting detection values are above the minimum detection limit of the methodology.

Sources of Anergy Markers

The polynucleotides and polypeptide markers of the invention may be isolated from any tissue or cell of a subject expressing the markers. In a preferred embodiment, the tissue is from blood, lymph nodes, spleen or lungs. However, it will be apparent to one skilled in the art that tissue samples, including bodily fluids, such as, for example, blood or lymph, may also serve as sources from which the markers of the invention may be assessed. The tissue samples containing one or more of the markers themselves may be useful in the methods of the invention, and one skilled in the art will be cognizant of the methods by which such samples may be conveniently obtained, stored and/or preserved.

Methods of Treatment of Immune Disorders

The present invention provides for both prophylactic and therapeutic methods of treating a subject at risk for, susceptible to or diagnosed with an immune disorder. The molecules of the invention, e.g., agents, described herein have therapeutic utilities. For example, these agents can be administered to cells in culture, e.g. in vitro or ex vivo, or in a subject, e.g., in vivo, to treat or diagnose a variety of disorders. As used herein, the term “subject” is intended to include human and non-human animals. Non-limiting examples of human subjects include human patients suffering from an immune disorder, which as used herein, includes the following conditions: unwanted immune response, e.g., autoimmune diseases, human patients prior, during, or after transplantation or grafting, and human subjects having a proliferative disorder, e.g., cancer. Other preferred human subjects include a subject in need of heightened immune surveillance, e.g., a patient suffering from a other or a subject suffering from a pathogenic infection, e.g., a viral, bacterial, or parasitic infection. The term “non-human animals” of the invention includes all vertebrates, e.g., mammals and non-mammals, such as non-human primates, sheep, dog, cow, chickens, amphibians, reptiles, etc.

As used herein, the term “immune disorder” refers to diseases affecting the immune system, e.g., T cell disorders, B cell disorders, autoimmune disease, infectious disorders, proliferative disorders, transplant rejection, and cancer.

The subject methods and compositions described herein can also be used to modulate (e.g., inhibit) the activity (e.g., proliferation, differentiation, survival) of an immune or hematopoietic cell (e.g., a cell of myeloid, lymphoid, erythroid lineages, or precursor cells thereof), and, thus, can be used to treat or prevent a variety of immune disorders. Non-limiting examples of the disorders that can be treated or prevented include, but are not limited to, transplant rejection, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, and allergy such as, atopic allergy.

As used herein, the terms “cancer,” “hyperproliferative,” “malignant,” and “neoplastic” are used interchangeably, and refer to those cells an abnormal state or condition characterized by rapid proliferation or neoplasm. The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth.

However, as used herein, the terms neoplasia and hyperplasia can be used interchangeably, as their context will reveal, referring generally to cells experiencing abnormal cell growth rates. Neoplasias and hyperplasias include “tumors,” which may be either benign, pre-malignant or malignant.

The subject method can be useful in treating malignancies of the various organ systems, such as those affecting lung, breast, lymphoid, gastrointestinal (e.g., colon), and genitourinary tract (e.g., prostate), pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The subject method can also be used to inhibit proliferative disorders, i.e., the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

As used herein, the terms “leukemia” or “leukemic cancer” refers to all cancers or neoplasias of the hematopoietic and immune systems (blood and lymphatic system). These terms refer to a progressive, malignant disease of the blood-forming organs, marked by distorted proliferation and development of leukocytes and their precursors in the blood and bone marrow. The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell.

Isolated Polynucleotides

One aspect of the invention pertains to isolated polynucleotide molecules comprising anergy markers (e.g., mRNA) of the invention, or polynucleotides which encode polypeptides corresponding to the anergy markers of the invention, or fragments thereof. Another aspect of the invention pertains to isolated polynucleotide fragments sufficient for use as hybridization probes to identify the polynucleotide molecules encoding the markers for the invention in a sample, as well as nucleotide fragments for use as PCR primers of the amplification or mutation of the nucleic acid molecules which encode the anergy markers of the invention.

A polynucleotide molecule of the present invention, e.g., a polynucleotide molecule having the nucleotide sequence of one of the anergy markers listed in Group I or Group II or Group III or Group IV, or homologs thereof, or a portion thereof, can be isolated using standard molecular biology techniques and the sequence information provided herein as well as sequence information known in the art. Using all or a portion of the polynucleotide sequence of one of the anergy markers listed in Group I or Group II or Group III or Group IV (or a homolog thereof) as a hybridization probe, an anergy marker polynucleotide of the invention or a polynucleotide molecule encoding an anergy marker polypeptide of the invention can be isolated using standard hybridization and cloning techniques.

A polynucleotide of the invention can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to anergy marker nucleotide sequences, or nucleotide sequences encoding a marker of the invention can be prepared by standard synthetic techniques, e.g., using an automated DNA synthesizer.

In another preferred embodiment, an isolated polynucleotide molecule of the invention comprises a polynucleotide molecule which is a complement of the nucleotide sequence of an anergy marker of the invention (e.g., a marker listed in Group I or Group II or Group III or Group IV, or homolog thereof), or a portion of any of these nucleotide sequences. A polynucleotide molecule which is complementary to such a nucleotide sequence is one which is sufficiently complementary to the nucleotide sequence such that it can hybridize to the nucleotide sequence, thereby forming a stable duplex.

The polynucleotide molecule of the invention, moreover, can comprise only a portion of the polynucleotide sequence of an anergy marker polynucleotide of the invention, or a gene encoding a polypeptide of the invention, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of an anergy marker polynucleotide, or a polynucleotide encoding an anergy marker polypeptide of the invention.

Probes based on the nucleotide sequence of an anergy marker or of a polynucleotide molecule encoding a marker polypeptide of the invention can be used to detect transcripts or genomic sequences corresponding to the marker polynucleotide(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polynucleotide or polypeptide of the invention, or which have greater or fewer copies of a marker gene of the invention. For example, a level of a marker in a sample of cells from a subject may be detected, the amount of polypeptide or mRNA transcript of a gene encoding a marker polypeptide may be determined, or the presence of mutations or deletions of a marker gene of the invention may be assessed.

The invention further encompasses polynucleotide molecules that differ from the polynucleotide sequences of the markers listed in Group I or Group II or Group III or Group IV due to degeneracy of the genetic code and which thus encode the same proteins as those encoded by the nucleic acids shown in Group I or Group II or Group III or Group IV.

The invention also specifically encompasses homologs of the markers listed in Group I or Group II or Group III or Group IV of other species. Homologs are well understood in the art and are available using databases or search engines such as the Pubmed-Entrez database available at <http:www.ncbi.nlm.nihigov/query.fcgi>.

The invention also encompasses polynucleotide molecules which are structurally different from the molecules described above (i.e. which have a slight altered sequence), but which have substantially the same properties as the molecules above (e.g., encoded amino acid sequences, or which are changed only in non-essential amino acid residues). Such molecules include allelic variants, and are described in greater detail in subsections herein.

In addition to the nucleotide sequences of the markers listed in Group I or Group II or Group III or Group IV, it will be appreciated by those skilled in the art that DNA sequence polymorphisms that lead to changes in the amino acid sequences of the proteins encoded by the markers listed in Group I or Group II or Group III or Group IV may exist within a population (e.g., the human population). Such genetic polymorphism in the markers listed in Group I or Group II or Group III or Group IV may exist among individuals within a population due to natural. An allele is one of a group of genes which occur alternatively at a given genetic locus. In addition it will be appreciated that DNA polymorphisms that affect RNA expression levels can also exist that may affect the overall expression level of that gene (e.g., by affecting regulation or degradation). As used herein, the phrase “allelic variant” includes a nucleotide sequence which occurs at a given locus or to a polypeptide encoded by the nucleotide sequence.

Polynucleotide molecules corresponding to natural allelic variants and homologs of the marker genes, or genes encoding the marker proteins of the invention can be isolated based on their homology to the markers listed in Group I or Group II or Group III or Group IV using the cDNAs disclosed herein, or a portion thereof, as a hybridization probe according to standard hybridization techniques under stringent hybridization conditions. Polynucleotide molecules corresponding to natural allelic variants and homologs of the markers of the invention can further be isolated by mapping to the same chromosome or locus as the markers or polynucleotides encoding the marker proteins of the invention.

In another embodiment, an isolated polynucleotide molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 or more nucleotides in length and hybridizes under stringent conditions to a polynucleotide molecule corresponding to a nucleotide sequence of a marker polynucleotide or polynucleotide encoding a marker protein of the invention. In certain embodiments, the hybridization under stringent conditions is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are described herein and additionally are known to those skilled in the art. Preferably, an isolated polynucleotide molecule of the invention that hybridizes under stringent conditions to the sequence of one of the markers set forth in Group I or Group II or Group III or Group IV corresponds to a naturally-occurring polynucleotide molecule.

In addition to naturally-occurring allelic variants of the marker polynucleotide and polynucleotide encoding a marker protein of the invention sequences that may exist in the population, the skilled artisan will further appreciate that changes can be introduced by mutation into the nucleotide sequences of the marker polynucleotides or polynucleotides encoding the marker proteins of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to polynucleotide molecules encoding a marker protein of the invention that contain changes in amino acid residues that are not essential for activity. Such proteins differ in amino acid sequence from the marker proteins encoded by the markers listed in Group I or Group II or Group III or Group IV yet retain biological activity. In one embodiment, the protein comprises an amino acid sequence at least about 60%, 65%, 70%, 75%, 80%, 85%, 90%, 95%, 98% or more homologous to a marker protein of the invention.

In yet other aspects of the invention, polynucleotides of an anergy marker may comprise one or more mutations. An isolated polynucleotide molecule encoding a protein with a mutation in an anergy marker protein of the invention can be created by introducing one or more nucleotide substitutions, additions or deletions into the nucleotide sequence of the gene encoding the marker protein, such that one or more amino acid substitutions, additions or deletions are introduced into the encoded protein. Such techniques are well known in the art. Mutations can be introduced into the anergy marker polynucleotides of the invention (e.g., a marker listed in Group I or Group II or Group III or Group IV) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of a coding sequence of an anergy gene of the invention, such as by saturation mutagenesis, and the resultant mutants can be screened for biological activity to identify mutants that retain activity. Following mutagenesis, the encoded protein can be expressed recombinantly and the activity of the protein can be determined.

Another aspect of the invention pertains to isolated polynucleotide molecules which are antisense to the anergy marker genes and genes encoding anergy marker proteins of the invention. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can hydrogen bond to a sense polynucleotide. The antisense polynucleotide can be complementary to an entire coding strand of a polynucleotide of the invention or to only a portion thereof. In one embodiment, an antisense polynucleotide molecule is antisense to a “coding region” of the coding strand of a nucleotide sequence of the invention. The term “coding region” includes the region of the nucleotide sequence comprising codons which are translated into amino acid. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a polynucleotide of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xantine, 4-acetylcytosine, 5-(carboxyhydroxyhnethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylesteri, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further herein).

The antisense polynucleotide molecules of the invention are typically administered to a subject or generated in situ such that they hybridize with or bind to cellular mRNA and/or genomic DNA encoding a marker protein of the invention to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention include direct injection at a tissue site (e.g., lymph node or blood). Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intracellular concentrations of the antisense molecules, vector constructs in which the antisense polynucleotide molecule is placed under the control of a strong pol II or pol III promoter are preferred.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue.

In still another embodiment, an antisense polynucleotide of the invention is a ribozyme. Ribozymes are catalytic RNA molecules with ribonuclease activity which are capable of cleaving a single-stranded polynucleotide, such as an mRNA, to which they have a complementary region. Thus, ribozymes (e.g., hammerhead ribozymes) can be used to catalytically cleave mRNA transcripts of the anergy marker polynucleotides of the invention (e.g., as set forth in Group I or Group II or Group III or Group IV) to thereby inhibit translation of said mRNA. A ribozyme having specificity for a marker protein-encoding polynucleotide can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. Alternatively, mRNA transcribed from a gene of the invention can be used to select a catalytic RNA having a specific ribonuclease activity from a pool of RNA molecules.

Alternatively, expression of an anergy marker polynucleotide of the invention can be inhibited by targeting nucleotide sequences complementary to the regulatory region of these genes (e.g., the promoter and/or enhancers) to form triple helical structures that prevent transcription of the gene in target cells.

Expression of the marker polynucleotides, and polynucleotides encoding marker proteins of the invention, can also be inhibited using RNA interference (“RNA₁”). This is a technique for post transcriptional gene silencing (“PTGS”), in which target gene activity is specifically abolished with cognate double-stranded RNA (“dsRNA”). RNAᵢ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNA₁ in mammalian systems is disclosed in PCT application WO 00/63364 which is incorporated by reference herein in its entirety. Basically, dsRNA of at least about 21 nucleotides, homologous to the target marker is introduced into the cell and a sequence specific reduction in gene activity is observed.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides. As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols according to techniques known in the art.

PNAs can be used in therapeutic and diagnostic applications. For example, PNAs can be used as antisense or antigene agents for sequence-specific modulation of marker polynucleotide expression by, for example, inducing transcription or translation arrest or inhibiting replication. PNAs of the polynucleotide molecules of the invention (e.g., set forth in Group I or Group II or Group III or Group IV, or homologs thereof) can also be used in the analysis of single base pair mutations in a gene, (e.g., by PNA-directed PCR clamping); as ‘artificial restriction enzymes’ when used in combination with other enzymes, (e.g., S1 nucleases); or as probes or primers for DNA sequencing or hybridization.

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation according to techniques known in the art. The synthesis of PNA-DNA chimeras is known in the art. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

In other embodiments, the oligonucleotide may include other appended groups such as peptides (e.g., for targeting host cell receptors in vivo), or agents facilitating transport across the cell membrane or the blood-kidney barrier. In addition, oligonucleotides can be modified with hybridization-triggered cleavage agents or intercalating agents. To this end, the oligonucleotide may be conjugated to another molecule, (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label (e.g., a molecular beacon).

Nucleic Acid Arrays

Arrays are useful molecular tools for characterizing a sample by multiple criteria. For example, an array having capture probes for one or more anergy polynucleotides of Group I or Group II or Group III or Group IV can be used to assess the anergic state of an immune cell. Arrays can have many addresses, e.g., locatable sites, on a substrate. The featured arrays can be configured in a variety of formats, non-limiting examples of which are described below.

Each anergy marker may be considered individually, although it is within the scope of the invention to provide combinations of two or more markers for use in the methods and compositions of the invention to increase the confidence of the analysis. In another aspect, the invention provides panels of the anergy markers of the invention. A panel may also comprise 2-5, 5-15, 15-35, 35-50, 50-100, 100-500, 500-1000, 1000-10000 or more than 10000 anergy markers.

In a preferred embodiment, these panels of markers are selected such that the markers within any one panel share certain features. For example, the markers of a first panel may each exhibit at least a two-fold increase in quantity or activity in an immune disorder sample, as compared to a sample which is substantially free of an immune disorder, from the same subject or a sample which is substantially free of an immune disorder from a different subject without an immune disorder. Alternatively, markers of a second panel may each exhibit differential regulation as compared to a first panel. Similarly, different panels of markers may be composed of markers from different functional categories (i.e., proteolysis, signal transduction, transcription, etc.) or samples (i.e., kidney, spleen, lymph node, brain, intestine, colon, heart or urine), or may be selected to represent different stages of an immune disorder. Panels of the anergy markers of the invention may be made by independently selecting markers from Group I or Group II or Group III or Group IV, and may further be provided on biochips or arrays, as discussed herein.

The array substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. Non-limiting examples of two-dimensional array substrates include glass slides, quartz (e.g., UV-transparent quartz glass), single crystal silicon, wafers (e.g., silica or plastic), mass spectroscopy plates, metal coated substrates (e.g., gold), membranes (e.g., nylon and nitrocellulose), plastics and polymers (e.g., polystyrene, polypropylene, polyinylidene difluoride, poly-tetrafluoroethylene, polycarbonate, PDMS, nylon, acrylic, and the like). Three-dimensional array substrates include porous matrices, e.g., gels or matrices. Potentially useful porous substrates include: agarose gels, acrylamide gels, sintered glass, dextran, meshed polymers (e.g., macroporous crosslinked dextran, sephacryl, and sepharose), and so forth.

The array can have a density of at least 2, 5, 10, 50, 100, 200, 500, 1 000, 2 000, 10⁴, 10⁵, 10⁶, 10⁷, 10⁸, or 10⁹ or more addresses per cm² and ranges between. In a preferred embodiment, the plurality of addresses includes at least 10, 100, 500, 1 000, 5 000, 10 000, or 50 000 addresses. In a preferred embodiment, the plurality of addresses includes less than 9, 99, 499, 999, 4 999, 9 999, or 49 999 addresses. Addresses in addition to the address of the plurality can be disposed on the array. The center to center distance can be 5 mm, 1 mm, 100 um, 10 um, 1 um or less. The longest diameter of each address can be 5 mm, 1 mm, 100 um, 10 um, 1 um or less. Each addresses can contain 0 ug, 1 ug, 100 ng, 10 ng, 1 ng, 100 pg, 10 pg, 1 pg, 0.1 pg, or less of a capture agent, i.e. the capture probe. For example, each address can contain 100, 10³, 10⁴, 10⁵, 10⁶, 10⁷, 10⁸, or 10⁹ or more molecules of the nucleic acid.

Arrays can be fabricated by a variety of methods, e.g., photolithographic methods, mechanical methods, pin based methods, and bead based techniques. The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe, i.e., the anergy marker, can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated nucleic acid is preferably mRNA that can be isolated by routine methods, e.g., including DNase treatment to remove genomic DNA and hybridization to an oligo-dT coupled solid substrate. The substrate is washed, and the mRNA is eluted.

The isolated mRNA can be reversed transcribed and optionally amplified, e.g., by rtPCR. The nucleic acid can be an amplification product, e.g., from PCR; rolling circle amplification (“RCA,”), isothermal RNA amplification or NASBA, and strand displacement amplification. The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

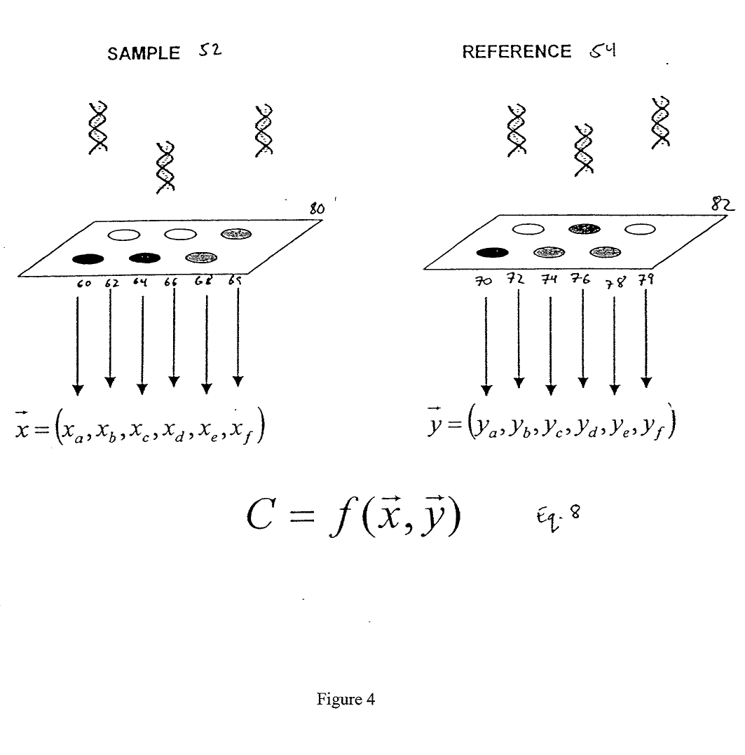

Referring to FIG. 4, a general scheme for producing and evaluating profiles is depicted. Nucleic acid is prepared from a sample 52, e.g., a sample of interest and hybridized to an array 80, e.g., with multiple addresses (60, 62, 64, 66, 68, and 69) of which six are shown. Hybridization of the nucleic acid to the array is detected. The extent of hybridization at an address is represented by a numerical value and stored, e.g., in a vector, a one-dimensional matrix, or one-dimensional array. The vector x has a value for each address of the array. For example, a numerical value for the extent of hybridization at address 60 is stored in variable xₐ. The numerical value can be adjusted, e.g., for local background levels, sample amount, and other variations. Nucleic acid is also prepared from a reference sample 54 and hybridized to an array 82 (e.g., the same or a different array), e.g., with multiple addresses (70, 72, 74, 76, 78, 79). The vector y is construct identically to vector x. The sample expression profile and the reference profile can be compared, e.g., using a mathematical equation 84 that is a function of the two vectors. The comparison can be evaluated as a scalar value, e.g., a score representing similarity of the two profiles. Either or both vectors can be transformed by a matrix in order to add weighting values to different nucleic acids detected by the array.

Computer readable media comprising anergy marker(s) of the present invention is also provided. As used herein, “computer readable media” includes a medium that can be read and accessed directly by a computer. Such media include, but are not limited to magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” includes a process for storing information on computer readable medium. Those skilled in the art can readily adopt any of the presently known methods for recording information on computer readable medium to generate manufactures comprising the markers of the present invention.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Nucleic acids that are similarly regulated during a change in T cell state, e.g., a change in NFAT activity, or induction of anergy, can be identified by clustering expression data to identify coregulated nucleic acids. Nucleic acids can be clustered using hierarchical clustering, Bayesian clustering, k-means clustering, and self-organizing maps.

Expression profiles obtained from nucleic acid expression analysis on an array can be used to compare samples and/or cells in a variety of states. In one embodiment, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or the Pearson correlation coefficient.

The similarity of a sample expression profile to a predictor expression profile (e.g., a reference expression profile that has associated weighting factors for each nucleic acid) can then be determined, e.g., by comparing the log of the expression level of the sample to the log of the predictor or reference expression value and adjusting the comparison by the weighting factor for all nucleic acids of predictive value in the profile.

For immune cells, expression profiles can include nucleic acids in addition to the anergy marker polynucleotides listed in Group I or Group II or Group III or Group IV. Nucleic acids can be classified based on their qualitative change in expression levels in the following two conditions (Ionomycin alone, “I alone”; Ionomycin+PMA, “I PMA”). Both conditions are compared relative to a third condition (Ionomycin+cyclosporin A; see Table 2).

TABLE 2

| Category | I alone | I + PMA |
|---|---|---|
| 1 | up | no change |
| 2 | no change | up |
| 3 | down | no change |
| 4 | no change | down |
| 5 | up | up |
| 6 | down | down |

Nucleic acids of all categories can be used to characterize a sample. In a preferred embodiment, the magnitude of change is determined and used for more sophisticated classification, e.g., with quantitative boundaries. As described above, such characterization is best determined using quantitative metrics and algorithms.

In one embodiment, the array can be used to assay gene expression in a tissue to ascertain tissue specificity of genes in the array. In this manner, up to about 12,000 genes can be simultaneously assayed for expression. This allows an expression profile to be developed showing a battery of polynucleotides specifically expressed in one or more tissues at a given point in time. In one embodiment the invention provides a kit comprising a brochure which comprises at least 5, more preferably 10, more preferably 25 or more anergy markers, and the same anergy markers in computer readable form.

In addition to such qualitative determination, the invention allows the quantitation of polynucleotide expression in the biochip. Thus, not only tissue specificity, but also the level of expression of a battery of markers in the tissue is ascertainable. Thus, markers can be grouped on the basis of their tissue expression per se and level of expression in that tissue. As used herein, a “normal level of expression” refers to the level of expression of a polynucleotide provided in a control sample, typically the control is taken from either a non-diseased animal or from a subject who has not suffered from an immune disorder. The determination of normal levels of expression is useful, for example, in ascertaining the relationship of polynucleotide expression between or among tissues. Thus, one tissue or cell type can be perturbed and the effect on polynucleotide expression in a second tissue or cell type can be determined. In this context, the effect of one cell type on another cell type in response to a biological stimulus can be determined. Such a determination is useful, for example, to know the effect of cell-cell interaction at the level of polynucleotide expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more polynucleotides in the array. This can occur in various biological contexts, as disclosed herein, for example development and differentiation, disease progression, in vitro processes, such as cellular transformation and activation.

The array is also useful for ascertaining the effect of the expression of a polynucleotide on the expression of other polynucleotides in the same cell or in different cells. This provides, for example, for a selection of alternate molecular targets for therapeutic intervention if the ultimate or downstream target cannot be regulated.

Importantly, the invention provides arrays useful for ascertaining differential expression patterns of one or more genes identified in diseased tissue versus non-diseased tissue. This provides a battery of polynucleotides that serve as a molecular target for diagnosis or therapeutic intervention. In particular, biochips can be made comprising arrays not only of the differentially expressed markers listed in Group I or Group II or Group III or Group IV, but of markers specific to subjects suffering from specific manifestations or degrees of an immune disease (i.e. c-myc for cancer; TNF-α in rheumatoid arthritis).

Polyeptide Arrays

The expression level of a polypeptide encoded by an anergy marker listed in Group I or Group II or Group III or Group IV can be determined using an antibody specific for the polypeptide (e.g., using a Western blot or an ELISA assay). Moreover, the expression levels of multiple polypeptides encoded by these anergy markers can be rapidly determined in parallel using a polypeptide array having antibody capture probes for each of the polypeptides. Antibodies specific for a polypeptide can be generated by a method described herein.

A low-density (96 well format) protein array has been developed in which proteins are spotted onto a nitrocellulose membrane. A high-density protein array (100,000 samples within 222×222 mm) used for antibody screening was formed by spotting proteins onto polyinylidene difluoride (PVDF). Polypeptides can be printed on a flat glass plate that contained wells formed by an enclosing hydrophobic Teflon mask. Also, polypeptide can be covalently linked to chemically derivatized flat glass slides in a high-density array (1600 spots per square centimeter). Known in the art is a method using a high-density array of 18,342 bacterial clones, each expressing a different single-chain antibody, in order to screen antibody-antigen interactions. These art-known methods and others can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

The anergy marker arrays and anergy polypeptide arrays of the invention can be used in wide variety of applications. For example, the arrays can be used to analyze a patient sample. The sample is compared to data obtained previously, e.g., known clinical specimens or other patient samples. Further, the arrays can be used to characterize a cell culture sample, e.g., to determine a cellular state after varying a parameter, e.g., exposing the cell culture to an antigen, a transgene, or a test compound.

Transactional Methods for Evaluating a Sample

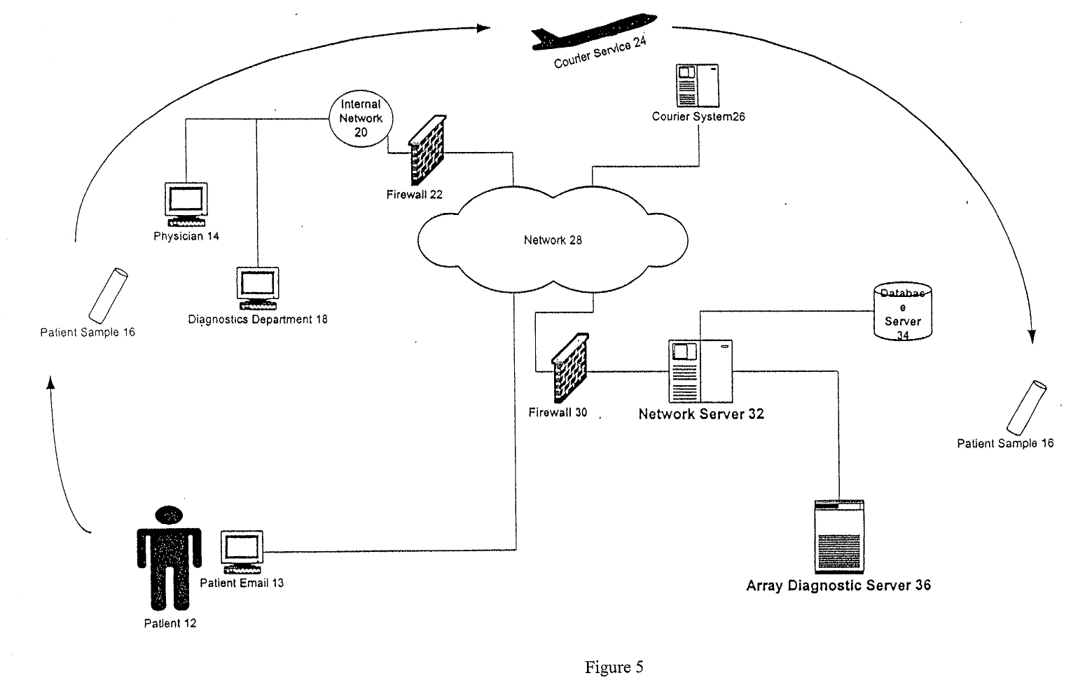

Referring to FIG. 5, a patient 12 is treated by a physician 14. The physician obtains a sample (i.e., “patient sample”) 16, e.g., a blood sample, from the patient. The patient sample can be delivered to a diagnostics department 18 which can collate information about the patient, the patient sample, and results of the evaluation. A courier service 24 can deliver the sample to a diagnostic service. Location of the sample is monitored by a courier computer system 26, and can be tracked by accessing the courier computer system, e.g., using a web page across the Internet. At the diagnostic service, the sample is processed to produce a sample expression profile. For example, nucleic acid is extracted from the sample, optionally amplified, and contacted to a nucleic acid microarray. Binding of the nucleic acid to the microarray is quantitated by a detector that streams data to the array diagnostic server 36. The array diagnostic server processes the microarray data, e.g., to correct for background, sample loading, and microarray quality. It can also compare the raw or processed data to a reference expression profile, e.g., to produce a difference profile. The raw profiles, processed profiles and/or difference profiles are stored in a database server 36. A network server 32 manages the results and information flow. In one embodiment, the network server encrypts and compresses the results for electronic delivery to the healthcare provider's internal network 20. The results can be sent across a computer network 26, e.g., the Internet, or a proprietary connection. For data security, the diagnostic systems and the healthcare provider systems can be located behind firewalls 22 & 30. In another embodiment, an indication that the results are available can also be sent to the healthcare provider and/or the patient 12, for example, by to an email client 13. The healthcare provider, e.g., the physician, can access the results, e.g., using the secure HTTP protocol (e.g., with secure sockets layer (SSL) encryption). The results can be provided by the network server as a web page (e.g., in HTML, XML, and the like) for viewing on the physician's browser.

Further communication between the physician and the diagnostic service can result in additional tests, e.g., a second expression profile can be obtained for the sample, e.g., using the same or a different microarray.

Nucleic Acids, Vectors and Host Cells

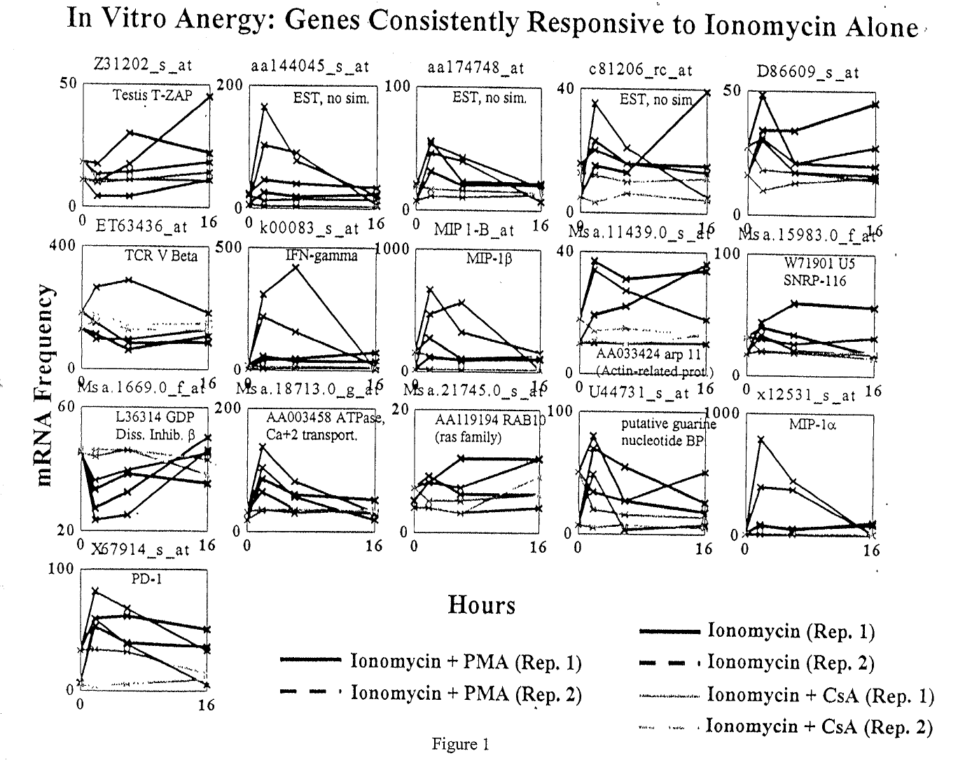

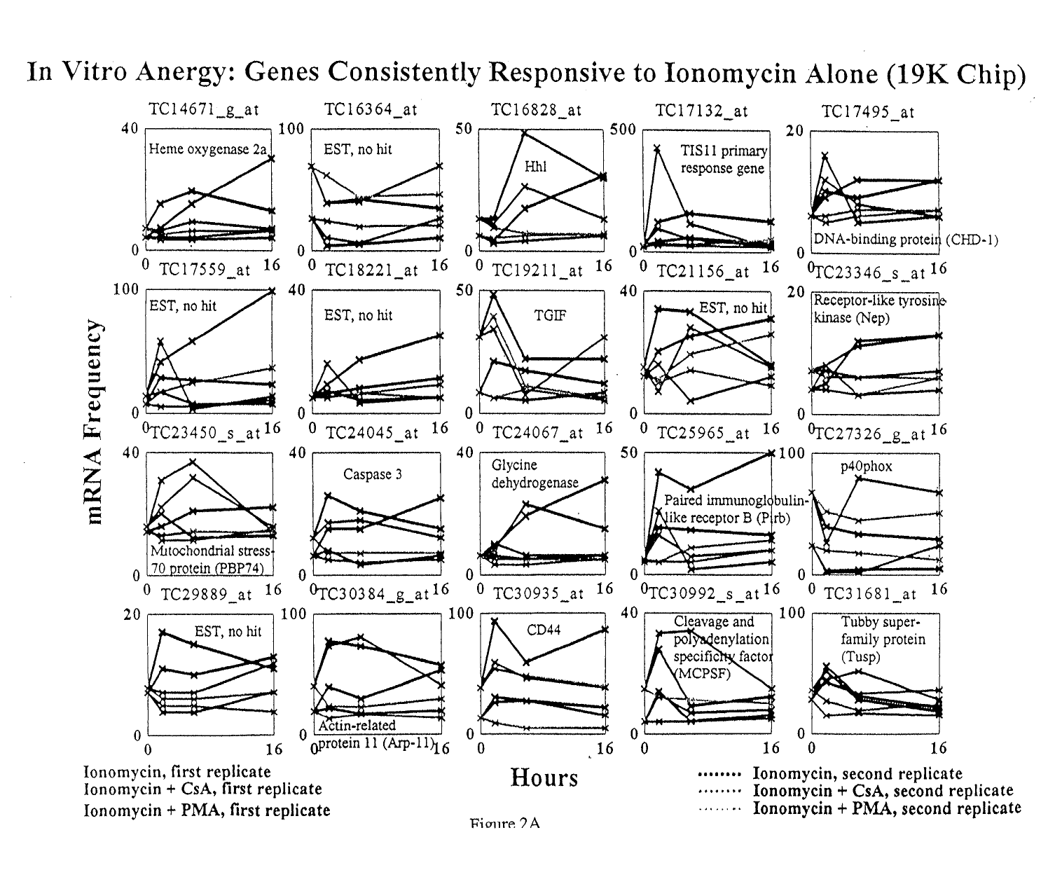

The anergy polynucleotides described herein include murine and human polynucleotides identified as nucleic acid components of the anergic nucleic acid expression program. The identity of these nucleic acids is documented in several ways. Murine sequences with “TC” identifiers are accompanied by a listing of their nucleotide sequences (Table 1). “TC” identifiers refer to the consensus sequence information as reported at the website for The Institute of Genetic Research. Other murine sequences and human sequences are identified by their UniGene reference number (e.g., beginning with the prefix “Mm.”). Still other sequences are identified by their Affymetrix reference number (e.g., beginning with the prefix “Msa.” or ending with the suffix “_at”). The corresponding GenBank EST identifier for many of these can be found in FIG. 1.

UniGene is a non-redundant collection of genetic loci with reference to EST, cDNA, and genomic DNA sequences that correspond to a given nucleic acid. The UniGene web resource (http://www.ncbi.nlm.nih.gov/UniGene/) allows for the rapid identification of additional sequences corresponding to the anergy marker, e.g., a capture probe can be made to any complex sequence region of the anergy marker. In addition, the UniGene web resource has links to the corresponding nucleic acid in other species, e.g., human or rat. A skilled artisan can rapidly identify sequences from other species and additional sequences of a given anergy marker in order to provide nucleic acids for aspects of the invention.

Another aspect of the invention pertains to isolated anergy markers listed in Group I or Group II or Group III or Group IV, or a fragment encoding a portion thereof, e.g., an immunogenic or biologically active portion of a protein encoded by an anergy marker listed in Group I or Group II or Group III or Group IV, as well as a vector and host cell compositions that can be used for expression of an anergy marker of the invention, e.g., an anergy marker listed in Group I or Group II or Group III or Group IV. The anergy marker can be used to express the polypeptide encoded by the marker, e.g., for a screening method described herein.

Particularly preferred polynucleotides of the present invention have a nucleotide sequence identical or sufficiently similar to the sequences described herein. The term “sufficiently identical” or “substantially identical” is used herein to refer to a first nucleotide sequence that contains a sufficient or minimum number of identical or equivalent (e.g., encoding an amino acid with a similar side chain) to a second nucleotide sequence such that the first and second nucleotide sequences encode polypeptides having a common structural domain or common functional activity. For example, nucleotide sequences that contain a common structural domain having at least about 60%, or 65% identity, likely 75% identity, more likely 85%, 90%. 91%, 92%, 93%, 94%, 95%, 96%, 97%, 98% or 99% identity are defined herein as sufficiently or substantially identical.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the amino acid sequence encoded by an anergy marker listed in Group I or Group II or Group III or Group IV). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller, which has been incorporated into the ALIGN program (version 2.0), using a PAM120 weight residue table, a gap length penalty of 12 and a gap penalty of 4.

The anergy markers and protein sequences encoded by the anergy markers described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to an anergy marker of the invention, such as, for example, those listed in Group I or Group II or Group III or Group IV. An example of the results of a BLAST search is shown in Table 3, which shows the qualifier, and the description from the BLAST search.

TABLE 3

| Qualifier | Description |
|---|---|
| TC14671_g_at | 1226/1259 (97%), AF054669 Mus musculus heme oxygenase 2a (HO- |
| | 2a) mRNA, complete cds. |
| TC16364_at | no hit |
| TC16828_at | 478/510 (93%), AF118565 Mus musculus Hhl mRNA, complete cds. |
| TC17132_at | 2652/2665 (99%), M58566 Mouse TIS11 primary response gene, |
| | complete cds. |
| TC17495_at | 5341/5347 (99%), L10410 Mouse DNA-binding protein (CHD-1) |
| | mRNA, complete eds. |
| TC17558_at | no hit |
| TC18221_at | not hit |
| TC19211_at | 1132/1189 (95%), X89749 M. musculus mRNA for mTGIF protein. |
| TC21156_at | no hit |
| TC23346_s_at | 1249/1269 (98%), AF026259 Mus musculus receptor-like tyrosine |
| | kinase (Nep) mRNA, complete cds. |
| TC23450_s_at | 2992/2999 (99%), D17556 Mouse mRNA for mitochondrial stress-70 |
| | protein (PBP74), complete cds. |
| TC24045_at | 1594/1604 (99%), U63720 Mus musculus CPP32 apoptotic protease |
| | mRNA, partial cds. AND 207 bp ORF match: 207/207 (100%), |
| | U49929 Mus musculus ICE-like cysteine protease (Lice) mRNA, |
| | complete cds. AND 207/207 (100%), Y13086 M. musculus mRNA |
| | for caspase-3. |
| TC24067_at | Blast P ID: Identities = 192/200 (96%), Positives = 196/200 (98%); |
| | glycine dehydrogenase (decarboxylating) (EC 1.4.4.2)-human |
| TC25965_at | Contig match: Identities = 35/42 (83%), Positives = 37/42 (87%); |
| | U84903 Mus musculus L23 mitochondrial-related protein (L23 mrp) |
| | gene, complete cds. AND Identities = 36/41 (87%). Positives = 37/41 |
| | (89%); AF038149 Mus musculus paired immunoglobulin-like receptor |
| | B (Pirb) gene, complete cds. |
| TC27326_g_at | 1422/1425 (99%), AB002665 Mus musculus mRNA for p40phox, |
| | complete cds. |
| TC29889_at | no hit |
| TC30384_g_at | 1549/1562 (99%), AF190797 Mus musculus actin-related protein 11 |
| | (Arp11) mRNA, complete cds. |
| TC30935_at | 2301/2379 (96%), AJ251594 Mus musculus mRNA for |
| | transmembrane glycoprotein (CD44 gene). |
| TC30992_s_at | 2195/2281 (96%), AF012822 Mus musculus cleavage and |
| | polyadenylation specificity factor (MCPSF) mRNA, complete cds. |
| TC31681_at | 2314/2335 (99%), AF219945 Mus musculus tubby super-family |
| | protein (Tusp) mRNA, complete cds. |
| TC3225_at | no hit |
| TC33206_at | 4078/4161 (98%), AJ131821 Mus musculus mRNA for |
| | sarco/endoplasmic reticulum Ca2+ATPase; SERCA2b. |
| TC33833_at | Blast P: Identities = 120/217 (55%), Positives = 149/217 (68%), Gaps = |
| | 36/217 (16%); Y57946 Human transmembrane protein HTMPN-70. |
| TC34186_at | TblastX: Identities = 24/26 (92%), Positives = 24/26 (92%); AF038149 |
| | Mus musculus paired immunoglobulin-like receptor B (Pirb) gene, |
| | complete cds. |
| TC36089_at | TblastX: Identities = 17/45 (37%), Positives = 24/45 (52%); Z80897 |
| | Human DNA sequence from clone LL22NC01-132D12 on |
| | chromosome 22 Contains the gene for a novel protein sharing domains |
| | with RAS-related protein, ESTs, STSs, GSSs and a putative CpG |
| | island, complete sequence. AND BlastP Identities = 33/93 (35%), |
| | Positives = 38/93 (40%), Gaps = 11/92 (11%); Q9vc21 ELA |
| | PROTEIN. |
| TC36583_at | TblastX: Identities = 15/21 (71%), Positives = 17/21 (80%); AF133300 |
| | Mus musculus MOR 3′Beta1, MOR 3′Beta2, and 3′Beta3 genes, |
| | complete cds. |
| TC37631_at | no hit |
| TC38094_at | no hit |
| TC38978_at | Identities = 24/31 (77%), Positives = 24/31 (77%); AL035682 Human |
| | DNA sequence from clone RP5-1009H6 on chromosome 20 Contains |
| | parts of isoforms B and C of the NFATC2 (nuclear factor of activated |
| | T-cells, cytoplasmic 2) gene, ESTs, STSs and GSSs, complete |
| | sequence. |
| TC39012_at | 1594/1604 (99%), U63720 Mus musculus CPP32 apoptotic protease |
| | mRNA, partial cds. AND 1465/1471 (99%). U49929 Mus musculus |
| | ICE-like cysteine protease (Lice) mRNA, complete cds. |
| TC39080_at | Identities = 26/32 (81%), Positives = 28/32 (87%); U80917 Homo |
| | sapiens transcription factor (NE-Atc/B) mRNA, complete cds. |
| TC39762_at | 215/238 (90%), X98051 R. norvegicus fos-related antigen DNA, exon |
| | 4. |
| TC40487_g_at | no hit |
| TC41014_at | no hit |

BLAST protein searches can be performed with the XBLAST program, score=50, wordlength=3 to obtain amino acid sequences homologous to protein molecules of the invention. Preferably, the homologous sequence has at least 60%, 70%, 80%, or 85% homology to the query sequence. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

In other embodiments, the term “sufficiently identical” or “substantially identical” refers to a nucleotide sequence which is capable of hybridizing under stringent conditions, e.g. highly strigent conditions, to an anergy marker listed in Group I or Group II or Group III or Group IV. As used herein, the term “hybridizes under stringent conditions” describes conditions for hybridization and washing. Stringent conditions are known to those skilled in the art and generally are described herein. Aqueous and nonaqueous methods are also known in the art and either can be used.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules or polynucleotides which include an open reading frame that is capable of encoding a protein or polypeptide of the invention, preferably a mammalian (e.g., murine or human) protein of the invention, after being transcribed and translated. Genes and recombinant genes can further include non-coding regulatory sequences, and introns. Any of the polypeptide sequences described herein may also be used to identify larger fragments or full-length coding sequences of the gene with which they are associated. Methods of isolating larger fragment sequences are known to those of skill in the art, some of which are described herein.

As used herein, the term, “transcribed” or “transcription” refers to the process by which genetic code information is transferred from one kind of nucleic acid to another, and refers in particular to the process by which a base sequence of mRNA is synthesized on a template of cDNA.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “gene product” includes an amino acid sequence (e.g., peptide or polypeptide) generated when a gene is transcribed and translated.

The anergy markers of the invention can be altered to have codons that are preferred or non-preferred, for a particular expression system. For example, the anergy marker can be one in which at least one codon, at preferably at least 10%, or 20% of the codons, has been altered. The alteration can render the sequence optimized for expression in E. coli, yeast, human, insect, or CHO cells.

In a preferred embodiment, the marker differs (e.g., differs by substitution, insertion, or deletion) from that of the sequences provided, e.g., as follows: by at least one but less than 10, 20, 30, or 40 nucleotides; at least one but less than 1%, 5%, 10% or 20% of the nucleotides in the subject marker. If necessary for this analysis, the sequences should be aligned for maximum homology. “Looped” out sequences from deletions or insertions, or mismatches, are considered differences. The differences are changes at nucleotides encoding a non-essential residue(s) or a conservative substitution(s).

Another aspect of the invention pertains to host cells into which a polynucleotide molecule of the invention is introduced, e.g., an anergy marker polynucleotide listed in Group I or Group II or Group III or Group IV, or homolog thereof, within a recombinant expression vector or a polynucleotide molecule of the invention containing sequences which allow it to homologously recombine into a specific site of the host cell's genome. The terms “host cell” and “recombinant host cell” are used interchangeably herein. Such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E coli, or eukaryotic, e.g., insect cells, yeast, or preferably mammalian cells (e.g., cultured cell or a cell line). Other suitable host cells are known to those skilled in the art.

Preferred mammalian host cells for expressing the polypeptides of the invention (e.g., polypeptidse encoded by anergy markers listed in Group I or Group II or Group III or Group IV) include Chinese Hamster Ovary (CHO cells) (including dhfr-CHO cells used with a DHFR selectable marker), lymphocytic cell lines, e.g., NS0 myeloma cells and SP2 cells, COS cells, and a cell from a transgenic animal, e.g., an NFAT−/− mouse e.g., a T cell or B cell from an NFAT−/− mouse.

In another aspect, the invention features a vector, e.g., a recombinant expression vector. The recombinant expression vectors of the invention can be designed for expression of the anergy markers listed in Group I or Group II or Group III or Group IV in prokaryotic or eukaryotic cells. For example, polypeptides of the invention can be expressed in E. coli, insect cells (e.g., using baculovirus expression vectors), yeast cells or mammalian cells. Suitable host cells are well-known in the art. Alternatively, the recombinant expression vector can be transcribed and translated in vitro, for example using T7 promoter regulatory sequences and T7 polymerase.

Vector DNA can be introduced into prokaryotic or eukaryotic cells via conventional transformation or transfection techniques. As used herein, the terms “transformation” and “transfection” are intended to refer to a variety of art-recognized techniques for introducing foreign polynucleotide (e.g., DNA) into a host cell, including calcium phosphate or calcium chloride co-precipitation, DAKD-dextran-mediated transfection, lipofection, or electoporation. Suitable methods for transforming or transferring host cells are well-known and can be found in laboratory manuals known in the art.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Polynucleotides encoding a selectable flag can be introduced into a host cell on the same vector as that encoding a marker protein or can be introduced on a separate vector. Cells stably transfected with the introduced polynucleotide can be identified by drug selection (e.g., cells that have incorporated the selectable flag gene will survive, while the other cells die).

A host cell of the invention, such as a prokaryotic or eukaryotic host cell in culture, can be used to produce (i.e., express) a marker protein. Accordingly, the invention further provides methods for producing an anergy marker protein using the host cells of the invention. In one embodiment, the method comprises culturing the host cell of invention (into which a recombinant expression vector encoding a marker protein has been introduced) in a suitable medium such that a marker protein of the invention is produced. In another embodiment, the method further comprises isolating a marker protein from the medium or the host cell.

Expression of proteins in prokaryotes is most often carried out in E coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in marker activity assays, (e.g., direct assays or competitive assays described in detail below), or to generate antibodies specific for marker proteins, for example.

In addition to the anergy marker polynucleotides, the recombinant expression vectors of the invention may carry regulatory sequences that are operatively linked and control the expression of the anergy markers in a host cell.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, “operably linked” means that the DNA sequences being linked are contiguous and, where necessary to join two protein coding regions, contiguous and in reading frame. For switch sequences, “operably linked” indicates that the sequences are capable of effecting switch recombination.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of nucleic acids to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “regulatory sequence” is intended to includes promoters, enhancers and other expression control elements (e.g., polyadenylation signals) that control the transcription or translation of the anergy markers. Such regulatory sequences are described, for example, in laboratory manuals known in the art. It will be appreciated by those skilled in the art that the design of the expression vector, including the selection of regulatory sequences may depend on such factors as the choice of the host cell to be transformed, the level of expression of protein desired, etc. The expression vectors of the invention can be introduced into host cells to thereby produce proteins or peptides, including fusion proteins or peptides, encoded by polynucleotides as described herein (e.g., marker proteins, mutant forms of marker proteins, fusion proteins, and the like). Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”).

Examples of suitable inducible non-fusion E coli expression vectors include pTrc and pET 11d. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the polynucleotide sequence of the polynucleotide to be inserted into an expression vector so that the individual codons for each amino acid are those preferentially utilized in E. coli. Such alteration of polynucleotide sequences of the invention can be carried out by standard DNA synthesis techniques.

In another embodiment, the anergy differential marker expression vector is a yeast expression vector. Examples of vectors for expression in yeast S. cerevisiae include pYepSec, pMFa, pJRY88, pYES2 (In Vitrogen Corporation, San Diego, Calif.), and picZ (In Vitrogen Corp, San Diego, Calif.).

Alternatively, marker proteins of the invention can be expressed in insect cells using baculovirus expression vectors. Baculovirus vectors available for expression of proteins in cultured insect cells (e.g., Sf 9 cells) include the pAc series and the pVL series.

In yet another embodiment, a polynucleotide of the invention is expressed in mammalian cells using a mammalian expression vector. Examples of mammalian expression vectors include pCDM8 and pMT2PC. When used in mammalian cells, the expression vector's control functions are often provided by viral regulatory elements. For example, commonly used promoters are derived from polyoma, Adenovirus 2, cytomegalovirus and Simian Virus 40. Other suitable expression systems for both prokaryotic and eukaryotic cells may be found in laboratory manuals known in the art. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

In another embodiment, the recombinant mammalian expression vector is capable of directing expression of the polynucleotide preferentially in a particular cell type (e.g., tissue-specific regulatory elements are used to express the polynucleotide). Tissue-specific regulatory elements are known in the art and may include epithelial cell-specific promoters. Other non-limiting examples of suitable tissue-specific promoters include the albumin promoter (liver-specific), lymphoid-specific promoters, in particular promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example, the marine hox promoters and the α-fetoprotein promoter. In certain preferred embodiments of the invention, the tissue-specific promoter is an epithelial cell-specific promoter.

In addition to the anergy marker polynucleotides and regulatory sequences, the recombinant expression vectors of the invention may carry additional sequences, such as sequences that regulate replication of the vector in host cells (e.g., origins of replication) and selectable flags as described above.

The invention further provides a recombinant expression vector comprising an anergy marker polynucleotide of the invention cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a marker gene of the invention (e.g., listed in Group I or Group II or Group III or Group IV). Regulatory sequences operatively linked to a polynucleotide cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region, the activity of which can be determined by the cell type into which the vector is introduced. For example, the antisense nucleic acid can be a synthetic oligonucleotide having a length of about 10, 15, 20, 30, 40, 50, 75, 90, 120 or more nucleotides in length.

An antisense nucleic acid can be synthesized chemically or produced using enzymatic reagents, e.g., a ligase. An antisense nucleic acid can also incorporate modified nucleotides, and artificial backbone structures, e.g., phosphorothioate derivative, and acridine substituted nucleotides.

The host cells of the invention can also be used to produce non-human transgenic animals, for example, a NFAT−/− knockout transgenic mouse. For example, in one embodiment, a host cell of the invention is a fertilized oocyte or an embryonic stem cell into which marker-protein-coding sequences have been introduced. Such host cells can then be used to create non-human transgenic animals in which exogenous sequences encoding a marker protein of the invention have been introduced into their genome or homologous recombinant animals in which endogenous sequences encoding the marker proteins of the invention have been altered. Such animals are useful for studying the function and/or activity of a marker protein and for identifying and/or evaluating modulators of marker protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous marker gene of the invention (e.g., listed in Group I or Group II or Group III or Group IV) has been altered by homologous recombination between the endogenous gene and an exogenous DNA molecule introduced into a cell of the animal, e.g., an embryonic cell of the animal, prior to development of the animal.

A transgenic animal of the invention can be created by introducing a marker-encoding polynucleotide into the mate pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a marker protein to particular cells. Methods for generating transgenic animals via embryo manipulation and microinjection, particularly animals such as mice, have become conventional in the art. Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a marker protein can further be bred to other transgenic animals carrying other transgenes.

To create a homologous recombinant animal, a vector is prepared which contains at least a portion of a gene of the invention into which a deletion, addition or substitution has been introduced to thereby alter, e.g., functionally disrupt, the gene. The gene can be a human gene, but more preferably, is a non-human homolog of a human gene of the invention (e.g., a homolog of a marker listed in Group I or Group II or Group III or Group IV). For example, a mouse gene can be used to construct a homologous recombination polynucleotide molecule, e.g., a vector, suitable far altering an endogenous gene of the invention in the mouse genome. In a preferred embodiment, the homologous recombination polynucleotide molecule is designed such that, upon homologous recombination, the endogenous gene of the invention is functionally disrupted (i.e., no longer encodes a functional protein; also referred to as a “knock out” vector). Alternatively, the homologous recombination polynucleotide molecule can be designed such that, upon homologous recombination, the endogenous gene is mutated or otherwise altered but still encodes functional protein (e.g., the upstream regulatory region can be altered to thereby alter the expression of the endogenous marker protein). In the homologous recombination polynucleotide molecule, the altered portion of the gene of the invention is flanked at its 5′ and 3′ ends by additional polynucleotide sequence of the gene of the invention to allow for homologous recombination to occur between the exogenous gene carried by the homologous recombination polynucleotide molecule and an endogenous gene in a cell, e.g., an embryonic stem cell. The additional flanking polynucleotide sequence is of sufficient length for successful homologous recombination with the endogenous gene.

Typically, several kilobases of flanking DNA (both at the 5′ and 3′ ends) are included in the homologous recombination polynucleotide molecule. The homologous recombination polynucleotide molecule is introduced into a cell, e.g., an embryonic stem cell line (e.g., by electroporation) and cells in which the introduced gene has homologously recombined with the endogenous gene are selected. The selected cells can then be injected into a blastocyst of an animal (e.g., a mouse) to form aggregation chimeras. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination polynucleotide molecules, e.g., vectors, or homologous recombinant animals are known in the art.

In another embodiment, transgenic non-human animals can be produced which contain selected systems which allow for regulated expression of the transgene. One example of such a system is the cre/loxP recombinase system of bacteriophage P1. Another example of a recombinase system is the FLP recombinase system of Saccharomyces cerevisiae. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to known methods. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated. In preferred embodiments of the invention, the non-human transgenic animals comprise an NFAT−/− mouse.

Reporter Nucleic Acid Assays

In another implementation, a reporter nucleic acid is utilized to monitor the expression of one or more anergy markers listed in Group I or Group II or Group III or Group IV. Such a reporter can be useful for high-throughput screens for agents that alter a T cell state.

The construction of a reporter for transcriptional regulation of a marker of the invention requires a regulatory sequence of marker, typically the promoter. The promoter can be obtained by a variety of routine methods. For example, a genomic library can be hybridized with a labeled probe consisting of the coding region of the nucleic acid to identify genomic library clones containing promoter sequences. The isolated clones can be sequenced to identify sequences upstream from the coding region. Another method is an amplification reaction using a primer that anneals to the 5′ end of the coding region of the marker polynucleotide. The amplification template can be, for example, restricted genomic nucleic acid to which bubble adaptors have been ligated.

To construct the reporter, the promoter of the selected nucleic acid can be operably linked to the reporter nucleic acid, e.g., without utilizing the reading frame of the selected nucleic acid. The nucleic acid construction is transformed into tissue culture cells, e.g., T cells, by a transfection protocol or lipofection to generate reporter cells.

In one embodiment, the reporter nucleic acid is green fluorescent protein. In a second implementation, the reporter is β-galactosidase. In still other embodiments, the reporter nucleic acid is alkaline phosphatase, β-lactamase, luciferase, or chloramphenicol acetyltransferase. The nucleic acid construction can be maintained on an episome or inserted into a chromosome, for example using targeted homologous recombination.

In the implementation utilizing green fluorescent protein (GFP) or enhanced GFP (eGFP) (Clontech, Palo Alto, Calif.) the reporter cells are grown in microtiter plates wherein each well is contacted with a unique agent to be tested. Following a desired treatment duration, e.g., 5 hours, 10 hours, 20 hours, 40 hours, or 80 hours, the microtiter plate is scanned under a microscope using UV lamp emitting light at 488 nm. A CCD camera and filters set to detect light at 509 nm is used to monitor the fluorescence of eGFP, the detected fluorescence being proportional to the amount of reporter produced.

In the implementation utilizing β-galactosidase, a substrate which produces a luminescent product in a reaction catalyzed by β-galactosidase is used. Again, reporter cells are grown in microtiter plates and contacted with compounds for testing. Following treatment, cells are lysed in the well using a detergent buffer and exposed to the substrate. Lysis and substrate addition is achieved in a single step by adding a buffer which contains a 1:40 dilution of Galacton-Star™ substrate (3-chloro-5-(4-methoxyspiro{1,2-dioxetane-3,2′-(4′chloro)-tricyclo-[3.3.1.1^{3,7}]decan}-4-yl)phenyl-B-D-galactopyranoside; Tropix, Inc., Cat.# GS100), a 1:5 dilution of Sapphire II™ luminescence signal enhancer (Tropix, Inc., Cat.#LAX250), 0.03% sodium deoxycholic acid, 0.053% CTAB, 250 mM NaCl, 300 mM HEPES, pH 7.5). The cells are incubated in the mixture at room temperature for approximately 2 hours prior to quantitation. β-galactosidase activity is monitored by the chemiluminescence produced by the product of β-galactosidase hydrolysis of the Galacton-Star™ substrate. A microplate reader fitted with a sensor is used to quantitate the light signal. Standard software, for example, Spotfire Pro version 4.0 data analysis software, is utilized to analyze the results. The mean chemilurninescent signal for untreated cells is determined. Compounds which exhibit a signal at least 2.5 standard deviations above the mean are candidates for further analysis and testing. Similarly, for alkaline phosphatase, β-lactamase, and luciferase, substrates are available which are fluorescent when converted to product by enzyme.

Isolated Polypeptides

Several aspects of the invention pertain to isolated anergy marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a protein encoded by a nucleic acid selected from Group I or II having less than about 30%, 20%, 10% and more preferably 5% (by dry weight), of non-protein of the invention (also referred to herein as a “contaminating protein”), or of chemical precursors. When a protein of the invention or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

Particularly preferred polypeptides of the present invention have an amino acid sequence sufficiently identical to the amino acid sequence of a polypeptide encoded by a nucleic acid selected from Group I or II. The term “sufficiently identical” or “substantially identical” or “substantially homologous” is used herein to refer to a first amino acid that contains a sufficient or minimum number of identical or equivalent (e.g., with a similar side chain) amino acid residues or nucleotides to a second amino acid sequence such that the first and second amino acid sequences have a common structural domain or common functional activity. For example, amino acid sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical. The invention also includes marker proteins that are substantially homologous to proteins encoded by the anergy markers listed in Group I or Group II or Group III or Group IV and differ in amino acid sequence due to natural and allelic variation or mutagenesis. Parameters for calculating percentage homology are described herein.

In other embodiments, the term “sufficiently identical” or “substantially identical” refers to a polypeptide sequence encoded by a nucleic which is capable of hybridizing under stringent conditions, e.g., highly strigent conditions, to a nucleic acid selected from Group I or Group II or Group III or Group IV. Preferred hybridization conditions are described herein.

In a non-limiting example, as used herein, proteins are referred to as “homologs” and “homlogous” where a first protein region and a second protein region are compared in term of identity. To determine the percent identity of two amino acid sequences or of two polynucleotide sequences, the sequences are aligned for optimal comparison purposes (e.g., gaps can be introduced in one or both of a first and a second amino acid or polynucleotide sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or polynucleotide “identity” is equivalent to amino acid or polynucleotide “homology”). The percent identity between the two sequences is a function of the number of identical positions shared by the sequences, taking into account the number of gaps, and the length of each gap, which need to be introduced for optimal alignment of the two sequences.

The invention also provides chimeric or fusion marker proteins. Within a marker fusion protein the polypeptide can correspond to all or a portion of a marker protein. In a preferred embodiment, a marker fusion protein comprises at least one biologically active portion of a marker protein. Within the fusion protein, the term “operatively linked” is intended to indicate that the marker polypeptide and the non-marker polypeptide are fused in-frame to each other. The non-marker polypeptide can be fused to the N-terminus or C-terminus of the marker polypeptide.

For example, in one embodiment, the fusion protein is a GST-marker fusion protein in which the marker sequences are fused to the C-terminus of the GST sequences. Such fusion proteins can facilitate the purification of recombinant marker proteins.

In another embodiment, the fusion protein is a marker protein containing a heterologous signal sequence at its N-terminus. In certain host cells (e.g., mammalian host cells), expression and/or secretion of marker proteins can be increased through use of a heterologous signal sequence. Such signal sequences are well known in the art.

The marker fusion proteins of the invention can be incorporated into pharmaceutical compositions and administered to a subject in vivo, as described herein. The marker fusion proteins can be used to affect the bioavailability of a marker protein substrate. Use of marker fusion proteins may be useful therapeutically for the treatment of or prevention of damage (e.g., colon damage resulting from cancer) caused by, for example, (i) aberrant modification or mutation of a polynucleotide encoding a marker protein; (ii) mis-regulation of the marker protein-encoding polynucleotide; and (iii) aberrant post-translational modification of a marker protein.

Moreover, the marker-fusion proteins of the invention can be used as immunogens to produce anti-marker protein antibodies in a subject, to purify marker protein ligands and in screening assays to identify molecules which inhibit the interaction of a marker protein with a marker protein substrate.

Preferably, a marker chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). A marker protein-encoding polynucleotide can be cloned into such an expression vector such that the fusion moiety is linked in-frame to the marker protein.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

The present invention also pertains to variants of the anergy marker proteins of the invention which function as either agonists or as antagonists to the marker proteins. In several embodiments of the invention antagonists or agonists of the anergy markers of the invention are therapeutic agents of the invention. For example, agonists of a down-regulated anergy marker can increase the activity or expression of such a marker and therefore ameliorate an immune disorder in a subject wherein said markers are abnormally decreased in level or activity. In one embodiment, the anergy marker GDP Dissociation Inhibitor Beta is abnormally decreased in activity or expression levels in a subject diagnosed with or suspected of having an immune disorder. In this embodiment, treatment of such a subject may comprise administering an agonist wherein such agonist provides increased activity or expression of GDP Dissociation Inhibitor Beta.

In another embodiment of the invention, the anergy marker GBP-3 is abnormally increased in activity or expression levels in a subject diagnosed with or suspected of having an immune disorder, or a decreased expression of normal levels of GBP-3 is desired. In this embodiment, treatment of such a subject may comprise administering an antagonist wherein such antagonist provides decreased activity or expression of GBP-3.

In other embodiments of the invention an agonist or antagonist of an anergy marker is a variant of a marker of the invention. Variants of the marker proteins can be generated by mutagenesis, e.g., discrete point mutation or truncation of a marker protein.

In certain embodiments, an agonist of the marker proteins can retain substantially the same, or a subset, of the biological activities of the naturally occurring form of a marker protein or may enhance an activity of a marker protein. In certain embodiments, an antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring forth of the protein has fewer side effects in a subject relative to treatment with the naturally occurring form of the marker protein. In another preferred embodiment, ionomycin serves as an agonist and an antagonist for anergy marker proteins of the invention depending on whether up- or down-regulation of a particular anergy marker protein of interest is required for treatment of an immune disorder.

Variants of a marker protein which function as either marker protein agonists or as marker protein antagonists can be identified by screening combinatorial libraries of mutants, e.g., truncation mutants, of a marker protein for marker protein agonist or antagonist activity. In one embodiment, a variegated library of anergy marker protein variants is generated by combinatorial mutagenesis at the polynucleotide level and is encoded by a variegated gene library. In certain embodiments, such protein may be used for example as a therapeutic protein of the invention. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods which can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art.

Methods and compositions for screening for protein inhibitors or activators are known in the art (see U.S. Pat. NoS. 4,980,281, 5,266,464, 5,688,635, and 5,877,007, which are incorporated herein by reference).

In addition, libraries of fragments of a protein coding sequence corresponding to an anergy marker protein of the invention can be used to generate a variegated population of marker protein fragments for screening and subsequent selection of variants of a marker protein. In one embodiment, a library of coding sequence fragments can be generated by treating a double stranded PCR fragment of a marker protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the marker protein.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify marker variants.

Screening Methods

The invention includes methods for screening for an agent that alters the activity of a polypeptide encoded by an anergy marker listed in Group I or Group II or Group III or Group IV. For example, the polypeptide can be nucleotide binding protein, e.g., a purine nucleotide binding protein, or a regulator thereof. Such polypeptides can be assayed for their ability to bind, hydrolyze, and release nucleotides. Similarly, a skilled artisan would be able to identify an activity for many polypeptides of the group, e.g., by doing homology searches, molecular modeling, and a variety of in vitro assays. The polypeptide can be purified, e.g., by fusing a nucleic acid encoding the polypeptide to an affinity tag (e.g., an epitope tag such as Flag, HA, or myc, glutathione-S-transferase, chitin binding protein, maltose binding protein, or dihydrofolate reductase). Alternatively, the polypeptide can be purified using standard purification techniques, such as for example, immunoaffinity chromatography, ammonium sulfate precipitation, ion exchange chromatography, filtration, electrophoresis, hydrophobic interaction chromotography, and others.

Also the invention provides methods (also referred to herein as “screening assays”) for identifying modulators, i.e., candidate or test compounds or agents comprising therapeutic moieties (e.g., peptides, peptidomimetics, peptoids, polynucleotides, small molecules or other drugs) which (a) bind to the marker, or (b) have a modulatory (e.g., stimulatory or inhibitory) effect on the activity of an anergy marker or, more specifically, (c) have a modulatory effect on the interactions of the marker with one or more of its natural substrates (e.g., peptide, protein, hormone, co-factor, or polynucleotide), or (d) have a modulatory effect on the expression of the marker. Such assays typically comprise a reaction between the marker and one or more assay components. The other components may be either the test compound itself, or a combination of test compound and a binding partner of the marker. As used herein, the terms “test compound” and “test agent” and “agent” are used interchangeably

As used herein, the term “binding partner” refers to a bioactive agent which serves as either a substrate for a protein encoded by an anergy marker of the invention, or alternatively, as a ligand having binding affinity to the protein for an anergy marker. As mentioned above, the bioactive agent may be any of a variety of naturally-occurring or synthetic compounds, biomolecules, proteins, peptides, oligopeptides, polysaccharides, nucleotides or polynucleotides.

The test compounds of the present invention are generally either small molecules or bioactive agents. Moreover, the test compounds of the present invention can be a large or small molecule, for example, an organic compound with a molecular weight of about 100 to 10000; 200 to 5000; 200 to 2000; or 200 to 1000 Daltons. A test compound can be any chemical compound, for example, a small organic molecule, a carbohydrate, a lipid, an amino acid, a polypeptide, a nucleoside, a nucleic acid, or a peptide nucleic acid. Small molecules include, but are not limited to, metabolites, metabolic analogues, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds). In one preferred embodiment the test compound is a small molecule. In another preferred embodiment, the test compound is a bioactive agent. Bioactive agents include but are not limited to naturally-occurring or synthetic compounds or molecules (“biomolecules”) having bioactivity in mammals, as well as proteins, peptides, oligopeptides, polysaccharides, nucleotides and polynucleotides. Preferably, the bioactive agent is a protein, polynucleotide or biomolecule. One skilled in the art will appreciate that the nature of the test compound may vary depending on the nature of the protein encoded by the marker of the invention. For example, if the marker encodes an orphan receptor having an unknown ligand, the test compound may be any of a number of bioactive agents which may act as cognate ligand, including but not limited to, cytokines, lipid-derived mediators, small biogenic amines, hormones, neuropeptides, or proteases.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds. Compounds and components for synthesis of compounds can be obtained from a commercial chemical supplier, e.g., Sigma-Aldrich Corp. (St. Louis, Mo.). The test compound or compounds can be naturally occurring, synthetic, or both. A test compound can be the only substance assayed by the method described herein. Alternatively, a collection of test compounds can be assayed either consecutively or concurrently by the methods described herein.

A high-throughput method, as described herein, can be used to screen large libraries of chemicals. Such libraries of candidate compounds can be generated or purchased e.g., from Chembridge Corp. (San Diego, Calif.). Libraries can be designed to cover a diverse range of compounds. For example, a library can include 10,000, 50,000, or 100,000 or more unique compounds. Merely by way of illustration, a library can be constructed from heterocycles including pyridines, indoles, quinolines, furans, pyrimidines, triazines, pyrroles, imidazoles, naphthalenes, benzimidazoles, piperidines, pyrazoles, benzoxazoles, pyrrolidines, thiphenes, thiazoles, benzothiazoles, and morpholines. Alternatively, a class or category of compounds can be selected to mimic the chemical structures of malate, oxaloacetate, amocarzine and suramin. A library can be designed and synthesized to cover such classes of chemicals.

In addition, libraries of compounds of the invention can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available. To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy results in a library of peptides, e.g., inhibitors, which can be used to prepare a library of test compounds of the invention. In another illustrative synthesis, a “diversomer library” is created by the method of DeWitt et al. (1993) Proc. Natl. Acad. Sci. USA. 90: 6909. Other synthesis methods, including the “tea-bag” technique of Houghten (see, e.g., Houghten et al., (1991) Nature 354: 84-86) can also be used to synthesize libraries of compounds according to the subject invention.

Libraries of compounds can be screened to determine whether any members of the library have a desired activity, and, if so, to identify the active species. Methods of screening combinatorial libraries have been described. Soluble compound libraries can be screened by affinity chromatography with an appropriate receptor to isolate ligands for a polypeptide encoded by a nucleic acid of Group I or II, followed by identification of the isolated ligands by conventional techniques (e.g., mass spectrometry, NMR, and the like). Immobilized compounds can be screened by contacting the compounds with a polypeptide encoded by a nucleic acid of Group I or II; preferably, the polypeptide is conjugated to a label (e.g., fluorophores, colorimetric enzymes, radioisotopes, luminescent compounds, and the like) that can be detected to indicate ligand binding. Alternatively, immobilized compounds can be selectively released and allowed to diffuse through a membrane to interact with a polypeptide. Exemplary assays useful for screening the libraries of the invention are described below.

In still another embodiment, large numbers of test compounds can be simultaneously tested for binding activity. For example, test compounds can be synthesized on solid resin beads in a “one bead-one compound” synthesis; the compounds can be immobilized on the resin support through a photolabile linker. A plurality of beads (e.g., as many as 100,000 beads or more) can then be combined with yeast cells and sprayed into a plurality of “nano-droplets”, in which each droplet includes a single bead (and, therefore, a single test compound). Exposure of the nano-droplets to UV light then results in cleavage of the compounds from the beads. It will be appreciated that this assay format allows the screening of large libraries of test compounds in a rapid format.

Combinatorial libraries of compounds can be synthesized with “tags” to encode the identity of each member of the library. In general, this method features the use of inert, but readily detectable, tags, that are attached to the solid support or to the compounds. When an active compound is detected (e.g., by one of the techniques described above), the identity of the compound is determined by identification of the unique accompanying tag. This tagging method permits the synthesis of large libraries of compounds which can be identified at very low levels. Such a tagging scheme can be useful, e.g., in the “nano-droplet” screening assay described herein, to identify compounds released from the beads.

In preferred embodiments, the libraries of transcriptional modulator compounds of the invention contain at least 30 compounds, more preferably at least 100 compounds, and still more preferably at least 500 compounds. In preferred embodiments, the libraries of transcriptional modulator compounds of the invention contain fewer than 10⁹ compounds, more preferably fewer than 10⁸ compounds, and still more preferably fewer than 10⁷ compounds.

Screening for Inhibitors of Immune Disorders

The invention provides methods of screening test compounds for inhibitors of immune disorders, and to the pharmaceutical compositions comprising the test compounds. The method of screening comprises obtaining samples from subjects diagnosed with or suspected of having an immune disorder, contacting each separate aliquot of the samples with one of a plurality of test compounds, and comparing expression of one or more anergy marker(s) in each of the aliquots to determine whether any of the test compounds provides: 1) a substantially decreased level of expression or activity of a up-regulated marker, or 2) a substantially increased level of expression or activity of a down-regulated, marker relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a protein encoded by the anergy markers of Group I or Group II or Group III or Group IV and a binding partner, by combining the test compound, protein, and binding partner together and determining whether binding of the binding partner and protein occurs. The test compound may be either small molecules or a bioactive agent. As discussed herein, test compounds may be provided from a variety of libraries well known in the art.

Modulators of an anergy marker expression, activity or binding ability are useful as thereapeutic compositions of the invention. Such modulators (e.g., antagonists or agonists) may be formulated as pharmaceutical compositions, as described herein below. Such modulators may also be used in the methods of the invention, for example, to diagnose, treat, or predict immune disorders.

High-Throughput Screening Assays

The invention provides methods of conducting high-throughput screening for test compounds capable of inhibiting activity or expression of a protein encoded by anergy markers of the invention. In one embodiment, the method of high-throughput screening involves combining test compounds and the marker protein and detecting the effect of the test compound on the encoded protein. Functional assays such as cytosensor microphysiometer, calcium flux assays such as FLIPR® (Molecular Devices Corp, Sunnyvale, Calif.), or the TUNEL assay may be employed to measure cellular activity, as discussed below.

Recent advancements have provided a number of methods to detect binding activity between bioactive agents. Common methods of high-throughput screening involve the use of of fluorescence-based technology, including but not limited, to BRET® or FRET® (both by Packard Instrument Co., Meriden, Conn.) which measure the detection signal provided by the proximity of bound fluorophores. By combining test compounds with proteins encoded by the markers of the invention and determining the binding activity between such, diagnostic analysis can be performed to elucidate the coupling systems. Generic assays using cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by TUNEL assay, which utilizes flow cytometry to detect free 3-OH termini resulting from cleavage of genomic DNA during apoptosis. As mentioned above, a variety of functional assays well-known in the art may be used in combination to screen and/or study the reactivity of different types of activating test compounds. Preferably, the high-throughput screening assay of the present invention utilizes label-free plasmon resonance technology as provided by BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit activity of a protein encoded by the markers listed in Group I or Group II or Group III or Group IV by combining the test compounds and the protein in high-throughput assays such as BIACORE®, or in fluorescence based assays such as BRET®. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

In a specific embodiment, the high-throughput screening assay detects the ability of a plurality of test compounds to bind to GBP-3. In another specific embodiment, the high-throughput screening assay detects the ability of a plurality of a test compound to inhibit a GBP-3 binding partner (such as a ligand) to bind to GBP-3.

Detection Methods

Detection and measurement of the relative amount of a polynucleotide or polypeptide marker of the invention may be by any method known in the art as described in well-known laboratory manuals.

Typical methodologies for detection of a transcribed polynucleotide include RNA extraction from a cell or tissue sample, followed by hybridization of a labeled probe (i.e., a complementary polynucleotide molecule) specific for the target RNA to the extracted RNA and detection of the probe (i.e. Northern blotting).

Typical methodologies for peptide detection include protein extraction from a cell or tissue sample, followed by binding of an antibody specific for the target protein to the protein sample, and detection of the antibody. For example, detection of GBP-3 may be accomplished using polyclonal anti-mouse GBP-3 antibody. Antibodies are generally detected by the use of a labeled secondary antibody. The label can be a radioisotope, a fluorescent compound, an enzyme, an enzyme co-factor, or ligand. Such methods are well understood in the art. In certain embodiments, antibodies specific for the anergy marker of interest are commercially available, for example, anti-caspase-3 antibodies are available from Santa Cruz Biotechnology (Catalog No. sc-1225).

In certain embodiments, the anergy marker polynucleotides themselves (i.e., the DNA or cDNA) may serve as markers for immune disorders. For example, an increase of polynucleotide corresponding to a marker (ie. an up-regulated anergy marker, such as, for example, GBP-3), such as by duplication of the gene, may also be correlated with an immune disorder. Similarly, a decrease of polynucleotide corresponding to a marker (ie. a down-regulated anergy marker, such as, for example, GDP Dissociation inhibitor Beta), such as by deletion of the gene, may also be correlated with an immune disorder.

Detection of specific polynucleotide molecules may also be assessed by gel electrophoresis, column chromatography, or direct sequencing, or quantitative PCR (in the case of polynucleotide molecules) among many other techniques well known to those skilled in the art.

Detection of the presence or number of copies of all or a part of an anergy marker polynucleotide of the invention may be performed using any method known in the art. Typically, it is convenient to assess the presence and/or quantity of a DNA or cDNA by Southern analysis, in which total DNA from a cell or tissue sample is extracted, is hybridized with a labeled probe (i.e. a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

In certain embodiments, the anergy marker proteins or polypeptides may serve as markers for immune disorders. For example, an aberrent increase in the polypeptide corresponding to a marker (i.e. an upregulated anergy marker, such as, for example, GBP-3), may also be correlated with immune disorders. Similarly, an aberrent decrease of a polypeptide corresponding to a marker (ie. a downregulated anergy marker, such as, for example, GDP Dissociation Inhibitor Beta) may also be correlated with immune disorders.

Detection of specific polypeptide molecules may also be assessed by gel electrophoresis, column chromatography, or direct sequencing, among many other techniques well known to those skilled in the art.

Pharmaceutical Compositions

The invention is further directed to pharmaceutical compositions comprising the test compound, or bioactive agent, or a marker modulator (ie. agonist or antagonist), which may further include a marker protein and/or polynucleotide of the invention (e.g., for those markers in Group I or Group II or Group III or Group IV) and can be formulated as described herein. Alternatively, these compositions may include an antibody which specifically binds to an anergy marker protein of the invention and/or an antisense polynucleotide molecule which is complementary to an anergy marker polynucleotide of the invention (e.g., for those markers which are increased in quantity) and can be formulated as described herein.

One or more of the anergy marker genes (listed in Group I or Group II or Group III or Group IV, such as, for example, GBP-3, PTP-1B, jumonji and GRG4) of the invention, fragments of marker genes, marker proteins, marker modulators, fragments of marker proteins, or anti-marker protein antibodies of the invention can be incorporated into pharmaceutical compositions suitable for administration.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The invention includes methods for preparing pharmaceutical compositions for modulating the expression or activity of a polypeptide or polynucleotide corresponding to a marker of the invention. Such methods comprise formulating a pharmaceutically acceptable carrier with an agent which modulates expression or activity of a polypeptide or polynucleotide corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or polynucleotide corresponding to a marker of the invention and one or more additional bioactive agents.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enmnerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on-the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The anergy polynucleotide molecules of the invention can be inserted into vectors and used as gene therapy vectors. Gene therapy vectors can be delivered to a subject by, for example, intravenous injection, local administration or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Therapeutic compositions can be administered with medical devices known in the art. For example, in a preferred embodiment, a therapeutic composition of the invention can be administered with a needleless hypodermic injection device. Examples of well-known implants and modules useful in the present invention include, but are not limited to the following devices known in the art: an implantable micro-infusion pump for dispensing medication at a controlled rate, a therapeutic device for administering medicants through the skin, a medication infusion pump for delivering medication at a precise infusion rate, a variable flow implantable infusion apparatus for continuous drug delivery, an osmotic drug delivery system having multi-chamber compartments, and an osmotic drug delivery system. Many other such implants, delivery systems, and modules are known to those skilled in the art.

Therapeutic Nucleic Acid Vectors

A vector can be designed for administration of an anergy marker to a subject, e.g., a mammal, such that a cell of the subject is able to express a therapeutic polypeptide, e.g., a encoded by an anergy maker listed in Group I or Group II or Group III or Group IV. In addition to the marker encoding the therapeutic polypeptide, the vector can contain regulatory elements, e.g., a 5′ regulatory element, an enhancer, a promoter, a 5′ untranslated region, a signal sequence, a 3′ untranslated region, a polyadenylation site, and a 3′ regulatory region. For example, the 5′ regulatory element, enhancer or promoter can regulate transcription of the DNA encoding the therapeutic polypeptide. The regulation can be tissue specific. For example, the regulation can restrict transcription of the desired marker to T cells, e.g., T cells of a particular developmental stage. Alternatively, regulatory elements can be included that respond to an exogenous drug, e.g., a steroid, tetracycline, or the like. Thus, the level and timing of expression of the therapeutic polypeptide can be controlled.

The vectors can be prepared for delivery as naked nucleic acid, as a component of a virus, or of an inactivated virus, or as the contents of a liposome or other gene delivery vehicle. Alternatively, the gene delivery vehicle, e.g., a viral vector, can be produced from recombinant cells. Appropriate viral vectors include retroviruses, e.g., Moloney retrovirus, poxyiruses, adenoviruses, adeno-associated viruses, and lentiviruses, e.g., Herpes simplex viruses (HSV).

The vector can be administered to a subject, for example, by intravenous injection, by local administration or by stereotactic injection. The vector agent can be further formulated, for example, to delay or prolong the release of the agent by means of a slow release matrix. For example, the vector can be retroviral vector and can be inserted into bone marrow cells harvested from a subject. The cells are infected and grown in culture. Meanwhile, the subject is irradiated to deplete the subject of bone marrow cells. The bone marrow of the subject is then replenished with the infected culture cells. The subject is monitored for recovery and for production of the therapeutic polypeptide.

Antibodies

Antibodies are useful reagents for many embodiments of the invention. An antibody against a polypeptide encoded by an anergy marker listed in Group I or Group II or Group III or Group IV can be used as 1) a reagent to detect the presence of the polypeptide and 2) a reagent to alter the activity or function of the polypeptide. Preferably the antibodies are monoclonal, and most preferably, the antibodies are humanized, as per the description of antibodies herein. In one embodiment, antibodies to the protein encoded by the anergy marker Guanylate Binding Protein-3 may be used in the invention. Other non-limiting examples of antibodies that may be useful in the invention, include, but are not limited to, antibodies that immunospecifically bind to proteins encoded by the anergy markers caspase-3, GDP Dissociation Inhibitor Beta.

An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat et al., (1991) Sequences of Proteins of Immunological Interest, Fifth Edition, U.S. Department of Health and Human Services, NIH Publication No. 91-3242, and Chothia, et al., (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by a nucleic acid of Group I or II). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The antibodies described herein can be human, rodent, humanized, or chimeric antibodies. Methods of producting antibodies are well known in the art. For example, a monoclonal antibody against a target (e.g., a polypeptide encoded by an anergy marker listed in Group I or Group II or Group III or Group IV) can be produced by a variety of techniques, including conventional monoclonal antibody methodology e.g., the standard somatic cell hybridization technique of Kohler and Milstein. Although somatic cell hybridization procedures are preferred, in principle, other techniques for producing monoclonal antibody can be employed e.g., viral or oncogenic transformation of B lymphocytes. The preferred animal system for preparing hybridomas is the murine system. Hybridoma production in the mouse is a very well-established procedure. In this method, a protein corresponding to (e.g., encoded by) an anergy marker of the invention is isolated (e.g., by purification from a cell in which it is expressed or by transcription and translation of a polynucleotide encoding the protein in vivo or in vitro using known methods). A vertebrate, preferably a mammal such as a mouse, rabbit or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

An isolated marker protein, or a portion or fragment thereof, can be used as an immunogen to generate antibodies that bind anergy marker proteins using standard techniques for polyclonal and monoclonal antibody preparation. A full-length marker protein can be used or, alternatively, the invention provides antigenic peptide fragments of these proteins for use as immunogens. The antigenic peptide of an anergy marker protein comprises at least 8 amino acid residues of an amino acid sequence encoded by a marker set forth in Group I or Group II or Group III or Group IV, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the marker protein that are located on the surface of the protein, e.g., hydrophilic regions, as well as regions with high antigenicity.

A marker protein immunogen typically is used to prepare antibodies by immunizing a suitable subject, (e.g., rabbit, goat, mouse or other mammal) with the immunogen. An appropriate immunogenic preparation can contain, for example, recombinantly expressed marker protein or a chemically synthesized marker polypeptide. The preparation can further include an adjuvant, such as Freund's complete or incomplete adjuvant, or similar immunostimulatory agent. Immunization of a suitable subject with an immunogenic marker protein preparation induces a polyclonal anti-marker protein antibody response. Techniques for preparing, isolating and using antibodies are well known in the art.

For example, antibodies to a polypeptide encoded by an anergy marker listed in Group I or Group II or Group III or Group IV can be raised, e.g., by immunization of rabbits with purified polypeptide or with peptides obtained by conventional methods of chemical synthesis, e.g., Merrifield solid phase synthesis. The antisera or monoclonal antibodies can be tested to determine whether they show the ability to discriminate between the polypeptide and other antigens, e.g., by dot immunoblotting or by ELISA. To select a high-affinity reagent with low background signal in the high-throughput screening assay, the candidate antiserum or monoclonal antibody can be further tested under the conditions to be used in the high-throughput screening assay.

Human monoclonal antibodies (mAbs) directed against human proteins can be generated using transgenic mice whose genomes include the human immunoglobulin loci instead of the murine loci. Splenocytes from these transgenic mice immunized with the antigen of interest are used to produce hybridomas that secrete human mAbs with specific affinities for epitopes from a human protein.

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. A similar strategy can also been used to amplify human heavy and light chain variable regions from human antibodies.

The amplified fragments can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in the literature. The fragments can also be variegated prior to expression, e.g., by random or directed mutagenesis or by DNA shuffling (Maxygen, Calif.).

Once displayed on the surface of a display package (e.g., filamentous phage), the antibody library is screened with the target antigen, or peptide fragment thereof, to identify and isolate packages that express an antibody having specificity for the target antigen. Nucleic acid encoding the selected antibody can be recovered from the display package (e.g., from the phage genome) and subcloned into other expression vectors by standard recombinant DNA techniques.

In certain embodiments, the V region domains of heavy and light chains can be expressed on the same polypeptide, joined by a flexible linker to form a single-chain Fv fragment, and the scFV nucleic acid subsequently cloned into the desired expression vector or phage genome. Generally, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

The Fv binding surface of a particular antibody molecule can be further engineered, e.g., on the basis of sequence data for V_{H} and V_{L} (the latter of which may be of the κ or λ chain type). Details of the protein surface that comprises the binding determinants can be obtained from antibody sequence information, by a modeling procedure using previously determined three-dimensional structures from other antibodies obtained from NMR studies or crytallographic data. Protein engineering by molecular modeling is one method for producing a modified antibody.

The term “modified antibody” is also intended to include antibodies, such as monoclonal antibodies, chimeric antibodies, and humanized antibodies which have been modified by, e.g., deleting, adding, or substituting portions of the antibody. For example, an antibody can be modified by deleting the hinge region, thus generating a monovalent antibody. Any modification is within the scope of the invention so long as the antibody has at least one antigen binding region specific.

Chimeric mouse-human monoclonal antibodies (i.e., chimeric antibodies) can be produced by recombinant DNA techniques known in the art. For example, a nucleic acid encoding the Fc constant region of a murine (or other species) monoclonal antibody molecule is digested with restriction enzymes to remove the region encoding the murine Fc, and the equivalent portion of a nucleic acid encoding a human Fc constant region is substituted.

The chimeric antibody can be further humanized by replacing sequences of the Fv variable region which are not directly involved in antigen binding with equivalent sequences from human Fv variable regions. Those methods include isolating, manipulating, and expressing the nucleic acid sequences that encode all or part of immunoglobulin Fv variable regions from at least one of a heavy or light chain. The recombinant DNA encoding the chimeric antibody, or fragment thereof, can then be cloned into an appropriate expression vector. Suitable humanized antibodies can alternatively be produced by CDR substitution.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method, which is capable of replacing at least a portion of a CDR of a human antibody with a CDR derived from a non-human antibody by techniques well-known in the art. The human CDRs may be replaced with non-human CDRs using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining anergy marker polynucleotide and/or polypeptide expression and/or activity, in the context of a biological sample (e.g., blood, serum, cells, tissue) to thereby determine whether an individual is at risk for developing an immune disorder associated with modulated marker expression or activity. The invention also provides for prognostic (or predictive) assays for determining whether an individual is at risk of developing an immune disorder associated with aberrant marker protein or polynucleotide expression or activity.

For example, the number of copies of a marker gene can be assayed in a biological sample. Such assays can be used for prognostic or predictive purposes to thereby prophylactically treat an individual prior to the onset of an immune disorder associated with aberrant marker protein, polynucleotide expression or activity.

Another aspect of the invention pertains to monitoring the influence of agents (e.g., drugs, compounds) on the expression or activity of marker in clinical trials.

Diagnostic Assays

An exemplary method for detecting the presence or absence of marker protein or polynucleotide of the invention in a biological sample involves obtaining a biological sample from a test subject and contacting the biological sample with a compound or an agent capable of detecting the protein or polynucleotide (e.g., mRNA, genomic DNA) that encodes the marker protein such that the presence of the marker protein or polynucleotide is detected in the biological sample. A preferred agent for detecting mRNA or genomic DNA corresponding to a marker gene or protein of the invention is a labeled polynucleotide probe capable of hybridizing to a mRNA or genomic DNA of the invention. In a most preferred embodiment, the polynucleotides to be screened are arranged on a GeneChip®. Suitable probes for use in the diagnostic assays of the invention are described herein. A preferred agent for detecting a marker protein of the invention is an antibody which specifically recognizes the marker.

The diagnostic assays may also be used to quantify the amount of expression or activity of an anergy marker in a biological sample. Such quantification is useful, for example, to determine the progression or severity of an immune disorder. Such quantification is also useful, for example, to determine the severity of an immune disorder following treatment.

Determining Severity of Immune Disorders

In the field of diagnostic assays, the invention also provides methods for determining the severity of an immune disorder by isolating a sample from a subject (e.g., a biopsy or blood draw), detecting the presence, quantity and/or activity of one or more markers of the invention in the sample relative to a second sample from a normal sample or control sample. In one embodiment, the levels of markers in the two samples are compared, and a modulation in one or more markers in the test sample indicates an immune disorder. In other embodiments the modulation of 2, 3, 4 or more markers indicate a severe case of an immune disorder.

In another aspect, the invention provides markers whose quantity or activity is correlated with different manifestations or severity or type of immune disorder. The subsequent level of expression may further be compared to different expression profiles of various stages of the disorder to confirm whether the subject has a matching profile. In yet another aspect, the invention provides anergy markers whose quantity or activity is correlated with a risk in a subject for developing immune disorders.

A preferred agent for detecting marker protein is an antibody capable of binding to a marker protein, preferably an antibody with a detectable label. Antibodies can be polyclonal, or more preferably, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect marker mRNA, protein, or genomic DNA in a biological sample in vitro as well as in vivo. For example, in vitro techniques for detection of marker mRNA include Northern hybridizations and in situ hybridizations. In vitro techniques for detection of marker protein include enzyme linked immunosorbent assays (ELISAs), Western blots, immunoprecipitations and immunofluorescence. In vitro techniques for detection of marker genomic DNA include Southern hybridizations. Furthermore, in vivo techniques for detection of marker protein include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject, e.g., a biopsy or blood draw.

In another embodiment, the methods further involve obtaining a control biological sample from a subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also encompasses kits for detecting the presence of an anergy marker in a biological sample. For example, the kit can comprise a labeled compound or agent capable of detecting marker protein or mRNA in a biological sample; means for determining the amount of marker in the sample; and means for comparing the amount of marker in the sample with a control or standard. The compound or agent can be packaged in a suitable container. The kit can further comprise instructions for using the kit to detect marker protein or polynucleotide.

Prognostic Assays

The diagnostic methods, described herein can furthermore be utilized to identify subjects having or at risk of developing immune disorders associated with aberrant marker expression or activity. In one embodiment of the present invention, as related to immune disorders, aberrant expression or activity of up-regulated anergy markers is typically correlated with an abnormal increase. In another embodiment of the present invention, as related to an immune disorder, aberrant expression or activity of down-regulated anergy markers is typically correlated with an abnormal decrease.

The assays described herein, such as the preceding or following assays, can be utilized to identify a subject having an immune disorder associated with an aberrant level of marker activity or expression. Alternatively, the prognostic assays can be utilized to identify a subject at risk for developing an immune disorder associated with aberrant levels of marker protein activity or polynucleotide expression. Thus, the present invention provides a method for identifying immune disorders associated with aberrant marker expression or activity in which a test sample is obtained from a subject and marker protein or polynucleotide (e.g., mRNA or genomic DNA) is detected, wherein the presence of marker protein or polynucleotide is diagnostic or prognostic for a subject having or at risk of developing immune disorders with aberrant marker expression or activity.

Furthermore, the prognostic assays described herein can be used to determine whether a subject can be administered an agent (e.g., an agonist, antagonist, peptidomimetic, protein, peptide, polynucleotide, small molecule, or other drug candidate) to treat or prevent immune disorders associated with aberrant marker expression or activity, such as, for example, Multiple Sclerosis. For example, such methods can be used to determine whether a subject can be effectively treated with an agent to inhibit immune disorders. Thus, the present invention provides methods for determining whether a subject can be effectively treated with an agent for immune disorders associated with increased marker expression or activity in which a test sample is obtained and marker protein or polynucleotide expression or activity is detected (e.g., wherein the abundance of marker protein or polynucleotide expression or activity is diagnostic for a subject that can be administered the agent to treat injury associated with aberrant marker expression or activity).

In relation to the field of immunology, prognostic assays can be devised to determine whether a subject undergoing treatment for such disorder has a poor outlook for long term survival or disease progression. In a preferred embodiment, prognosis can be determined shortly after diagnosis, i.e., within a few days. By establishing expression profiles of different stages of immune disorders, from onset to acute disease, an expression pattern may emerge to correlate a particular expression profile to increased likelihood of a poor prognosis. The prognosis may then be used to devise a more aggressive treatment program to avert chronic immune disorders and enhance the likelihood of long-term survival and well being.

The methods of the invention can also be used to detect genetic alterations in a marker gene, thereby determining if a subject with the altered gene is at risk for damage characterized by aberrant regulation in marker protein activity or polynucleotide expression. In preferred embodiments, the methods include detecting, in a sample of cells from the subject, the presence or absence of a genetic alteration characterized by at least one alteration affecting the integrity of a gene encoding a marker protein, or the aberrant expression of the marker gene. For example, such genetic alterations can be detected by ascertaining the existence of at least one 1) deletion of one or more nucleotides from a marker gene; 2) addition of one or more nucleotides to a marker gene; 3) substitution of one or more nucleotides of a marker gene, 4) a chromosomal rearrangement of a marker gene; 5) alteration in the level of a messenger RNA transcript of a marker gene, 6) aberrant modification of a marker gene, such as of the methylation pattern of the genomic DNA, 7) the presence of a non-wild type splicing pattern of a messenger RNA transcript of a marker gene, 8) non-wild type level of a marker-protein, 9) allelic loss of a marker gene, and 10) inappropriate post-translational modification of a marker-protein. As described herein, there are a large number of assays known in the art which can be used for detecting alterations in a marker gene. A preferred biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be particularly useful for detecting point mutations in the marker-gene. This method can include the steps of collecting a sample of cells from a subject, isolating polynucleotide (e.g., genomic, mRNA or both) from the cells of the sample, contacting the polynucleotide sample with one or more primers which specifically hybridize to a marker gene under conditions such that hybridization and amplification of the marker gene (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self sustained sequence replication, transcriptional amplification system, Q-Beta Replicase, or any other polynucleotide amplification method, followed by the detection of the amplified molecules using techniques well known to those of skill in the art. These detection schemes are especially useful for the detection of polynucleotide molecules if such molecules are present in very low numbers.

In an alternative embodiment, mutations in an anergy marker gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

In other embodiments, genetic mutations in an anergy marker gene or a gene encoding an anergy marker protein of the invention can be identified by hybridizing a sample and control polynucleotides, e.g., DNA or RNA, to high density arrays containing hundreds or thousands of oligonucleotides probes. For example, genetic mutations in marker can be identified in two dimensional arrays containing light generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the marker gene and detect mutations by comparing the sequence of the sample marker with the corresponding wild-type (control) sequence. Examples of sequencing reactions include those based on techniques developed by Maxam and Gilbert. It is also contemplated that any of a variety of automated sequencing procedures can be utilized when performing the diagnostic assays, including sequencing by mass spectrometry.

Other methods for detecting mutations in an anergy marker gene or gene encoding a marker protein of the invention include methods in which protection from cleavage agents is used to detect mismatched bases in RNA/RNA or RNA/DNA heteroduplexes. In general, the art technique of “mismatch cleavage” starts by providing heteroduplexes by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches. According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like.

In other embodiments, alterations in electrophoretic mobility will be used to identify mutations in marker genes or genes encoding a marker protein of the invention. For example, single strand conformation polymorphism (SSCP) may be used to detect differences in electrophoretic mobility between mutant and wild type polynucleotides. Single-stranded DNA fragments of sample and control marker polynucleotides will be denatured and allowed to renature. The secondary structure of single-stranded polynucleotides varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility.

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing gradient to identify differences in the mobility of control and sample DNA.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) or at the extreme 3′ end of one primer where, under appropriate conditions, mismatch can prevent, or reduce polymerase extension. In addition it may be desirable to introduce a novel restriction site in the region of the mutation to create cleavage-based detection. It is anticipated that in certain embodiments amplification may also be performed using Taq ligase for amplification. In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe polynucleotide or antibody reagent described herein, which may be conveniently used, e.g., in clinical settings to diagnose subjects exhibiting symptoms or family history of a disease or illness involving a marker gene. In a specific embodiment of the invention, a mutation is detected in GBP-3 polynucleotide or GBP-3 polypeptide. In a further specific embodiment, a mutation in GDP Dissociation Inhibitor Beta is correlated with the prognosis or susceptibility of a subject to immune disorders, including Multiple Sclerosis or Type I Diabetes.

Furthermore, any cell type or tissue in which an anergy marker is expressed may be utilized in the prognostic or diagnostic assays described herein.

Monitoring Effects During Clinical Trials

Monitoring the influence of agents (e.g., drugs, small molecules, proteins, nucleotides) on the expression or activity of a marker protein (e.g., the modulation of an anergy marker involved in an immune disorder) can be applied not only in basic drug screening, but also in clinical trials. For example, the effectiveness of an agent determined by a screening assay, as described herein to decrease marker gene expression, protein levels, or down-regulate marker activity, can be monitored in clinical trials of subjects exhibiting increased marker gene expression, protein levels, or up-regulated marker activity. Similarly, the effectiveness of an agent to increase marker gene expression, protein levels, or up-regulate marker activity can be monitored in clinical trials of subjects exhibiting decreased marker polynucleotide expression, protein levels or down-regulated marker activity. In such clinical trials, the expression or activity of a marker gene can be used as a “read out” of the phenotype of a particular tissue.

For example, and not by way of limitation, genes, including marker genes and genes encoding a marker protein of the invention, that are modulated in tissues by treatment with an agent which modulates marker activity (e.g., identified in a screening assay as described herein) can be identified. Thus, to study the effect of agents on marker-associated damage (e.g., the size of tumors), for example, in a clinical trial, cells can be isolated and RNA prepared and analyzed for the levels of expression of marker. The levels of gene expression (e.g., a gene expression pattern) can be quantified by northern blot analysis, RT-PCR or GeneChip® as described herein, or alternatively by measuring the amount of protein produced, by one of the methods as described herein, or by measuring the levels of activity of marker. In this way, the gene expression pattern can serve as a read-out, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, polynucleotide, small molecule, or other drug candidate identified by the screening assays described herein) including the steps of (i) obtaining a pre-administration sample from a subject prior to administration of the agent; (ii) detecting the level of expression of an anergy marker protein or mRNA in the pre-administration sample; (iii) obtaining one or more post-administration samples from the subject; (iv) detecting the level of expression or activity of the marker protein or mRNA in the post-administration samples; (v) comparing the level of expression or activity of the marker protein or mRNA in the pre-administration sample with the marker protein or mRNA the post administration sample or samples; and (vi) altering the administration of the agent to the subject accordingly. For example, decreased administration of the agent may be desirable to decrease expression or activity of marker to lower levels than detected, i.e., to decrease the effectiveness of the agent. According to such an embodiment, marker expression or activity may be used as an indicator of the effectiveness of an agent, even in the absence of an observable phenotypic response.

Prophylactic Methods

In one aspect, the invention provides a method for preventing in a subject, an immune disorder associated with aberrant anergy marker expression or activity, by administering to the subject a marker protein or an agent which modulates marker protein expression or activity.

Subjects at risk for an immune disorder which is caused or contributed to by aberrant marker expression or activity can be identified by, for example, any or a combination of diagnostic or prognostic assays as described herein.

Administration of a prophylactic agent can occur prior to the manifestation of symptoms characteristic of the differential marker protein expression, such that an immune disorder is prevented or, alternatively, delayed in its progression. Depending on the type of marker aberrancy (e.g., typically a modulation outside the normal standard deviation), for example, a marker protein, marker agonist or antagonist agent can be used for treating the subject. The appropriate agent can be determined based on screening assays described herein. Therapeutic Methods

Another aspect of the invention pertains to methods of modulating marker protein expression or activity for therapeutic purposes. Accordingly, in an exemplary embodiment, the modulatory method of the invention involves contacting a cell with an anergy marker (such as, for example, GBP-3 and GDP Dissociation Inhibitor Beta), an anergy marker protein or a test compound that modulates one or more of the activities of a marker protein activity associated with the cell. A test compound that modulates marker protein activity can be a test compound as described herein, such as a polynucleotide or a protein, a naturally-occurring target molecule of a marker protein (e.g., a marker protein substrate), a marker protein antibody, a marker modulator (e.g., agonist or antagonist), a peptidomimetic of a marker protein agonist or antagonist, or other small molecule.

In one embodiment, the test compound stimulates one or more marker protein activities. Examples of such stimulatory test compounds include active marker protein and a polynucleotide molecule encoding a marker protein that has been introduced into the cell.

In another embodiment, the test compound inhibits one or more marker protein activities. Examples of such inhibitory test compounds include antisense marker nucleic acid molecules, anti-marker protein antibodies, and marker protein inhibitors. In a specific embodiment, an inhibitory test compound is an antisense caspase-3 polynucleotide. In another specific embodiment, an inhibitory test compound is an antisense GBP-3 polynucleotide.

These modulatory methods can be performed in vitro (e.g., by culturing the cell with the agent) or, alternatively, in vivo (e.g., by administering the agent to a subject). As such, the present invention provides methods of treating an individual diagnosed with or at risk for an immune disorder characterized by aberrant expression or activity of one or more anergy marker proteins or polynucleotide molecules. In one embodiment, the method involves administering a test compound (e.g., a test compound identified by a screening assay described herein), or combination of test compounds that modulate (e.g., up-regulates or down-regulates) marker protein expression or activity. In another embodiment, the method involves administering a marker protein or polynucleotide molecule as therapy to compensate for reduced or aberrant marker protein expression or activity.

The invention further provides methods of modulating a level of expression of an anergy marker of the invention, comprising administration to a subject having an immune disorder, a variety of compositions which correspond to the markers of Group I or Group II or Group III or Group IV including proteins or antisense oligonucleotides. The protein may be provided by further providing a vector comprising a polynucleotide encoding the protein to the cells. Alternatively, the expression levels of the markers of the invention may be modulated by providing an antibody, a plurality of antibodies or an antibody conjugated to a therapeutic moiety. Treatment with the antibody may further be localized to the tissue comprising an immune disorder. In another aspect, the invention provides methods for localizing a therapeutic moiety to diseased or afflicted tissue or cells comprising exposing the tissue or cells to an antibody which is specific to a protein encoded from the markers of the invention. This method may therefore provide a means to inhibit or enhance expression of a specific polynucleotide corresponding to a marker listed in Group I or Group II or Group III or Group IV. Where the gene is up-regulated as a result of an immune disorder, it is likely that inhibition or prevention of the disorder would involve inhibiting expression of the up-regulated polynucleotide. Where the gene is down-regulated as a result of an immune disorder, it is likely that inhibition or prevention of the disorder would involve enhancing expression of the down-regulated polynucleotide.

Determining Efficacy of a Test Compound or Therapy

The invention also provides methods of assessing the efficacy of a test compound or therapy for inhibiting an immune disorder in a subject. These methods involve isolating samples from a subject suffering from an immune disorder, who is undergoing treatment or therapy, and detecting the presence, quantity, and/or activity of one or more markers of the invention in the first sample relative to a second sample. Where a test compound is administered, the first and second samples are preferably sub-portions of a single sample taken from the subject, wherein the first portion is exposed to the test compound and the second portion is not. In one aspect of this embodiment, the anergy marker is expressed at a substantially decreased level in the first sample, relative to the second. Most preferably, the level of expression in the first sample approximates (i.e., is less than the standard deviation for normal samples) the level of expression in a third control sample, taken from a control sample of normal tissue. In another aspect of this embodiment, the anergy marker is expressed at a substantially increased level in the first sample, relative to the second. Most preferably, the level of expression in the first sample approximates (i.e., is less than the standard deviation for normal samples) the level of expression in a third control sample, taken from a control sample of normal tissue.

In certain embodiments, the normal sample is a non-diseased or non-afflicted cell. In other embodiments the normal sample is derived from a tissue substantially free of an immune disorder.

Where the efficacy of a therapy is being assessed, the first sample obtained from the subject is preferably obtained prior to provision of at least a portion of the therapy, whereas the second sample is obtained following provision of the portion of the therapy. The levels of markers in the samples are compared, preferably against a third control sample as well, and correlated with the presence, or risk of presence, of an immune disorder. Most preferably, the level of markers in the second sample approximates the level of expression of a third control sample. In the present invention, a substantially decreased level of expression of a marker indicates that the therapy is efficacious for treating an immune disorder.

Pharmacogenomics

The marker protein and polynucleotide molecules of the present invention, as well as agents, inhibitors or modulators which have a stimulatory or inhibitory effect on an anergy marker as identified by a screening assay described herein, can be administered to individuals to treat (prophylactically or therapeutically) immune disorders associated with aberrant marker protein activity.

In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a marker molecule or marker modulator as well as tailoring the dosage and/or therapeutic regimen of treatment with a marker molecule or marker modulator.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants.) Such a high-resolution genetic map can be compared to a map of the genome of each of a statistically substantial number of subjects taking part in a Phase II/III drug trial to identify genes associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals. Thus, mapping of the markers of the invention to SNP maps of patients afflicted with an immune disorder may allow easier identification of these genes according to the genetic methods described herein.

Alternatively, a method termed the “candidate gene approach,” can be utilized to identify genes that predict drug response. According to this method, if a gene that encodes a drug target is known (e.g., an anergy differential marker protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabilizer phenotypes is different among different populations. For example, the gene coding for CYP2D6 is highly polymorphic and several mutations have been identified in poor metabilizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabilizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a marker or marker modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a marker or marker modulator, such as a modulator identified by one of the exemplary screening assays described herein.

Kits

The invention also provides kits for determining the prognosis for long term survival in a subject having an immune disorder, the kit comprising reagents for assessing expression of the markers of the invention. Preferably, the reagents may be an antibody or fragment thereof, wherein the antibody or fragment thereof specifically binds with a protein corresponding to a marker from Group I or Group II or Group III or Group IV. For example, antibodies of interest may be prepared by methods known in the art. Optionally, the kits may comprise a polynucleotide probe wherein the probe specifically binds with a transcribed polynucleotide corresponding to an anergy marker listed in Group I or Group II or Group III or Group IV. The kits may also include an array of anergy markers arranged on a biochip, such as, for example, a GeneChip®.

The invention further provides kits for assessing the suitability of each of a plurality of compounds for Inhibiting an immune disorder in a subject. Such kits include a plurality of compounds to be tested, and a reagent (ie. antibody specific to corresponding proteins, or a probe or primer specific to corresponding polynucleotides) for assessing expression of an anergy marker listed in Group I or Group II or Group III or Group IV.

Modifications to the above-described compositions and methods of the invention, according to standard techniques, will be readily apparent to one skilled in the art and are meant to be encompassed by the invention.

This invention is further illustrated by the following examples which should not be construed as limiting. The contents of all references, patents and published patent applications cited throughout this application, as well as the Figures and Tables are incorporated herein by reference.

### EXAMPLES

### Example 1.0 Mice

Mice were maintained in pathogen-free conditions in a barrier facility. BALB/cJ DO11.10 TCR transgenic mice (Murphy et al., (1990) Science 250:1720-1723) were bred with NFAT1−/− mice (Xanthoudakis et al., (1996) Science 272: 892-895) or their isogenic wildtype controls to obtain NFAT1−/− or wildtype DO11.10 TCR transgenic mice.

### Example 1.1 Cell Culture

The murine Th1 cell clone D5 (Ar-5) was cultured as previously described (Agarwal and Rao, (1998) Immunity 9: 765-775). Primary CD4+T cells were isolated from lymph nodes and spleen of NFAT1−/− or wild type DO11.10 transgenic mice using magnetic beads (Dynal), and differentiated in vitro by stimulating for one week with irradiated APC and 1 μg/ml OVA as previously described (Agarwal and Rao, (1998) Immunity 9: 765-775). Jurkat and Phoenix Ecotropic cells were cultured in Dulbecco's modified Eagle's medium supplemented with 10% fetal calf serum, 10 mM HEPES and 2 mM glutamine.

### Example 1.2 Rnase Protection Assay

Total cellular RNA was purified from resting cells or cells stimulated with APC and antigen or anti-CD3 plus anti-CD28 for 4 h, using Ultraspec reagent (Biotecx) and analyzed using the RiboQuant multiprobe RNase protection kit and specific multi-set probes (Pharmingen) according to the manufacturer's instructions. Jurkat cells were transfected by electroporation in serum-free medium with pulses of 250 V and 960 μF with 10 mg/10⁶ cells of pEGFPN1 (Clontech) or pNLS-NFAT1 (ST2+5+8) (Okamura et al., (2000) Mol. Cell. 6: 539-550). RNA from transiently transfected Jurkat cells was obtained after selecting transfected cells for expression of a cotransfected murine CD4 plasmid using magnetic beads (Dynal).

### Example 1.3 Electrophoretic Mobility Shift Assays (EMSAs)

Nuclear extracts were prepared from Th1 cells, unstimulated or stimulated for 60 minutes with 500 nM ionomycin or 20 nM PMA plus 500 nM ionomycin. Binding reactions were performed as previously described (Macian et al., (2000) EMBO J. 19: 4783-1795) using NFAT, AP-1 and NF-KB specific probes (Goldfeld et al., (1993) J. Exp. Med. 178: 1365-1379).

### Example 1.4 Retroviral Infections

Three different retroviral vectors were used, the MSCV containing GFP-KV-DV (Genetics Institute) that expresses GFP from an IRES sequence, GFP-KV-DV-CA-NFAT1 and GFP-KV-DV-CA-RIT-NFAT1 (the last two vectors were constructed by subcloning the DNAs encoding murine constitutively active (ST2+5+8) NFAT1 ((Okamura et al., (2000) Mol. Cell. 6: 539-550) with or without a R468A/1469A/T535G mutation (Macian et al., (2000) EMBO J. 19: 4783-4795) into GFP-KV-DV, respectively). Twenty-four and 48 hours after stimulation with 1 μg/ml plate bound anti-CD3ε and 5 μg/ml anti-CD28 (Pharmingen) in media supplemented with 20 U/ml of IL-2, Th1 cells were infected by spin infection at 1000 g for 90 minutes with retrovirus-containing supernatants derived from the Phoenix Ecotropic packaging cell line (kindly provided by G. P. Nolan), previously transfected using Calcium/phosphate with the corresponding retroviral vectors. Eight μg/ml of polybrene was added to the supernatants during infection. Sevety-two hours post-infection, cells were analyzed and, if necessary, sorted for GFP expression. Infection efficiencies were similar for all three retroviruses, ranging between 10% and 40% in different experiments. Protein expression was confirmed by Western analysis; the levels of CA-NFAT1 proteins in the infected cells tended to be lower than levels of endogenous NFAT1 in wildtype Th1 cells.

### Example 1.5 ELISA

Supernatants from activated cells were collected 24 hours after activation and IL-2 levels were measured in a sandwich ELISA using two different monoclonal anti-mouse IL-2 antibodies (one of them biotinylated) that recognized different epitopes on the IL-2 protein (Pharmingen).

### Example 1.6 Protease Inhibitors Assays

Th1 cells were anergised by pretreatment with 1 μg/ml plate-bound antiCD3ε in the presence of 100 μM Z-VAD.fink (Clabiochem), 5 μM NLVS (Calbiochem) and/or 10 μM lactacystein (Calbiochem) for 16 hours. After that, cells were washed three times and left resting for 48-72 hours. Production of IL-2 and/or IFN-γ in response to stimulation with APC plus antigen or anti-CD3/anti-CD28 was determined by RPA or ELISA.

### Example 1.7 Immunoblotting

Total cellular extracts were prepared by boiling cell pellets directly in SDS containing loading buffer to prevent proteolysis during cell lysis. Antibodies against caspase-3 (Santa Cruz) and active caspase-3 (Cell Signaling) were used. The polyclonal antibody 67.1 against NFAT1 has been described (Okamura et al., (2000) Mol. Cell. 6: 539-550).

### Example 1.8 Proliferation Assay

D5 or primary T cells were stimulated with APC and antigen and pulsed for 24 hours with 10 μCi/ml ³H-thymidine. DNA was collected using a cell harvester and the amount of radioactivity incorporated was measured in a β-counter.

### Example 1.9 RNA Samples and DNA Array Procedures

D5 or primary T cells from NFAT1−/− or wild type DO11.10 transgenic mice were stimulated for 2, 6 or 16 hours with 500 nM ionomycin, 20 nM PMA plus 500 nM ionomycin, or 1 μM CsA plus 500 nM ionomycin. Total RNA was isolated with an RNeasy kit (QIAGEN). Ten μg of total RNA was quantitatively amplified and biotin-labeled as described (Byrne et al., (2000) “Preparation of mRNA for Expression Monitoring” Current Protocols in Molecular Biology (John Wiley & Sons, Inc.) pp. 22.22.21-22.22.13). Hybridization to GeneChips® (Affymetrix) displaying probes for 11,000 mouse genes/ESTs was performed at 40° C. overnight in a mix that included 10 μg fragmented RNA, 6× SSPE, 0.005% Triton X-100 and 100 μg/ml herring sperm DNA in a total volume of 200 μl. Chips were washed, stained with SA-PE and read using an Affymetrix GeneChip® scanner and accompanying gene expression software. Labeled bacterial RNAs of known concentration were spiked into each chip hybridization mix to generate an internal standard curve, allowing normalization between chips and conversion of raw hybridization intensity values to mRNA frequency (mRNA molecules per million).

### Example 1.10 Tunel Assay

Apoptosis was detected by the Tunel method using the In situ Cell Death Detection kit (Boheringer) following manufacture's instructions. Stained cells were analyzed on a FACSCAN (Beckton-Dickinson).

### Example 1.11 Intracellular Cytokine Stains

T cells were stimulated for 4 hours with 1 μg/ml plate-bound anti-CD3ε and 5 μg/ml anti-CD28. For the last two hours, Brefeldin A was added at 10 μg/ml to promote intracellular accumulation of IL-2. After stimulation, cells were fixed in 4% paraphormaldehyde and permeabilized in PBS/1% BSA/0.5% saponin. Cells were then washed and incubated for 10 minutes with Fc-block (Pharmingen) and then for 30 more minutes with 10 μg/ml PE-conjugated anti-mouse IL-2 antibody (Pharmingen) to detect intracellular IL-2. Stained cells were analyzed on a FACSCAN (Beckton-Dickinson). Example 1.12 Quantitative Real Time (RT)-PCR

Total RNA was prepared from resting or stimulated T cells using Ultraspec reagent (Biotecx). cDNA was synthesized using oligo-dT primers and Superscript polymerase (Invitrogen) following the manufacturer's recommendations. Quantitative real-time PCR was performed in an I-Cycler (BioRad) using a SYBR Green PCR kit from Applied Biosystems and specific primers to amplify 100-200 bp fragments from the different genes analyzed. A threshold was set in the linear part of the amplification curve (fluorescence=f[cycle number]) and the number of cycles needed to reach it calculated for every gene. Melting curves as well as agarose gel electrophoresis was performed to ascertain the purity of the amplified band. Normalization was achieved by including a sample with primers for L32.

### Example 2.0, Results: Anergy Marker Expression Profiles

Nucleic acid expression profiles were obtained from a T cell line at multiple time points and in various T cell states. The D5 Th1 T cell line was stimulated in three different ways: 1) ionomycin alone (e.g., causing the regulatory factor NFAT to move from the cytoplasm to the nucleus); 2) ionomycin+PMA (e.g., causing both NFAT and the transcription factor AP-1 to enter the nucleus); 3) ionomycin +cyclosporin A (CsA) (e.g., preventing NFAT from entering the nucleus in response to ionomycin). The ionomycin+CsA condition is a control to insure that the observed changes are not a result of a cellular response to ionomycin unrelated to T cell anergy. Cells were stimulated for 0, 2, 6, and 16 hours. RNA was isolated and analyzed on a nucleic acid chip with probes that monitor the expression of 11,000 nucleic acids located at unique addresses. The experiment was done twice (i.e., with two replicates, Rep. 1 and Rep. 2). Nucleic acid expression at the 2, 6 and 16 hour timepoints was compared to nucleic acid expression at 0 hours.

The nucleic acid expression data was stored in a computer database. A database query was used to identify all nucleic acids that were deemed “increasing” or “decreasing” in response to ionomycin (relative to the 0 hour sample) in at least one timepoint in Rep. 1, and the nucleic acids were also deemed “increasing” or “decreasing” in response to ionomycin in Rep. 2. This query returned expression information on 205 nucleic acids/ESTs out of the 11,000 being monitored. Expression information under multiple conditions was then scrutinized for all 205 nucleic acids: ionomycin alone, ionomycin+PMA, ionomycin+CsA. Expression patterns for each of the 205 nucleic acids was plotted individually. From these 205 nucleic acids, 18 were identified as consistently regulated. The anergy markers of the invention were culled from the 205 nucleic acids described above.

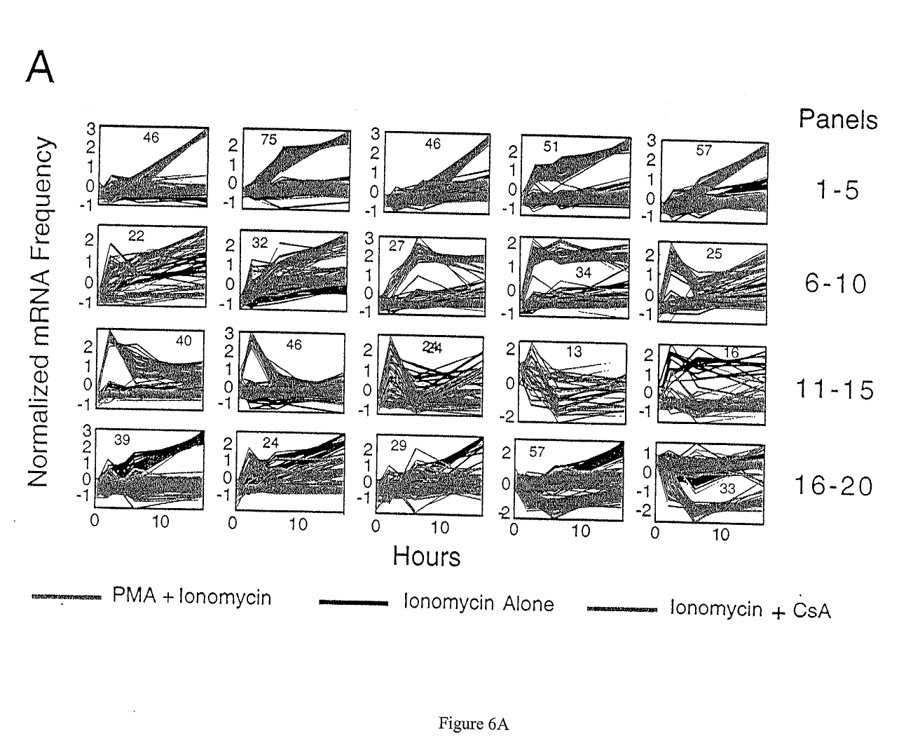

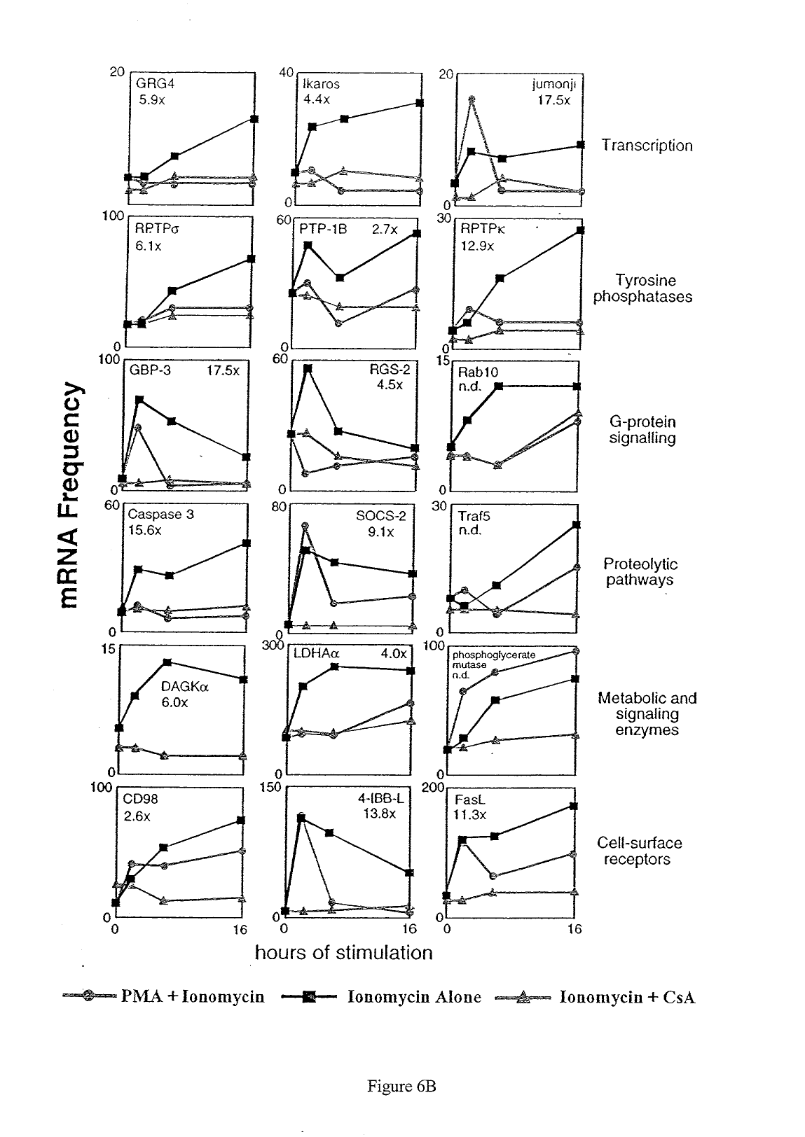

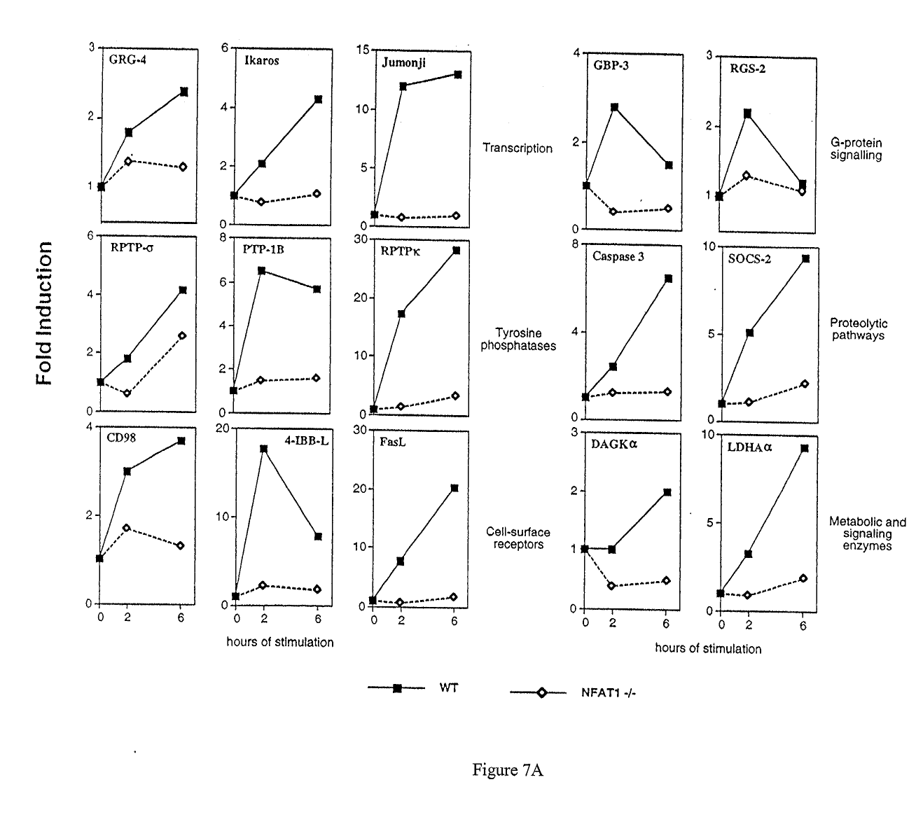

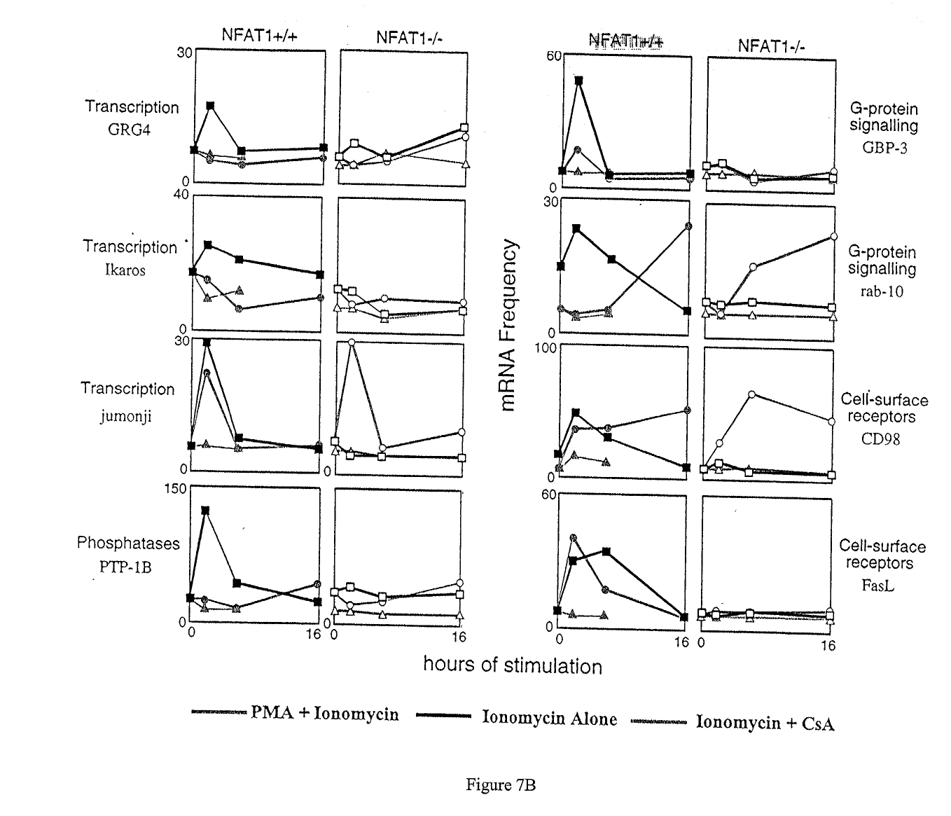

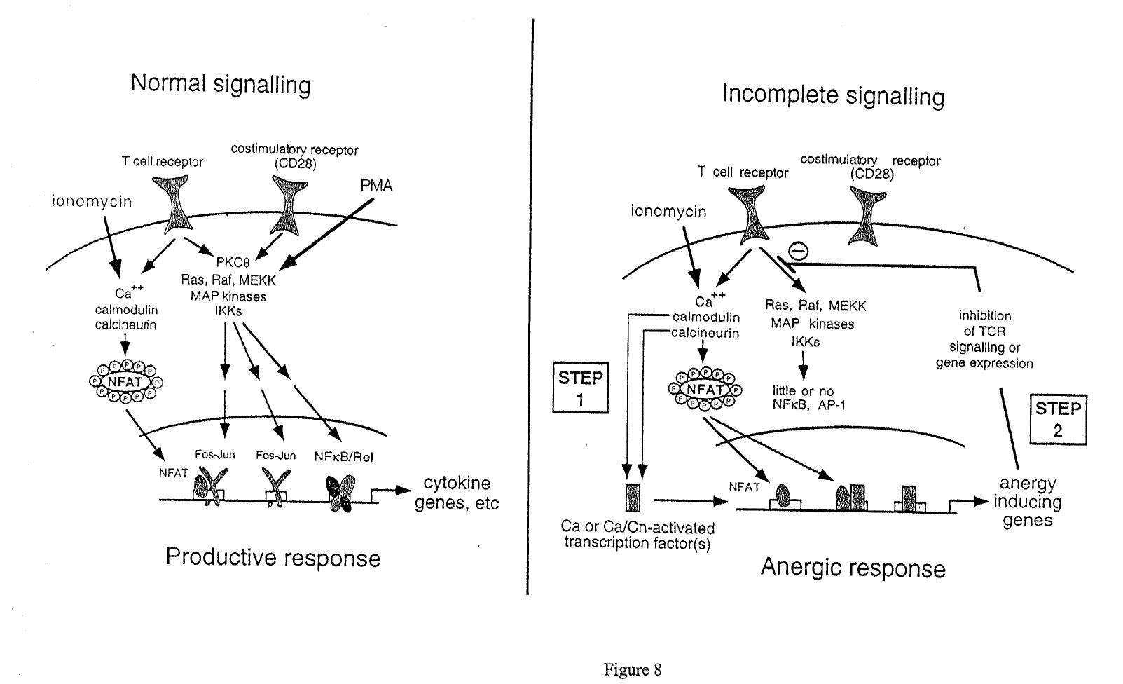

Referring to FIG. 6B, graphs detail the expression levels of 18 nucleic acids at various times after treatment with lonomycin alone, lonomycin+PMA, and lonomycin+CsA. Nucleic acids include the cytokine Interferon-gamma (D5 is a Th1 line) and the chemokines MIP-1α (x12531_s_at) and MIP-1p. In addition, two small nucleotide binding proteins, Msa.21745.0_s_at (also Mm. 21985 and corresponding human Hs. 129764) and U44731_{—}_at (also Mm. 1909 and corresponding human Hs. 240849)), and a potential nucleotide binding protein regulator, Msa.1669.0_f_at (also Mm. 19123 and GenBank PID:g2853176), were modulated in response to ionomycin alone, as was the inhibitory receptor PD-1 (X67914_s_at).

Nucleotide binding proteins such as Rab10 (Msa.21745.0_s_at /Mn. 21985/Hs. 129764) function in the cell as molecular switches. They predominantly adopt one of two conformations, a GTP-bound form and a GDP-bound form (see, e.g., Bourne et al. (1991) Nature 349:117-27). Interaction with signaling molecules such as GTPase exchange factors stimulate the release of the GDP bound by the second form, and its replacement with GTP. Interaction with a GTPase activating factor protein stimulates the hydrolysis of the bound GTP for GDP. The two conformations trigger different downstream events, e.g., through the effector loop near the bound guanine nucleotide. Hence, these nucleotide binding proteins are frequently critical regulators of signaling cascade. Compounds which alter the rate of hydrolysis, GDP release, and GTP binding can, thus, affect signaling events in the cell, particular those related to T cell physiology. Compounds can also affect the ability of the nucleotide binding protein to interact with effectors and with regulators to the same ends. Mutations in the nucleotide binding region are known to perturb the function of nucleotide binding proteins. Such mutations can be introduced by artifice to study the function of the polypeptide, and to introduce a hyper- or hypo-active allele encoding the polypeptide into a cell in culture or a cell of a subject. Further, the alleles encoding the polypeptide can be isolated from a subject and analyzed to identify if mutations are present and associated with an immunological disorder.

U44731_s_at (Mm. 1909 and corresponding human 240849)) is related to guanylate binding proteins. These proteins can bind to GMP in addition to GDP and GTP, and can lack the N(T)KXD consensus motif of canonical G-proteins. Such polypeptide can hydrolyze GTP to GMP (Schwemmle and Staeheli (1994) J. Biol. Chem. 269:11299-305).

Msa.1669.0_f_at (also Mm. 19123 and GenBank PID:g2853176) is a regulator of nucleotide binding proteins. It is a GDP dissociation inhibitor, and hence binds to nucleotide binding protein switches and stabilizes the GDP bound state. The activity of a GDP dissociation inhibitor is critical to the rate of cycling and the state of a G-protein signaling system.

Interferon-gamma, MIP-1α, MIP-1β and EST aa144045_s_at were induced to a larger extent with ionomycin+PMA relative to ionomycin alone. Interestingly, other nucleic acids were modulated similarly or to a lesser extent with ionomycin +PMA relative to ionomycin alone.

Referring to FIG. 3, the induction of caspase-3 with ionomycin is illustrated. Data is from an assay of a sample on a custom nucleic acid array that monitors the expression of approximately 350 nucleic acids with immunological function (left panel). Induction of caspase-3 was detected in a second sample on a nucleic acid chip monitoring 11,000 nucleic acids (right panel).

### Example 2.1, Results: Ionomycin Pretreatment of T Cells Attenuates the Transcription of Specific Inducible Genes

The murine antigen-specific Th1 clone D5 was used to set up the model of clonal anergy developed by Schwartz and coworkers (Schwartz, (1996) J. Exp. Med. 184: 1-8). As previously reported for other T cell clones (Jenkins et al., (1987) Proc. Natl. Acad. Sci. USA 84: 5409-5413), pretreatment of D5 T cells with ionomycin greatly diminished their subsequent proliferative response to antigen or anti-CD3. As expected (Beverly et al., (1992) Int. Immunol. 4: 661-671), anergy was overcome by exposure to IL-2. lonomycin-treated D5 cells showed markedly decreased transcription of several inducible genes, including IL-2, IFN-γ, TNF-α, GM-CSF and MIP-1α, in response to a second stimulation with anti-CD3/anti-CD28 or antigen/antigen presenting cells (APC).

Ionomycin pretreatment also reduced cytokine gene transcription by primary differentiated T cells. In Th1 cells, ionomycin pretreatment led to a pronounced decrease in induction of IL-2, IFN-γ, IL-10, TNF-α and MIP-1α mRNAs upon subsequent stimulation with antigen, with less effect on Fas-L mRNA. lonomycin pretreatment was also effective at inducing anergy in Th2 cells, eliciting approximately 70% reduction in mRNA levels of IL-4, IL-5 and IL-13. Notably, IL-10 transcript levels were not affected by ionomycin pretreatment of Th2 cells, although they were greatly diminished by ionomycin pretreatment of Th1 cells. Thus the net effect of a tolerising stimulus on T cells is to skew the cytokine response towards production of the immunosuppressive cytokine L-10, while down-regulating production of multiple other cytokines and chemokines associated with a productive immune response.

Anergy induction required calcineurin and was correlated with NFAT activation. Overnight treatment of DO 11.10 Th1 cells with the calcineurin inhibitor cyclosporine A (CsA) did not significantly attenuate 1L-2 production in response to subsequent antigen stimulation, when a 3-day washout period was included. However CsA strongly impaired the ability of immobilized anti-CD3 to induce a long-lasting anergic state, implicating calcineurin in anergy induction. In T cells, calcineurin regulates not only NFAT activation but also induction of AP-1 and NFKB (Aramburu et al., (2000) Curr. Top. Cell. Regul. 36: 237-295); however, ionomycin-induced anergy correlated only with activation of NFAT. Nuclear extracts of ionomycin-stimulated Th1 cells showed increased NFAT DNA-binding activity, but no increase in AP-1 or NFκB DNA-binding activity, whereas combined stimulation with PMA and ionomycin induced the cooperative NFAT:AP-1 complex, the AP-1 complex, and the p50/p65 NFκB complex.

### Example 2.2, Results: NFAT−/− T Cells are Less Readily Anergised than Wildtype T Cells

The involvement of NFAT proteins in anergy induction was examined. NFAT1 constitutes 85-90% of total NFAT in resting murine T cells, as assessed by electrophoretic mobility shift assays of total NFAT DNA binding activity in nuclear extracts from stimulated wildtype and NFAT1−/− T cells (Xanthoudakis et al., (1996) Science 272: 892-895). Western analysis with antisera specific for individual NFAT proteins has confirmed that NFAT1 is the predominant NFAT protein in resting human peripheral blood T cells (Lyakh et al., (1997) Mol. Cell. Biol. 17: 2475-2484). NFAT1−/− T cells do not show compensatory increases in other NFATs (Xanthoudakis et al., (1996) Science 272: 892-895); thus these cells not only lack all NFAT1, but also contain only about 10-15% of the normal levels of total NFAT. NFAT1−/− mice were bred to DO 11.10 TcR transgenic mice, and evaluated the ability of tolerising stimuli to induce unresponsiveness in Th1 cells derived from these mice.

Wildtype DO11.10 Th1 T cells that had been pretreated with ionomycin showed markedly decreased induction of L-2 and IFN-γ mRNA. NFAT1−/− Th1 cells showed somewhat lower induction of both cytokines relative to wildtype Th1 cells, presumably because of their lower levels of total NFAT; however they were less susceptible to anergy induction than wildtype Th1 cells, showing perceptible induction of IL-2 and IFN-γ mRNA even after ionomycin pretreatment. The anergised cells were fully responsive to PMA and ionomycin, stimuli which bypass the membrane-proximal steps of TCR signal transduction. NFAT1−/− Th1 cells also showed no anergisation in response to anti-CD3 pretreatment, compared to wildtype T cells which were effectively anergised under these conditions, again supporting a role for NFAT proteins in anergy.

### Example 2.3, Results: A Genetic Program Activated by Calcium Calcineurin and NFAT

To test the hypothesis that NFAT in the absence of AP-1 and NFKB induced a genetic program of anergy, genes induced in D5 T cells by ionomycin stimulation alone were evaluated (FIGS. 1, 2 and 6). Samples from D5 cells stimulated with ionomycin plus CsA, which blocks anergy induction, and ionomycin plus PMA, which pharmacologically mimic complete stimulation through the TCR and CD28 were included. RNA was prepared from unstimulated D5 T cells and from cells stimulated for 2, 6 or 16 h under these three conditions, and gene transcription profiles were evaluated using Affymetrix oligonucleotide arrays (FIG. 6A). For 1349 genes whose expression was altered at least 3-fold by any of the treatments at one or more time points, the results were clustered into 36 panels based on patterns of expression. Twenty of these panels (736 genes) are depicted in FIG. 6A.

Most genes were induced more strongly by PMA/ionomycin stimulation than by ionomycin stimulation alone (FIG. 6A panels 1-12 and three panels not shown; 571 genes total), including essentially all the genes known to be characteristic of the productive immune response (e.g IL-2, IFN-γ, GM-CSF, etc.). Considerably fewer genes were induced more strongly by ionomycin than by PMA/ionomycin (FIG. 6A panels 15-19; 165 genes): genes in panel 15 were rapidly induced, achieving near-maximal levels by 2-6 h of ionomycin stimulation; while those in panels 16-19 were induced more slowly, with higher expression levels at 16 h compared to 2 or 6 h of ionomycin stimulation. Forty genes were equivalently induced by ionomycin and by PMA/ionomycin; these are considered to be ionomycin-induced genes on which PMA had no additional effect (FIG. 6A panels 13, 14 plus a few in panel 6). Panel 20 exemplifies an interesting category of genes that showed no change in expression in response to ionomycin alone but were downregulated in response to PMA/ionomycin, thus achieving differential expression under these two conditions. Thirteen panels (553 genes) are not shown; they were either down-regulated in both ionomycin- and PMA/ionomycin-treated cells, or displayed profiles resembling (but less strikingly) those of panel 20. For almost all genes, alterations in expression were abolished by CsA, consistent with previous findings using human T cells (Feske et al., (2001) Nat. Immunol. 2: 316-324).

By repeating the DNA arrays with RNA prepared from primary Th1 cells, the focus was on approximately 70 genes and ESTs that were equivalently or more strongly induced by ionomycin than by PMA plus ionomycin in both D5 and primary cells. Among them were many ESTs with no assigned function; a single cytokine gene, M-CSF; genes in other functional categories as shown in FIG. 6B; but no other genes normally associated with a productive immune response. FIG. 6B presents DNA array data from D5 T cells for 18 known genes selected from among the strongest of the approximately 70 ionomycin-induced genes. To validate the array data, expression of 15 of the 18 genes were evaluated by quantitative real-time RT-PCR, and in every case ionomycin-mediated induction was confirmed (see numbers within panels of FIG. 6B). To determine whether induction of these genes was dependent on NFAT, gene expression was compared in wildtype and NFAT 1-deficient Th1 cells (FIG. 7). As shown in FIG. 7, 16 of the 18 genes were NFAT1-dependent, based on significantly stronger expression in wildtype Th1 cells following ionomycin stimulation (FIG. 7A, real time RT-PCR data; FIG. 7B, DNA array data). Overall, about 35 of the approximately 70 ionomycin-induced genes showed strong NFAT1-dependent expression, consistent with participation in an NFAT-dependent anergy program, while 20-25 genes were equivalently induced, suggesting participation of transcription factors other than NFAT1 (e.g. different NFAT family members or other calcium/calcineurin-dependent transcriptional activators). Together the results implicate NFAT proteins, directly or indirectly, in a substantial proportion of ionomycin-induced gene transcription in T cells.

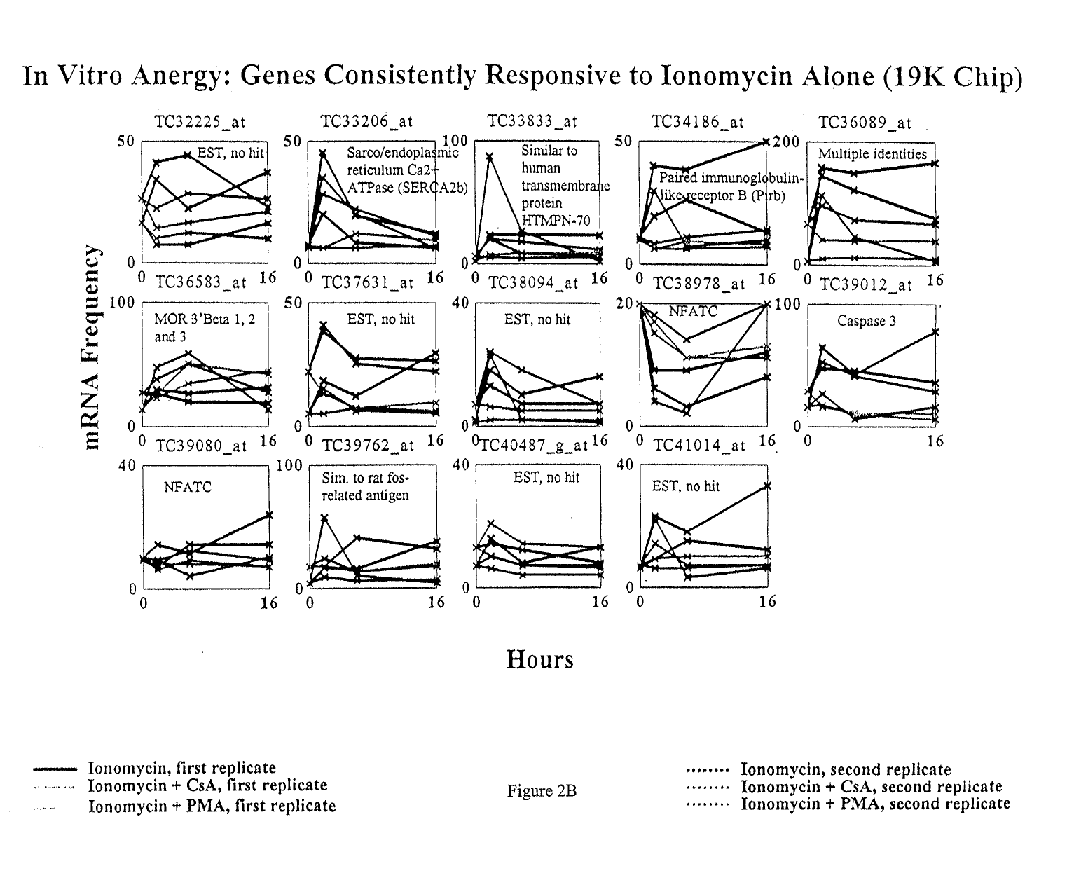

Tables 4-7 show in tabular form the individual data values for the points graphed in FIGS. 1 and 2. For example, Table 4 shows the tabular data from the tests conducted on the 11K chip, which served as the first replicate in FIG. 1. Table 5 shows the tabular data from the tests conducted on the 19K chip, which served as the second replicate in FIG. 2. Similarly, Table 6 shows the tabular data from the tests conducted on the 11K chip, which served as the first replicate in FIG. 2; and Table 7 shows the tabular data from the tests conducted on the 19K chip, which served as the second replicate in FIG. 2.

TABLE 4

| | Ionomycin | | | | Ionomycin + CsA | | | Ionomycin + PMA | | |
|---|---|---|---|---|---|---|---|---|---|---|
| Qualifier | 0 h | 2 h | 6 h | 16 h | 2 h | 6 h | 16 h | 2 h | 6 h | 16 h |
| Z31202_s_at | 18 | 17 | 30 | 22 | 11 | 11 | 11 | 13 | 14 | 22 |
| aa144045_s_at | 20 | 46 | 39 | 33 | 11 | 14 | 13 | 166 | 76 | 87 |
| aa174748_at | 18 | 55 | 23 | 22 | 16 | 15 | 14 | 53 | 43 | 27 |
| c81206_rc_at | 13 | 23 | 16 | 15 | 12 | 10 | 11 | 20 | 16 | 14 |
| D86609_s_at | 27 | 48 | 21 | 20 | 18 | 17 | 14 | 31 | 21 | 28 |
| ET63436_at | 185 | 149 | 82 | 86 | 180 | 146 | 151 | 262 | 287 | 246 |
| k00083_s_at | 17 | 54 | 32 | 35 | 12 | 13 | 13 | 220 | 156 | 112 |
| MIP1-B_at | 154.5 | 265.5 | 102 | 119 | 127.5 | 83 | 118 | 665.5 | 316.5 | 254.5 |
| Msa.11439.0_s_at | 18 | 37 | 31 | 34 | 14 | 15 | 13 | 34 | 27 | 33 |
| Msa.15983.0_f_at | 31 | 40 | 33 | 17 | 19 | 18 | 14 | 30 | 26 | 18 |
| Msa.1669.0_f_at | 45 | 33 | 38 | 35 | 44 | 46 | 38 | 36 | 39 | 35 |
| Msa.18713.0_g_at | 31 | 63 | 35 | 28 | 35 | 28 | 35 | 135 | 80 | 64 |
| Msa.21745.0_s_at | 8 | 9 | 6 | 6 | 5 | 5 | 6 | 8 | 7 | 11 |
| U44731_s_at | 50 | 80 | 26 | 17 | 20 | 15 | 13 | 33 | 26 | 17 |
| x12531_s_at | 6 | 81 | 34 | 97 | 4 | 4 | 5 | 781 | 435 | 365 |
| X67914_s_at | 33 | 59 | 62 | 51 | 34 | 32 | 14 | 82 | 68 | 82 |

TABLE 5

| | Ionomycin | | | | Ionomycin + CsA | | | Ionomycin + PMA | | |
|---|---|---|---|---|---|---|---|---|---|---|
| Qualifier | 0 h | 2 h | 6 h | 16 h | 2 h | 6 h | 16 h | 2 h | 6 h | 16 h |
| Z31202_s_at | 14 | 10 | 17 | 45 | 9 | 11 | 14 | 4 | 4 | 12 |
| aa144045_s_at | 5 | 24 | 17 | 22 | 4 | 4 | 4 | 103 | 90 | 164 |
| aa174748_at | 7 | 31 | 19 | 21 | 11 | 10 | 12 | 46 | 40 | 44 |
| c81206_rc_at | 5 | 15 | 13 | 39 | 3 | 6 | 4 | 35 | 21 | 33 |
| D86609_s_at | 16 | 34 | 34 | 45 | 10 | 13 | 15 | 30 | 17 | 40 |
| ET63436_at | 128 | 113 | 60 | 107 | 156 | 128 | 127 | 93 | 94 | 248 |
| k00083_s_at | 3 | 41 | 45 | 72 | 4 | 9 | 8 | 307 | 421 | 296 |
| MIP1-B_at | 13.5 | 110.5 | 85.5 | 97.5 | 13 | 16 | 16 | 467 | 559 | 423 |
| Msa.11439.0_s_at | 13 | 19 | 22 | 36 | 11 | 10 | 13 | 10 | 10 | 23 |
| Msa.15983.0_f_at | 17 | 44 | 60 | 56 | 21 | 18 | 19 | 33 | 21 | 36 |
| Msa.1669.0_f_at | 46 | 27 | 32 | 50 | 46 | 46 | 43 | 23 | 25 | 44 |
| Msa.18713.0_g_at | 17 | 83 | 58 | 49 | 33 | 34 | 33 | 102 | 54 | 106 |
| Msa.21745.0_s_at | 10 | 8 | 12 | 12 | 4 | 3 | 9 | 4 | 3 | 8 |
| U44731_s_at | 7 | 69 | 54 | 25 | 5 | 7 | 5 | 48 | 3 | 5 |
| x12531_s_at | 8 | 68 | 49 | 84 | 2 | 2 | 8 | 386 | 363 | 373 |
| X67914_s_at | 11 | 53 | 39 | 36 | 4 | 6 | 9 | 59 | 38 | 72 |

TABLE 6

| | Ionomycin + CsA | | | | Ionomycin | | | Ionomycin + PMA | | |
|---|---|---|---|---|---|---|---|---|---|---|
| Qualifier | 0 h | 2 h | 6 h | 16 h | 2 h | 6 h | 16 h | 2 h | 6 h | 16 h |
| TC14671_g_at | 7 | 5 | 6 | 6 | 15 | 19 | 13 | 6 | 9 | 14 |
| TC16364_at | 69 | 62 | 44 | 46 | 38 | 42 | 34 | 38 | 40 | 40 |
| TC16828_at | 13 | 9 | 7 | 7 | 13 | 48 | 30 | 11 | 26 | 29 |
| TC17132_at | 22 | 22 | 25 | 20 | 41 | 54 | 32 | 93 | 50 | 48 |
| TC17495_at | 6 | 6 | 7 | 7 | 10 | 9 | 12 | 12 | 8 | 7 |
| TC17558_at | 7 | 5 | 5 | 10 | 28 | 26 | 23 | 16 | 7 | 7 |
| TC18221_at | 5 | 5 | 6 | 5 | 6 | 8 | 11 | 7 | 4 | 5 |
| TC19211_at | 8 | 6 | 9 | 5 | 21 | 17 | 12 | 6 | 5 | 5 |
| TC21156_at | 15 | 10 | 14 | 9 | 34 | 33 | 16 | 7 | 28 | 11 |
| TC23346_s_at | 7 | 6 | 6 | 6 | 8 | 11 | 13 | 7 | 6 | 4 |
| TC23450_s_at | 16 | 11 | 11 | 15 | 20 | 12 | 13 | 22 | 32 | 16 |
| TC24045_at | 12 | 7 | 7 | 7 | 26 | 21 | 15 | 17 | 18 | 15 |
| TC24067_at | 6 | 5 | 5 | 5 | 8 | 23 | 15 | 6 | 5 | 4 |
| TC25965_at | 6 | 5 | 5 | 10 | 19 | 18 | 16 | 16 | 7 | 10 |
| TC27326_g_at | 68 | 52 | 45 | 51 | 40 | 33 | 29 | 26 | 79 | 41 |
| TC29889_at | 8 | 6 | 6 | 7 | 17 | 15 | 11 | 7 | 7 | 11 |
| TC30384_g_at | 41 | 24 | 23 | 29 | 77 | 73 | 57 | 73 | 81 | 59 |
| TC30935_at | 13 | 9 | 5 | 5 | 30 | 27 | 22 | 26 | 27 | 26 |
| TC30992_s_at | 4 | 4 | 4 | 5 | 14 | 4 | 6 | 13 | 7 | 10 |
| TC31681_at | 36 | 27 | 19 | 25 | 52 | 28 | 18 | 43 | 33 | 16 |
| TC3225_at | 26 | 14 | 16 | 21 | 41 | 44 | 23 | 22 | 28 | 21 |
| TC33206_at | 7 | 6 | 6 | 7 | 20 | 8 | 6 | 28 | 22 | 15 |
| TC33833_at | 6 | 5 | 4 | 5 | 19 | 8 | 6 | 20 | 17 | 8 |
| TC34186_at | 11 | 6 | 6 | 7 | 19 | 26 | 13 | 8 | 11 | 14 |
| TC36089_at | 67 | 41 | 38 | 39 | 145 | 122 | 75 | 95 | 72 | 49 |
| TC36583_at | 27 | 23 | 34 | 45 | 26 | 20 | 18 | 37 | 50 | 26 |
| TC37631_at | 22 | 13 | 7 | 9 | 38 | 27 | 26 | 41 | 25 | 29 |
| TC38094_at | 7 | 6 | 5 | 5 | 13 | 7 | 7 | 24 | 18 | 18 |
| TC38978_at | 20 | 17 | 11 | 13 | 9 | 9 | 12 | 18 | 14 | 17 |
| TC39012_at | 28 | 15 | 11 | 10 | 47 | 45 | 35 | 52 | 41 | 37 |
| TC39080_at | 9 | 6 | 9 | 7 | 7 | 14 | 14 | 9 | 12 | 12 |
| TC39762_at | 17 | 17 | 13 | 19 | 17 | 41 | 31 | 24 | 13 | 14 |
| TC40487_g_at | 13 | 10 | 7 | 6 | 14 | 12 | 8 | 21 | 14 | 7 |
| TC41014_at | 7 | 6 | 6 | 7 | 9 | 15 | 12 | 14 | 7 | 7 |

TABLE 7

| | Ionomycin + CsA | | | | Ionomycin | | | Ionomycin + PMA | | |
|---|---|---|---|---|---|---|---|---|---|---|
| Qualifier | 0 h | 2 h | 6 h | 16 h | 2 h | 6 h | 16 h | 2 h | 6 h | 16 h |
| TC14671_g_at | 6 | 4 | 4 | 6 | 7 | 15 | 30 | 3 | 3 | 4 |
| TC16364_at | 26 | 24 | 20 | 21 | 4 | 5 | 10 | 10 | 6 | 7 |
| TC16828_at | 6 | 5 | 6 | 7 | 4 | 17 | 31 | 3 | 4 | 14 |
| TC17132_at | 21 | 29 | 31 | 51 | 121 | 155 | 126 | 426 | 115 | 124 |
| TC17495_at | 6 | 5 | 6 | 7 | 9 | 12 | 12 | 16 | 5 | 6 |
| TC17558_at | 13 | 16 | 24 | 36 | 41 | 57 | 98 | 57 | 3 | 4 |
| TC18221_at | 6 | 5 | 6 | 9 | 9 | 17 | 25 | 16 | 3 | 3 |
| TC19211_at | 31 | 39 | 11 | 6 | 48 | 22 | 22 | 34 | 7 | 5 |
| TC21156_at | 12 | 11 | 19 | 26 | 20 | 25 | 31 | 16 | 4 | 4 |
| TC23346_s_at | 6 | 4 | 3 | 6 | 5 | 12 | 13 | 8 | 3 | 4 |
| TC23450_s_at | 14 | 13 | 14 | 14 | 16 | 21 | 22 | 31 | 37 | 38 |
| TC24045_at | 6 | 5 | 4 | 5 | 15 | 15 | 25 | 8 | 3 | 5 |
| TC24067_at | 6 | 3 | 3 | 5 | 9 | 19 | 31 | 10 | 6 | 6 |
| TC25965_at | 5 | 5 | 11 | 14 | 42 | 35 | 50 | 26 | 2 | 2 |
| TC27326_g_at | 24 | 19 | 17 | 12 | 3 | 4 | 5 | 2 | 2 | 4 |
| TC29889_at | 7 | 5 | 5 | 4 | 11 | 10 | 13 | 4 | 4 | 4 |
| TC30384_g_at | 19 | 13 | 16 | 13 | 39 | 29 | 53 | 22 | 17 | 34 |
| TC30935_at | 38 | 54 | 48 | 40 | 93 | 60 | 86 | 60 | 46 | 46 |
| TC30992_s_at | 15 | 12 | 11 | 10 | 28 | 9 | 12 | 33 | 34 | 11 |
| TC31681_at | 28 | 15 | 17 | 15 | 56 | 31 | 22 | 45 | 52 | 50 |
| TC3225_at | 16 | 10 | 12 | 10 | 34 | 22 | 37 | 7 | 7 | 13 |
| TC33206_at | 6 | 6 | 12 | 9 | 45 | 19 | 12 | 35 | 20 | 31 |
| TC33833_at | 2 | 7 | 8 | 9 | 23 | 23 | 23 | 87 | 26 | 32 |
| TC34186_at | 10 | 6 | 9 | 8 | 40 | 38 | 50 | 30 | 7 | 15 |
| TC36089_at | 5 | 9 | 12 | 9 | 156 | 149 | 165 | 113 | 45 | 75 |
| TC36583_at | 13 | 26 | 50 | 42 | 29 | 26 | 31 | 47 | 58 | 69 |
| TC37631_at | 5 | 5 | 7 | 6 | 18 | 12 | 29 | 15 | 6 | 17 |
| TC38094_at | 2 | 2 | 2 | 2 | 18 | 10 | 16 | 23 | 2 | 4 |
| TC38978_at | 20 | 15 | 11 | 11 | 6 | 3 | 8 | 4 | 2 | 3 |
| TC39012_at | 15 | 17 | 9 | 6 | 64 | 42 | 77 | 26 | 6 | 13 |
| TC39080_at | 10 | 8 | 8 | 7 | 14 | 11 | 24 | 9 | 4 | 8 |
| TC39762_at | 4 | 9 | 6 | 7 | 16 | 15 | 37 | 57 | 10 | 8 |
| TC40487_g_at | 7 | 6 | 4 | 4 | 16 | 8 | 13 | 10 | 7 | 9 |
| TC41014_at | 6 | 9 | 10 | 10 | 23 | 18 | 33 | 22 | 3 | 3 |

Like the calcium influx-dependent genes previously described (Feske et al., (2001) Nat. Immunol. 2: 316-324), the ionomycin-induced genes shown in FIG. 7B displayed diverse patterns of gene expression, indicating diverse mechanisms of regulation by PMA-and ionomycin-induced signaling pathways (FIG. 7B). Grg4, Ikaros, PTP-1B and GBP-3 were more strongly induced by ionomycin compared to PMA/ionomycin; jumonji, CD98 and FasL were similarly induced by these two stimuli; Rab-10 was markedly induced by ionomycin at early times, but also showed late, high-level induction in response to PMA/ionomycin (FIGS. 6B, 7B). lonomycin-mediated gene induction required NFAT1 (FIGS. 7A, B), consistent with the fact that NFAT1 is the predominant NFAT protein in resting and ionomycin-stimulated cells (Lyakh et al., (1997) Mol Cell. Biol. 17: 2475-2484); Xanthoudakis et al., (1996) Science 272: 892-895). For jumonji, Rab10 and CD98, PMA/ionomycin-mediated induction was not NFAT1-dependent (FIG. 7B); this response may be mediated by inducible isoforms of NFAT2, which are only synthesised following PMA/ionomycin stimulation (Chuvpilo et al., (1999) Immunity 10: 261-269; Lyakh et al., (1997) Mol. Cell. Biol. 17: 2475-2484).

### Example 2.4, Results: Proteolytic Pathways Involved in Anergy Induction

Of particular interest was the fact that several of the ionomycin-induced genes encoded proteins involved (or potentially involved) in protein degradation. Candidate genes in this category included caspase-3, SOCS-2 and Traf5 (FIG. 6B). Caspase-3 is an enzyme for which commercial reagents are readily available. Tests were conducted to determine whether caspase-3 was activated and functional in ionomycin-pretreated T cells. RNase protection assays confirmed that caspase-3 was upregulated at the mRNA level in ionomycin-treated but not PMA/ionomycin-treated D5 T cells, and that the induction was sensitive to CsA. Likewise, primary Th1 cells showed marked induction of caspase-3 mRNA when stimulated with anti-CD3 alone, but much less induction when simultaneously stimulated with anti-CD28. Western blotting showed that caspase-3 was induced at the protein level and activated in ionomycin-treated T cells: induction of the inactive precursor procaspase 3 was observed by 6 h, while the partially-processed p20 and fully-processed, active p17 forms were observed by 6 h and persisted for 16-18 h. Ionomycin-treated D5 cells also showed increased expression of Traf5 and a Cb1/Cb1-b related protein. Caspase-3 activation was not associated with cell death, since several independent assays showed no detectable apoptosis of ionomycin-stimulated D5 or primary T cells. Western blots of ionomycin-treated D5 cells showed minor cleavage of Vav-1, Gads and PKC-theta, three established caspase-3 substrates implicated in T cell signaling (Berry et al., (2001) Oncogene 20: 1203-1211; Datta et al., (1997) J. Biol. Chem. 272: 20317-20320; Hofmann et al., (2000) Oncogene 19: 1153-1163; Yankee et al., (2001) Proc. Natl. Acad. Sci., USA 98: 6789-6793); proteolysis was not indiscriminate, however, since several other signaling proteins were unaffected. Further work will be necessary to establish whether proteolysis occurs in specific intracellular compartments relevant to TCR signal transduction or whether other signaling proteins are substrates for proteolysis in anergic cells.

Whether anergy could be prevented by including caspase and proteasome inhibitors in the first step of pretreatment with ionomycin or anti-CD3 was tested to evaluate the functional importance of proteolytic pathways in anergy induction. Treatment with combinations of caspase and proteasome inhibitors (ZVAD and NLVS, or ZVAD and lactacystin) during the first anergy-inducing step invariably resulted in significant recovery of antigen responses by primary Th1 cells, as assessed by ELISAs or RNase protection assays for IL-2 and IFN-Y. Recovery of IL-2 production ranged from 18 to 87%, while recovery of IFN-γ production ranged from 45 to 115%. The variability likely reflects the fact that the proteasome inhibitors are toxic, and needed to be washed away hours to days before the assay stimuli (antigen/APC or anti-CD3/anti-CD28) were applied. In two experiments, substantial recovery of IL-2 production (90% and 112%, respectively) was observed in primary Th1 cells treated with the single inhibitors ZVAD and NLVS, while in two other experiments the recovery with single inhibitors ranged between 5 and 21%. Similar results were obtained with the D5 T cell clone. Thus directed proteolysis of signaling proteins may be one of several mechanisms that cooperate to maintain lymphocyte anergy.

### Example 2.5, Results: Anergy Is Induced BY NFAT In The Absence Of AP-1

To determine whether NFAT1 is sufficient to impose the anergic state, a constitutively-active version of NFAT1, termed CA-NFAT1, was used (Okamura et al., (2000) Mol. Cell. 6: 539-550). This protein bears alanine substitutions in 12 phosphorylated serines whose dephosphorylation is required for nuclear localization; it is constitutively nuclear under conditions where endogenous NFAT proteins are fully localized to the cytoplasm (Okamura et al., (2000) Mol. Cell. 6: 539-550). CA-NFAT1 was shown to act positively to induce the transcription of endogenous cytokine genes. The protein was introduced by transient transfection into Jurkat cells, the transfected population was left unstimulated or stimulated with PMA alone (neither condition permits activation of endogenous NFAT proteins), and cytokine expression was assessed by RNase protection assay. Untransfected Jurkat cell populations showed no cytokine expression, as expected from the lack of activation of endogenous NFAT, while cells transfected with the CA-NFAT1 plasmid showed perceptible basal induction of the TNFα gene, an NFAT-dependent gene that can be transcribed in the absence of NFAT-AP-1 cooperation, as well as strong PMA-stimulated induction of the IL-3, GM-CSF and MIP-1α genes, which require the cooperative interaction of NFAT and AP-1 (Macian et al., (2000) EMBO J. 19: 4783-4795).

Despite its ability to activate cytokine transcription, CA-NFAT1 paradoxically reduced antigen responsiveness when expressed in unstimulated T cells. The protein was retrovirally expressed in NFAT1-deficient Th1 cells; an empty IRES-GFP retrovirus was used as the control. Five to 7 days after infection, the ability of the brightest GFP+cells to produce IL-2 in response to anti-CD3/anti-CD28 stimulation was assessed by intracellular cytokine staining. In four independent experiments, T cells expressing CA-NFAT1 showed markedly decreased IL-2 production following anti-CD3/anti-CD28 stimulation, compared to control T cells expressing GFP alone. Thus continuous expression of an NFAT protein induces an anergic state, in which T cells are significantly less capable of producing IL-2 in response to TCR stimulation.

The transcription factor AP-1 (Fos/Jun) is an established partner of NFAT in productively-stimulated T cells (Chen et al., (1998) Nature 392: 42-48; Macian et al., (2000) EMBO J. 19: 4783-4795; Rao et al., (1997) Ann. Rev. Immunol. 15: 707-747). To determine whether the ability of NFAT proteins to impose anergy involved cooperation with Fos-Jun proteins basally present in the nucleus of resting cells, CA-RIT-NFAT1, a CA-NFAT1 derivative engineered to be incapable of cooperation with AP-1, was used. In addition to the serine>alanine substitutions present in CA-NFAT1, this protein contains three point mutations in its DNA-binding domain which abrogate Fos-Jun interaction (R468A/I469A/T535G; (Macian et al., (2000) EMBO J. 19: 4783-4795)). In three independent experiments, CA-RIT-NFAT1 was as efficient as CA-NFAT1 at inducing the anergic state, implying that NFAT:AP-1 cooperation is not required for anergy induction.

Constitutive expression of CA-NFAT1 sufficed for basal transcription of mRNAs encoding caspase-3 and certain other anergy-associated genes. CA-NFAT1 was retrovirally expressed in primary NFAT1−/− Th1 cells, GFP+ cells were isolated by cell sorting, RNA was prepared from the unstimulated cells, and expression of 15 anergy-associated genes that were known to be NFAT1-dependent (see FIG. 7A) was assessed by real-time RT-PCR. Only a subset of the 15 genes showed increased basal expression in CA-NFAT1-expressing cells. RPA analysis confirmed that caspase-3 could be induced by CA-NFAT1 as well as CA-RIT-NFAT1 in resting cells, indicating that its expression was NFAT-dependent but independent of NFAT1-AP-1 cooperation. Thus NFAT1 is both necessary and sufficient for expression of certain anergy-associated genes, while expression of others requires additional signaling pathways or transcriptional partners induced by calcium mobilization.

### Example 3.0, Discussion and Implications

The data are consistent with the model of tolerance induction depicted in FIG. 8. It is believed that NFAT plays a central role, not only in productive activation of lymphocytes but also in tolerance induction. Balanced NFAT-AP-1 activation is required for transcription of most genes in the productive immune response (FIG. 8, productive response), while tolerance induction is associated with unbalanced activation of the calcium arm of the TCR signal transduction pathway relative to the PKC/IKK/Ras/MAP kinase arm (FIG. 8, anergic response). Under these conditions of unbalanced activation of NFAT relative to its cooperating transcription factor AP-1 (Fos-Jun), NFAT is diverted towards transcription of an alternate set of anergy-associated genes, whose products together impose the tolerant state (FIG. 8, anergic response). This model does not exclude the participation of non-transcriptional mechanisms dependent on calcium signaling, or participation of calcium-regulated transcriptional modulators other than NFAT.

The model is consistent with essentially all previous data on tolerance induction, both in vivo and ex vivo. Experimental data supporting this model are as follows: First, T cells 8iilacking NFAT1, the major NFAT protein expressed in resting cells, are much more resistant than wildtype T cells to anergy induction ex vivo, consistent with previous findings of T and B cell hyperproliferation in mice lacking NFAT1 (Heyer et al., (1997) Immunobiol. 198: 162-169; Hodge et al., (1996) Immunity 4: 397-405; Schuh et al., (1998) Eur. J. Immunol. 28: 2456-2466; Xanthoudakis et al., (1996) Science 272: 892-895) or both NFAT1 and NFAT2 (Peng et al., (2001) Immunity 14: 13-20). Second, T cells anergised with ionomycin show selective NFAT activation as well as induction of a novel set of anergy-associated genes; these genes are distinct from those activated during the productive immune response, and encode diverse categories of proteins that could plausibly impose an anergic state. Third, a substantial number of anergy-associated genes are direct or indirect targets of NFAT, since they are expressed at significantly lower levels in NFAT1−/− T cells following ionomycin stimulation. Fourth, the anergy-associated genes include genes encoding caspase-3 and putative E3 ligases; caspase-3 is an AP-1-independent NFAT target gene in T cells, and experiments with caspase and proteasome inhibitors suggest that directed proteolysis of signaling proteins contributes to T cell anergy. Fifth, constitutively active versions of NFAT, even if they cannot cooperate productively with AP-1, are capable of inducing caspase-3 expression and down-regulating IL-2 production when retrovirally introduced into NFAT1-deficient Th1 cells.

The model provides a molecular explanation for the observation that anergy is often associated with the apoptotic process of activation-induced cell death (AICD) (Kamradt and Mitchison, (2001) N. Engl. J. Med. 344: 655-664; Kruisbeek and Amsen, (1996) Curr. Opin. Immunol 8: 233-244; Li et al., (2000) Curr. Opin. Immunol. 12: 522-527). Mice injected with superantigens (proteins that interact simultaneously with MHC Class II and the VP region of the TCR) or high doses of soluble antigen delete large numbers of reactive cells, but the surviving cells are tolerant to subsequent stimulation (Garside and Mowat, (2001) Semin. Immunol. 13: 177-185; Kruisbeek and Amsen, (1996) Curr. Opin. Immunol. 8: 233-244). Similarly in the B cell HEL model, there is evidence for early deletion of B cells bearing the anti-HEL BCR (Fang et al., (1998) Immunity 9: 35-45). It has been previously shown that NFAT:AP-1 cooperation is required for AICD (Macian et al., (2000) EMBO J. 19: 4783-4795); consistent with this finding, T cells anergised with ionomycin alone showed no evidence of cell death in our experiments, despite increased expression of active caspase-3 protein and FasL mRNA. It is believed that both NFAT and AP-I are induced early in response to high circulating concentrations of antigen or superantigen, but individual cells show varying ratios of NFAT to AP-1. This results in activation-induced death of cells with the highest NFAT:AP-1 ratios, but establishment of the anergic state in cells with the lowest levels of AP-1.

One question is how the anergic state is prevented when cells encounter a high-affinity foreign antigen to which a rapid and productive response is needed. NFAT-containing transcription complexes are in a dynamic state of reversible dissociation, since ongoing NFAT-dependent gene activation is rapidly reversed by CsA (Timmerman et al., (1996) Nature 383: 837-840; Umlauf et al., (1995). Moreover, the affinity with which cooperative NFAT:AP-1 complexes form on DNA is significantly higher than that exhibited by NFAT binding independently to DNA (approximately 20-fold difference on the murine distal 1L-2 promoter ARRE-2 site; G. Powers and PG Hogan, unpublished; Jain et al., (1993). Thus when cells encounter a strong stimulus with engagement of both antigen and costimulatory receptors, the resulting up-regulation and activation of Fos-Jun proteins would divert NFAT from the lower-affinity sites likely to be characteristic of anergy-associated genes, to the high-affinity NFAT:AP-1 sites observed in genes induced in the productive immune response.

The data support the existence of two distinct mechanisms of tolerance induction in lymphocytes. The first is simple interference with signaling pathways coupled to antigen receptors (Boussiotis et al., (1997) Science 278: 124-128; Fields et al., (1996) Science 271: 1276-1278; Healy et al., (1997) Immunity 6: 419-428; Li et al., (1996) Science 271: 1272-1276). This process could be mediated by the protein products of several anergy-associated genes, including soluble and receptor tyrosine phosphatases (Germain and Stefanova, (1999) Ann. Rev. Immunol. 17: 467-522); diacylglycerol kinase alpha (Sanjuan et al., (2001) J. Cell. Biol. 153: 207-220); and the cell-surface receptor CD98, which is coupled to increased GTP loading of the small G protein Rap1 (Suga et al., (2001) FEBS Lett. 489: 249-253). Rap1 activation has been linked to impaired activation of the ERK MAP kinase pathway in anergic T cells (Boussiotis et al., (1997) Science 278: 124-128; Fields et al, (1996) Science 271: 1276-1278; Li et al, (1996) Science 271: 1272-1276; reviewed in Bos, (1998) EMBO J. 17: 6776-6782). The data suggest that proteolytic mechanisms contribute to anergy induction: treatment of T cells with caspase and proteasome inhibitors during the first phase of anergy induction reproducibly led to recovery of the cytokine response. Caspase-3 has been implicated in modulating lymphocyte responses under conditions where its activation does not appear to be associated with cell death; its targets in the T cell activation pathway include Vavl, PKC-theta, the adapter protein Gads, and the zeta chain of the TCR/CD3 complex (Berry et al., (2001) Oncogene 20: 1203-121 1; Datta et al., (1997) J. Biol. Chem. 272: 203 17-20320; Gastman et al., (1999) Cancer Res. 59: 1422-1427; Hofmann et al., (2000) Oncogene 19: 1153-1163; Yankee et al., (2001) Proc. Natl. Acad. Sci., USA 98: 6789-6793). Like the related proteins SOCS-1 and Traf6 (Kamizono et al., (2001) J. Biol. Chem. 276: 12530-12538; Wang et al., (2001) Nature 412: 346-351), SOCS-2 and Traf5 may be E3 ligases involved in protein degradation. Mice lacking the E3 ligases Itch and Cb1-b show a striking autoimmune phenotype (Bachmaier et al., (2000) Nature 403: 211-216; Chiang et al., (2000) Nature 403: 216-220); Perry et al., (1998) Nat. Genet. 18: 143-148), emphasizing that proteolytic pathways play a role in tolerance induction. Directed proteolysis of specific signaling components in anergic T cells could explain the long-lasting nature of anergy in vivo and ex vivo (Lanoue et al., (1997) J. Exp. Med 185: 405-414; Schwartz, (1996) J. Exp. Med. 184: 1-8), as well as the finding that anergy is dominant in somatic cell fusion experiments (Telander et al., (1999) J. Immunol. 162: 1460-1465).

The data also support a mechanism of selective transcriptional modulation which blocks essentially all cytokine production by Th1 cells, while skewing the cytokine profile of Th2 cells away from IL-4 transcription and towards IL-10 production. Indeed in an in vivo model of T cell tolerance, self-reactive T cells resident in lymphoid organs produced primarily IL-10 (Buer et al., (1998) J. Exp. Med 187: 177-183). Preferential IL-10 production by anergic T cells provides a link between the two current models of how peripheral tolerance is maintained: the cell-intrinsic mechanism of anergy induction would attenuate the antigen responsiveness of differentiated effector T cells, while the bias towards L-10 production by Th2 cells would lead to some immunosuppression by itself but would also result, over the longer term, in generation of IL-10-producing regulatory T cells capable of suppressing any remaining productive response (Maloy and Powrie, (2001) Nat. Immunol 2: 816-822). Since the transcriptional skewing is both celltype- and cytokine-specific, it is likely to be imposed in the nucleus by transcriptional modulators which act on specific genes, rather than in the cytoplasm by global interference with the TCR signaling complex. Candidate transcriptional modulators emerging from the screens include Ikaros, a family of proteins implicated in gene silencing (Sabbattini et al., (2001) EMBO J. 20: 2812-2822; Brown et al, (1997) Cell 91: 845-854); the Groucho-related protein Grg4 (Eberhard et al., (2000) EMBO J. 19: 2292-2303; and the DNA-binding protein jumonji that negatively regulates cell proliferation (Toyoda et al., (2000) Biochem. Biophys. Res. Commun. 274: 332-336).

The hypothesis that anergy is induced by NFAT in the absence of Fos and Jun has practical implications. Potentially, a long-lasting tolerant state could be induced at any time, even in the presence of ongoing immune stimulation, merely by disrupting the interaction of NFAT with Fos and Jun. This should eliminate or severely disrupt transcription of genes involved in the productive immune response for which NFAT-AP-1 cooperation is essential, while at the same time switching the cell's genetic program towards transcription of the distinct set of anergy-inducing genes that are activated by NFAT in the absence of Fos and Jun. Availability of the detailed molecular structure of the NFAT-Fos-Jun-DNA complex (Chen et al., (1998) Nature 392: 42-48) will facilitate identification of peptide and small molecule inhibitors that selectively disrupt cooperative NFAT:Fos:Jun complexes on composite NFAT-AP-1 sites, without affecting independent binding of NFAT or Fos/Jun to noncomposite sites.

The present invention has been described in some detail by way of example for the purpose of clarity and understanding. It will be apparent to those of ordinary skill in the art that certain changes and modifications may be made to the present invention without departing from either the spirit or the scope of the claims. Although the present invention has been described with reference to the presently preferred embodiments, it is understood that various modifications may be made without departing from the spirit of the invention. Accordingly, the invention is not to be limited except as specifically set forth in the following claims.

What is claimed is:

1. A method of screening for test compounds capable of modulating the activity of an anergy marker protein encoded by an anergy marker listed in Group I or Group II or Group III or Group IV, the method comprising:
a) contacting the anergy marker protein with a plurality of test compounds;
b) detecting binding of one of the test compounds to the anergy marker protein, relative to other test compounds; and
c) correlating the amount of binding of the test compound to the anergy marker protein with the ability of the test compound to modulate the activity of the anergy marker protein, wherein binding indicates that the test compound is capable of modulating the activity of the anergy marker protein and wherein the nucleic acid sequence of the anergy marker is 75% homologous to the anergy marker listed in Group I or Group II or Group III or Group IV.

2. The method of claim 1, wherein the method of screening is high-throughput screening.

3. The method of claim 1, wherein the test compound is from a library selected from a group of libraries consisting of spatially addressable parallel solid phase or solution phase libraries or synthetic libraries made from deconvolution, ‘one-bead one-compound’ methods and by affinity chromatography selection.

4. The method of claim 1, wherein the selected test compound prevents binding of the anergy marker protein with a bioactive agent selected from the group consisting of naturally-occurring compounds, biomolecules, proteins, peptides, oligopeptides, polysaccharides, nucleotides and polynucleotides.

5. The method of claim 1, wherein the test compound is a bioactive agent selected from the group consisting of naturally-occurring compounds, biomolecules, proteins, peptides, oligopeptides, polysaccharides, nucleotides and polynucleotides.

6. The method of claim 1, wherein the test compound is a small molecule.

7. The method of claim 1, wherein the anergy marker is selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176.

8. The method of claim 1, wherein the anergy marker is selected from the group consisting of Mm. 116802, Hs. 248037, Mm. 10085 and Hs. 96149.

9. The method of claim 1, wherein the anergy marker is selected from the group ET63436_at, k00083_s_at, MIP1-B_at, Msa.11439.0_s_at, Msa.15983.0_f_at, Msa.1669.0_f_at Msa.18713.0_g_at, U44731_s_at, x12531 s_at and x67914_s_at.

10. The method of claim 1, wherein the anergy marker is selected from the group consisting of GRG4, jumonji, RPTPσ, PTP-1B, RPTPκ, GBP-3, Rab10, SOCS-2, Traf5, DAGKα, LDHAα, phosphoglycerate mutase, CD98, 4-IBB-L, and FasL.

11. The method of claim 1, wherein the anergy marker is GBP-3.

12. A method of screening for test compounds capable of modulating the level of expression of an anergy marker, the method comprising the steps of comparing:
a) a level of expression of an anergy marker listed in Group I or Group II or Group III or Group IV in a first sample of cells prior to providing a test compound to the first sample of cells; and
b) a level of expression of the same anergy marker in a second sample of cells after providing the test compound to the second sample of cells,
wherein a substantially modulated level of expression of the anergy marker in the second sample, relative to the first sample, is an indication that the test compound is capable of modulating the level of expression.

13. The method of claim 12, wherein the test compound is from a library selected from a group of libraries consisting of spatially addressable parallel solid phase or solution phase libraries or synthetic libraries made from deconvolution, ‘one-bead one-compound’ methods and by affinity chromatography selection.

14. The method of claim 12, wherein the cell is an immune cell.

15. The method of claim 12, further comprising the step of stimulating the cells prior to providing the test compound.

16. The method of claim 15, wherein the step of stimulating the cells includes contacting the cells with a stimulant selected from the group consisting of an antigen, an antigen presenting cell, an activator of NFAT-NFAT ligand signaling, a combination of anti-CD3 and anti-CD28 antibodies, and a combination of anti-TCR and anti-CD28 antibodies.

17. The method of claim 16, wherein the activator of NFAT-NFAT ligand signaling is selected from the group consisting of ionomycin and PMA.

18. The method of claim 12, wherein the anergy marker is selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176.

19. The method of claim 12, wherein the anergy marker is selected from the group consisting of Mm. 116802, Hs. 248037, Mm. 10085 and Hs. 96149.

20. The method of claim 12, wherein the anergy marker is selected from the group consisting of Z31202_s_at, aa144045_s_at, aa174748_at, c81206_rc_at, D86609_s_at, ET63436_at, k00083_s_at, MIP1-B_at, Msa.11439.0_s_at, Msa.15983.0_f_at, Msa.1669.0_f_at, Msa.18713.0_g_at, U44731_s_at, x12531_s_at, and x67914_s_at.

21. The method of claim 12, wherein the anergy marker is selected from the group consisting of GRG4, jumonji, RPTPσ, PTP-1B, RPTPκ, GBP-3, Rab10, caspase-3, SOCS-2, Traf5, DAGKα, LDHAα, phosphoglycerate mutase, CD98, 4-IBB-L, and FasL.

22. The method of claim 12, wherein the anergy marker is GBP-3.

23. A method of screening for test compounds capable of inhibiting an immune disorder, the method comprising:
a) contacting a panel of anergy marker proteins with a plurality of test compounds, wherein the panel of anergy marker proteins comprise at least 2 anergy marker proteins encoded by anergy markers listed in Group I or Group II or Group III or Group IV;
b) detecting binding of one of the test compounds to the panel of anergy marker proteins, relative to other test compounds; and
c) correlating the amount of binding of the test compound to the panel of anergy marker proteins with the ability of the test compound to inhibit an immune disorder, wherein binding indicates that the test compound is capable of inhibiting an immune disorder.

24. The method of claim 23, wherein the method of screening is high-throughput screening.

25. The method of claim 23, wherein the test compound is from a library selected from a group of libraries consisting of spatially addressable parallel solid phase or solution phase libraries or synthetic libraries made from deconvolution, ‘one-bead one-compound’ methods and by affinity chromatography selection.

26. The method of claim 23, wherein the selected test compound prevents binding of the anergy marker protein with a bioactive agent selected from the group consisting of naturally-occurring compounds, biomolecules, proteins, peptides, oligopeptides, polysaccharides, nucleotides and polynucleotides.

27. The method of claim 23, wherein the test compound is a bioactive agent selected from the group consisting of naturally-occurring compounds, biomolecules, proteins, peptides, oligopeptides, polysaccharides, nucleotides and polynucleotides.

28. The method of claim 23, wherein the test compound is a small molecule.

29. The method of claim 23, wherein the anergy marker is selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176.

30. The method of claim 23, wherein the anergy marker is selected from the group consisting of Mm. 116802, Hs. 248037, Mm. 10085 and Hs. 96149.

31. The method of claim 23, wherein the anergy marker is selected from the group consisting of Z31202_s_at, aa144045_s_at, aa174748_at c81206_rc_at, D86609_s_at, ET63436_at k00083_s_at, MIP1-B_at Msa.11439.0_s_at, Msa.15983.0_f_at, Msa.1669.0_f_at, Msa.18713.0_g_at, U44731_s_at, x12531_s_at, and x67914_s_at.

32. The method of claim 23, wherein the anergy marker is selected from the group consisting of GRG4, jumonji, RPTPσ, PTP-1B, RPTPκ, GBP-3, Rab10, caspase-3, SOCS-2, Traf5, DAGKα, LDHAα, phosphoglycerate mutase, CD98, 4-IBB-L, and FasL.

33. The method of claim 23, wherein the anergy marker is GBP-3.

34. The method of claim 23, wherein the immune disorder is selected from the group consisting of T cell disorders, B cell disorders, autoimmune disorders, infectious disorders, proliferative disorders, transplant rejection and cancer.

35. The method of claim 23, wherein the immune disorder is selected from the group consisting of diabetes mellitus, rheumatoid arthritis, juvenile rheumatoid arthritis, osteoarthritis, psoriatic arthritis, multiple sclerosis, encephalomyelitis, myasthenia gravis, systemic lupus erythematosis, autoimmune thyroiditis, atopic dermatitis eczematous dermatitis, psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, graft-versus-host disease, and allergy.

36. The method of claim 23, wherein the immune disorder is selected from the group consisting of diabetes mellitus, rheumatoid arthritis, multiple sclerosis, Crohn's disease, asthma, allergic asthma, graft-versus-host disease, and allergy.

37. The method of claim 23, wherein the cancer is selected from the group consisting of lung cancer, breast cancer, lymphoid cancer, gastrointestinal cancer, genitourinary tract cancer, pharynx cancer, colon cancer, renal-cell carcinoma, prostate cancer, testicular cancer, non-small cell carcinoma of the lung, cancer of the small intestine, cancer of the esophagus, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

38. The method of claim 23, wherein the cancer is selected from the group consisting of breast cancer, renal cell carcinoma, melanoma, lymphoma, and multiple myeloma.

39. A method of screening test compounds for inhibitors of an immune disorder in a subject, the method comprising the steps of:
a) obtaining a sample comprising cells;
b) contacting an aliquot of the sample with one of a plurality of test compounds;
c) comparing a level of expression of an anergy marker listed in Group I or Group II or Group III or Group IV; and
d) selecting one of the test compounds which substantially modulates the level of expression of the anergy marker in the aliquot containing that test compound, relative to other test compounds.

40. The method of claim 39, wherein the test compound is from a library selected from a group of libraries consisting of spatially addressable parallel solid phase or solution phase libraries or synthetic libraries made from deconvolution, ‘one-bead one-compound’ methods and by affinity chromatography selection.

41. The method of claim 39, wherein the anergy marker is selected from the group consisting of Msa.21745.0_s_at (also Mm. 21985), Hs. 129764, U44731_s_at (also Mm. 1909), Hs. 240849, Msa.1669.0_f_at (also Mm. 19123), and GenBank PID:g2853176.

42. The method of claim 39, wherein the anergy marker is selected from the group consisting of Z31202_s_at, aa144045_s_at, aa174748_at, c81206_rc_at, D86609_s_at, ET63436_at, k00083_s_at, MIP1-B_at, Msa.11439.0_s_at, Msa.15983.0_f_at, Msa.1669.0_f_at, Msa.18713.0_g_at, U44731_s_at, x12531_s_at, and x67914_s_at.

43. The method of claim 39, wherein the anergy marker is selected from the group consisting of Mm. 116802, Hs. 248037, M. 10085 and Hs. 96149.

44. The method of claim 39, wherein the anergy marker is selected from the group consisting of GRG4, jumonji, RPTPσ, PTP-1B, RPTPκ, GBP-3, Rab10, caspase-3, SOCS-2, Traf5, DAGKα, LDHAα, phosphoglycerate mutase, CD98, 4-IBB-L, and FasL.

45. The method of claim 39, wherein the anergy marker is GBP-3.
